(19) United States
(12) Patent Application Publication
(10) Pub. No.: US 2023/0242628 A1
(43) Pub. Date: Aug. 3, 2023

(54) BROADLY NEUTRALIZING AND POTENT ANTIBODIES AGAINST HIV

(71) Applicants: University of Maryland, Baltimore, Baltimore, MD (US); U.S. Department of Veterans Affairs, Washington, DC (US); The United States of America,as represented by the Secretary,Department of Health and Human Services, Bethesda, MD (US)

(72) Inventors: Mohammad Sajadi, Cockeysville, MD (US); Paolo Lusso, Rockville, MD (US); Qingbo Liu, Rockville, MD (US)

(21) Appl. No.: 17/778,536

(22) PCT Filed: Nov. 27, 2020
(86) PCT No.: PCT/US2020/062493

§ 371 Date: May 20, 2022

Related U.S. Application Data
(60) Provisional application No. 62/940,572, filed on Nov. 26, 2019.

(51) Int. Cl. C07K 16/10; C12N 15/85; A61K 45/06; A61P 31/18

(52) U.S. Cl. CPC C07K 16/1063; C12N 15/85; A61K 45/06; A61P 31/18; A61K 2039/505

(57) ABSTRACT

The present invention provides broadly neutralizing and potent antibodies against HIV, compositions comprising the same and methods of use thereof.

### CROSS-REFERENCE TO RELATED APPLICATIONS

This application claims the benefit of U.S. Provisional Appl. No. 62/940,572, filed Nov. 26, 2019, the contents of which are hereby incorporated by reference in their entirety.

### STATEMENT OF FEDERALLY SPONSORED RESEARCH AND DEVELOPMENT

This invention was made with government support under Grant No. AI110259 awarded by the National Institutes of Health and Grant No. BX002358 awarded by the US Department of Veterans Affairs. The government has certain rights in the invention.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 979,248 Byte ASCII (Text) file named “Sequence_Listing_ST25.txt,” created on Nov. 28, 2022.

### FIELD OF THE INVENTION

The field of the invention generally relates to medicine, infectious disease and in particular anti-HIV monoclonal antibodies which have enhanced therapeutic neutralizing activity and potency for treating or preventing HIV infection in a mammalian subject.

### BACKGROUND OF THE INVENTION

HIV is an integrating retrovirus that rapidly establishes chronic infection in CD+4 T cells with subsequent depletion of the T cell arm of immunity. This fundamental characteristic means that prevention of HIV infection largely depends on humoral responses and associated effector mechanisms directed against HIV envelope proteins (gp120 and gp41) that drive viral attachment and entry. Humoral anti-envelope responses in a minority of HIV-infected persons comprise neutralizing activity against diverse viral variants. It is widely held that these broadly neutralizing responses can be used to guide the development of effective HIV vaccines and/or other immune-based prevention measures.

We have previously found that multiple HIV-infected subjects harbor broad and potent neutralizing activities with highly shared biochemical determinants. These broadly neutralizing antibodies were isolated from two test subjects, N60 and N49. The broadly neutralizing antibodies in N49 plasma fell into two lineages distinguished by different light chain gene usage. The N49 mAbs all exhibited basic pIs and VH1-2 gene usage. However, all of the N49 mAbs used gamma light chain genes, while also containing deletions in CDRL1 and CDRL3. The binding characteristics of this N49 lineage also matched N60 neutralizing antibodies, reflecting anti-CD4BS specificity. Members of the N49P series of broadly neutralizing antibodies are the broadest and most potent naturally occurring anti-gp120 antibodies described to date capable of neutralizing viruses that are missed by other broadly neutralizing mAbs (including N6, DH411-2 and 10E8).

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

In one aspect, the invention provides an anti-HIV antibody that is derived from a N49P series antibody, wherein the N49P series antibody is modified whereby a part or all of the framework 3 region of the heavy chain is replaced with an amino acid sequence selected from the group consisting of QLSQDPDDPDWG (SEQ ID NO:541) and LFSQDLYYPDRG (SEQ ID NO:542). In some embodiments, the framework 3 region in the N49P series antibody is fully deleted or missing, and in those cases either SEQ ID NO: 541 or 542 is inserted therein.

In some embodiments, the N49P series of antibodies to be modified are selected from the natural antibody sequences 1-38 as shown in Table 1 below. In some embodiments, the N49P series of antibodies to be modified comprises variants of these natural antibodies. In some embodiments, the variants that can be further modified are selected from antibodies N49P6, N49P6.2, N49P7, N49P7.1, N49P7A, N49P7S, N49P7F, N49P7Y, N49P7.54TY, N49P7-LS1, N49P7-LS2, N49P7/6L, N49P7/11L, R49P7,N49P7.2, N49P11, N49P18, N49P18.2, N49P18.1, N49P19, N49P37, N49P38, N49P38.1, N49P55, N49P56, N49P57, N49P58, N49P59, N49P73, N49P74, N49P75, N49P9, N49P9.1, N49P9.2, N49P9i7, N49P22, N49P23, N49P9.3, N49P9.4, N49P9.6, N49P9.6-54W, N49P9.6-54F, N49P51, N49P52, N49P53, N49P54, N49P60, N49P61, N49P62, N49P63, N49P64, N49P65, N49P66, N49P67, N49P68, N49P69, N49P70, N49P71, and N49P72. These variants are described in Table 3, below.

In another aspect, the invention provides an anti-HIV antibody, wherein the antibody comprises the VH and VL regions of antibody N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE.

In another aspect, the invention provides an anti-HIV antibody, wherein the antibody is N49P7-FR or an antigen binding fragment thereof. The amino acid sequence of the heavy chain of N49P7-FR is SEQ ID NO:501 and the nucleotide sequence is SEQ ID NO:502. The amino acid sequence of the light chain of N49P7-FR is SEQ ID NO:503 and the nucleotide sequence is SEQ ID NO:504.

In another aspect, the invention provides an anti-HIV antibody, wherein the antibody is N49P9-FR or an antigen binding fragment thereof. The amino acid sequence of the heavy chain of N49P9-FR is SEQ ID NO:505 and the nucleotide sequence is SEQ ID NO:506. The amino acid sequence of the light chain of N49P9-FR is SEQ ID NO:295 and the nucleotide sequence is SEQ ID NO:296.

In another aspect, the invention provides an anti-HIV antibody, wherein the antibody is N49P9.3-FR or an antigen binding fragment thereof. The amino acid sequence of the heavy chain of N49P9.3-FR is SEQ ID NO:507 and the nucleotide sequence is SEQ ID NO:508. The amino acid sequence of the light chain of N49P9.3-FR is SEQ ID NO:327 and the nucleotide sequence is SEQ ID NO:328.

In another aspect, the invention provides an anti-HIV antibody, wherein the antibody is N49P9.6-FR or an antigen binding fragment thereof. The amino acid sequence of the heavy chain of N49P9.6-FR is SEQ ID NO:509 and the nucleotide sequence is SEQ ID NO:510. The amino acid sequence of the light chain of N49P9.6-FR is SEQ ID NO:511 and the nucleotide sequence is SEQ ID NO:512.

In another aspect, the invention provides an anti-HIV antibody, wherein the antibody is N49P9.6-FR-54W or an antigen binding fragment thereof. The amino acid sequence of the heavy chain of N49P9.6-FR-54W is SEQ ID NO:513 and the nucleotide sequence is SEQ ID NO:514. The amino acid sequence of the light chain of N49P9.6-FR-54W is SEQ ID NO:515 and the nucleotide sequence is SEQ ID NO:516.

In another aspect, the invention provides an anti-HIV antibody, wherein the antibody is N49P9.6-FR-54F or an antigen binding fragment thereof. The amino acid sequence of the heavy chain of N49P9.6-FR-54F is SEQ ID NO:517 and the nucleotide sequence is SEQ ID NO:518. The amino acid sequence of the light chain of N49P9.6-FR-54F is SEQ ID NO:519 and the nucleotide sequence is SEQ ID NO:520.

In another aspect, the invention provides an anti-HIV antibody, wherein the antibody is N49P9.6-FR3-06 or an antigen binding fragment thereof. The amino acid sequence of the heavy chain of N49P9.6-FR3-06 is SEQ ID NO:521 and the nucleotide sequence is SEQ ID NO:522. The amino acid sequence of the light chain of N49P9.6-FR3-06 is SEQ ID NO:523 and the nucleotide sequence is SEQ ID NO:524.

In another aspect, the invention provides an anti-HIV antibody, wherein the antibody is N49P9.6-FR1-D or an antigen binding fragment thereof. The amino acid sequence of the heavy chain of N49P9.6-FR1-D is SEQ ID NO:525 and the nucleotide sequence is SEQ ID NO:526. The amino acid sequence of the light chain of N49P9.6-FR1-D is SEQ ID NO:527 and the nucleotide sequence is SEQ ID NO:528.

In another aspect, the invention provides an anti-HIV antibody, wherein the antibody is N49P9.6-FR1-D-I or an antigen binding fragment thereof. The amino acid sequence of the heavy chain of N49P9.6-FR1-D-I is SEQ ID NO:529 and the nucleotide sequence is SEQ ID NO:530. The amino acid sequence of the light chain of N49P9.6-FR1-D-I is SEQ ID NO:531 and the nucleotide sequence is SEQ ID NO:532.

In another aspect, the invention provides an anti-HIV antibody, wherein the antibody is N49P9.6-FR-LS or an antigen binding fragment thereof. The amino acid sequence of the heavy chain of N49P9.6-FR-LS is SEQ ID NO:533 and the nucleotide sequence is SEQ ID NO:534. The amino acid sequence of the light chain of N49P9.6-FR-LS is SEQ ID NO:535 and the nucleotide sequence is SEQ ID NO:536.

In another aspect, the invention provides an anti-HIV antibody, wherein the antibody is N49P9.6-FR-YTE or an antigen binding fragment thereof. The amino acid sequence of the heavy chain of N49P9.6-FR-YTE is SEQ ID NO:537 and the nucleotide sequence is SEQ ID NO:538. The amino acid sequence of the light chain of N49P9.6-FR-YTE is SEQ ID NO:539 and the nucleotide sequence is SEQ ID NO:540.

In another aspect, the invention provides an anti-HIV antibody, wherein the antibody comprises the heavy chain CDR and light chain CDR sequences of the antibodies N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE, wherein the antibody comprises a framework 3 region of the heavy chain comprising an amino acid sequence selected from QLSQDPDDPDWG (SEQ ID NO:541) and LFSQDLYYPDIRG (SEQ ID NO:542).

In some embodiments, the anti-HIV antibody neutralizes at least 95%, at least 96%, at least 97%, at least 98%, at least 99% or 100% of the HIV pseudoviruses listed Table 4 (see also FIG. 1) with an IC50 value of less than 50 μg/mL.

In some embodiments, the anti-HIV antibody neutralizes at least 50%, at least 55%, at least 60%, at least 65%, at least 70%, at least 75%, at least 80%, at least 85%, at least 90%, at least 95%, or at least 99% of the HIV pseudoviruses listed in Table 4 with an IC50 value of less than about 1 μg/ml, between about 1-5 μg/ml or greater than about 5 μg/ml.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Neutralization activity of N49 plasma and P series mAbs. A panel of 117 HIV-1 pseudovirus Tier 1-3 isolates (individual viruses listed on the left column) were tested against N49 plasma, N49P7, and N4P9.6-FR. IC₅₀ values are color-coded according to the color key on the left: the greater the neutralization, the darker red the color; white represents no neutralization (IC₅₀>50 ug/ml). N49P7 exhibited 100% breadth, while N49P9.6 and N49P9.6-FR exhibited 97% breadth, with extreme potency. IC50=Inhibitory Concentration 50 in ug/ml. T/F=transmitted founder. NC=not classified.

FIG. 2. IC80 values of “FR” variants of N49 P series monoclonal antibodies compared to earlier variants. Anti-HIV-1 monoclonal antibodies N49P7, N49P9, N49P9.3, and N49P9.6 were engineered to include an extra heavy chain framework 3 loop to increase binding to the HIV-1 trimer. In all four cases, IC80 was able to be improved.

FIG. 3. Germline and natural heavy chains. A) Variable region. IMGT numbering system of germline 1-2 heavy chain shown, as well as alignment of germline 1-2 to natural sequences 2, 18, and 21. B) Constant region. IMGT numbering system of CH1-3 of IgG1 shown as well as alignment of natural sequences that have point mutations in the constant region.

FIG. 4. Germline and natural light chain variable region. A) IMGT numbering system of germline IgL2-11 shown, as well as alignment of IgL2-11 with J3 to natural sequence 2. B) IMGT numbering system of germline IgL2-23 shown, as well as alignment of IgL2-23 with J3 shown to natural sequences 18 and 21.

FIG. 5. Germline and natural light chain constant region. A) IMGT numbering system of germline LC2 as well as alignment to natural sequences 1-17 that use this gene with point mutation shown. B) IMGT numbering system of germline LC7, as well as alignment to natural sequences 18-38 that use this gene, with point mutation shown.

FIG. 6. Crystal structure of N49P9.3 Fab-gp120_{993TH057} coreₑ complex. (A) Ribbon diagram of complex with the complementarity-determining regions (CDRs) of Fab contributing to the gp120 binding. An expanded view shows details of interaction of Light chain N terminus with Loop E and CDR H2 with Loop V5 (B) Structural comparison of gp120 antigen binding modes of N49P9.3 to N49P7, two bNAbs from donor N49 that come from separate families. Fab-gp120_{993TH057} coreₑ complexes were superimposed based on gp120 sequence. Expanded views show differences in how light chain N-terminus (top panel) and CDR H3 interact with gp120 antigen.

FIG. 7. Suppression of viremia by bNAb P9.3 in NGS mice reconstituted with HIV+PBL. There were 5 animals in each group. Samples with no readings above the lower limit of detection were assigned an arbitrary value of 10 copies/ml.

FIG. 8. Prevention of cell-free HIV BaL infection by bNAb P9.6 in NGS mice reconstituted with human PBL. Mean values are shown; bars equal standard error. Samples with no readings above the lower limit of detection were assigned an arbitrary value of 10 copies/ml.

FIG. 9. Acute HIV treatment with VRC01 and N49P9.6. Hu-CD34 mice were generated and used as described in the text. On Day 0, HIV infection was established with 8000TCID50 of HIV-Bal virus 2 weeks prior to challenge. Two weeks after HIV injection, cART was started (tenofovir and emtricitibine) to induce partial viral suppression. Two weeks after start of cART a single dose (10 mg/kg) of VRC01, N49P9.6, or Synagis (anti-RSV mAb) was injected IP. Ten days after mAb injection, cART was stopped (timed so concentrations of antibody and cART would be decreased by at least 4 half-lives by Day 30). Samples with no readings above the lower limit of detection were assigned an arbitrary value of 10 copies/ml.

FIG. 10. IC80 values of N49 parental antibodies and their “FR” variants. Each bNAb shown was engineered to insert a heavy chain framework 3 loop from VRC03 where there was a deletion as described in the narrative, resulting in improved potency.

FIG. 11. Neutralization characteristics of single bNAbs in a global, cross Clade, cross-Tier panel of pseudoviruses.

FIG. 12. Neutralization characteristics of triple bNAb combinations in a global, cross-clade, cross-Tier panel of pseudoviruses.

FIG. 13. Neutralization characteristics of triple bNAb combinations in Clade C pseudoviruses.

FIG. 14. Suppression of viremia by bNAb P9.3 in NGS mice reconstituted with HIV+PBL. There were 5 animals in each group. Samples with no readings above the lower limit of detection were assigned an arbitrary value of 10 copies/ml.

FIG. 15. Prevention of cell-free HIV BaL infection by bNAb P9.6 in NGS mice reconstituted with human PBL. Mean values are shown; bars equal standard error. Samples with no readings above the lower limit of detection were assigned an arbitrary value of 10 copies/ml.

FIG. 16. Acute HIV treatment with VRC01 and N49P9.6. Hu-CD34 mice were generated and used as described in the text. On Day 0, HIV infection was established with 8000TCID50 of HIV-Bal virus 2 weeks prior to challenge. Two weeks after HIV injection, cART was started (tenofovir and emtricitibine) to induce partial viral suppression. Two weeks after start of cART a single dose (10 mg/kg) of VRC01, N49P9.6, or Synagis (anti-RSV mAb) was injected IP. Ten days after mAb injection, cART was stopped (timed so concentrations of antibody and cART would be decreased by at least 4 half-lives by Day 30). Samples with no readings above the lower limit of detection were assigned an arbitrary value of 10 copies/ml.

### DETAILED DESCRIPTION OF THE INVENTION

From the N49P series of mAbs there is a need to develop the most broadly neutralizing and potent antibodies for clinical/therapeutic applications. Further modifications to certain of the N49P series of mAbs have been made and are herein described in detail and form the basis of this invention and the next generation of broadly neutralizing anti-HIV monoclonal antibodies.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)). Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

As used herein, the term “about” means plus or minus 10% of the numerical value of the number with which it is being used.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments, dual affinity retargeting antibodies (DART)), single chain Fv (scFv) mutants, multispecific antibodies such as bispecific and trispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

In some embodiments, an antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the α and γ chains and four C_{H} domains for μ, and ε isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ) respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antigen binding fragment” or antibody fragment refers to a portion of an intact antibody and comprises the antigenic determining variable regions of an intact antibody. Examples of antigen binding fragment include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)).

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommie, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles (Ruiz, M. and Lefranc, M.-P., Immunogenetics, 53, 857-883 (2002)/Kaas, Q. and Lefranc, M.-P., Current Bioinformatics, 2, 21-30 (2007)), and in 3D structures in IMGT/3Dstructure-DB (Kaas, Q., Ruiz, M. and Lefranc, M.-P., T cell receptor and MHC structural data. Nucl. Acids. Res., 32, D208-D210 (2004)).

In some embodiments, CDRs are determined based on cross-species sequence variability (i.e., Kabat et al. Sequences of Proteins of Immunological Interest, (5th ed., 1991, National Institutes of Health, Bethesda Md.)). In some embodiments, CDRs are determined based on crystallographic studies of antigen-antibody complexes (Al-lazikani et al (1997) J. Molec. Biol. 273:927-948)). In addition, combinations of these two approaches can be used to determine CDRs. In some embodiments, the CDRs are determined based on AHo (Honegger and Pluckthun, J. Mol. Biol. 309(3):657-670; 2001). In some embodiments, CDRs are determined based on the IMGT system.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

A “neutralizing antibody” may inhibit the entry of HIV-1 virus for example SF162 and/or JR-CSF with a neutralization index >1.5 or >2.0. (Kostrikis L G et al. J Virol. 1996; 70(1): 445-458.). By “broad and potent neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. A broad neutralizing antibody may neutralize multiple HIV-1 species belonging to at least 2, 3, 4, 5, or 6 different clades. In some embodiments, the \concentration of the monoclonal antibody able to neutralize at 50% of the input virus in the neutralization assay can be less than about 50 μg/ml.

An “intact” antibody is one that comprises an antigen-binding site as well as a C_{L} and at least heavy chain constant domains, C_{H1}, C_{H2} and C_{H3}. The constant domains may be native sequence constant domains (e.g., human native sequence constant domains) or amino acid sequence variants thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

The antibodies herein also include antibodies in which a portion of the heavy and/or light chain is identical with or homologous to corresponding sequences in antibodies belonging to a particular antibody class or subclass, while the remainder of the chain(s) is identical with or homologous to corresponding sequences in antibodies derived from another species or belonging to another antibody class or subclass, as well as fragments of such antibodies.

In some embodiments, the antibody comprises variable region antigen-binding sequences derived from human antibodies (e.g., CDRs) and containing one or more sequences derived from a non-human antibody, e.g., an FR or C region sequence. In some embodiments, the antibody includes those comprising a human variable region antigen binding sequence of one antibody class or subclass and another sequence, e.g., FR or C region sequence, derived from another antibody class or subclass.

In some embodiments, chimeric antibodies may comprise residues that are not found in the recipient antibody or in the donor antibody. In some embodiments, modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Low-affinity antibodies generally bind antigen slowly and tend to dissociate readily, whereas high-affinity antibodies generally bind antigen faster and tend to remain bound longer.

The affinity or avidity of an antibody for an antigen can be determined experimentally using any suitable method well known in the art, e.g. flow cytometry, enzyme-linked immunoabsorbent assay (ELISA), or radioimmunoassay (RIA), or kinetics (e.g., BIACORE™analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kₒₙ, K_{off}) are made with standardized solutions of antibody and antigen, and a standardized buffer, as known in the art and such as the buffer described herein.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., Kd values). The difference between said two values is less than about 500%, less than about 40%, less than about 300%, less than about 200%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature. A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the invention and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating one or more biological activities of the polypeptide as described herein and/or using any of a number of techniques well known in the art. or can be produced by recombinant or synthetic means.

As used herein, “substantially pure” refers to material which is at least 50% pure (i.e., free from contaminants), at least 90% pure, at least 95% pure, at least 98% pure, or at least 99% pure.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Administration “in combination with” one or more further therapeutic agents includes simultaneous (concurrent) and consecutive administration in any order.

The term “pharmaceutical formulation” refers to a preparation which is in such form as to permit the biological activity of the active ingredient to be effective, and which contains no additional components which are unacceptably toxic to a subject to which the formulation would be administered. Such formulation can be sterile.

An “effective amount” of an antibody as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” or prevent a disease or disorder in a subject or mammal.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100.times.(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman. Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In certain embodiments, identity exists over a region of the sequences that is at least about 10, about 20, about 40-60 residues in length or any integral value therebetween, or over a longer region than 60-80 residues, at least about 90-100 residues, or the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the invention do not abrogate the binding of the polypeptide or antibody containing the amino acid sequence, to the antigen(s), i.e., the gp120 to which the polypeptide or antibody binds. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

### Anti-HIV Antibodies

HIV-1 is among the most genetically diverse viral pathogens. Of the three main branches of the HIV-1 phylogenetic tree, the M (main), N (new), and O (outlier) groups, group M viruses are the most widespread, accounting for over 99% of global infections. This group is presently divided into nine distinct genetic subtypes, or clades (A through K), based on full-length sequences. Env is the most variable HIV-1 gene, with up to 35% sequence diversity between clades, 20% sequence diversity within clades, and up to 10% sequence diversity in a single infected person (Shankarappa, R. et al. 1999. J. Virol. 73:10489-10502). Clade B is dominant in Europe, the Americas, and Australia. Clade C is common in southern Africa, China, and India and presently infects more people worldwide than any other clade (McCutchan, F E. 2000. Understanding the genetic diversity of HIV-1. AIDS 14(Suppl. 3):531-S44). Clades A and D are prominent in central and eastern Africa.

In some embodiments, the invention provides antibodies that are broadly neutralizing and potent antibodies against HIV. The antibodies are modified from the N49P series of antibodies. The N49P series of antibodies are detailed and described in WO 2018/237357, filed on Jun. 22, 2018, which is hereby incorporated by reference in its entirety. The N49P series of antibodies comprises natural antibodies as well as engineered variants of the natural antibodies.

In some embodiments, the antibody is derived from a N49P series antibody, wherein the N49P series antibody is modified whereby a part or all of the framework 3 region of the heavy chain is replaced with an amino acid sequence selected from the group consisting of QLSQDPDDPDWG (SEQ ID NO:541) and LFSQDLYYPDRG (SEQ ID NO:542). In some embodiments, the framework 3 region in the N49P series antibody is already deleted or missing, and in those cases either SEQ ID NO: 541 or 542 is inserted therein in the framework 3 region.

In some embodiments, the N49P series of antibodies to be modified are selected from the natural antibody sequences 1-38 as shown in Table 1 below. In some embodiments, the N49P series of antibodies to be modified comprises variants of these natural antibodies. In some embodiments, the variants that can be further modified are selected from antibodies N49P6, N49P6.2, N49P7, N49P7.1, N49P7A, N49P7S, N49P7F, N49P7Y, N49P7.54TY, N49P7-LS1, N49P7-LS2, N49P7/6L, N49P7/11L, R49P7,N49P7.2, N49P11, N49P18, N49P18.2, N49P18.1, N49P19, N49P37, N49P38, N49P38.1, N49P55, N49P56, N49P57, N49P58, N49P59, N49P73, N49P74, N49P75, N49P9, N49P9.1, N49P9.2, N49P9i7, N49P22, N49P23, N49P9.3, N49P9.4, N49P9.6, N49P9.6-54W, N49P9.6-54F, N49P51, N49P52, N49P53, N49P54, N49P60, N49P61, N49P62, N49P63, N49P64, N49P65, N49P66, N49P67, N49P68, N49P69, N49P70, N49P71, and N49P72. These variants are described in Table 3, below.

Without being bound by theory, modification of the framework 3 region of the heavy chain in the N49P antibodies to encode amino acid sequence motifs selected from QLSQDPDDPDWG (SEQ ID NO:541) and LFSQDLYYPDRG (SEQ ID NO:542) enable the modified N49P antibodies to make additional contacts to improve binding to the CD4-binding site of HIV, resulting in increased potency of neutralization. The binding of the N49P antibodies with the CD4-binding site of HIV is described in WO 2018/237357, which is incorporated by reference herein.

In some embodiments, the modified N49P series antibody is antibody N49P7-FR or an antigen binding fragment thereof. The amino acid sequence of the heavy chain of N49P7-FR is SEQ ID NO:501 and the nucleotide sequence is SEQ ID NO:502. The amino acid sequence of the light chain of N49P7-FR is SEQ ID NO:503 and the nucleotide sequence is SEQ ID NO:504.

In some embodiments, the modified N49P series antibody is antibody N49P9-FR or an antigen binding fragment thereof. The amino acid sequence of the heavy chain of N49P9-FR is SEQ ID NO:505 and the nucleotide sequence is SEQ ID NO:506. The amino acid sequence of the light chain of N49P9-FR is SEQ ID NO:295 and the nucleotide sequence is SEQ ID NO:296.

In some embodiments, the modified N49P series antibody is antibody N49P9.3-FR or an antigen binding fragment thereof. The amino acid sequence of the heavy chain of N49P9.3-FR is SEQ ID NO:507 and the nucleotide sequence is SEQ ID NO:508. The amino acid sequence of the light chain of N49P9.3-FR is SEQ ID NO:327 and the nucleotide sequence is SEQ ID NO:328.

In some embodiments, the modified N49P series antibody is antibody N49P9.6-FR or an antigen binding fragment thereof. The amino acid sequence of the heavy chain of N49P9.6-FR is SEQ ID NO:509 and the nucleotide sequence is SEQ ID NO:510. The amino acid sequence of the light chain of N49P9.6-FR is SEQ ID NO:511 and the nucleotide sequence is SEQ ID NO:512.

In some embodiments, the modified N49P series antibody is antibody N49P9.6-FR-54W or an antigen binding fragment thereof. The amino acid sequence of the heavy chain of N49P9.6-FR-54W is SEQ ID NO:513 and the nucleotide sequence is SEQ ID NO:514. The amino acid sequence of the light chain of N49P9.6-FR-54W is SEQ ID NO:515 and the nucleotide sequence is SEQ ID NO:516.

In some embodiments, the modified N49P series antibody is antibody N49P9.6-FR-54F or an antigen binding fragment thereof. The amino acid sequence of the heavy chain of N49P9.6-FR-54F is SEQ ID NO:517 and the nucleotide sequence is SEQ ID NO:518. The amino acid sequence of the light chain of N49P9.6-FR-54F is SEQ ID NO:519 and the nucleotide sequence is SEQ ID NO:520.

In some embodiments, the modified N49P series antibody is antibody N49P9.6-FR3-06 or an antigen binding fragment thereof. The amino acid sequence of the heavy chain of N49P9.6-FR3-06 is SEQ ID NO:521 and the nucleotide sequence is SEQ ID NO:522. The amino acid sequence of the light chain of N49P9.6-FR3-06 is SEQ ID NO:523 and the nucleotide sequence is SEQ ID NO:524.

In some embodiments, the modified N49P series antibody is antibody N49P9.6-FR1-D or an antigen binding fragment thereof. The amino acid sequence of the heavy chain of N49P9.6-FR1-D is SEQ ID NO:525 and the nucleotide sequence is SEQ ID NO:526. The amino acid sequence of the light chain of N49P9.6-FR1-D is SEQ ID NO:527 and the nucleotide sequence is SEQ ID NO:528.

In some embodiments, the modified N49P series antibody is antibody N49P9.6-FR1-D-I or an antigen binding fragment thereof. The amino acid sequence of the heavy chain of N49P9.6-FR1-D-I is SEQ ID NO:529 and the nucleotide sequence is SEQ ID NO:530. The amino acid sequence of the light chain of N49P9.6-FR1-D-I is SEQ ID NO:531 and the nucleotide sequence is SEQ ID NO:532.

In some embodiments, the modified N49P series antibody is antibody N49P9.6-FR-LS or an antigen binding fragment thereof. The amino acid sequence of the heavy chain of N49P9.6-FR-LS is SEQ ID NO:533 and the nucleotide sequence is SEQ ID NO:534. The amino acid sequence of the light chain of N49P9.6-FR-LS is SEQ ID NO:535 and the nucleotide sequence is SEQ ID NO:536.

In some embodiments, the modified N49P series antibody is antibody N49P9.6-FR-YTE or an antigen binding fragment thereof. The amino acid sequence of the heavy chain of N49P9.6-FR-YTE is SEQ ID NO:537 and the nucleotide sequence is SEQ ID NO:538. The amino acid sequence of the light chain of N49P9.6-FR-YTE is SEQ ID NO:539 and the nucleotide sequence is SEQ ID NO:540.

In some embodiments, the antibody comprises the VH and VL regions of antibody N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE as shown below.

In some embodiments, the antibody comprises the heavy chain CDR and light chain CDR sequences of the antibodies N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE as shown below, wherein the antibody comprises a framework 3 region of the heavy chain comprising an amino acid sequence selected from QLSQDPDDPDWG (SEQ ID NO:541) and LFSQDLYYPDRG (SEQ ID NO:542).

The naturally occurring N49P series antibodies are shown below in Table 1 (antibody sequences 1-38), and the related variants are indicated.

TABLE 1
Amino acid sequence of natural antibodies. Heavy chain is shown first (above dotted
line), followed by the light chain (below dotted line). Sequences of CDR H1, CDR H2, CDR
H3, CDR L1, CDR L2, and CDR L3 are in bold. Note that there is a predicted blank CDR in
the light chain for Sequences 25, 34 and 36.

| Natural antibody | Related variants | Amino Acid sequence |
|---|---|---|
| Sequence 1 | N49P6,N49P6.2 | AGLMQSGAVMKNSGASVRVSCQADGYDFIDYVIHWFRQRRGEGLEW |
| | | LGWMNPSGGGTNYPRPFQGKVTMTRDTSTETAYLDVRGLTYDDTAV |
| | | YYCVRDRANGSGRRRFESVNWFLDLWGRGTQITVVSPSTKGPSVFPL |
| | | APSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | -------------------------------------------------- |
| | | --------------------- |
| | | SEQ ID NO: 1 |
| | | QSALTQPRSVSASPGQSVTISCTGTHNYVSWCQQKPGQAPKLLIYDFNK |
| | | RPSGVSDRFSGSTSGNTASLTISGLQADDEGHYFCWAFENIGGGTKLTV |
| | | LRQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTV |
| | | EKTVAPTECS SEQ ID NO: 2 |
| Sequence 2 | N49P7, N49P7.1 | ADLVQSGAVVKKPGDSVRISCEAQGYRFPDYIIHWIRRAPGQGPEWMG |
| | N49P7S | WMNPMGGQVNIPWKFQGRVSMTRDTSIETAFLDLRGLKSDDTAVYY |
| | N49P7F | CVRDRSNGSGKRFESSNWFLDLWGRGTAVTIQSSSTKGPSVFPLAPSS |
| | N49P7Y | KSTSGGTAALGCLVKDYFPEPVTVSWNSGALTSGVHTFPAVLQSSGLY |
| | N49P7.54TY | SLSSVVTVPSSSLGTQTYICNVNHKPSNTKVDKKVEPKSCDKTHTCPPC |
| | N49P7-LS1 | PAPELLGGPSVFLFPPKPKDTLMISRTPEVTCVVVDVSHEDPEVKFNWY |
| | N49P7-LS2 | VDGVEVHNAKTKPREEQYNSTYRVVSVLTVLHQDWLNGKEYKCKVS |
| | N49P7/6L | NKALPAPIEKTISKAKGQPREPQVYTLPPSRDELTKNQVSLTCLVKGFYP |
| | N49P7/11L | SDIAVEWESNGQPENNYKTTPPVLDSDGSFFLYSKLTVDKSRWQQGNV |
| | R49P7 | FSCSVMHEALHNHYTQKSLSLSPGK |
| | N49P7-FR | SEQ ID NO: 3 |
| | N49P7A | -------------------------------------------------- |
| | | --------------------- |
| | | ALTQPRSVSASPGQSVTISCTGTHNLVSWCQHQPGRAPKLLIYDFNKRP |
| | | SGVPDRFSGSGSGGTASLTITGLQDDDDAEYFCWAYEAFGGGTKLTVL |
| | | RQPKAAPSVTLFPPSSEELQANKATLVCLISDFYPGAVTVAWKADSSPV |
| | | KAGVETTTPSKQSNNKYAASSYLSLTPEQWKSHRSYSCQVTHEGSTVE |
| | | KTVAPTECS |
| | | SEQ ID NO: 4 |
| Sequence 3 | N49P7.2 | ADLVQSGAVVKKPGDSVRISCEAQGYKFPDYIIHWIRRAPGQGLEWM |
| | | GWINPMGGQVNIPWQFQGRVSMTRDTSIETAFLDLRGLKSDDTALYY |
| | | CVRDRSNGSGRRFESSNWFLDLWGRGTAVTVHSPSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNH |
| | | YTQKSLSLSPGK |
| | | SEQ ID NO: 5 |
| | | -------------------------------------------------- |
| | | --------------------- |
| | | QSALTQPRSVSASPGQSVTISCTGTHNLVSWCQHHPGRAPKLLIYDFNK |
| | | RPSGVPDRFSGSGSGGTASLTISGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 6 |
| Sequence 4 | N49P11 | SAELVQSGAVVKKPGTSVKVSCQAYGYTFTDYLIHWLRQAPGQGLEW |
| | | MGWMNPVYGQVNYAQNFQGRVSMTRDIYRETAFLEVRDLKTDDTGT |
| | | YYCVRDTGDGSRRHFDSINWFLDLWGRGTWIRVAPASTKGPSVFPLA |
| | | PSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 7 |
| | | -------------------------------------------------- |
| | | --------------------- |
| | | QCVLTQPRSVSGSPGQSVTISCTGTHNYVSWCQHHPGNAPKLLLYDFD |
| | | KRPSGISDRFSGSRSGNTASLTISGLQPEDEADYFCWAFEAFGGGTKVL |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 8 |
| Sequence 5 | N49P18 | ADLVQSGAVMKKPGDSVRISCEARGYTFTDYVIHWIRRAPGQGLEWM |
| | N49P18.2 | GWIDPPYGQVNIPWNFQGRVSMTRDTSIETAFLDLRGLKSDDTGLYYC |
| | | VRDRSNGWGKRFESSNWFLDLWGRGTVVTVHSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 9 |
| | | -------------------------------------------------- |
| | | --------------------- |
| | | QSALTQPRSVSASPGQSVTISCTGTHNLVSWCQHHPGRAPKLLIYDFNK |
| | | RPSGVPDRFSGSGSGGTASLTISGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 10 |
| Sequence 6 | N49P18.1 | ADLVQSGAVVKKPGDSVRISCEAQGYTFTDYVIHWIRRAPGQGLEWM |
| | | GWINPGYGQVNIPWNFQGRVSMTRDTSIETAFLDLRGLKSDDTGLYYC |
| | | VRDRSNGWGKRFESSNWFLDLWGRGTVVTVHSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 11 |
| | | -------------------------------------------------- |
| | | --------------------- |
| | | QSALTQPRSMSASPGQSVTISCTGTHNLVSWCQHHPGRPPKLLIYDFNK |
| | | RPSGVPDRFSGSGSGGTASLTISGLQDDDDAEYICWAYEAFGGGTKLTV |
| | | LRQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTV |
| | | EKTVAPTECS |
| | | SEQ ID NO: 12 |
| Sequence 7 | N49P19 | ADLVQSGAVVKNAGASVRVSCEAYGYTFVDYFIHWVRQAPGQGFEW |
| | | MGYMDPLNGRPNIARKFQGRLSLSRDRSSETSFLDLSGLRSDDSAVYY |
| | | CVRDKSNGSGRRFDSSNWFLDLWGRGTRVSIFSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 13 |
| | | -------------------------------------------------- |
| | | --------------------- |
| | | QSALTQPRSVSATPGQSVTISCTGTHNYVSWCQQHPGRAPKLLIYDFNK |
| | | RPSGVPDRFSGSGSGGTASLTITGLQDDDEADYFCWAYDAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 14 |
| Sequence 8 | N49P37 | ADLVQSGAVVKKPGDSVRVSCEAYGYTFSDYIIHWIRRAPGRGLEWM |
| | | GWMNPMGGQVNIPWNFQGRVSMTRDTSIETAFLDLRGLRSDDTAVY |
| | | YCVRDRSNGSGKRFESSNWFLDLWGRGTAVTISSPSTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| | | SEQ ID NO: 15 |
| | | -------------------------------------------------- |
| | | --------------------- |
| | | QSALTQPRSVSAAPGQSVTISCTGTHNLVSWCQHHPGRAPKLLIYDFNK |
| | | RPSGVPDRFSGSGSGGTASLTITGLQDDDEAEYFCWAYEVFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 16 |
| Sequence 9 | N49P38 | ADLVQSGAVVKTPGASVRVSCEAYGYTFIDYIIHWVRQAPGQGFEWL |
| | N49P38.1 | GYIDPMNGRPNIARKFQGRLSLSRDTSIETSFLDLSGLRSDDSAVYYCV |
| | | RDKSNGSGKRFDSSNWFLDLWGRGTRVSISSASTKGPSVFPLAPSSKS |
| | | TSGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| | | SEQ ID NO: 17 |
| | | -------------------------------------------------- |
| | | --------------------- |
| | | QSALTQPRSVSAAPGQSVTISCTGTHNYVSWCQQHPGRAPKLLIYDFNK |
| | | RPSGVPDRFSGSGSGGTASLTITRLQDDDDADYFCWAYDAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 18 |
| Sequence 10 | N49P55 | ADLVQSGAVVKKPGASVRVSCEAYGYTFTDYIIHWIRQAPGQGLEWM |
| | | GWMNPMGGRTNIPWKFQGRVSMTRDTSIETAFLDLSGLTSDDTAVYY |
| | | CVRDKSNGSGKRFDSSNWFLDLWGRGTPVTISSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 19 |
| | | -------------------------------------------------- |
| | | --------------------- |
| | | QSALTQPRSVSAAPGQSVTISCTGTHNLVSWCQQHPGRAPKLLIYDFNK |
| | | RPSGVPDRFSGSGSGGTASLSITGLQDDDEAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 20 |
| Sequence 11 | N49P56 | ADLVQSGAVVKKPGASVRVSCEAYGYTFVDYLIHWVRQAPGQGFEW |
| | | MGYMDPMNGRPNIARKFQGRLSLSRDTSIETSFLDLSGLRSDDSAVYY |
| | | CVRDKSGGSGKLFDSSNWFLDLWGRGTRVSISSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 21 |
| | | -------------------------------------------------- |
| | | --------------------- |
| | | QSALTQPRSVSAAPGQSVTISCTGTHNYVSWCQQHPGRAPKLLIYDFNK |
| | | RPSGVPDRFSGSGSGGTASLTITGLQDDDDADYFCWAYDAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 22 |
| Sequence 12 | N49P57 | ADLVQSGAVVKKPGDSVRISCEAQGYTFTDYVIHWIRRAPGQGLEWM |
| | | GWINPGYGQVNIPWNFQGRVSMTRDTSIETAFLELRGLKSDDTGLYYC |
| | | VRDRSNGWGKRFESSNWFLDLWGRGTVITVHSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 23 |
| | | -------------------------------------------------- |
| | | --------------------- |
| | | QSALTQPRSMSASPGQSVTISCTGTHNLVSWCQHHPGRPPKLLIYDFNK |
| | | RPSGVPDRFSGSGSGGTASLTITGLQDDDDAEYICWAYEAFGGGTKLTI |
| | | LRQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTV |
| | | EKTVAPTECS |
| | | SEQ ID NO: 24 |
| Sequence 13 | N49P58 | ADLVQSGAVVKKPGDSVRISCEAQGYTFTDYVIHWIRRAPGQGLEWM |
| | | GWMDPSYGQVNIPRNFQGRVSMTRDTFRETAYLELRGLQSDDKGLYY |
| | | CVRDRSHGSGRQFESSNWFLDLWGRGTVVNVQSPSTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| | | SEQ ID NO: 25 |
| | | -------------------------------------------------- |
| | | --------------------- |
| | | QSALTQPRSVSASPGQSVTISCTGTHNLVSWCQHHPGRPPKLLIYDFNK |
| | | RASGVPDRFSGSGSGGTASLTISGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 26 |
| Sequence 14 | N49P59 | ADLVQSGAVVKKPGDSLRISCEAQGYTFTDYVIHWIRRAPGQGLEWM |
| | | GWMDPSFGQMNIPRNFQGRVSMTRDMYIETAFLDLRGLKSDDTGLYY |
| | | CVRDRSHGSGRLFESSNWFLDLWGRGTVVTVQSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 27 |
| | | -------------------------------------------------- |
| | | --------------------- |
| | | QSALTQPRSVSASPGQSVTISCTGTHNLVSWCQHHPGRPPKLLIYDFNK |
| | | RASGVPDRFSGSGSGGTASLTISGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 28 |
| Sequence 15 | N49P73 | ADLVQSGAVVKKPGDSVRISCEAQGYRFTDYVIHWIRRAPGQGLEWM |
| | | GLMDPSFGRMNIPRKFQGRVSMTRDTSMETAFLDFRGLNFDDTGLYY |
| | | CVRDRSHGSGRLFESSNWFLDLWGRGTVVTVQSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQGNV |
| | | FSCSVMHEALHNHYTQKSLSLSPGK |
| | | SEQ ID NO: 29 |
| | | -------------------------------------------------- |
| | | --------------------- |
| | | QSALTQPRSVSASPGQSVTISCTGTHNLVSWCQHHPGRPPKLLIYDFNK |
| | | RASGVPDRFSGSGSGGTASLTISGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 30 |
| Sequence 16 | N49P74 | ADLVQSGAVVKKPGDSVRISCEAQGYTFIDYVIHWIRRAPGQGLEWM |
| | | GLMDPTYGRMNIPRKFQGRVSMTRDTSIETAFLDLRGLKSDDTGLYYC |
| | | VRDRSHGSGRLFESSNWFLDLWGRGTVVTVQSPSTKGPSVFPLAPSSK |
| | | STSGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| | | SEQ ID NO: 31 |
| | | -------------------------------------------------- |
| | | --------------------- |
| | | QSALTQPRSVSASPGQSVTISCTGTHNLVSWCQHHPGRPPKLLIYDFNK |
| | | RASGVPDRFSGSGSGGTASLTISGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 32 |
| Sequence 17 | N49P75 | ADLVQSGAVVKKPGDSVRISCEAQGYRFLDYIIHWIRRAPGQGLEWM |
| | | GWMNPMGGQVNIPWNFQGRVSMTRDTSIETAFLDLRGLKSDDTAVY |
| | | YCVRDRSNGSGKRFESSNWFLDLWGRGTAVTIHSPSTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| | | SEQ ID NO: 33 |
| | | -------------------------------------------------- |
| | | --------------------- |
| | | QSALTQPRSVSASPGQSVTISCTGTHNLVSWCQHHPGRAPKLLIYDFNK |
| | | RPSGVPDRFSGSGSGGTASLTITGLQDDDDAEYFCWAYEAFGGGTKLT |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGST |
| | | VEKTVAPTECS |
| | | SEQ ID NO: 34 |
| Sequence 18 | N49P9 | HVQLVQSGGGVKKIGAAVRISCEVTGYKFMDQLINWVRQAPGQGLE |
| | N49P9.1 | WMGWMNPTYGQVNYSWRFEGRVTMTRDMDTETAFMELRGLRVDDT |
| | N49P9.2 | AVYYCARGPSGENYPFHYWGQGVRVVVSSPSTKGPSVFPLAPSSKSTS |
| | N49P9i7 | GGTAALGCLVKDYFPEPVTVSWNSGALTSGVHTFPAVLQSSGLYSLSS |
| | N49P9-FR | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| | | SEQ ID NO: 35 |
| | | -------------------------------------------------- |
| | | --------------------- |
| | | ASALTQPASMSASPGQSVTISCSGTRHIISAWFQQYPGKPPKLIIFDDDK |
| | | RPSGVPSRFSASRPGDTASLTISNVQPEDEATYICNTYEFFGGGTRLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 36 |
| Sequence 19 | N49P22 | HIQLLQSGPQVKKSGDTVRISCETSGYNFVDSRIHWVRQTPEKRLRWM |
| | | GWINPLQGGVNYAPEFQGRIRMTRDTFIDTVYVDLSGLTPADTAYYYC |
| | | ARGIDGKSYPFHFWGHGTRVTVFSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| | | SEQ ID NO: 37 |
| | | -------------------------------------------------- |
| | | --------------------- |
| | | RFALTQPASVSGSPGQTITITCAGGSVSWFHFPPGKTPRLIIYESSKRPSG |
| | | VSPRFSGSQSGSTASLIISGLQSDDEGTYFCSILEFFGRGTLVTVLSQPKA |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVAP |
| | | AECS |
| | | SEQ ID NO: 38 |
| Sequence 20 | N49P23 | QVRLVQSGAGARKTGASMKLSCSTSGYTFTTHHGHFINWVRQARGQ |
| | | GLEWMGWMNPMTGQMNIEGKFQGRVTLTRDIYSDTAYMEMTRLTT |
| | | GDTGTYYCARGDFGQNYPFHYWGQGSLVIVSSASTKGPSVFPLAPSSK |
| | | STSGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| | | SEQ ID NO: 39 |
| | | -------------------------------------------------- |
| | | --------------------- |
| | | LSALTQPASVSGSPGQSVTISCSGTNRYLVSWYQQHPDKAPKLIIYDDN |
| | | KRPSGISDRFSASRPDDTASLTISGLQTGDEATYWCASYERFGGGTRLT |
| | | VLSQPKAAPSVTLFPPSSEELQANKATLVCLVSDFYPGAVTVAWKADG |
| | | SPVKVGVETTKPSKQSNNKYAASSYLSLTPEQWKSHRSYSCRVTHEGS |
| | | TVEKTVAPAECS |
| | | SEQ ID NO: 40 |
| Sequence 21 | N49P9.3 | HVQLVQSGGGVKKIGAAVRISCEVSGYNFMDQFINWVRQAPGQGLEW |
| | N49P9.4 | MGWMNPIYGQVNYSWRFQGRVTMTRDMYTDTAFMELRGLRVDDTA |
| | N49P9.6 | VYYCARGPSGENYPFHYWGQGVRVVVSSPSTKGPSVFPLAPSSKSTSG |
| | N49P9.3-FR | GTAALGCLVKDYFPEPVTVSWNSGALTSGVHTFPAVLQSSGLYSLSSV |
| | N49P9.6-FR | VTVPSSSLGTQTYICNVNHKPSNTKVDKKVEPKSCDKTHTCPPCPAPEL |
| | N49P9.6-FR- | LGGPSVFLFPPKPKDTLMISRTPEVTCVVVDVSHEDPEVKFNWYVDGV |
| | 54W | EVHNAKTKPREEQYNSTYRVVSVLTVLHQDWLNGKEYKCKVSNKALP |
| | N49P9.6-FR-54- | APIEKTISKAKGQPREPQVYTLPPSRDELTKNQVSLTCLVKGFYPSDIAV |
| | F | EWESNGQPENNYKTTPPVLDSDGSFFLYSKLTVDKSRWQQGNVFSCSV |
| | N49P9.6-54W | MHEALHNHYTQKSLSLSPGK |
| | N49P9.6-54F | SEQ ID NO: 41 |
| | N49P9.6-FR3-06 | -------------------------------------------------- |
| | N49P9.6-FR1-D | --------------------- |
| | N49P9.6-FR1-D-I | LTQPASMSASPGQSVTISCSGTRHIISAWFQQYPGKPPKLIIFDDDKRPSG |
| | | VPSRFSASRPGDTASLTISNVQPEDEATYICNTYEFFGGGTKLTVLSQPK |
| | | AAPSVTLFPPSSEELQANKATLVCLVSDFYPGAVTVAWKADGSPVKVG |
| | | VETTKPSKQSNNKYAASSYLSLTPEQWKSHRSYSCRVTHEGSTVEKTV |
| | | APAECS |
| | | SEQ ID NO: 42 |
| Sequence 22 | N49P51 | HIQLLQSGPQVKKSGDTVRISCETSGYNFVDSRIHWVRQTPEKRLRWM |
| | | GWINPLHGGVNYAPEFQGRIRMTRDTFIDTVYVDLSGLTPADTAYYYC |
| | | ARGIDGKSYPFHFWGHGTRVTVFSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| | | SEQ ID NO: 43 |
| | | -------------------------------------------------- |
| | | --------------------- |
| | | RFALTQPASVSGSPGQTITITCAGGSVSWFHFPPGKTPRLIIYESSKRPSG |
| | | VSPRFSGSQSGSTASLIISGLQSDDEGTYFCSILEFFGRGTLVTVLSQPKA |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVAP |
| | | AECS |
| | | SEQ ID NO: 44 |
| Sequence 23 | N49P52 | RVTLQQSGAIVRQPGASVTVSCETSGYTFTKYFIYWVRQAPGQGLEWL |
| | | GRIHPRTGAVKYAPRFQGRLSMTRDWSLDTAYLGLTGLTLGDTALYF |
| | | CARGAFEADSYGSSYPFHHWGQGTLVTVSSASTKGPSVFPLAPSSKST |
| | | SGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| | | SEQ ID NO: 45 |
| | | -------------------------------------------------- |
| | | --------------------- |
| | | SWALTQPASVSASPGQSVTMSCTGFGNYNPDSWYQQYPGKAPKLIIYE |
| | | DNKRPSGVSDRFSASRLGSTSSLTISNVQAADDAHYVCASFEFFGGGTK |
| | | LTVLSQPKAAPSVTLFPPSSEELQANKATLVCLVSDFYPGAVTVAWKA |
| | | DGSPVKVGVETTKPSKQSNNKYAASSYLSLTPEQWKSHRSYSCRVTHE |
| | | GSTVEKTVAPAECS |
| | | SEQ ID NO: 46 |
| Sequence 24 | N49P53 | RVTLQQSGATVKQPGASVTVSCETSGYTFTKYTIHWVRQAPGQGLQW |
| | | VGRIHPRTGAVKYAPIFQGKVSMSRDLSRDTAYLGLTRLTLADTALFF |
| | | CARGAFEADLSGPTYPFHHWGQGTLVIVSAASTKGPSVFPLAPSSKST |
| | | SGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIA |
| | | VMHEALHNHYTQKSLSLSPGK |
| | | SEQ ID NO: 47 |
| | | -------------------------------------------------- |
| | | --------------------- |
| | | SWALTQPASVSASPGQSVTMSCTGFGNYNPDSWYQQYPGKAPKLIIYE |
| | | DNKRPSGVSNRFSASRLGSTSSLTISNVQAADDAHYVCASFEFFGGGTK |
| | | LIVLSQPKAAPSVTLFPPSSEELQANKATLVCLVSDFYPGAVTVAWKAD |
| | | GSPVKVGVETTKPSKQSNNKYAASSYLSLTPEQWKSHRSYSCRVTHEG |
| | | STVEKTVAPAECS |
| | | SEQ ID NO: 48 |
| Sequence 25 | N49P54 | NVQLMQSGTEVKKSGASVTISCETAGFNFIDSVIHWLRQAPGGGFQWM |
| | | GWIKPLRGAVNYPQFLQGRVSMTRDLSTDTVYMVLNGLTPDDTGLYY |
| | | CAKGAFRGGSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| | | SEQ ID NO: 49 |
| | | -------------------------------------------------- |
| | | --------------------- |
| | | QSALSQPVSVSGSPGESITISCTGATTWYQQLPGRPPKLIIYDVTNRPSGI |
| | | SSRFSGSTSGHTASLTISGLQVDDEGLYHCASREFFGGGTKLTVLSQPK |
| | | AAPSVTLFPPSSEELQANKATLVCLVSDFYPGAVTVAWKADGSPVKVG |
| | | VETTKPSKQSNNKYAASSYLSLTPEQWKSHRSYSCRVTHEGSTVEKTV |
| | | APAECS |
| | | SEQ ID NO: 50 |
| Sequence 26 | N49P60 | QVRLVQSGPQVKKTGASVRVSCETSGYTFTSYFIHWLRLGPGEGLQW |
| | | MGWINPLHGAVNYENKFRGRVTITRDTSTDTVYLDMSRLTPDDTAVY |
| | | FCTRGIVADGWPYGHWGQGTQVTVSPASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| | | SEQ ID NO: 51 |
| | | -------------------------------------------------- |
| | | --------------------- |
| | | SWALTQPASVSGSPGQSVAISCAGGSVSWYQVLPGRAPKLIIYEGAKRP |
| | | SGVSARFSGSQSGNTAYLTISDLQTEDEGIYFCSSLQFFGGGTKLTVLSQ |
| | | PKAAPSVTLFPPSSEELQANKATLVCLVSDFYPGAVTVAWKADGSPVK |
| | | VGVETTKPSKQSNNKYAASSYLSLTPEQWKSHRSYSCRVTHEGSTVEK |
| | | TVAPAECS |
| | | SEQ ID NO: 52 |
| Sequence 27 | N49P61 | QVRLQQSGVVVRKPGASVRISCETSGFTFIDHIVHWVRRAPGRGFEWM |
| | | GWIKPLRGAVDYAPQLRGRISLTRDIYSETVFIDVSRLTSGDTAIYFCCK |
| | | AAAPEEAFPLQYWGQGTQLIVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| | | SEQ ID NO: 53 |
| | | -------------------------------------------------- |
| | | --------------------- |
| | | QAALTQPASVSGSPGQSVTISCLYANVDICWYQLHPGRAPKLLIVDNNK |
| | | RPSGVSPRFSGSKSGTTASLTISGLQADDEAEYHCSSRTFFGGGTKLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 54 |
| Sequence 28 | N49P62 | QVRLQQSGVVVRKPGASVRLSCETSGFKFIDHIVNWVRRAPGRGFEW |
| | | MGWIKPLGGVADYAPQHRGRISLTRDIYTETVFIDLSRLTSGDTAIYFC |
| | | CKAAAPDEAFPLEYWGQGTQLIVSPASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| | | SEQ ID NO: 55 |
| | | -------------------------------------------------- |
| | | --------------------- |
| | | QAALTQPASVSGSPGQSVTISCLYANVDICWYQIQPGRLPKLLIVDNNR |
| | | RPSGVSPRFSGSKSGTTASLTISGLQADDEAEYHCSSTTFFGGGTKLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 56 |
| Sequence 29 | N49P63 | QVRLVQSGPVMRKPGASVRISCETSGFAFLDHIVHWVRRAPGRGFEW |
| | | MGWVKTIGGVVDYAPHLRGRISVTRDVFSETVFLDLSRLTSGDTAMYF |
| | | CSKAAAPDEAFPLEFWGQGTQVIVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| | | SEQ ID NO: 57 |
| | | -------------------------------------------------- |
| | | --------------------- |
| | | QAALTQPASVSGSPGQSVTISCLYANVDICWYQLHPGRAPKLLILDNNK |
| | | RPSGVSSRFSGSKSGTTASLTISDLQADDEAEYHCSSTTFFGGGTRLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 58 |
| Sequence 30 | N49P64 | QVRLVQSGPVVRKPGTSVRISCETSGFAFLDHIVHWVRRAPGRGFEWM |
| | | GWVKTIGGVVDYAPHLRGRISVTRDVFSEIVFMELSRLTSGDTAMYFC |
| | | SKAAAPDEAFPLEFWGQGTQVIVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| | | SEQ ID NO: 59 |
| | | -------------------------------------------------- |
| | | --------------------- |
| | | QAALTQPASVSGSPGQSVTISCLYANVDICWYQLHPGRAPKLLIVDNNK |
| | | RPSGVSSRFSGSKSGTTASLTISDLQADDEAEYHCSSTTFFGGGTRLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 60 |
| Sequence 31 | N49P65 | QVQLVQSGAGVKKPGASVRVSCETSGFKFTEYFIHFLRQAPGQGLEW |
| | | MGWLNPLRGAVNYPRKFQGRVTLTRDIYTTTVYMQLNGLTPDDTAV |
| | | YYCARAVFNEAFPFDYWGQGSLVTVSSASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| | | SEQ ID NO: 61 |
| | | -------------------------------------------------- |
| | | --------------------- |
| | | SWAQTQPASVSGSPGQSITISCAGIVSDAWYQQYPGRPPRLILYDGDKR |
| | | PSGVSPRFSASRAGKTASLTISGLQADDEAYYHCASREFFGGVTKLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 62 |
| Sequence 32 | N49P66 | QVRLQQSGVVVRKPGASVRLSCETSGFKFIDHIVNWVRRAPGRGFEW |
| | | MGWIKPLGGVADYAPQHRGRISLTRDIYTETVFIDLSRLTSGDTAIYFC |
| | | CKAAAPDEAFPLEYWGQGTQLIVSPASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| | | SEQ ID NO: 63 |
| | | -------------------------------------------------- |
| | | --------------------- |
| | | QAALTQPASVSGSPGQSVTISCLYANVDICWYQIQPGRLPKLLIVDNDR |
| | | RPSGVSPRFSGSKSGTTASLTISGLQADDEAEYHCSSTTFFGGGTKLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 64 |
| Sequence 33 | N49P67 | QVRLVQSGPVMRKPGASVRISCETSGFAFLDHIVHWVRRAPGRGFEW |
| | | MGWVKTIGGVVDYAPHLRGRISVTRDVFSETVFLDLSRLTSGDTAMYF |
| | | CSKAAAPDEAFPLEFWGQGTQVIVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| | | SEQ ID NO: 65 |
| | | -------------------------------------------------- |
| | | --------------------- |
| | | QAALTQPASVSGSPGQSVTISCLYANVDICWYQLHPGRAPKLLILDNNK |
| | | RPSGVSSRFSGSKSGTTASLTISDLQADDEAEYHCSSTTFFGGGTRLTVL |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 66 |
| Sequence 34 | N49P68 | HVQLRQSGTEAKKSGASVTISCETAGFNFIDSVIHWLRQAPGGGFQWM |
| | | GWIKPLRGGVNYPHYLQGRISMTRDLSSDTVYMVLNRLTPADTGLYY |
| | | CAKGAFGGSSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| | | SEQ ID NO: 67 |
| | | -------------------------------------------------- |
| | | --------------------- |
| | | QSALSQPVSVSGSPGESITISCTEATTWYQQLPGKPPKLIIYDVTNRPSGI |
| | | SSRFSGSMSGRTASLTISGLQVDDEGLYHCASREFFGGGTKLTVLSQPK |
| | | AAPSVTLFPPSSEELQANKATLVCLVSDFYPGAVTVAWKADGSPVKVG |
| | | VETTKPSKQSNNKYAASSYLSLTPEQWKSHRSYSCRVTHEGSTVEKTV |
| | | APAECS |
| | | SEQ ID NO: 68 |
| Sequence 35 | N49P69 | HVQLMQSGTQAKKSGASVTISCIGAGFKFIDSVIHWLRQAPGGGIQW |
| | | MGWIKPLGGAVNYPPYLQGRISLTRDLSTDTIYMVLNGLTPADTGFYY |
| | | CAKGAFGGGSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| | | SEQ ID NO: 69 |
| | | -------------------------------------------------- |
| | | --------------------- |
| | | QSALSQPVSVSGSPGDSITISCFGATTWYQQLPGRPPKLIIYDVTNRPSGI |
| | | SGRFSGSMSGQKASLTISGLQVDDEGLYHCASREFFGGGTKLTVLSQPK |
| | | AAPSVTLFPPSSEELQANKATLVCLVSDFYPGAVTVAWKADGSPVKVG |
| | | VETTKPSKQSNNKYAASSYLSLTPEQWKSHRSYSCRVTHEGSTVEKTV |
| | | APAECS |
| | | SEQ ID NO: 70 |
| Sequence 36 | N49P70 | HVQLRQSGTEAKKSGASVTISCETAGFNFIDSVIHWLRQAPGGGFQWM |
| | | GWIKPLRGGVNYPHYLQGRISMTRDLSSDTVYMVLNRLTPDDTGLYY |
| | | CAKGAFGGSSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | NGQPENNYKTTPPVLDSDGSFFLYSKLTVDKSRWQQGNVFSCSVMHEA |
| | | LHNHYTQKSLSLSPGK |
| | | SEQ ID NO: 71 |
| | | -------------------------------------------------- |
| | | --------------------- |
| | | QSALSQPVSVSGSPGESITISCTEATTWYQQLPGRSPKLIIYDVTNRPSGIS |
| | | SRFSGSMSGRTASLTISGLQVDDEGLYHCASREFFGGGTKLTVLSQPKA |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVAP |
| | | AECS |
| | | SEQ ID NO: 72 |
| Sequence 37 | N49P71 | RVTLQQSGATVRQPGASVTVSCETSGFTFIKYTIHWVRQAPGQGLQW |
| | | VGRIHPRTGAVKFAPIFQGKFSMSRDLSRDTAYLGLTRLTLADTALFFC |
| | | ARGAFEADLYGPTYPFHHWGQGTQVTVSAASTKGPSVFPLAPSSKSTS |
| | | GGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| | | SEQ ID NO: 73 |
| | | -------------------------------------------------- |
| | | --------------------- |
| | | SWALTQPASVSASPGQSVTMSCTGFGSYNPDSWYQQYPGKAPKLIIYD |
| | | DNKRPSGVSDRFSASRLGSTSSLTISNVQAADDAHYVCASFEFFGGGTK |
| | | LTVLSQPKAAPSVTLFPPSSEELQANKATLVCLVSDFYPGAVTVAWKA |
| | | DGSPVKVGVETTKPSKQSNNKYAASSYLSLTPEQWKSHRSYSCRVTHE |
| | | GSTVEKTVAPAECS |
| | | SEQ ID NO: 74 |
| Sequence 38 | N49P72 | HIQLLQSGPQVKKSGDTVRISCETSGYNFVDSLIHWVRQTPEKRLRWM |
| | | GWINPLQGGVNYAPEFQGRIRMTRDTFIDTVYVDLSGLTPADTAYYYC |
| | | ARGIDGNSYPFHFWGHGTRVTVFSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| | | SEQ ID NO: 75 |
| | | -------------------------------------------------- |
| | | --------------------- |
| | | RFALTQPASVSGSPGQTITITCAGGSVSWFHFPPGKTPRLIIYESSKRPGS |
| | | VSPRFSGSQSGSTASLIISGLQSDDEGTYFCSILEFFGRGTLLTVLSQPKA |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVAP |
| | | AECS |
| | | SEQ ID NO: 76 |

TABLE 2
Nucleotide sequences of natural antibodies. Heavy chain is shown first (above dotted line),
followed by the light chain (below dotted line).

| Natural | Related | |
|---|---|---|
| antibody | variants | Nucleotide sequence |
| Sequence 1 | N49P6, | gcgggactgatgcagtctggggctgtgatgaagaattcgggggcctcagtgagggtctcttgtcaggctgatggatacgacttcatt |
| | N49P6.2 | gactatgtcattcactggtttcgacaaagacgtggagaaggtcttgagtggctgggatggatgaatccctcgggaggcggcacaa |
| | N49P6- | actatccgcgaccatttcagggcaaagtcaccatgaccagggacacgtccaccgagacagcctatttagatgtcagaggacttaca |
| | LS1 | tatgacgacacggccgtctattattgtgtgagagacagggccaacggttcgggaagaagacgttttgagtcggtgaattggttcctg |
| | N49P6- | gatctgtggggccgcggcacccaaataacagtcgtctcgccctccaccaagggcccatcggtcttccccctggcaccctcctcca |
| | LS2 | agagcacctctgggggcacagcggccctgggctgcctggtcaaggactacttccccgaaccggtgacggtgtcgtggaactcag |
| | N49P6A | gcgccctgaccagcggcgtgcacaccttcccggctgtcctacagtcctcaggactctactccctcagcagcgtggtgaccgtgcc |
| | N49P6S | ctccagcagcttgggcacccagacctacatctgcaacgtgaatcacaagcccagcaacaccaaggtggacaagaaagttgagcc |
| | N49P6F | caaatcttgtgacaaaactcacacatgcccaccgtgcccagcacctgaactcctggggggaccgtcagtcttcctcttccccccaa |
| | N49P6Y | aacccaaggacaccctcatgatctcccggacccctgaggtcacatgcgtggtggtggacgtgagccacgaagaccctgaggtca |
| | N49P6.54 | agttcaactggtacgtggacggcgtggaggtgcataatgccaagacaaagccgcgggaggagcagtacaacagcacgtaccg |
| | TY | ggtggtcagcgtcctcaccgtcctgcaccaggactggctgaatggcaaggagtacaagtgcaaggtctccaacaaagccctccc |
| | | agcccccatcgagaaaaccatctccaaagccaaagggcagccccgagaaccacaggtgtacaccctgcccccatcccgggatg |
| | | agctgaccaagaaccaggtcagcctgacctgcctggtcaaaggcttctatcccagcgacatcgccgtggagtgggagagcaatg |
| | | ggcagccggagaacaactacaagaccacgcctcccgtgctggactccgacggctccttcttcctctacagcaagctcaccgtgga |
| | | caagagcaggtggcagcaggggaacgtcttctcatgctccgtgatgcatgaggctctgcacaaccactacacgcagaagagcct |
| | | ctccctgtctccgggtaaa |
| | | SEQ ID NO: 77 |
| | | ----------------------------------------------------------------------------------------- |
| | | cagtctgccctaactcagcctcgctcagtgtccgcatctcctggtcagtcagtcaccatctcctgcactggaacacacaattatgtgtc |
| | | ctggtgtcaacagaaaccgggccaagcccccaaattattaatttacgatttcaataaacggccctcaggggtctctgatcgcttctct |
| | | ggctccacgtctggcaacacggcctccctgaccatctctggactccaggctgacgatgagggtcattatttttgttgggcgtttgaaa |
| | | atatcggcggagggaccaagctgaccgtcctgcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| | | SEQ ID NO: 78 |
| Sequence 2 | N49P7, | gcggacttggtgcagtctggggctgtggtgaagaagcctggggactcagtgaggatctcctgtgaggctcaaggatatagatttcc |
| | N49P7.1 | tgactacatcattcactggattcgacgggcccctggacaaggccctgaatggatgggatggatgaatccaatgggcggacaagta |
| | N49P7A | aatattccatggaaatttcagggtagggtctccatgacccgggacacgtccatcgaaacagcatttctggacttaagaggactaaag |
| | N49P7S | tctgacgacacggccgtctattattgcgtgagagatcgcagtaatggatcgggaaagcgattcgagtcctccaattggttcctcgatc |
| | N49P7F | tgtggggccgtgggactgcggtcacaattcaatca |
| | N49P7Y | tcctccaccaagggcccatcggtcttccccctggcaccctcctccaagagcacctctgggggcacagcggccctgggctgcctg |
| | N49P7.54 | gtcaaggactacttccccgaaccggtgacggtgtcgtggaactcaggcgccctgaccagcggcgtgcacaccttcccggctgtc |
| | TY | ctacagtcctcaggactctactccctcagcagcgtggtgaccgtgccctccagcagcttgggcacccagacctacatctgcaacgt |
| | N49P7- | gaatcacaagcccagcaacaccaaggtggacaagaaagttgagcccaaatcttgtgacaaaactcacacatgcccaccgtgccc |
| | LS1 | agcacctgaactcctggggggaccgtcagtcttcctcttccccccaaaacccaaggacaccctcatgatctcccggacccctgag |
| | N49P7- | gtcacatgcgtggtggtggacgtgagccacgaagaccctgaggtcaagttcaactggtacgtggacggcgtggaggtgcataat |
| | LS2 | gccaagacaaagccgcgggaggagcagtacaacagcacgtaccgggtggtcagcgtcctcaccgtcctgcaccaggactggc |
| | N49P7/6L | tgaatggcaaggagtacaagtgcaaggtctccaacaaagccctcccagcccccatcgagaaaaccatctccaaagccaaaggg |
| | N49P7/11 | cagccccgagaaccacaggtgtacaccctgcccccatcccgggatgagctgaccaagaaccaggtcagcctgacctgcctggt |
| | L | caaaggcttctatcccagcgacatcgccgtggagtgggagagcaatgggcagccggagaacaactacaagaccacgcctcccg |
| | R49P7 | tgctggactccgacggctccttcttcctctacagcaagctcaccgtggacaagagcaggtggcagcaggggaacgtcttctcatgc |
| | N49P7-FR | tccgtgatgcatgaggctctgcacaaccactacacgcagaagagcctctccctgtctccgggtaaa |
| | | SEQ ID NO: 79 |
| | | ----------------------------------------------------------------------------------------- |
| | | gccctgactcagcctcgctcagtgtccgcatctcctgggcagtccgtcaccatttcctgcactggaacccacaatttggtctcttggt |
| | | gtcaacatcagccaggcagagcccccaaattattaatttatgacttcaataagaggccctcaggggtccctgatcgcttctctggctc |
| | | cgggtctggcggcacggcctccctgaccatcactggactccaggatgacgatgacgccgaatatttttgttgggcgtatgaagcttt |
| | | tggcggagggaccaagttgaccgttcttcgtcagcccaaggctgccccctcggtcactctgttcccgccctcctctgaggagcttca |
| | | agccaacaaggccacactggtgtgtctcataagtgacttctacccgggagccgtgacagtggcttggaaagcagatagcagcccc |
| | | gtcaaggcgggagtggagaccaccacaccctccaaacaaagcaacaacaagtacgcggccagcagctatctgagcctgacgc |
| | | ctgagcagtggaagtcccacagaagctacagctgccaggtcacgcatgaagggagcaccgtggagaagacagtggcccctac |
| | | agaatgttca |
| | | SEQ ID NO: 80 |
| Sequence 3 | N49P7.2 | gcggacttggtgcagtctggggctgtggtgaagaagcctggggactcagtgaggatctcctgtgaggctcaaggatacaaatttcc |
| | | tgactacatcattcactggattcgacgggcccctggacaaggccttgagtggatggggtggattaatccaatgggcggacaagtaa |
| | | acattccatggcagtttcagggcagggtctccatgacccgggacacgtccatcgaaacagcatttctggacttaagaggactaaag |
| | | tctgacgacacggccctctattattgcgtgagagatcgaagtaatggatcgggaaggcgattcgagtcctccaattggttcctcgatc |
| | | tgtggggccgcggcactgcggtcactgttcattcaccctccaccaagggcccatcggtcttccccctggcaccctcctccaagagc |
| | | acctctgggggcacagcggccctgggctgcctggtcaaggactacttccccgaaccggtgacggtgtcgtggaactcaggcgcc |
| | | ctgaccagcggcgtgcacaccttcccggctgtcctacagtcctcaggactctactccctcagcagcgtggtgaccgtgccctccag |
| | | cagcttgggcacccagacctacatctgcaacgtgaatcacaagcccagcaacaccaaggtggacaagaaagttgagcccaaatc |
| | | ttgtgacaaaactcacacatgcccaccgtgcccagcacctgaactcctggggggaccgtcagtcttcctcttccccccaaaaccca |
| | | aggacaccctcatgatctcccggacccctgaggtcacatgcgtggtggtggacgtgagccacgaagaccctgaggtcaagttca |
| | | actggtacgtggacggcgtggaggtgcataatgccaagacaaagccgcgggaggagcagtacaacagcacgtaccgggtggt |
| | | cagcgtcctcaccgtcctgcaccaggactggctgaatggcaaggagtacaagtgcaaggtctccaacaaagccctcccagcccc |
| | | catcgagaaaaccatctccaaagccaaagggcagccccgagaaccacaggtgtacaccctgcccccatcccgggatgagctga |
| | | ccaagaaccaggtcagcctgacctgcctggtcaaaggcttctatcccagcgacatcgccgtggagtgggagagcaatgggcagc |
| | | cggagaacaactacaagaccacgcctcccgtgctggactccgacggctccttcttcctctacagcaagctcaccgtggacaagag |
| | | caggtggcagcaggggaacgtcttctcatgctccgtgatgcatgaggctctgcacaaccactacacgcagaagagcctctccctg |
| | | tctccgggtaaa |
| | | SEQ ID NO: 81 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagagcccccaaattattaatttatgacttcaataagaggccctcaggggtccctgatcgcttctct |
| | | ggctccgggtctggcggcacggcctccctgaccatcagtggactccaggatgacgatgacgccgaatatttttgttgggcgtatga |
| | | agcttttggcggagggaccaagttgaccgtacttcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| | | SEQ ID NO: 82 |
| Sequence 4 | N49P11 | tcggcggaattggtgcaatctggggctgtggtgaagaagcctgggacctccgtgaaggtctcttgtcaggcttatggatacactttta |
| | | ccgactaccttattcattggcttcgacaggcccctggacaaggacttgaatggatgggatggatgaatcctgtttatggacaagtaaa |
| | | ttatgcccaaaactttcagggcagggtctccatgaccagggacatttacagggaaacagcatttctagaggtgcgcgacctgaaga |
| | | ctgacgacacaggcacttattattgtgtgagagacacaggcgacggttcgcggagacactttgactccatcaattggtttctcgatctt |
| | | tggggccgcgggacatggataagggtcgccccagcctccaccaagggcccatcggtcttccccctggcaccctcctccaagag |
| | | cacctctgggggcacagcggccctgggctgcctggtcaaggactacttccccgaaccggtgacggtgtcgtggaactcaggcgc |
| | | cctgaccagcggcgtgcacaccttcccggctgtcctacagtcctcaggactctactccctcagcagcgtggtgaccgtgccctcca |
| | | gcagcttgggcacccagacctacatctgcaacgtgaatcacaagcccagcaacaccaaggtggacaagaaagttgagcccaaat |
| | | cttgtgacaaaactcacacatgcccaccgtgcccagcacctgaactcctggggggaccgtcagtcttcctcttccccccaaaaccc |
| | | aaggacaccctcatgatctcccggacccctgaggtcacatgcgtggtggtggacgtgagccacgaagaccctgaggtcaagttc |
| | | aactggtacgtggacggcgtggaggtgcataatgccaagacaaagccgcgggaggagcagtacaacagcacgtaccgggtgg |
| | | tcagcgtcctcaccgtcctgcaccaggactggctgaatggcaaggagtacaagtgcaaggtctccaacaaagccctcccagccc |
| | | ccatcgagaaaaccatctccaaagccaaagggcagccccgagaaccacaggtgtacaccctgcccccatcccgggatgagctg |
| | | accaagaaccaggtcagcctgacctgcctggtcaaaggcttctatcccagcgacatcgccgtggagtgggagagcaatgggcag |
| | | ccggagaacaactacaagaccacgcctcccgtgctggactccgacggctccttcttcctctacagcaagctcaccgtggacaaga |
| | | gcaggtggcagcaggggaacgtcttctcatgctccgtgatgcatgaggctctgcacaaccactacacgcagaagagcctctccct |
| | | gtctccgggtaaa |
| | | SEQ ID NO: 83 |
| | | ----------------------------------------------------------------------------------------- |
| | | cagtgtgtcttgactcagcctcgctcagtgtccggatctcctggacaatcagtcaccatctcctgcactggaactcacaattatgtctc |
| | | ctggtgtcaacaccacccaggcaacgcccccaaattattactttatgatttcgacaagcggccctcaggaatctctgatcgcttctctg |
| | | gctctaggtctggcaacacggcctccctgaccatctctggcctccagcctgaggatgaggccgattacttttgttgggcctttgaagc |
| | | ctttggcggagggaccaaggtgctcgtccttcgtcagcccaaggctgccccctcggtcactctgttcccgccctcctctgaggagc |
| | | ttcaagccaacaaggccacactggtgtgtctcataagtgacttctacccgggagccgtgacagtggcttggaaagcagatagcag |
| | | ccccgtcaaggcgggagtggagaccaccacaccctccaaacaaagcaacaacaagtacgcggccagcagctatctgagcctg |
| | | acgcctgagcagtggaagtcccacagaagctacagctgccaggtcacgcatgaagggagcaccgtggagaagacagtggccc |
| | | ctacagaatgttca |
| | | SEQ ID NO: 84 |
| Sequence 5 | N49P18 | gcggacttggtgcagtctggggctgtgatgaagaagcctggggactcagtgagaatctcctgtgaggctcgaggatacacattca |
| | N49P18.2 | ctgactacgtcattcactggattcgacgggcccctggacaaggccttgaatggatggggtggattgatccaccttatggacaagtaa |
| | | atattccatggaattttcagggcagggtctccatgacccgggacacgtccatcgaaacagcatttctggacttaagaggtctaaagtc |
| | | tgacgacacgggcctctattattgcgtgagagatcgaagtaatggatggggaaagcgattcgagtcctccaattggttcctcgatct |
| | | gtggggccgcggcactgtggtcactgttcactcaccctccaccaagggcccatcggtcttccccctggcaccctcctccaagagc |
| | | acctctgggggcacagcggccctgggctgcctggtcaaggactacttccccgaaccggtgacggtgtcgtggaactcaggcgcc |
| | | ctgaccagcggcgtgcacaccttcccggctgtcctacagtcctcaggactctactccctcagcagcgtggtgaccgtgccctccag |
| | | cagcttgggcacccagacctacatctgcaacgtgaatcacaagcccagcaacaccaaggtggacaagaaagttgagcccaaatc |
| | | ttgtgacaaaactcacacatgcccaccgtgcccagcacctgaactcctggggggaccgtcagtcttcctcttccccccaaaaccca |
| | | aggacaccctcatgatctcccggacccctgaggtcacatgcgtggtggtggacgtgagccacgaagaccctgaggtcaagttca |
| | | actggtacgtggacggcgtggaggtgcataatgccaagacaaagccgcgggaggagcagtacaacagcacgtaccgggtggt |
| | | cagcgtcctcaccgtcctgcaccaggactggctgaatggcaaggagtacaagtgcaaggtctccaacaaagccctcccagcccc |
| | | catcgagaaaaccatctccaaagccaaagggcagccccgagaaccacaggtgtacaccctgcccccatcccgggatgagctga |
| | | ccaagaaccaggtcagcctgacctgcctggtcaaaggcttctatcccagcgacatcgccgtggagtgggagagcaatgggcagc |
| | | cggagaacaactacaagaccacgcctcccgtgctggactccgacggctccttcttcctctacagcaagctcaccgtggacaagag |
| | | caggtggcagcaggggaacgtcttctcatgctccgtgatgcatgaggctctgcacaaccactacacgcagaagagcctctccctg |
| | | tctccgggtaaa |
| | | SEQ ID NO: 85 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagagcccccaagttattaatttatgacttcaataagaggccctcaggggtccctgatcgcttctct |
| | | ggctccgggtctggcggcacggcctccctaaccatcagtggactccaggatgacgatgacgccgaatatttttgttgggcatatga |
| | | agctttcggcggagggaccaagttgactgtacttcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| | | SEQ ID NO: 86 |
| Sequence 6 | N49P18.1 | gcggacttggtgcagtctggggctgtggtgaagaagcctggggactcagtgagaatctcctgtgaggctcaaggatacacattca |
| | | ctgactacgtcattcactggattcgacgggcccctggacaaggccttgaatggatggggtggattaatccaggttatggacaagtaa |
| | | atattccatggaactttcagggcagggtctccatgacccgagacacgtccatcgaaacagcatttctggacttaagaggtctaaagt |
| | | ctgacgacacgggcctctattattgcgtgagagatcgaagtaatggatggggaaagcgattcgagtcctccaattggttcctcgatc |
| | | tgtggggccgcggcactgtggtcactgttcactcaccctccaccaagggcccatcggtcttccccctggcaccctcctccaagagc |
| | | acctctgggggcacagcggccctgggctgcctggtcaaggactacttccccgaaccggtgacggtgtcgtggaactcaggcgcc |
| | | ctgaccagcggcgtgcacaccttcccggctgtcctacagtcctcaggactctactccctcagcagcgtggtgaccgtgccctccag |
| | | cagcttgggcacccagacctacatctgcaacgtgaatcacaagcccagcaacaccaaggtggacaagaaagttgagcccaaatc |
| | | ttgtgacaaaactcacacatgcccaccgtgcccagcacctgaactcctggggggaccgtcagtcttcctcttccccccaaaaccca |
| | | aggacaccctcatgatctcccggacccctgaggtcacatgcgtggtggtggacgtgagccacgaagaccctgaggtcaagttca |
| | | actggtacgtggacggcgtggaggtgcataatgccaagacaaagccgcgggaggagcagtacaacagcacgtaccgggtggt |
| | | cagcgtcctcaccgtcctgcaccaggactggctgaatggcaaggagtacaagtgcaaggtctccaacaaagccctcccagcccc |
| | | catcgagaaaaccatctccaaagccaaagggcagccccgagaaccacaggtgtacaccctgcccccatcccgggatgagctga |
| | | ccaagaaccaggtcagcctgacctgcctggtcaaaggcttctatcccagcgacatcgccgtggagtgggagagcaatgggcagc |
| | | cggagaacaactacaagaccacgcctcccgtgctggactccgacggctccttcttcctctacagcaagctcaccgtggacaagag |
| | | caggtggcagcaggggaacgtcttctcatgctccgtgatgcatgaggctctgcacaaccactacacgcagaagagcctctccctg |
| | | tctccgggtaaa |
| | | SEQ ID NO: 87 |
| | | ----------------------------------------------------------------------------------------- |
| | | cagtctgccctgactcagcctcgctcaatgtccgcatctcctgggcagtccgtcaccatttcctgcactggaacccacaatttggtct |
| | | cttggtgtcaacatcacccaggcagaccccccaaattattaatttatgacttcaataagaggccctcaggggtccctgatcgcttctct |
| | | ggctccgggtctggcggcacggcctccctgaccatcagtggactccaggatgacgatgacgccgaatacatttgttgggcatatg |
| | | aagctttcggcggagggaccaagttgaccgtacttcgtcagcccaaggctgccccctcggtcactctgttcccgccctcctctgag |
| | | gagcttcaagccaacaaggccacactggtgtgtctcataagtgacttctacccgggagccgtgacagtggcttggaaagcagata |
| | | gcagccccgtcaaggcgggagtggagaccaccacaccctccaaacaaagcaacaacaagtacgcggccagcagctatctgag |
| | | cctgacgcctgagcagtggaagtcccacagaagctacagctgccaggtcacgcatgaagggagcaccgtggagaagacagtg |
| | | gcccctacagaatgttca |
| Sequence 7 | N49P19 | SEQ ID NO: 88 |
| | | gcggacttggtgcagtctggggctgtggtgaaaaatgctggggcctcagtgagggtctcctgtgaggcttatggatacacattcgt |
| | | ggactacttcattcattgggtccgacaggcccctggacaaggctttgaatggatgggatacatggatcccttgaacgggcgcccaa |
| | | acattgcgcgaaaatttcagggcaggctctccctgagtcgagataggtccagcgaaacttcatttctggacttaagtggactgaggt |
| | | ctgacgactcggccgtctattattgtgtgagagacaagagtaatggatcgggcagacggtttgactcgtctaattggtttctcgatctg |
| | | tggggccgtggaacccgggtcagtattttctcagcctccaccaagggcccatcggtcttccccctggcaccctcctccaagagcac |
| | | ctctgggggcacagcggccctgggctgcctggtcaaggactacttccccgaaccggtgacggtgtcgtggaactcaggcgccct |
| | | gaccagcggcgtgcacaccttcccggctgtcctacagtcctcaggactctactccctcagcagcgtggtgaccgtgccctccagc |
| | | agcttgggcacccagacctacatctgcaacgtgaatcacaagcccagcaacaccaaggtggacaagaaagttgagcccaaatctt |
| | | gtgacaaaactcacacatgcccaccgtgcccagcacctgaactcctggggggaccgtcagtcttcctcttccccccaaaacccaa |
| | | ggacaccctcatgatctcccggacccctgaggtcacatgcgtggtggtggacgtgagccacgaagaccctgaggtcaagttcaa |
| | | ctggtacgtggacggcgtggaggtgcataatgccaagacaaagccgcgggaggagcagtacaacagcacgtaccgggtggtc |
| | | agcgtcctcaccgtcctgcaccaggactggctgaatggcaaggagtacaagtgcaaggtctccaacaaagccctcccagccccc |
| | | atcgagaaaaccatctccaaagccaaagggcagccccgagaaccacaggtgtacaccctgcccccatcccgggatgagctgac |
| | | caagaaccaggtcagcctgacctgcctggtcaaaggcttctatcccagcgacatcgccgtggagtgggagagcaatgggcagcc |
| | | ggagaacaactacaagaccacgcctcccgtgctggactccgacggctccttcttcctctacagcaagctcaccgtggacaagagc |
| | | aggtggcagcaggggaacgtcttctcatgctccgtgatgcatgaggctctgcacaaccactacacgcagaagagcctctccctgt |
| | | ctccgggtaaa |
| | | SEQ ID NO: 89 |
| | | ----------------------------------------------------------------------------------------- |
| | | cagtctgccctgactcagcctcgctcagtgtccgcaactcctggacagtcagtcaccatctcctgcactggaacccacaattatgtct |
| | | cttggtgtcaacaacatccaggcagagcccccaaattactaatttatgacttcaataagaggccctcaggggtcccggatcgcttctc |
| | | tggctccggatctggcggcacggcctccctaaccatcactggactccaggatgacgatgaagcggactatttttgttgggcctatga |
| | | tgcttttggcggagggaccaagttgaccgtcctgcgtcagcccaaggctgccccctcggtcactctgttcccgccctcctctgagg |
| | | agcttcaagccaacaaggccacactggtgtgtctcataagtgacttctacccgggagccgtgacagtggcttggaaagcagatag |
| | | cagccccgtcaaggcgggagtggagaccaccacaccctccaaacaaagcaacaacaagtacgcggccagcagctatctgagc |
| | | ctgacgcctgagcagtggaagtcccacagaagctacagctgccaggtcacgcatgaagggagcaccgtggagaagacagtgg |
| | | cccctacagaatgttca |
| | | SEQ ID NO: 90 |
| Sequence 8 | N49P37 | gcggacttggtgcagtctggggctgtggtgaagaagcctggggactcagtgagggtctcctgtgaggcttatggatacacattcag |
| | | tgactacatcattcattggattcgacgggcccctggacgaggccttgaatggatgggatggatgaatccgatgggcggacaagtg |
| | | aatattccgtggaactttcaggggagagtctccatgacccgggacacgtccatcgaaacagcatttctggacttaagaggactgag |
| | | gtctgacgacacggccgtctattactgtgtgagagatcgcagcaatggatcgggcaagcgatttgagtcctccaattggttcctcga |
| | | tctgtggggccgcgggaccgcggtcactatttcctcaccctccaccaagggcccatcggtcttccccctggcaccctcctccaaga |
| | | gcacctctgggggcacagcggccctgggctgcctggtcaaggactacttccccgaaccggtgacggtgtcgtggaactcaggc |
| | | gccctgaccagcggcgtgcacaccttcccggctgtcctacagtcctcaggactctactccctcagcagcgtggtgaccgtgccctc |
| | | cagcagcttgggcacccagacctacatctgcaacgtgaatcacaagcccagcaacaccaaggtggacaagaaagttgagccca |
| | | aatcttgtgacaaaactcacacatgcccaccgtgcccagcacctgaactcctggggggaccgtcagtcttcctcttccccccaaaa |
| | | cccaaggacaccctcatgatctcccggacccctgaggtcacatgcgtggtggtggacgtgagccacgaagaccctgaggtcaag |
| | | ttcaactggtacgtggacggcgtggaggtgcataatgccaagacaaagccgcgggaggagcagtacaacagcacgtaccgggt |
| | | ggtcagcgtcctcaccgtcctgcaccaggactggctgaatggcaaggagtacaagtgcaaggtctccaacaaagccctcccagc |
| | | ccccatcgagaaaaccatctccaaagccaaagggcagccccgagaaccacaggtgtacaccctgcccccatcccgggatgagc |
| | | tgaccaagaaccaggtcagcctgacctgcctggtcaaaggcttctatcccagcgacatcgccgtggagtgggagagcaatgggc |
| | | agccggagaacaactacaagaccacgcctcccgtgctggactccgacggctccttcttcctctacagcaagctcaccgtggacaa |
| | | gagcaggtggcagcaggggaacgtcttctcatgctccgtgatgcatgaggctctgcacaaccactacacgcagaagagcctctcc |
| | | ctgtctccgggtaaa |
| | | SEQ ID NO: 91 |
| | | ----------------------------------------------------------------------------------------- |
| | | cagtctgccctgactcagcctcgctcagtgtccgcagctcctggacagtccgtcaccatttcctgcactggaacccacaatttggttt |
| | | cttggtgtcaacatcacccaggcagagcccccaagttattaatttatgacttcaataagagaccctcaggtgtccctgatcgtttctctg |
| | | gctccgggtctggcggcacggcctccctaaccatcactggactccaggatgacgatgaggctgaatatttttgttgggcgtatgaa |
| | | gtttttggcggagggaccaagttgaccgtgcttcgtcagcccaaggctgccccctcggtcactctgttcccgccctcctctgaggag |
| | | cttcaagccaacaaggccacactggtgtgtctcataagtgacttctacccgggagccgtgacagtggcttggaaagcagatagca |
| | | gccccgtcaaggcgggagtggagaccaccacaccctccaaacaaagcaacaacaagtacgcggccagcagctatctgagcct |
| | | gacgcctgagcagtggaagtcccacagaagctacagctgccaggtcacgcatgaagggagcaccgtggagaagacagtggcc |
| | | cctacagaatgttca |
| | | SEQ ID NO: 92 |
| Sequence 9 | N49P38 | gcggacttggtgcagtctggggctgtggtgaagacgcctggggcctcagtgagggtctcctgtgaggcttatggatacacattcatt |
| | | gactacatcattcattgggtccgacaggcccctggacaaggttttgaatggctgggatacatcgatcctatgaacgggcgcccaaa |
| | | cattgcgcgaaaatttcagggcaggctctccctgagccgggatacgtccatcgaaacatcatttctggacttaagtggactgaggtc |
| | | tgacgactcggccgtctattattgtgtgagagacaagagtaatggatcgggcaaacgatttgactcctctaattggtttctcgatctgt |
| | | ggggccgtggaacgcgggtcagcatttcttcagcctccaccaagggcccatcggtcttccccctggcaccctcctccaagagcac |
| | | ctctgggggcacagcggccctgggctgcctggtcaaggactacttccccgaaccggtgacggtgtcgtggaactcaggcgccct |
| | | gaccagcggcgtgcacaccttcccggctgtcctacagtcctcaggactctactccctcagcagcgtggtgaccgtgccctccagc |
| | | agcttgggcacccagacctacatctgcaacgtgaatcacaagcccagcaacaccaaggtggacaagaaagttgagcccaaatctt |
| | | gtgacaaaactcacacatgcccaccgtgcccagcacctgaactcctggggggaccgtcagtcttcctcttccccccaaaacccaa |
| | | ggacaccctcatgatctcccggacccctgaggtcacatgcgtggtggtggacgtgagccacgaagaccctgaggtcaagttcaa |
| | | ctggtacgtggacggcgtggaggtgcataatgccaagacaaagccgcgggaggagcagtacaacagcacgtaccgggtggtc |
| | | agcgtcctcaccgtcctgcaccaggactggctgaatggcaaggagtacaagtgcaaggtctccaacaaagccctcccagccccc |
| | | atcgagaaaaccatctccaaagccaaagggcagccccgagaaccacaggtgtacaccctgcccccatcccgggatgagctgac |
| | | caagaaccaggtcagcctgacctgcctggtcaaaggcttctatcccagcgacatcgccgtggagtgggagagcaatgggcagcc |
| | | ggagaacaactacaagaccacgcctcccgtgctggactccgacggctccttcttcctctacagcaagctcaccgtggacaagagc |
| | | aggtggcagcaggggaacgtcttctcatgctccgtgatgcatgaggctctgcacaaccactacacgcagaagagcctctccctgt |
| | | ctccgggtaaa |
| | | SEQ ID NO: 93 |
| | | ----------------------------------------------------------------------------------------- |
| | N49P38.1 | cagtctgccctgactcagcctcgctcagtgtccgcagctcctggacagtcagtcaccatctcctgcactggaacccacaattatgtct |
| | | cttggtgtcaacaacatccaggcagagcccccaaattactaatttatgacttcaataagaggccctcaggggtcccggatcgcttctc |
| | | tggctccggatctggcggcacggcctccctaaccatcactagactccaggatgacgatgacgctgactatttttgttgggcgtatgat |
| | | gcttttggcggagggaccaagttgaccgtcctgcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| | | SEQ ID NO: 94 |
| Sequence | N49P55 | gcggacttggtgcagtctggggctgtggtgaagaagcctggggcctcagtgagggtctcctgtgaggcttatggatacacattcac |
| 10 | | tgactacatcattcattggattcgacaggcccctggacaaggccttgaatggatgggatggatgaatcctatgggcgggcgcacaa |
| | | atattccgtggaaatttcagggcagggtctccatgacccgggacacgtccatcgaaacagcatttctggacttaagtggactaacgt |
| | | ctgacgacacggccgtctattattgcgtgagagacaagagtaatggatcgggcaaacgatttgactcctctaattggttcctcgatct |
| | | gtggggccgcggaaccccggtcactatttcctcaccctccaccaagggcccatcggtcttccccctggcaccctcctccaagagc |
| | | acctctgggggcacagcggccctgggctgcctggtcaaggactacttccccgaaccggtgacggtgtcgtggaactcaggcgcc |
| | | ctgaccagcggcgtgcacaccttcccggctgtcctacagtcctcaggactctactccctcagcagcgtggtgaccgtgccctccag |
| | | cagcttgggcacccagacctacatctgcaacgtgaatcacaagcccagcaacaccaaggtggacaagaaagttgagcccaaatc |
| | | ttgtgacaaaactcacacatgcccaccgtgcccagcacctgaactcctggggggaccgtcagtcttcctcttccccccaaaaccca |
| | | aggacaccctcatgatctcccggacccctgaggtcacatgcgtggtggtggacgtgagccacgaagaccctgaggtcaagttca |
| | | actggtacgtggacggcgtggaggtgcataatgccaagacaaagccgcgggaggagcagtacaacagcacgtaccgggtggt |
| | | cagcgtcctcaccgtcctgcaccaggactggctgaatggcaaggagtacaagtgcaaggtctccaacaaagccctcccagcccc |
| | | catcgagaaaaccatctccaaagccaaagggcagccccgagaaccacaggtgtacaccctgcccccatcccgggatgagctga |
| | | ccaagaaccaggtcagcctgacctgcctggtcaaaggcttctatcccagcgacatcgccgtggagtgggagagcaatgggcagc |
| | | cggagaacaactacaagaccacgcctcccgtgctggactccgacggctccttcttcctctacagcaagctcaccgtggacaagag |
| | | caggtggcagcaggggaacgtcttctcatgctccgtgatgcatgaggctctgcacaaccactacacgcagaagagcctctccctg |
| | | tctccgggtaaa |
| | | SEQ ID NO: 95 |
| | | ----------------------------------------------------------------------------------------- |
| | | cagtctgccctgactcagcctcgctcagtgtccgcagctcctggacagtccgtcaccatttcctgcactggaacccacaatttggtct |
| | | cttggtgtcaacaacacccaggcagagcccccaaattattaatttatgacttcaataagaggccctcaggggtccctgatcgcttctc |
| | | tggctccgggtctggcggcacggcctccctaagtatcactggactccaggatgacgatgaagctgaatatttttgttgggcgtatga |
| | | agcttttggcggagggaccaagttgaccgtccttcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| | | SEQ ID NO: 96 |
| Sequence | N49P56 | gcggacttggtgcagtctggggctgtggtgaagaagcctggggcctcagtgcgggtctcctgtgaggcttatggatatacattcgtt |
| 11 | | gactacctcattcattgggtccgacaggcccccggacaaggttttgaatggatgggatacatggatcctatgaacgggcgcccaaa |
| | | tattgcgcgaaaatttcagggcaggctctccctgagccgagatacgtccatcgaaacatcatttctggacttaagtggactgaggtct |
| | | gacgactcggccgtctattattgtgtgagagacaagagtggtggatcgggcaaactatttgactcctctaattggtttctcgatctgtg |
| | | gggccgtggaacccgggtcagcatttcttcagcctccaccaagggcccatcggtcttccccctggcaccctcctccaagagcacc |
| | | tctgggggcacagcggccctgggctgcctggtcaaggactacttccccgaaccggtgacggtgtcgtggaactcaggcgccctg |
| | | accagcggcgtgcacaccttcccggctgtcctacagtcctcaggactctactccctcagcagcgtggtgaccgtgccctccagca |
| | | gcttgggcacccagacctacatctgcaacgtgaatcacaagcccagcaacaccaaggtggacaagaaagttgagcccaaatctt |
| | | gtgacaaaactcacacatgcccaccgtgcccagcacctgaactcctggggggaccgtcagtcttcctcttccccccaaaacccaa |
| | | ggacaccctcatgatctcccggacccctgaggtcacatgcgtggtggtggacgtgagccacgaagaccctgaggtcaagttcaa |
| | | ctggtacgtggacggcgtggaggtgcataatgccaagacaaagccgcgggaggagcagtacaacagcacgtaccgggtggtc |
| | | agcgtcctcaccgtcctgcaccaggactggctgaatggcaaggagtacaagtgcaaggtctccaacaaagccctcccagccccc |
| | | atcgagaaaaccatctccaaagccaaagggcagccccgagaaccacaggtgtacaccctgcccccatcccgggatgagctgac |
| | | caagaaccaggtcagcctgacctgcctggtcaaaggcttctatcccagcgacatcgccgtggagtgggagagcaatgggcagcc |
| | | ggagaacaactacaagaccacgcctcccgtgctggactccgacggctccttcttcctctacagcaagctcaccgtggacaagagc |
| | | aggtggcagcaggggaacgtcttctcatgctccgtgatgcatgaggctctgcacaaccactacacgcagaagagcctctccctgt |
| | | ctccgggtaaa |
| | | SEQ ID NO: 97 |
| | | ----------------------------------------------------------------------------------------- |
| | | cagtctgccctgactcagcctcgctcagtgtccgcagctcctggacagtcagtcaccatctcctgcaccggaactcacaattatgtct |
| | | cttggtgtcaacaacatccaggcagagcccccaaattactaatttatgacttcaataagaggccctcaggggtcccggatcgcttctc |
| | | tggctccggatctggcggcacggcctccctaaccatcactggactccaggatgacgatgacgctgattatttttgttgggcgtatgat |
| | | gcttttggcggagggaccaagttgaccgtcctgcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| Sequence | N49P57 | SEQ ID NO: 98 |

12

| | | |
|---|---|---|
| | | gcggacttggtgcagtctggggctgtggtgaagaagcctggggactcagtgagaatctcctgtgaggctcaaggatacacattca |
| | | ctgactacgtcattcactggattcgacgggcccctggacaaggccttgaatggatggggtggattaatccaggttatggacaagtaa |
| | | atattccatggaactttcagggcagggtctccatgacccgagacacgtccatcgaaacagcttttctggagttaagaggtctaaagtc |
| | | tgacgacacgggcctctattattgcgtgagagatcgaagtaatggatggggaaagcgattcgagtcctccaattggttcctcgatct |
| | | gtggggccgcggcactgtgattactgttcactcaccctccaccaagggcccatcggtcttccccctggcaccctcctccaagagca |
| | | cctctgggggcacagcggccctgggctgcctggtcaaggactacttccccgaaccggtgacggtgtcgtggaactcaggcgccc |
| | | tgaccagcggcgtgcacaccttcccggctgtcctacagtcctcaggactctactccctcagcagcgtggtgaccgtgccctccagc |
| | | agcttgggcacccagacctacatctgcaacgtgaatcacaagcccagcaacaccaaggtggacaagaaagttgagcccaaatctt |
| | | gtgacaaaactcacacatgcccaccgtgcccagcacctgaactcctggggggaccgtcagtcttcctcttccccccaaaacccaa |
| | | ggacaccctcatgatctcccggacccctgaggtcacatgcgtggtggtggacgtgagccacgaagaccctgaggtcaagttcaa |
| | | ctggtacgtggacggcgtggaggtgcataatgccaagacaaagccgcgggaggagcagtacaacagcacgtaccgggtggtc |
| | | agcgtcctcaccgtcctgcaccaggactggctgaatggcaaggagtacaagtgcaaggtctccaacaaagccctcccagccccc |
| | | atcgagaaaaccatctccaaagccaaagggcagccccgagaaccacaggtgtacaccctgcccccatcccgggatgagctgac |
| | | caagaaccaggtcagcctgacctgcctggtcaaaggcttctatcccagcgacatcgccgtggagtgggagagcaatgggcagcc |
| | | ggagaacaactacaagaccacgcctcccgtgctggactccgacggctccttcttcctctacagcaagctcaccgtggacaagagc |
| | | aggtggcagcaggggaacgtcttctcatgctccgtgatgcatgaggctctgcacaaccactacacgcagaagagcctctccctgt |
| | | ctccgggtaaa |
| | | SEQ ID NO: 99 |
| | | ----------------------------------------------------------------------------------------- |
| | | cagtctgccctgactcagcctcgctcaatgtccgcatctcctgggcagtccgtcaccatttcctgcactggaacccacaatttggtct |
| | | cttggtgtcaacatcacccaggcagaccccccaaattattaatttatgacttcaataagaggccctcaggggtccctgatcgcttctct |
| | | ggctccgggtctggcggcacggcctccctgaccatcactggactccaggatgacgatgacgccgaatacatttgttgggcatatg |
| | | aagctttcggcggagggaccaagttgaccatacttcgtcagcccaaggctgccccctcggtcactctgttcccgccctcctctgag |
| | | gagcttcaagccaacaaggccacactggtgtgtctcataagtgacttctacccgggagccgtgacagtggcttggaaagcagata |
| | | gcagccccgtcaaggcgggagtggagaccaccacaccctccaaacaaagcaacaacaagtacgcggccagcagctatctgag |
| | | cctgacgcctgagcagtggaagtcccacagaagctacagctgccaggtcacgcatgaagggagcaccgtggagaagacagtg |
| | | gcccctacagaatgttca |
| | | SEQ ID NO: 100 |
| Sequence | N49P58 | gcggacttggtgcagtctggggctgtggtgaagaagcctggggactcagtgagaatctcctgtgaggctcagggatatacattcac |
| 13 | | cgactacgtcattcattggattcgacgggcccctggacaaggccttgaatggatggggtggatggatccaagttatggacaagtca |
| | | atattccacggaactttcagggcagggtctccatgacccgggacacgttcagggaaacagcatatctggaattaagaggtctacag |
| | | tctgacgacaagggcctctattattgtgtgagagatcgaagtcacggatcgggaaggcaattcgagtcctccaactggttcctcgat |
| | | ctgtggggccgcggcactgtggtcaatgttcagtcaccctccaccaagggcccatcggtcttccccctggcaccctcctccaagag |
| | | cacctctgggggcacagcggccctgggctgcctggtcaaggactacttccccgaaccggtgacggtgtcgtggaactcaggcgc |
| | | cctgaccagcggcgtgcacaccttcccggctgtcctacagtcctcaggactctactccctcagcagcgtggtgaccgtgccctcca |
| | | gcagcttgggcacccagacctacatctgcaacgtgaatcacaagcccagcaacaccaaggtggacaagaaagttgagcccaaat |
| | | cttgtgacaaaactcacacatgcccaccgtgcccagcacctgaactcctggggggaccgtcagtcttcctcttccccccaaaaccc |
| | | aaggacaccctcatgatctcccggacccctgaggtcacatgcgtggtggtggacgtgagccacgaagaccctgaggtcaagttc |
| | | aactggtacgtggacggcgtggaggtgcataatgccaagacaaagccgcgggaggagcagtacaacagcacgtaccgggtgg |
| | | tcagcgtcctcaccgtcctgcaccaggactggctgaatggcaaggagtacaagtgcaaggtctccaacaaagccctcccagccc |
| | | ccatcgagaaaaccatctccaaagccaaagggcagccccgagaaccacaggtgtacaccctgcccccatcccgggatgagctg |
| | | accaagaaccaggtcagcctgacctgcctggtcaaaggcttctatcccagcgacatcgccgtggagtgggagagcaatgggcag |
| | | ccggagaacaactacaagaccacgcctcccgtgctggactccgacggctccttcttcctctacagcaagctcaccgtggacaaga |
| | | gcaggtggcagcaggggaacgtcttctcatgctccgtgatgcatgaggctctgcacaaccactacacgcagaagagcctctccct |
| | | gtctccgggtaaa |
| | | SEQ ID NO: 101 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagacctcccaaattattaatttatgacttcaataagagggcttcaggggtccctgatcgcttctct |
| | | ggctccgggtctggcggcacggcctccctgaccattagtggactccaggatgacgatgacgccgaatatttttgttgggcatatga |
| | | agctttcggcggagggaccaagttgaccgtacttcgtcagcccaaggctgccccctcggtcactctgttcccgccctcctctgagg |
| | | agcttcaagccaacaaggccacactggtgtgtctcataagtgacttctacccgggagccgtgacagtggcttggaaagcagatag |
| | | cagccccgtcaaggcgggagtggagaccaccacaccctccaaacaaagcaacaacaagtacgcggccagcagctatctgagc |
| | | ctgacgcctgagcagtggaagtcccacagaagctacagctgccaggtcacgcatgaagggagcaccgtggagaagacagtgg |
| | | cccctacagaatgttca |
| | | SEQ ID NO: 102 |
| Sequence | N49P59 | gcggacttggtgcagtctggggctgtggtgaagaagcctggggactcactgagaatctcctgtgaggctcaaggatacacattca |
| 14 | | ctgactacgtcattcactggattcgacgggcccctggacaaggccttgaatggatgggatggatggatccaagttttggacaaatga |
| | | acattccacggaactttcagggcagggtctccatgacccgtgacatgtacatcgaaacagcatttctggacttaagaggtctaaagt |
| | | ctgacgacacgggcctctattattgcgtgagagatcgaagtcatggatcgggaaggctattcgagtcctccaattggttcctcgatct |
| | | gtggggccgcggcactgtggtcactgttcagtcaccctccaccaagggcccatcggtcttccccctggcaccctcctccaagagc |
| | | acctctgggggcacagcggccctgggctgcctggtcaaggactacttccccgaaccggtgacggtgtcgtggaactcaggcgcc |
| | | ctgaccagcggcgtgcacaccttcccggctgtcctacagtcctcaggactctactccctcagcagcgtggtgaccgtgccctccag |
| | | cagcttgggcacccagacctacatctgcaacgtgaatcacaagcccagcaacaccaaggtggacaagaaagttgagcccaaatc |
| | | ttgtgacaaaactcacacatgcccaccgtgcccagcacctgaactcctggggggaccgtcagtcttcctcttccccccaaaaccca |
| | | aggacaccctcatgatctcccggacccctgaggtcacatgcgtggtggtggacgtgagccacgaagaccctgaggtcaagttca |
| | | actggtacgtggacggcgtggaggtgcataatgccaagacaaagccgcgggaggagcagtacaacagcacgtaccgggtggt |
| | | cagcgtcctcaccgtcctgcaccaggactggctgaatggcaaggagtacaagtgcaaggtctccaacaaagccctcccagcccc |
| | | catcgagaaaaccatctccaaagccaaagggcagccccgagaaccacaggtgtacaccctgcccccatcccgggatgagctga |
| | | ccaagaaccaggtcagcctgacctgcctggtcaaaggcttctatcccagcgacatcgccgtggagtgggagagcaatgggcagc |
| | | cggagaacaactacaagaccacgcctcccgtgctggactccgacggctccttcttcctctacagcaagctcaccgtggacaagag |
| | | caggtggcagcaggggaacgtcttctcatgctccgtgatgcatgaggctctgcacaaccactacacgcagaagagcctctccctg |
| | | tctccgggtaaa |
| | | SEQ ID NO: 103 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagacctcccaaattattaatttatgacttcaataagagggcttcaggggtccctgatcgcttctct |
| | | ggctccgggtctggcggcacggcctccctgaccattagtggactccaggatgacgatgacgccgaatatttttgttgggcatatga |
| | | agctttcggcggagggaccaagttgaccgtacttcgtcagcccaaggctgccccctcggtcactctgttcccgccctcctctgagg |
| | | agcttcaagccaacaaggccacactggtgtgtctcataagtgacttctacccgggagccgtgacagtggcttggaaagcagatag |
| | | cagccccgtcaaggcgggagtggagaccaccacaccctccaaacaaagcaacaacaagtacgcggccagcagctatctgagc |
| | | ctgacgcctgagcagtggaagtcccacagaagctacagctgccaggtcacgcatgaagggagcaccgtggagaagacagtgg |
| | | cccctacagaatgttca |
| | | SEQ ID NO: 104 |
| Sequence | N49P73 | gcggacttggtgcagtctggggctgtggtgaagaagcctggggactcagtgagaatctcctgtgaggctcaaggatacagattca |
| 15 | | ctgactacgtcattcattggattcgacgggcccctggacaaggccttgaatggatggggttgatggatccaagttttggacgaatga |
| | | atattccacggaaatttcagggcagggtctccatgacccgggacacgtccatggaaacagcatttctggacttcagaggtctaaattt |
| | | tgacgacacgggcctctattattgcgtgagagatcgaagtcatggatcgggaagactattcgagtcctccaattggttcctcgatctg |
| | | tggggccgcggcactgtggtcactgttcagtcaccctccaccaagggcccatcggtcttccccctggcaccctcctccaagagca |
| | | cctctgggggcacagcggccctgggctgcctggtcaaggactacttccccgaaccggtgacggtgtcgtggaactcaggcgccc |
| | | tgaccagcggcgtgcacaccttcccggctgtcctacagtcctcaggactctactccctcagcagcgtggtgaccgtgccctccagc |
| | | agcttgggcacccagacctacatctgcaacgtgaatcacaagcccagcaacaccaaggtggacaagaaagttgagcccaaatctt |
| | | gtgacaaaactcacacatgcccaccgtgcccagcacctgaactcctggggggaccgtcagtcttcctcttccccccaaaacccaa |
| | | ggacaccctcatgatctcccggacccctgaggtcacatgcgtggtggtggacgtgagccacgaagaccctgaggtcaagttcaa |
| | | ctggtacgtggacggcgtggaggtgcataatgccaagacaaagccgcgggaggagcagtacaacagcacgtaccgggtggtc |
| | | agcgtcctcaccgtcctgcaccaggactggctgaatggcaaggagtacaagtgcaaggtctccaacaaagccctcccagccccc |
| | | atcgagaaaaccatctccaaagccaaagggcagccccgagaaccacaggtgtacaccctgcccccatcccgggatgagctgac |
| | | caagaaccaggtcagcctgacctgcctggtcaaaggcttctatcccagcgacatcgccgtggagtgggagagcaatgggcagcc |
| | | ggagaacaactacaagaccacgcctcccgtgctggactccgacggctccttcttcctctacagcaagctcaccgtggacaagagc |
| | | aggtggcagcaggggaacgtcttctcatgctccgtgatgcatgaggctctgcacaaccactacacgcagaagagcctctccctgt |
| | | ctccgggtaaa |
| | | SEQ ID NO: 105 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagacctcccaaattattaatttatgacttcaataagagggcatcaggggtccctgatcgcttctct |
| | | ggctccgggtctggcggcacggcctccctgaccatcagtggactccaagatgacgatgacgccgaatatttttgttgggcatatga |
| | | agctttcggcggagggaccaagttgaccgtacttcgtcagcccaaggctgccccctcggtcactctgttcccgccctcctctgagg |
| | | agcttcaagccaacaaggccacactggtgtgtctcataagtgacttctacccgggagccgtgacagtggcttggaaagcagatag |
| | | cagccccgtcaaggcgggagtggagaccaccacaccctccaaacaaagcaacaacaagtacgcggccagcagctatctgagc |
| | | ctgacgcctgagcagtggaagtcccacagaagctacagctgccaggtcacgcatgaagggagcaccgtggagaagacagtgg |
| | | cccctacagaatgttca |
| | | SEQ ID NO: 106 |
| Sequence | N49P74 | ccggacttggtgcagtctggggctgtggtgaagaagcctggggactcagtgagaatttcctgtgaggctcaaggatacacattcatt |
| 16 | | gactacgtcattcactggattcgacgggcccctggacaaggccttgaatggatggggttgatggatccaacttatggacgaatgaat |
| | | attccacggaagtttcagggcagggtctccatgacccgggacacgtccatcgaaacagcatttctggacttaagaggtctaaaatct |
| | | gacgacacgggcctctattattgcgtgagagatcgaagtcatggatcgggaaggctattcgagtcctccaactggttcctggatctg |
| | | tggggccgcggcactgtggtcactgttcagtcaccctccaccaagggcccatcggtcttccccctggcaccctcctccaagagca |
| | | cctctgggggcacagcggccctgggctgcctggtcaaggactacttccccgaaccggtgacggtgtcgtggaactcaggcgccc |
| | | tgaccagcggcgtgcacaccttcccggctgtcctacagtcctcaggactctactccctcagcagcgtggtgaccgtgccctccagc |
| | | agcttgggcacccagacct |
| | | acatctgcaacgtgaatcacaagcccagcaacaccaaggtggacaagaaagttgagcccaaatcttgtgacaaaactcacacatg |
| | | cccaccgtgcccagcacctgaactcctggggggaccgtcagtcttcctcttccccccaaaacccaaggacaccctcatgatctccc |
| | | ggacccctgaggtcacatgcgtggtggtggacgtgagccacgaagaccctgaggtcaagttcaactggtacgtggacggcgtgg |
| | | aggtgcataatgccaagacaaagccgcgggaggagcagtacaacagcacgtaccgggtggtcagcgtcctcaccgtcctgcac |
| | | caggactggctgaatggcaaggagtacaagtgcaaggtctccaacaaagccctcccagcccccatcgagaaaaccatctccaaa |
| | | gccaaagggcagccccgagaaccacaggtgtacaccctgcccccatcccgggatgagctgaccaagaaccaggtcagcctga |
| | | cctgcctggtcaaaggcttctatcccagcgacatcgccgtggagtgggagagcaatgggcagccggagaacaactacaagacc |
| | | acgcctcccgtgctggactccgacggctccttcttcctctacagcaagctcaccgtggacaagagcaggtggcagcaggggaac |
| | | gtcttctcatgctccgtgatgcatgaggctctgcacaaccactacacgcagaagagcctctccctgtctccgggtaaa |
| | | SEQ ID NO: 107 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagacctcccaaattattaatttatgacttcaataagagggcttcaggggtccctgatcgcttctct |
| | | ggctccgggtctggcggcacggcctccctgaccatcagtggactccaagatgacgatgacgccgaatatttttgttgggcatatga |
| | | agctttcggcggagggaccaagttgaccgtacttcgtcagcccaaggctgccccctcggtcactctgttcccgccctcctctgagg |
| | | agcttcaagccaacaaggccacactggtgtgtctcataagtgacttctacccgggagccgtgacagtggcttggaaagcagatag |
| | | cagccccgtcaaggcgggagtggagaccaccacaccctccaaacaaagcaacaacaagtacgcggccagcagctatctgagc |
| | | ctgacgcctgagcagtggaagtcccacagaagctacagctgccaggtcacgcatgaagggagcaccgtggagaagacagtgg |
| | | cccctacagaatgttca |
| | | SEQ ID NO: 108 |
| Sequence | N49P75 | gcggacttggtgcagtctggggctgtggtgaagaagcctggggactcagtgaggatctcctgtgaggctcaaggatacagatttct |
| 17 | | tgactacatcattcactggattcgacgggcccctggacaaggccttgaatggatgggatggatgaatccaatgggcggacaagta |
| | | aacattccatggaactttcagggtagggtctccatgacccgggacacgtccatcgaaacagcatttctggacttaagaggactaaa |
| | | gtctgacgacacggccgtctattattgcgtgagagatcgcagtaatggatcgggaaagcgattcgagtcctccaattggttcctcga |
| | | tctgtggggccgcgggactgcggtcactattcattcaccctccaccaagggcccatcggtcttccccctggcaccctcctccaaga |
| | | gcacctctgggggcacagcggccctgggctgcctggtcaaggactacttccccgaaccggtgacggtgtcgtggaactcaggc |
| | | gccctgaccagcggcgtgcacaccttcccggctgtcctacagtcctcaggactctactccctcagcagcgtggtgaccgtgccctc |
| | | cagcagcttgggcacccagacctacatctgcaacgtgaatcacaagcccagcaacaccaaggtggacaagaaagttgagccca |
| | | aatcttgtgacaaaactcacacatgcccaccgtgcccagcacctgaactcctggggggaccgtcagtcttcctcttccccccaaaa |
| | | cccaaggacaccctcatgatctcccggacccctgaggtcacatgcgtggtggtggacgtgagccacgaagaccctgaggtcaag |
| | | ttcaactggtacgtggacggcgtggaggtgcataatgccaagacaaagccgcgggaggagcagtacaacagcacgtaccgggt |
| | | ggtcagcgtcctcaccgtcctgcaccaggactggctgaatggcaaggagtacaagtgcaaggtctccaacaaagccctcccagc |
| | | ccccatcgagaaaaccatctccaaagccaaagggcagccccgagaaccacaggtgtacaccctgcccccatcccgggatgagc |
| | | tgaccaagaaccaggtcagcctgacctgcctggtcaaaggcttctatcccagcgacatcgccgtggagtgggagagcaatgggc |
| | | agccggagaacaactacaagaccacgcctcccgtgctggactccgacggctccttcttcctctacagcaagctcaccgtggacaa |
| | | gagcaggtggcagcaggggaacgtcttctcatgctccgtgatgcatgaggctctgcacaaccactacacgcagaagagcctctcc |
| | | ctgtctccgggtaaa |
| | | SEQ ID NO: 109 |
| | | ----------------------------------------------------------------------------------------- |
| | | cagtctgccctgactcagcctcgctcagtgtccgcatctcctgggcagtccgtcaccatttcctgcactggaacccacaatttggtct |
| | | cttggtgtcaacatcacccaggcagagcccccaaattattaatttatgacttcaataagaggccctcaggggtccctgatcgcttctct |
| | | ggctccgggtctggcggcacggcctccctgaccatcactggactccaggatgacgatgacgccgaatatttttgttgggcgtatga |
| | | agcttttggcggagggaccaagttgaccgtacttcgtcagcccaaggctgccccctcggtcactctgttcccgccctcctctgagga |
| | | gcttcaagccaacaaggccacactggtgtgtctcataagtgacttctacccgggagccgtgacagtggcttggaaagcagatagc |
| | | agccccgtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagacagtggc |
| | | ccctacagaatgttca |
| | | SEQ ID NO: 110 |
| Sequence | N49P9 | cacgtccaattggtgcagtctggaggtggggtgaagaagattggggccgctgtgaggatctcctgcgaggtgactggatataaatt |
| 18 | N49P9.1 | catggaccaactcataaactgggtgcggcaggcccccggtcagggccttgagtggatgggatggatgaatccaacatatggaca |
| | N49P9.2 | agtaaattattcatggagatttgaaggaagggtcaccatgaccagggacatggacaccgagacggccttcatggagttgagagga |
| | N49P9i7 | ctgagagtggacgacacggccgtctattattgcgcgagggggccctctggggaaaattatccttttcactattggggccagggtgtc |
| | N49P9-FR | cgagtggtcgtctcgtcaccctccaccaagggcccatcggtcttccccctggcaccctcctccaagagcacctctgggggcacag |
| | | cggccctgggctgcctggtcaaggactacttccccgaaccggtgacggtgtcgtggaactcaggcgccctgaccagcggcgtgc |
| | | acaccttcccggctgtcctacagtcctcaggactctactccctcagcagcgtggtgaccgtgccctccagcagcttgggcacccag |
| | | acctacatctgcaacgtgaatcacaagcccagcaacaccaaggtggacaagaaagttgagcccaaatcttgtgacaaaactcaca |
| | | catgcccaccgtgcccagcacctgaactcctggggggaccgtcagtcttcctcttccccccaaaacccaaggacaccctcatgatc |
| | | tcccggacccctgaggtcacatgcgtggtggtggacgtgagccacgaagaccctgaggtcaagttcaactggtacgtggacggc |
| | | gtggaggtgcataatgccaagacaaagccgcgggaggagcagtacaacagcacgtaccgggtggtcagcgtcctcaccgtcct |
| | | gcaccaggactggctgaatggcaaggagtacaagtgcaaggtctccaacaaagccctcccagcccccatcgagaaaaccatctc |
| | | caaagccaaagggcagccccgagaaccacaggtgtacaccctgcccccatcccgggatgagctgaccaagaaccaggtcagc |
| | | ctgacctgcctggtcaaaggcttctatcccagcgacatcgccgtggagtgggagagcaatgggcagccggagaacaactacaag |
| | | accacgcctcccgtgctggactccgacggctccttcttcctctacagcaagctcaccgtggacaagagcaggtggcagcagggg |
| | | aacgtcttctcatgctccgtgatgcatgaggctctgcacaaccactacacgcagaagagcctctccctgtctccgggtaaa |
| | | SEQ ID NO: 111 |
| | | ----------------------------------------------------------------------------------------- |
| | | gcatctgccctgactcagcctgcctccatgtctgcgtcccctggacagtcggtaaccatctcgtgctctggaaccagacacataatct |
| | | ctgcttggttccaacaatatccaggcaaaccacccaaactcataatttttgacgacgataagcgtccctctggagttcctagtcgcttc |
| | | tctgcctccaggcctggcgacacggcctccctgacaatctctaatgttcaacctgaggacgaggcgacgtacatttgcaatacatat |
| | | gaattctttggcggagggaccagattgaccgtcctaagtcagcccaaggctgccccctcggtcactctgttcccaccctcctctgag |
| | | gagcttcaagccaacaaggccacactggtgtgtctcgtaagtgacttctacccgggagccgtgacagtggcctggaaggcagatg |
| | | gcagccccgtcaaggtgggagtggagaccaccaaaccctccaaacaaagcaacaacaagtatgcggccagcagctacctgag |
| | | cctgacgcccgagcagtggaagtcccacagaagctacagctgccgggtcacgcatgaagggagcaccgtggagaagacagtg |
| | | gcccctgcagaatgctct |
| | | SEQ ID NO: 112 |
| Sequence | N49P22 | cacattcagttgctacagtcggggcctcaagttaagaagtctggggacacagtgagaatctcctgtgagacctctggatataacttc |
| 19 | | gtcgactcccgtatccactgggtccgacagaccccggaaaaacgtctcagatggatgggctggatcaatcctctccaaggtggtgt |
| | | gaattacgcgccggaatttcagggcagaatcaggatgaccagggacacatttatagacacagtttacgtggacctgagcggactg |
| | | acaccggccgacacggcctattattactgcgcgcgagggatcgatggcaagtcttacccctttcatttctggggccacggaacccg |
| | | ggtcaccgtcttctcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgaccagcggcgtgcac |
| | | accttcccggctgtcctacagtcctcaggactctactccctcagcagcgtggtgaccgtgccctccagcagcttgggcacccagac |
| | | ctacatctgcaacgtgaatcacaagcccagcaacaccaaggtggacaagaaagttgagcccaaatcttgtgacaaaactcacaca |
| | | tgcccaccgtgcccagcacctgaactcctggggggaccgtcagtcttcctcttccccccaaaacccaaggacaccctcatgatctc |
| | | ccggacccctgaggtcacatgcgtggtggtggacgtgagccacgaagaccctgaggtcaagttcaactggtacgtggacggcgt |
| | | ggaggtgcataatgccaagacaaagccgcgggaggagcagtacaacagcacgtaccgggtggtcagcgtcctcaccgtcctgc |
| | | accaggactggctgaatggcaaggagtacaagtgcaaggtctccaacaaagccctcccagcccccatcgagaaaaccatctcca |
| | | aagccaaagggcagccccgagaaccacaggtgtacaccctgcccccatcccgggatgagctgaccaagaaccaggtcagcct |
| | | gacctgcctggtcaaaggcttctatcccagcgacatcgccgtggagtgggagagcaatgggcagccggagaacaactacaaga |
| | | ccacgcctcccgtgctggactccgacggctccttcttcctctacagcaagctcaccgtggacaagagcaggtggcagcagggga |
| | | acgtcttctcatgctccgtgatgcatgaggctctgcacaaccactacacgcagaagagcctctccctgtctccgggtaaa |
| | | SEQ ID NO: 113 |
| | | ----------------------------------------------------------------------------------------- |
| | | cgatttgccctgactcaacctgcctccgtgtctgggtctcctggacagacgatcaccataacctgcgctggaggcagcgtctcctgg |
| | | tttcatttccctccaggcaaaacccccagactcattatttatgagtcttctaagcgaccctctggggtctctcctcgattctctgggtccc |
| | | agtctggcagcacggcctcccttataatttctggcctccagtctgatgacgaagggacatacttctgttctattcttgaatttttcggcag |
| | | agggactcttgtcaccgtcctgagtcagcccaaggctgccccctcggtcactctgttcccaccctcctctgaggagcttcaagccaa |
| | | caaggccacactggtgtgtctcgtaagtgacttctacccgggagccgtgacagtggcctggaaggcagatggcagccccgtcaa |
| | | ggtgggagtggagaccaccaaaccctccaaacaaagcaacaacaagtatgcggccagcagctacctgagcctgacgcccgag |
| | | cagtggaagtcccacagaagctacagctgccgggtcacgcatgaagggagcaccgtggagaagacagtggcccctgcagaat |
| | | gctct |
| | | SEQ ID NO: 114 |
| Sequence | N49P23 | caggtgcgcttggtgcagtctggggctggggcgaggaagactggggcctcaatgaaactttcctgctcgacctctggatacacctt |
| 20 | | caccactcatcacggccacttcataaattgggtgcgacaggcccgtggacaagggcttgagtggatggggtggatgaatcccatg |
| | | actgggcagatgaatattgaggggaaatttcagggcagagtcaccctcactcgagacatatacagtgacacggcttacatggaaat |
| | | gaccagactgacaactggcgacacgggcacttattactgtgcgcgaggcgatttcggacagaattatccctttcattattggggcca |
| | | gggaagcctggtcatcgtctcctcggcctccaccaagggcccatcggtcttccccctggcaccctcctccaagagcacctctggg |
| | | ggcacagcggccctgggctgcctggtcaaggactacttccccgaaccggtgacggtgtcgtggaactcaggcgccctgaccag |
| | | cggcgtgcacaccttcccggctgtcctacagtcctcaggactctactccctcagcagcgtggtgaccgtgccctccagcagcttgg |
| | | gcacccagacctacatctgcaacgtgaatcacaagcccagcaacaccaaggtggacaagaaagttgagcccaaatcttgtgaca |
| | | aaactcacacatgcccaccgtgcccagcacctgaactcctggggggaccgtcagtcttcctcttccccccaaaacccaaggacac |
| | | cctcatgatctcccggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtggtcagcgtcc |
| | | tcaccgtcctgcaccaggactggctgaatggcaaggagtacaagtgcaaggtctccaacaaagccctcccagcccccatcgaga |
| | | aaaccatctccaaagccaaagggcagccccgagaaccacaggtgtacaccctgcccccatcccgggatgagctgaccaagaac |
| | | caggtcagcctgacctgcctggtcaaaggcttctatcccagcgacatcgccgtggagtgggagagcaatgggcagccggagaa |
| | | caactacaagaccacgcctcccgtgctggactccgacggctccttcttcctctacagcaagctcaccgtggacaagagcaggtgg |
| | | cagcaggggaacgtcttctcatgctccgtgatgcatgaggctctgcacaaccactacacgcagaagagcctctccctgtctccggg |
| | | taaa |
| | | SEQ ID NO: 115 |
| | | ----------------------------------------------------------------------------------------- |
| | | ctgtctgccctgactcagcctgcctccgtgtctgggtctcctgggcagtcggtcaccatctcctgctctggaacgaaccgttaccttgt |
| | | ctcctggtatcaacaacaccctgacaaagcccccaaactcatcatttatgacgacaataagcggccctcaggaatttctgatcgcttc |
| | | tcagcctccaggcctgacgacacggcctccctgacaatctctggactccagactggggacgaggctacttattggtgtgcctcatat |
| | | gaacgttttggcggcgggacgaggctgaccgtccttagtcagcccaaggctgccccctcggtcactctgttcccaccctcctctga |
| | | ggagcttcaagccaacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagcagctacctga |
| | | gcctgacgcccgagcagtggaagtcccacagaagctacagctgccgggtcacgcatgaagggagcaccgtggagaagacagt |
| | | ggcccctgcagaatgctct |
| | | SEQ ID NO: 116 |
| Sequence | N49P9.3 | cacgtccaattggtgcagtctggaggtggggtgaagaagattggggccgctgtgaggatctcctgcgaggtgtctggatacaactt |
| 21 | N49P9.4 | catggaccaattcataaattgggtgcgacaggcccccggtcagggccttgagtggatgggatggatgaacccaatatatggacaa |
| | N49P9.3- | gtaaattattcatggagatttcaaggaagggtcaccatgaccagggacatgtacaccgacacggccttcatggagttgagaggact |
| | FR | gagagtggacgacacggccgtctattattgcgcgagggggccctctggggaaaattatccttttcactattggggccagggtgtcc |
| | N49P9.6- | gagtggtcgtctcgtcaccctccaccaagggcccatcggtcttccccctggcaccctcctccaagagcacctctgggggcacagc |
| | FR | ggccctgggctgcctggtcaaggactacttccccgaaccggtgacggtgtcgtggaactcaggcgccctgaccagcggcgtgca |
| | N49P9.6- | caccttcccggctgtcctacagtcctcaggactctactccctcagcagcgtggtgaccgtgccctccagcagcttgggcacccaga |
| | FR-54W | cctacatctgcaacgtgaatcacaagcccagcaacaccaaggtggacaagaaagttgagcccaaatcttgtgacaaaactcacac |
| | N49P9.6- | atgcccaccgtgcccagcacctgaactcctggggggaccgtcagtcttcctcttccccccaaaacccaaggacaccctcatgatct |
| | FR-54-F | cccggacccctgaggtcacatgcgtggtggtggacgtgagccacgaagaccctgaggtcaagttcaactggtacgtggacggc |
| | N49P9.6- | gtggaggtgcataatgccaagacaaagccgcgggaggagcagtacaacagcacgtaccgggtggtcagcgtcctcaccgtcct |
| | 54W | gcaccaggactggctgaatggcaaggagtacaagtgcaaggtctccaacaaagccctcccagcccccatcgagaaaaccatctc |
| | N49P9.6- | caaagccaaagggcagccccgagaaccacaggtgtacaccctgcccccatcccgggatgagctgaccaagaaccaggtcagc |
| | 54F | ctgacctgcctggtcaaaggcttctatcccagcgacatcgccgtggagtgggagagcaatgggcagccggagaacaactacaag |
| | N49P9.6- | accacgcctcccgtgctggactccgacggctccttcttcctctacagcaagctcaccgtggacaagagcaggtggcagcagggg |
| | FR3-06 | aacgtcttctcatgctccgtgatgcatgaggctctgcacaaccactacacgcagaagagcctctccctgtctccgggtaaa |
| | N49P9.6- | SEQ ID NO: 117 |
| | FR1-D | ----------------------------------------------------------------------------------------- |
| | N49P9.6 | ctgactcagcctgcctccatgtctgcgtcccctggacagtcggtaaccatctcgtgctctggaaccagacacataatctctgcttggtt |
| | N49P9.6- | ccaacaatatccaggcaaaccacccaaactcataatttttgacgacgataagcgtccctctggagttcctagtcgcttctctgcctcca |
| | FRI-D-I | ggcctggcgacacggcctccctgacaatctctaatgttcaacctgaggacgaggcgacatacatttgcaatacatatgaattctttgg |
| | | cggagggaccaaattgaccgtcctaagtcagcccaaggctgccccctcggtcactctgttcccaccctcctctgaggagcttcaag |
| | | ccaacaaggccacactggtgtgtctcgtaagtgacttctacccgggagccgtgacagtggcctggaaggcagatggcagccccg |
| | | tcaaggtgggagtggagaccaccaaaccctccaaacaaagcaacaacaagtatgcggccagcagctacctgagcctgacgccc |
| | | gagcagtggaagtcccacagaagctacagctgccgggtcacgcatgaagggagcaccgtggagaagacagtggcccctgcag |
| | | aatgctct |
| | | SEQ ID NO: 118 |
| Sequence | N49P51 | cacattcagttgctacagtcggggcctcaagttaagaagtctggggacacagtgagaatctcctgtgagacctctggatataacttc |
| 22 | | gtcgactcccgtatccactgggtccgacagaccccggaaaaacgtctcagatggatgggctggatcaatcctctccacggtggtgt |
| | | gaattacgcgccggaatttcagggcagaatcaggatgaccagggacacatttatagacacagtttacgtggacctgagcggactg |
| | | acaccggccgacacggcctattattactgcgcgcgagggatcgatggcaagtcttacccctttcatttctggggccacggaacccg |
| | | ggtcaccgtcttctcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgaccagcggcgtgcac |
| | | accttcccggctgtcctacagtcctcaggactctactccctcagcagcgtggtgaccgtgccctccagcagcttgggcacccagac |
| | | ctacatctgcaacgtgaatcacaagcccagcaacaccaaggtggacaagaaagttgagcccaaatcttgtgacaaaactcacaca |
| | | tgcccaccgtgcccagcacctgaactcctggggggaccgtcagtcttcctcttccccccaaaacccaaggacaccctcatgatctc |
| | | ccggacccctgaggtcacatgcgtggtggtggacgtgagccacgaagaccctgaggtcaagttcaactggtacgtggacggcgt |
| | | ggaggtgcataatgccaagacaaagccgcgggaggagcagtacaacagcacgtaccgggtggtcagcgtcctcaccgtcctgc |
| | | accaggactggctgaatggcaaggagtacaagtgcaaggtctccaacaaagccctcccagcccccatcgagaaaaccatctcca |
| | | aagccaaagggcagccccgagaaccacaggtgtacaccctgcccccatcccgggatgagctgaccaagaaccaggtcagcct |
| | | gacctgcctggtcaaaggcttctatcccagcgacatcgccgtggagtgggagagcaatgggcagccggagaacaactacaaga |
| | | ccacgcctcccgtgctggactccgacggctccttcttcctctacagcaagctcaccgtggacaagagcaggtggcagcagggga |
| | | acgtcttctcatgctccgtgatgcatgaggctctgcacaaccactacacgcagaagagcctctccctgtctccgggtaaa |
| | | SEQ ID NO: 119 |
| | | ----------------------------------------------------------------------------------------- |
| | | cgatttgccctgactcaacctgcctccgtgtctgggtctcctggacagacgatcaccataacctgcgctggaggcagcgtctcctgg |
| | | tttcatttccctccaggcaaaacccccagactcattatttatgagtcttctaagcgaccctctggggtctctcctcgattctctgggtccc |
| | | agtctggcagcacggcctccctcataatttctggcctccagtctgatgacgaagggacatacttctgttctattcttgaatttttcggca |
| | | gagggactcttgtcaccgtcctgagtcagcccaaggctgccccctcggtcactctgttcccaccctcctctgaggagcttcaagcca |
| | | acaaggccacactggtgtgtctcgtaagtgacttctacccgggagccgtgacagtggcctggaaggcagatggcagccccgtca |
| | | aggtgggagtggagaccaccaaaccctccaaacaaagcaacaacaagtatgcggccagcagctacctgagcctgacgcccga |
| | | gcagtggaagtcccacagaagctacagctgccgggtcacgcatgaagggagcaccgtggagaagacagtggcccctgcagaa |
| | | tgctct SEQ ID NO: 120 |
| Sequence | N49P52 | cgtgttacattacaacaatctggggctatagtgaggcagcctggggcctcagtgaccgtctcctgcgagacttctggatatactttca |
| 23 | | ccaagtatttcatctactgggtgcgacaggcccctggacagggtcttgagtggctgggcagaatacacccccgaaccggtgccgt |
| | | gaagtatgcaccgagatttcagggtagactgtccatgaccagagactggtcactcgacacagcctacctcggattgaccggactg |
| | | acactcggcgacacggctctatatttctgtgcgaggggggcctttgaggcagattcatatgggtcaagttatccctttcaccactggg |
| | | gccagggaaccctagtcaccgtctccgcggcctccaccaagggcccatcggtcttccccctggcaccctcctccaagagcacctc |
| | | tgggggcacagcggccctgggctgcctggtcaaggactacttccccgaaccggtgacggtgtcgtggaactcaggcgccctga |
| | | ccagcggcgtgcacaccttcccggctgtcctacagtcctcaggactctactccctcagcagcgtggtgaccgtgccctccagcag |
| | | cttgggcacccagacctacatctgcaacgtgaatcacaagcccagcaacaccaaggtggacaagaaagttgagcccaaatcttgt |
| | | gacaaaactcacacatgcccaccgtgcccagcacctgaactcctggggggaccgtcagtcttcctcttccccccaaaacccaagg |
| | | acaccctcatgatctcccggacccctgaggtcacatgcgtggtggtggacgtgagccacgaagaccctgaggtcaagttcaactg |
| | | gtacgtggacggcgtggaggtgcataatgccaagacaaagccgcgggaggagcagtacaacagcacgtaccgggtggtcagc |
| | | gtcctcaccgtcctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggatgagctgaccaa |
| | | gaaccaggtcagcctgacctgcctggtcaaaggcttctatcccagcgacatcgccgtggagtgggagagcaatgggcagccgg |
| | | agaacaactacaagaccacgcctcccgtgctggactccgacggctccttcttcctctacagcaagctcaccgtggacaagagcag |
| | | gtggcagcaggggaacgtcttctcatgctccgtgatgcatgaggctctgcacaaccactacacgcagaagagcctctccctgtctc |
| | | cgggtaaa |
| | | SEQ ID NO: 121 |
| | | ----------------------------------------------------------------------------------------- |
| | | tcctgggccctgactcaacccgcctccgtgtctgcgtctcctgggcagtcggtcaccatgtcctgcactggattcggaaattataacc |
| | | ctgactcctggtaccaacaatacccaggcaaagcccccaaactcatcatttatgaagacaataaaagaccctcgggggtctctgat |
| | | cgcttctctgcctccagacttggcagcacgtcttccctgacaatctctaacgtccaggctgcggacgacgcccattatgtctgcgcct |
| | | cctttgaatttttcggcggagggaccaagctgaccgtcctgagtcagcccaaggctgccccctcggtcactctgttcccaccctcct |
| | | ctgaggagcttcaagccaacaaggccacactggtgtgtctcgtaagtgacttctacccgggagccgtgacagtggcctggaaggc |
| | | agatggcagccccgtcaaggtgggagtggagaccaccaaaccctccaaacaaagcaacaacaagtatgcggccagcagctac |
| | | ctgagcctgacgcccgagcagtggaagtcccacagaagctacagctgccgggtcacgcatgaagggagcaccgtggagaaga |
| | | cagtggcccctgcagaatgctct |
| | | SEQ ID NO: 122 |
| Sequence | N49P53 | cgtgtgacattacaacaatctggggctacagtgaagcagcctggggcctcagtgaccgtctcctgcgagacttctggatacactttc |
| 24 | | accaagtataccattcactgggtgcgacaggcccctggacagggtcttcagtgggtgggcagaatacacccccgaaccggtgcc |
| | | gtgaagtatgcaccgatatttcagggtaaagtgtccatgagtcgagacttgtcacgcgacacagcctacctcggattgaccagactg |
| | | acgctcgccgacacggctctatttttctgtgcgaggggggcctttgaggcagatttaagtgggccaacttacccctttcaccactgg |
| | | ggccaaggaaccctagtcatcgtctccgcggcctccaccaagggcccatcggtcttccccctggcaccctcctccaagagcacct |
| | | ctgggggcacagcggccctgggctgcctggtcaaggactacttccccgaaccggtgacggtgtcgtggaactcaggcgccctg |
| | | accagcggcgtgcacaccttcccggctgtcctacagtcctcaggactctactccctcagcagcgtggtgaccgtgccctccagca |
| | | gcttgggcacccagacctacatctgcaacgtgaatcacaagcccagcaacaccaaggtggacaagaaagttgagcccaaatctt |
| | | gtgacaaaactcacacatgcccaccgtgcccagcacctgaactcctggggggaccgtcagtcttcctcttccccccaaaacccaa |
| | | ggacaccctcatgatctcccggacccctgaggtcacatgcgtggtggtggacgtgagccacgaagaccctgaggtcaagttcaa |
| | | ctggtacgtggacggcgtggaggtgcataatgccaagacaaagccgcgggaggagcagtacaacagcacgtaccgggtggtc |
| | | agcgtcctcaccgtcctgcaccaggactggctgaatggcaaggagtacaagtgcaaggtctccaacaaagccctcccagccccc |
| | | atcgagaaaaccatctccaaagccaaagggcagccccgagaaccacaggtgtacaccctgcccccatcccgggatgagctgac |
| | | caagaaccaggtcagcctgacctgcctggtcaaaggcttctatcccagcgacatcgccgtggagtgggagagcaatgggcagcc |
| | | ggagaacaactacaagaccacgcctcccgtgctggactccgacggctccttcttcctctacagcaagctcaccgtggacaagagc |
| | | aggtggcagcaggggaacgtcttctcatgctccgtgatgcatgaggctctgcacaaccactacacgcagaagagcctctccctgt |
| | | ctccgggtaaa |
| | | SEQ ID NO: 123 |
| | | ----------------------------------------------------------------------------------------- |
| | | tcctgggccctgactcaacccgcctccgtgtctgcgtctcctgggcagtcggtcaccatgtcctgcactggattcggaaattataacc |
| | | ctgactcctggtaccaacaatacccaggcaaagcccccaaactcatcatttatgaggacaataaaagaccctcgggagtctctaatc |
| | | gcttctctgcctccagacttggcagcacgtcttccctgacaatctctaacgtccaggccgctgacgacgcccattatgtctgcgcctc |
| | | ctttgaatttttcggcggagggaccaagctgatcgtcctgagtcagcccaaggctgccccctcggtcactctgttcccaccctcctct |
| | | gaggagcttcaagccaacaaggccacactggtgtgtctcgtaagtgacttctacccgggagccgtgacagtggcctggaaggca |
| | | gatggcagccccgtcaaggtgggagtggagaccaccaaaccctccaaacaaagcaacaacaagtatgcggccagcagctacct |
| | | gagcctgacgcccgagcagtggaagtcccacagaagctacagctgccgggtcacgcatgaagggagcaccgtggagaagac |
| | | agtggcccctgcagaatgctct |
| | | SEQ ID NO: 124 |
| Sequence | N49P54 | aacgtgcagttgatgcagtctgggactgaggtgaagaagtctggggcctcggtgacaatctcttgtgagaccgctggattcaacttc |
| 25 | | atcgactccgtcatacactggctgcgccaggcccctggaggaggatttcagtggatggggtggatcaagcctcttagaggtgccg |
| | | tcaattatccacagtttttgcagggcagggtctccatgacccgggacttgtccaccgacacggtgtacatggtcttgaatggactgac |
| | | acctgacgacacaggcctttattactgcgcgaaaggggcctttagagggggttctccctttggcttctggggccagggaactctgct |
| | | caccgtctccccagcctccaccaagggcccatcggtcttccccctggcaccctcctccaagagcacctctgggggcacagcggc |
| | | cctgggctgcctggtcaaggactacttccccgaaccggtgacggtgtcgtggaactcaggcgccctgaccagcggcgtgcacac |
| | | cttcccggctgtcctacagtcctcaggactctactccctcagcagcgtggtgaccgtgccctccagcagcttgggcacccagacct |
| | | acatctgcaacgtgaatcacaagcccagcaacaccaaggtggacaagaaagttgagcccaaatcttgtgacaaaactcacacatg |
| | | cccaccgtgcccagcacctgaactcctggggggaccgtcagtcttcctcttccccccaaaacccaaggacaccctcatgatctccc |
| | | ggacccctgaggtcacatgcgtggtggtggacgtgagccacgaagaccctgaggtcaagttcaactggtacgtggacggcgtgg |
| | | aggtgcataatgccaagacaaagccgcgggaggagcagtacaacagcacgtaccgggtggtcagcgtcctcaccgtcctgcac |
| | | caggactggctgaatggcaaggagtacaagtgcaaggtctccaacaaagccctcccagcccccatcgagaaaaccatctccaaa |
| | | gccaaagggcagccccgagaaccacaggtgtacaccctgcccccatcccgggatgagctgaccaagaaccaggtcagcctga |
| | | cctgcctggtcaaaggcttctatcccagcgacatcgccgtggagtgggagagcaatgggcagccggagaacaactacaagacc |
| | | acgcctcccgtgctggactccgacggctccttcttcctctacagcaagctcaccgtggacaagagcaggtggcagcaggggaac |
| | | gtcttctcatgctccgtgatgcatgaggctctgcacaaccactacacgcagaagagcctctccctgtctccgggtaaa |
| | | SEQ ID NO: 125 |
| | | ----------------------------------------------------------------------------------------- |
| | | cagtctgccctgtctcagcctgtctccgtgtctgggtctcctggagagtcgatcaccatttcctgtactggagccaccacctggtatca |
| | | acaactcccaggcagaccccccaaactcatcatttatgacgtcactaaccggccctcaggcatttctagtcgtttctctggctccacg |
| | | tctggccacacggcctccctgacaatctccggtctccaggttgacgacgagggtctgtatcactgcgcctcacgtgaatttttcggc |
| | | ggagggaccaagctgaccgtcctgagtcagcccaaggctgccccctcggtcactctgttcccaccctcctctgaggagcttcaag |
| | | ccaacaaggccacactggtgtgtctcgtaagtgacttctacccgggagccgtgacagtggcctggaaggcagatggcagccccg |
| | | tcaaggtgggagtggagaccaccaaaccctccaaacaaagcaacaacaagtatgcggccagcagctacctgagcctgacgccc |
| | | gagcagtggaagtcccacagaagctacagctgccgggtcacgcatgaagggagcaccgtggagaagacagtggcccctgcag |
| | | aatgctct |
| | | SEQ ID NO: 126 |
| Sequence | N49P60 | caggtgcgactggtgcagtctgggcctcaggtgaagaagactggggcctcagtgagggtctcctgcgaaacctctggatacacgt |
| 26 | | tcacctcctacttcatccattggttacgactgggccccggagaggggcttcagtggatggggtggatcaaccctttacatggtgccg |
| | | tgaattatgaaaacaaatttaggggcagggtcacaatcaccagggacacgtccacagacacagtgtatttggacatgagcagactg |
| | | acccctgacgacacggccgtctatttctgcacaagaggaatcgttgctgatgggtggccctatggccactggggccagggaaccc |
| | | aagtcaccgtctccccggcctccaccaagggcccatcggtcttccccctggcaccctcctccaagagcacctctgggggcacag |
| | | cggccctgggctgcctggtcaaggactacttccccgaaccggtgacggtgtcgtggaactcaggcgccctgaccagcggcgtgc |
| | | acaccttcccggctgtcctacagtcctcaggactctactccctcagcagcgtggtgaccgtgccctccagcagcttgggcacccag |
| | | acctacatctgcaacgtgaatcacaagcccagcaacaccaaggtggacaagaaagttgagcccaaatcttgtgacaaaactcaca |
| | | catgcccaccgtgcccagcacctgaactcctggggggaccgtcagtcttcctcttccccccaaaacccaaggacaccctcatgatc |
| | | tcccggacccctgaggtcacatgcgtggtggtggacgtgagccacgaagaccctgaggtcaagttcaactggtacgtggacggc |
| | | gtggaggtgcataatgccaagacaaagccgcgggaggagcagtacaacagcacgtaccgggtggtcagcgtcctcaccgtcct |
| | | gcaccaggactggctgaatggcaaggagtacaagtgcaaggtctccaacaaagccctcccagcccccatcgagaaaaccatctc |
| | | caaagccaaagggcagccccgagaaccacaggtgtacaccctgcccccatcccgggatgagctgaccaagaaccaggtcagc |
| | | ctgacctgcctggtcaaaggcttctatcccagcgacatcgccgtggagtgggagagcaatgggcagccggagaacaactacaag |
| | | accacgcctcccgtgctggactccgacggctccttcttcctctacagcaagctcaccgtggacaagagcaggtggcagcagggg |
| | | aacgtcttctcatgctccgtgatgcatgaggctctgcacaaccactacacgcagaagagcctctccctgtctccgggtaaa |
| | | SEQ ID NO: 127 |
| | | ----------------------------------------------------------------------------------------- |
| | | tcctgggccctgactcagcctgcctccgtgtctgggtctcctggacagtcggtcgccatctcctgcgctggcggcagcgtctcctg |
| | | gtaccaggtgctcccaggcagagcccccaaactcatcatttatgagggcgctaagcgaccctcaggggtttctgctcgcttctctgg |
| | | ctcccagtctggcaacacggcttacctgacaatttctgacctccagactgaggacgagggcatctacttctgctcttcacttcaattctt |
| | | cggcggagggaccaaactgaccgtcctaagtcagcccaaggctgccccctcggtcactctgttcccaccctcctctgaggagctt |
| | | caagccaacaaggccacactggtgtgtctcgtaagtgacttctacccgggagccgtgacagtggcctggaaggcagatggcagc |
| | | cccgtcaaggtgggagtggagaccaccaaaccctccaaacaaagcaacaacaagtatgcggccagcagctacctgagcctgac |
| | | gcccgagcagtggaagtcccacagaagctacagctgccgggtcacgcatgaagggagcaccgtggagaagacagtggcccct |
| | | gcagaatgctct |
| | | SEQ ID NO: 128 |
| Sequence | N49P61 | caggtgcgacttcagcagtctggtgtcgtggtgaggaagcctggggcctcagtgagaatttcctgcgagacttctggattcaccttc |
| 27 | | atcgaccacattgtccattgggtgcggcgggcccctggacgaggctttgaatggatgggttggatcaagcctcttaggggtgccgt |
| | | agattatgcaccccaacttcggggcaggatctccctgacgagggacatttacagtgaaaccgtctttatagacgtgagtcgactgac |
| | | gtctggcgacacggcgatatacttttgttgtaaggccgccgcccctgaagaagcattcccccttcaatactggggccaggggaccc |
| | | aacttatcgtctcctcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgaccagcggcgtgcac |
| | | accttcccggctgtcctacagtcctcaggactctactccctcagcagcgtggtgaccgtgccctccagcagcttgggcacccagac |
| | | ctacatctgcaacgtgaatcacaagcccagcaacaccaaggtggacaagaaagttgagcccaaatcttgtgacaaaactcacaca |
| | | tgcccaccgtgcccagcacctgaactcctggggggaccgtcagtcttcctcttccccccaaaacccaaggacaccctcatgatctc |
| | | ccggacccctgaggtcacatgcgtggtggtggacgtgagccacgaagaccctgaggtcaagttcaactggtacgtggacggcgt |
| | | ggaggtgcataatgccaagacaaagccgcgggaggagcagtacaacagcacgtaccgggtggtcagcgtcctcaccgtcctgc |
| | | accaggactggctgaatggcaaggagtacaagtgcaaggtctccaacaaagccctcccagcccccatcgagaaaaccatctcca |
| | | aagccaaagggcagccccgagaaccacaggtgtacaccctgcccccatcccgggatgagctgaccaagaaccaggtcagcct |
| | | gacctgcctggtcaaaggcttctatcccagcgacatcgccgtggagtgggagagcaatgggcagccggagaacaactacaaga |
| | | ccacgcctcccgtgctggactccgacggctccttcttcctctacagcaagctcaccgtggacaagagcaggtggcagcagggga |
| | | acgtcttctcatgctccgtgatgcatgaggctctgcacaaccactacacgcagaagagcctctccctgtctccgggtaaa |
| | | SEQ ID NO: 129 |
| | | ----------------------------------------------------------------------------------------- |
| | | caggctgccctgactcagcccgcctccgtgtccggctctcctggacagtcggtcaccatttcctgcctttatgccaatgtagatatct |
| | | gctggtatcaactacacccgggcagagcccccaaacttctaattgttgacaataataagcggccctcaggagtctctcctcgcttctc |
| | | tggctccaagtctggcaccacggcctccctgacaatctctggacttcaggctgacgacgaggctgaatatcactgctcttcaagaac |
| | | attttttggcggggggaccaagttgaccgtcctgagtcagcccaaggctgccccctcggtcactctgttcccaccctcctctgagga |
| | | gcttcaagccaacaaggccacactggtgtgtctcgtaagtgacttctacccgggagccgtgacagtggcctggaaggcagatggc |
| | | agccccgtcaaggtgggagtggagaccaccaaaccctccaaacaaagcaacaacaagtatgcggccagcagctacctgagcct |
| | | gacgcccgagcagtggaagtcccacagaagctacagctgccgggtcacgcatgaagggagcaccgtggagaagacagtggc |
| | | ccctgcagaatgctct |
| | | SEQ ID NO: 130 |
| Sequence | N49P62 | caggtgcgacttcagcagtctggtgtcgtggtgaggaagcctggggcctcagtgagactttcctgcgagacgtctggattcaaattc |
| 28 | | atcgaccacattgtcaactgggtgcggcgggcccctggacgaggctttgaatggatgggttggatcaagcctcttgggggtgtcg |
| | | ctgattatgcaccccaacatcggggcaggatctcactgacgagggacatttacactgaaaccgtctttatagacctgagtcgactga |
| | | cgtctggcgacacggcgatttatttctgttgtaaggccgccgcccctgatgaagcattcccccttgaatactggggccaggggacc |
| | | caacttatcgtctccccggcctccaccaagggcccatcggtcttccccctggcaccctcctccaagagcacctctgggggcacag |
| | | cggccctgggctgcctggtcaaggactacttccccgaaccggtgacggtgtcgtggaactcaggcgccctgaccagcggcgtgc |
| | | acaccttcccggctgtcctacagtcctcaggactctactccctcagcagcgtggtgaccgtgccctccagcagcttgggcacccag |
| | | acctacatctgcaacgtgaatcacaagcccagcaacaccaaggtggacaagaaagttgagcccaaatcttgtgacaaaactcaca |
| | | catgcccaccgtgcccagcacctgaactcctggggggaccgtcagtcttcctcttccccccaaaacccaaggacaccctcatgatc |
| | | tcccggacccctgaggtcacatgcgtggtggtggacgtgagccacgaagaccctgaggtcaagttcaactggtacgtggacggc |
| | | gtggaggtgcataatgccaagacaaagccgcgggaggagcagtacaacagcacgtaccgggtggtcagcgtcctcaccgtcct |
| | | gcaccaggactggctgaatggcaaggagtacaagtgcaaggtctccaacaaagccctcccagcccccatcgagaaaaccatctc |
| | | caaagccaaagggcagccccgagaaccacaggtgtacaccctgcccccatcccgggatgagctgaccaagaaccaggtcagc |
| | | ctgacctgcctggtcaaaggcttctatcccagcgacatcgccgtggagtgggagagcaatgggcagccggagaacaactacaag |
| | | accacgcctcccgtgctggactccgacggctccttcttcctctacagcaagctcaccgtggacaagagcaggtggcagcagggg |
| | | aacgtcttctcatgctccgtgatgcatgaggctctgcacaaccactacacgcagaagagcctctccctgtctccgggtaaa |
| | | SEQ ID NO: 131 |
| | | ----------------------------------------------------------------------------------------- |
| | | caggctgccctgactcagcccgcctccgtgtccggctctcctggacagtcggtcaccatttcctgcctttatgccaatgtagatatct |
| | | gctggtatcaaatacagccgggcagattacccaaacttctgattgttgacaataataggcgaccctcaggagtctctcctcgcttctct |
| | | ggctccaagtctggcaccacggcctccctgacaatctctggacttcaggctgacgacgaggctgaatatcactgctcttcaacaac |
| | | attttttggcggggggaccaagttgaccgtcctcagtcagcccaaggctgccccctcggtcactctgttcccaccctcctctgagga |
| | | gcttcaagccaacaaggccacactggtgtgtctcgtaagtgacttctacccgggagccgtgacagtggcctggaaggcagatggc |
| | | agccccgtcaaggtgggagtggagaccaccaaaccctccaaacaaagcaacaacaagtatgcggccagcagctacctgagcct |
| | | gacgcccgagcagtggaagtcccacagaagctacagctgccgggtcacgcatgaagggagcaccgtggagaagacagtggc |
| | | ccctgcagaatgctct |
| | | SEQ ID NO: 132 |
| Sequence | N49P63 | caggtgcgacttgtgcagtctggtcccgtgatgagaaagcctggggcctcagtgagaatttcttgcgagacatctggattcgccttct |
| 29 | | tggaccacattgtccactgggtgcggcgggcccctggacgcggctttgaatggatgggttgggttaagaccattgggggtgtcgtt |
| | | gattatgcaccccaccttaggggcaggatctccgtgacgagagacgtctttagtgaaaccgtctttctggacttgagtcgactgacgt |
| | | ctggcgacacggcgatgtatttttgttctaaggccgccgcccctgacgaagccttcccccttgaattttggggccaggggacccaa |
| | | gtcatcgtctcctcggcctccaccaagggcccatcggtcttccccctggcaccctcctccaagagcacctctgggggcacagcgg |
| | | ccctgggctgcctggtcaaggactacttccccgaaccggtgacggtgtcgtggaactcaggcgccctgaccagcggcgtgcaca |
| | | ccttcccggctgtcctacagtcctcaggactctactccctcagcagcgtggtgaccgtgccctccagcagcttgggcacccagacc |
| | | tacatctgcaacgtgaatcacaagcccagcaacaccaaggtggacaagaaagttgagcccaaatcttgtgacaaaactcacacat |
| | | gcccaccgtgcccagcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtacgtggacggcgtg |
| | | gaggtgcataatgccaagacaaagccgcgggaggagcagtacaacagcacgtaccgggtggtcagcgtcctcaccgtcctgca |
| | | ccaggactggctgaatggcaaggagtacaagtgcaaggtctccaacaaagccctcccagcccccatcgagaaaaccatctccaa |
| | | agccaaagggcagccccgagaaccacaggtgtacaccctgcccccatcccgggatgagctgaccaagaaccaggtcagcctg |
| | | acctgcctggtcaaaggcttctatcccagcgacatcgccgtggagtgggagagcaatgggcagccggagaacaactacaagac |
| | | cacgcctcccgtgctggactccgacggctccttcttcctctacagcaagctcaccgtggacaagagcaggtggcagcaggggaa |
| | | cgtcttctcatgctccgtgatgcatgaggctctgcacaaccactacacgcagaagagcctctccctgtctccgggtaaa |
| | | SEQ ID NO: 133 |
| | | ----------------------------------------------------------------------------------------- |
| | | caggctgccctgactcagcccgcctccgtgtccggctctcctggacagtcggtcaccatttcttgcctttatgccaatgtggatatctg |
| | | ctggtatcaacttcacccgggcagagcccccaaacttcttattcttgacaataataaacggccctcaggagtctctagtcgcttctccg |
| | | gttccaagtctggcaccacggcctccctaaccatctctgaccttcaggctgacgacgaggctgaatatcactgctcttcaacaacatt |
| | | ttttggcggggggaccaggttgaccgtcctgagtcagcccaaggctgccccctcggtcactctgttcccaccctcctctgaggagc |
| | | ttcaagccaacaaggccacactggtgtgtctcgtaagtgacttctacccgggagccgtgacagtggcctggaaggcagatggcag |
| | | ccccgtcaaggtgggagtggagaccaccaaaccctccaaacaaagcaacaacaagtatgcggccagcagctacctgagcctga |
| | | cgcccgagcagtggaagtcccacagaagctacagctgccgggtcacgcatgaagggagcaccgtggagaagacagtggccc |
| | | ctgcagaatgctct |
| | | SEQ ID NO: 134 |
| Sequence | N49P64 | caggtgcgacttgtgcagtctggtcccgtggtgagaaagcctgggacctcagtgagaatttcttgcgagacatctggattcgccttct |
| 30 | | tggaccacattgtccactgggtgcggcgggcccctggacgcggctttgaatggatgggttgggttaagaccattgggggtgtcgtt |
| | | gattatgcaccccaccttaggggcaggatctccgtgacgagggacgtatttagtgaaatcgtctttatggagttgagtcgactgacgt |
| | | ctggcgacacggcgatgtatttttgttctaaggccgccgcccctgacgaagccttcccccttgaattttggggccaggggacccaa |
| | | gtcatcgtctcctcggcctccaccaagggcccatcggtcttccccctggcaccctcctccaagagcacctctgggggcacagcgg |
| | | ccctgggctgcctggtcaaggactacttccccgaaccggtgacggtgtcgtggaactcaggcgccctgaccagcggcgtgcaca |
| | | ccttcccggctgtcctacagtcctcaggactctactccctcagcagcgtggtgaccgtgccctccagcagcttgggcacccagacc |
| | | tacatctgcaacgtgaatcacaagcccagcaacaccaaggtggacaagaaagttgagcccaaatcttgtgacaaaactcacacat |
| | | gcccaccgtgcccagcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtacgtggacggcgtg |
| | | gaggtgcataatgccaagacaaagccgcgggaggagcagtacaacagcacgtaccgggtggtcagcgtcctcaccgtcctgca |
| | | ccaggactggctgaatggcaaggagtacaagtgcaaggtctccaacaaagccctcccagcccccatcgagaaaaccatctccaa |
| | | agccaaagggcagccccgagaaccacaggtgtacaccctgcccccatcccgggatgagctgaccaagaaccaggtcagcctg |
| | | acctgcctggtcaaaggcttctatcccagcgacatcgccgtggagtgggagagcaatgggcagccggagaacaactacaagac |
| | | cacgcctcccgtgctggactccgacggctccttcttcctctacagcaagctcaccgtggacaagagcaggtggcagcaggggaa |
| | | cgtcttctcatgctccgtgatgcatgaggctctgcacaaccactacacgcagaagagcctctccctgtctccgggtaaa |
| | | SEQ ID NO: 135 |
| | | ----------------------------------------------------------------------------------------- |
| | | caggctgccctgactcagcccgcctccgtgtccggctctcctggacagtcggtcaccatttcctgcctttatgccaatgtggatatct |
| | | gctggtatcaacttcacccgggcagagcccccaaacttctaattgttgacaataataagcggccctcaggagtctctagtcgcttctc |
| | | tggttccaagtctggcaccacggcctccctaacaatctctgatcttcaggctgacgacgaggctgaatatcactgctcttcaacaaca |
| | | ttttttggcggggggaccaggttgaccgtcctgagtcagcccaaggctgccccctcggtcactctgttcccaccctcctctgaggag |
| | | cttcaagccaacaaggccacactggtgtgtctcgtaagtgacttctacccgggagccgtgacagtggcctggaaggcagatggca |
| | | gccccgtcaaggtgggagtggagaccaccaaaccctccaaacaaagcaacaacaagtatgcggccagcagctacctgagcctg |
| | | acgcccgagcagtggaagtcccacagaagctacagctgccgggtcacgcatgaagggagcaccgtggagaagacagtggcc |
| | | cctgcagaatgctct |
| | | SEQ ID NO: 136 |
| Sequence | N49P65 | caagtgcaactggtgcagtctggggctggggtgaagaagcctggggcctcagtgagggtctcctgcgagacatccggattcaag |
| 31 | | ttcaccgagtactttatccactttttacgacaggcccctggacaagggcttgagtggatgggatggctcaaccctctcagaggtgcc |
| | | gtcaactatccacggaagtttcagggcagagtcactttgaccagggacatctacaccaccaccgtctacatgcaacttaacggtctg |
| | | acccctgacgacacggccgtctactactgtgccagagcggtctttaatgaagctttcccctttgactactggggccagggaagcctg |
| | | gtcaccgtctcctcagcctccaccaagggcccatcggtcttccccctggcaccctcctccaagagcacctctgggggcacagcgg |
| | | ccctgggctgcctggtcaaggactacttccccgaaccggtgacggtgtcgtggaactcaggcgccctgaccagcggcgtgcaca |
| | | ccttcccggctgtcctacagtcctcaggactctactccctcagcagcgtggtgaccgtgccctccagcagcttgggcacccagacc |
| | | tacatctgcaacgtgaatcacaagcccagcaacaccaaggtggacaagaaagttgagcccaaatcttgtgacaaaactcacacat |
| | | gcccaccgtgcccagcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtacgtggacggcgtg |
| | | gaggtgcataatgccaagacaaagccgcgggaggagcagtacaacagcacgtaccgggtggtcagcgtcctcaccgtcctgca |
| | | ccaggactggctgaatggcaaggagtacaagtgcaaggtctccaacaaagccctcccagcccccatcgagaaaaccatctccaa |
| | | agccaaagggcagccccgagaaccacaggtgtacaccctgcccccatcccgggatgagctgaccaagaaccaggtcagcctg |
| | | acctgcctggtcaaaggcttctatcccagcgacatcgccgtggagtgggagagcaatgggcagccggagaacaactacaagac |
| | | cacgcctcccgtgctggactccgacggctccttcttcctctacagcaagctcaccgtggacaagagcaggtggcagcaggggaa |
| | | cgtcttctcatgctccgtgatgcatgaggctctgcacaaccactacacgcagaagagcctctccctgtctccgggtaaa |
| | | SEQ ID NO: 137 |
| | | ----------------------------------------------------------------------------------------- |
| | | tcctgggcccagactcagcctgcctccgtgtctgggtctcctggacagtcgatcaccatctcctgcgctggaatcgtcagtgatgcc |
| | | tggtaccagcaatacccaggcagaccccccagactcatcctttatgacggcgataagcggccctcaggggtttctcctcgtttttctg |
| | | cctccagggccggcaagacggcctccctgacaatttctgggctgcaggctgacgacgaggcttattatcactgcgcgtcaaggga |
| | | attttttggaggcgtgaccaagttgaccgtcctaagtcagcccaaggctgccccctcggtcactctgttcccaccctcctctgaggag |
| | | cttcaagccaacaaggccacactggtgtgtctcgtaagtgacttctacccgggagccgtgacagtggcctggaaggcagatggca |
| | | gccccgtcaaggtgggagtggagaccaccaaaccctccaaacaaagcaacaacaagtatgcggccagcagctacctgagcctg |
| | | acgcccgagcagtggaagtcccacagaagctacagctgccgggtcacgcatgaagggagcaccgtggagaagacagtggcc |
| | | cctgcagaatgctct |
| | | SEQ ID NO: 138 |
| Sequence | N49P66 | caggtgcgacttcagcagtctggtgtcgtggtgaggaagcctggggcctcagtgagactttcctgcgagacgtctggcttcaaattc |
| 32 | | atcgaccacattgtcaactgggtgcggcgggcccctggacgaggctttgaatggatgggttggatcaagcctcttgggggtgtcg |
| | | ctgattatgcaccccaacatcggggcaggatctcactgacgagggacatttacactgaaaccgtctttatagacctgagtcgactga |
| | | cgtctggcgacacggcgatttatttttgttgtaaggccgccgcccctgatgaagcattcccccttgaatactggggccaggggaccc |
| | | aacttatcgtctccccggcctccaccaagggcccatcggtcttccccctggcaccctcctccaagagcacctctgggggcacagc |
| | | ggccctgggctgcctggtcaaggactacttccccgaaccggtgacggtgtcgtggaactcaggcgccctgaccagcggcgtgca |
| | | caccttcccggctgtcctacagtcctcaggactctactccctcagcagcgtggtgaccgtgccctccagcagcttgggcacccaga |
| | | cctacatctgcaacgtgaatcacaagcccagcaacaccaaggtggacaagaaagttgagcccaaatcttgtgacaaaactcacac |
| | | atgcccaccgtgcccagcacctgaactcctggggggaccgtcagtcttcctcttccccccaaaacccaaggacaccctcatgatct |
| | | cccggacccctgaggtcacatgcgtggtggtggacgtgagccacgaagaccctgaggtcaagttcaactggtacgtggacggc |
| | | gtggaggtgcataatgccaagacaaagccgcgggaggagcagtacaacagcacgtaccgggtggtcagcgtcctcaccgtcct |
| | | gcaccaggactggctgaatggcaaggagtacaagtgcaaggtctccaacaaagccctcccagcccccatcgagaaaaccatctc |
| | | caaagccaaagggcagccccgagaaccacaggtgtacaccctgcccccatcccgggatgagctgaccaagaaccaggtcagc |
| | | ctgacctgcctggtcaaaggcttctatcccagcgacatcgccgtggagtgggagagcaatgggcagccggagaacaactacaag |
| | | accacgcctcccgtgctggactccgacggctccttcttcctctacagcaagctcaccgtggacaagagcaggtggcagcagggg |
| | | aacgtcttctcatgctccgtgatgcatgaggctctgcacaaccactacacgcagaagagcctctccctgtctccgggtaaa |
| | | SEQ ID NO: 139 |
| | | ----------------------------------------------------------------------------------------- |
| | | caggctgccctgactcagcccgcctccgtgtccggctctcctggacagtcggtcaccatttcctgcctttatgccaatgtagatatct |
| | | gctggtatcaaatacagccgggcagattacccaaacttctgattgttgacaatgataggcgaccctcaggagtctctcctcgcttctct |
| | | ggctccaagtctggcaccacggcctccctgacaatctctggacttcaggctgacgacgaggctgaatatcactgctcttcaacaac |
| | | attttttggcggggggaccaagttgaccgtcctcagtcagcccaaggctgccccctcggtcactctgttcccaccctcctctgagga |
| | | gcttcaagccaacaaggccacactggtgtgtctcgtaagtgacttctacccgggagccgtgacagtggcctggaaggcagatggc |
| | | agccccgtcaaggtgggagtggagaccaccaaaccctccaaacaaagcaacaacaagtatgcggccagcagctacctgagcct |
| | | gacgcccgagcagtggaagtcccacagaagctacagctgccgggtcacgcatgaagggagcaccgtggagaagacagtggc |
| | | ccctgcagaatgctct |
| | | SEQ ID NO: 140 |
| Sequence | N49P67 | caggtgcgacttgtgcagtctggtcccgtgatgagaaagcctggggcctcagtgagaatttcttgcgagacatctggattcgccttct |
| 33 | | tggaccacattgtccactgggtgcggcgggcccctggacgcggctttgaatggatgggttgggttaagaccattgggggtgtcgtt |
| | | gattatgcaccccaccttaggggcaggatctccgtgacgagagacgtctttagtgaaaccgtctttctggacttgagtcgactgacgt |
| | | ctggcgacacggcgatgtatttttgttctaaggccgccgcccctgacgaagccttcccccttgaattttggggccaggggacccaa |
| | | gtcatcgtctcctcggcctccaccaagggcccatcggtcttccccctggcaccctcctccaagagcacctctgggggcacagcgg |
| | | ccctgggctgcctggtcaaggactacttccccgaaccggtgacggtgtcgtggaactcaggcgccctgaccagcggcgtgcaca |
| | | ccttcccggctgtcctacagtcctcaggactctactccctcagcagcgtggtgaccgtgccctccagcagcttgggcacccagacc |
| | | tacatctgcaacgtgaatcacaagcccagcaacaccaaggtggacaagaaagttgagcccaaatcttgtgacaaaactcacacat |
| | | gcccaccgtgcccagcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtacgtggacggcgtg |
| | | gaggtgcataatgccaagacaaagccgcgggaggagcagtacaacagcacgtaccgggtggtcagcgtcctcaccgtcctgca |
| | | ccaggactggctgaatggcaaggagtacaagtgcaaggtctccaacaaagccctcccagcccccatcgagaaaaccatctccaa |
| | | agccaaagggcagccccgagaaccacaggtgtacaccctgcccccatcccgggatgagctgaccaagaaccaggtcagcctg |
| | | acctgcctggtcaaaggcttctatcccagcgacatcgccgtggagtgggagagcaatgggcagccggagaacaactacaagac |
| | | cacgcctcccgtgctggactccgacggctccttcttcctctacagcaagctcaccgtggacaagagcaggtggcagcaggggaa |
| | | cgtcttctcatgctccgtgatgcatgaggctctgcacaaccactacacgcagaagagcctctccctgtctccgggtaaa |
| | | SEQ ID NO: 141 |
| | | ----------------------------------------------------------------------------------------- |
| | | caggctgccctgactcagcccgcctccgtgtccggctctcctggacagtcggtcaccatttcttgcctttatgccaatgtggatatctg |
| | | ctggtatcaacttcacccgggcagagcccccaaacttctaattcttgacaataataaacggccctcaggagtctctagtcgcttctcc |
| | | ggttccaagtctggcaccacggcctccctaaccatctctgaccttcaggctgacgacgaggctgaatatcactgctcttcaacaactt |
| | | tttttggcggggggaccaggttgaccgtcctgagtcagcccaaggctgccccctcggtcactctgttcccaccctcctctgaggag |
| | | cttcaagccaacaaggccacactggtgtgtctcgtaagtgacttctacccgggagccgtgacagtggcctggaaggcagatggca |
| | | gccccgtcaaggtgggagtggagaccaccaaaccctccaaacaaagcaacaacaagtatgcggccagcagctacctgagcctg |
| | | acgcccgagcagtggaagtcccacagaagctacagctgccgggtcacgcatgaagggagcaccgtggagaagacagtggcc |
| | | cctgcagaatgctct |
| | | SEQ ID NO: 142 |
| Sequence | N49P68 | cacgtgcagttgaggcagtctgggactgaggcgaagaagtctggggcctcggtgacaatctcttgtgagaccgctggattcaactt |
| 34 | | catcgactccgtcatacactggctgcgccaggcccctggtgggggatttcagtggatggggtggatcaagcctcttagaggtggc |
| | | gtcaattatccacattatttgcagggcagaatctccatgacccgggacttgtccagtgacacggtttacatggtcttaaatagactgac |
| | | acctgccgacacaggcctttattactgcgcgaaaggggcctttggggggagttctccctttggcttctggggccagggaactctgct |
| | | caccgtctccccagcctccaccaagggcccatcggtcttccccctggcaccctcctccaagagcacctctgggggcacagcggc |
| | | cctgggctgcctggtcaaggactacttccccgaaccggtgacggtgtcgtggaactcaggcgccctgaccagcggcgtgcacac |
| | | cttcccggctgtcctacagtcctcaggactctactccctcagcagcgtggtgaccgtgccctccagcagcttgggcacccagacct |
| | | acatctgcaacgtgaatcacaagcccagcaacaccaaggtggacaagaaagttgagcccaaatcttgtgacaaaactcacacatg |
| | | cccaccgtgcccagcacctgaactcctggggggaccgtcagtcttcctcttccccccaaaacccaaggacaccctcatgatctccc |
| | | ggacccctgaggtcacatgcgtggtggtggacgtgagccacgaagaccctgaggtcaagttcaactggtacgtggacggcgtgg |
| | | aggtgcataatgccaagacaaagccgcgggaggagcagtacaacagcacgtaccgggtggtcagcgtcctcaccgtcctgcac |
| | | caggactggctgaatggcaaggagtacaagtgcaaggtctccaacaaagccctcccagcccccatcgagaaaaccatctccaaa |
| | | gccaaagggcagccccgagaaccacaggtgtacaccctgcccccatcccgggatgagctgaccaagaaccaggtcagcctga |
| | | cctgcctggtcaaaggcttctatcccagcgacatcgccgtggagtgggagagcaatgggcagccggagaacaactacaagacc |
| | | acgcctcccgtgctggactccgacggctccttcttcctctacagcaagctcaccgtggacaagagcaggtggcagcaggggaac |
| | | gtcttctcatgctccgtgatgcatgaggctctgcacaaccactacacgcagaagagcctctccctgtctccgggtaaa |
| | | SEQ ID NO: 143 |
| | | ----------------------------------------------------------------------------------------- |
| | | cagtctgccctgtctcagcctgtctccgtgtctgggtctcctggagagtcgatcaccatttcctgtactgaagccaccacctggtatca |
| | | acaactcccaggcaaaccccccaaactcatcatttatgacgtgaccaaccggccctcaggcatttcaagtcgtttctctggctccatg |
| | | tctggtcgcacggcctccctgacaatctccggtctccaggttgacgacgagggtctctatcactgtgcctcacgtgaatttttcggcg |
| | | gggggaccaagctgaccgtcctgagtcagcccaaggctgccccctcggtcactctgttcccaccctcctctgaggagcttcaagc |
| | | caacaaggccacactggtgtgtctcgtaagtgacttctacccgggagccgtgacagtggcctggaaggcagatggcagccccgt |
| | | caaggtgggagtggagaccaccaaaccctccaaacaaagcaacaacaagtatgcggccagcagctacctgagcctgacgccc |
| | | gagcagtggaagtcccacagaagctacagctgccgggtcacgcatgaagggagcaccgtggagaagacagtggcccctgcag |
| | | aatgctct |
| | | SEQ ID NO: 144 |
| Sequence | N49P69 | cacgtgcaattgatgcagtctgggactcaggcgaagaagtctggggcctcggtgacaatttcttgtgagaccgctggattcaagttc |
| 35 | | atcgactccgtcatacactggctgcgccaggcccctggagggggatttcagtggatggggtggatcaagcctcttggaggtgccg |
| | | tcaactatccaccctatttgcagggcaggatctccttgacccgtgacttgtccaccgacacaatttacatggtcttgaatggactgaca |
| | | cctgccgacacaggcttttattactgcgccaaaggggcctttggggggggttctccctttggcttctggggccaggggactctgctc |
| | | accgtctccccagcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagcggcgtgcacacc |
| | | ttcccggctgtcctacagtcctcaggactctactccctcagcagcgtggtgaccgtgccctccagcagcttgggcacccagaccta |
| | | catctgcaacgtgaatcacaagcccagcaacaccaaggtggacaagaaagttgagcccaaatcttgtgacaaaactcacacatgc |
| | | ccaccgtgcccagcacctgaactcctggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacggcgtgga |
| | | ggtgcataatgccaagacaaagccgcgggaggagcagtacaacagcacgtaccgggtggtcagcgtcctcaccgtcctgcacc |
| | | aggactggctgaatggcaaggagtacaagtgcaaggtctccaacaaagccctcccagcccccatcgagaaaaccatctccaaag |
| | | ccaaagggcagccccgagaaccacaggtgtacaccctgcccccatcccgggatgagctgaccaagaaccaggtcagcctgac |
| | | ctgcctggtcaaaggcttctatcccagcgacatcgccgtggagtgggagagcaatgggcagccggagaacaactacaagacca |
| | | cgcctcccgtgctggactccgacggctccttcttcctctacagcaagctcaccgtggacaagagcaggtggcagcaggggaacg |
| | | tcttctcatgctccgtgatgcatgaggctctgcacaaccactacacgcagaagagcctctccctgtctccgggtaaa |
| | | SEQ ID NO: 145 |
| | | ----------------------------------------------------------------------------------------- |
| | | cagtctgccctgtctcagcctgtctccgtgtctgggtctcctggagactcgatcaccatttcttgttttggagccaccacctggtatcaa |
| | | caactcccaggcagaccccccaaactcatcatttatgacgtgactaaccggccctcaggcatttcaggtcgtttctctggctccatgt |
| | | ctggtcaaaaggcctccctgacaatctccggtctccaggttgacgacgagggtctctatcactgcgcctcacgtgaatttttcggcg |
| | | gggggaccaaactgaccgtcctgagtcagcccaaggctgccccctcggtcactctgttcccaccctcctctgaggagcttcaagc |
| | | caacaaggccacactggtgtgtctcgtaagtgacttctacccgggagccgtgacagtggcctggaaggcagatggcagccccgt |
| | | caaggtgggagtggagaccaccaaaccctccaaacaaagcaacaacaagtatgcggccagcagctacctgagcctgacgccc |
| | | gagcagtggaagtcccacagaagctacagctgccgggtcacgcatgaagggagcaccgtggagaagacagtggcccctgcag |
| | | aatgctct |
| | | SEQ ID NO: 146 |
| Sequence | N49P70 | cacgtgcagttgaggcagtctgggactgaggcgaagaagtctggggcctcggtgacaatctcttgtgagaccgctggattcaactt |
| 36 | | catcgactccgtcatacactggctgcgccaggcccctggtgggggatttcagtggatggggtggatcaagcctcttagaggtggc |
| | | gtcaattatccacattatttgcagggcagaatctccatgacccgggacttgtccagtgacacggtttacatggtcttaaatagactgac |
| | | acctgacgacacaggcctttactactgcgcgaaaggggcctttggggggagttctccctttggcttctggggccagggaactctgc |
| | | tcaccgtctccccagcctccaccaagggcccatcggtcttccccctggcaccctcctccaagagcacctctgggggcacagcgg |
| | | ccctgggctgcctggtcaaggactacttccccgaaccggtgacggtgtcgtggaactcaggcgccctgaccagcggcgtgcaca |
| | | ccttcccggctgtcctacagtcctcaggactctactccctcagcagcgtggtgaccgtgccctccagcagcttgggcacccagacc |
| | | tacatctgcaacgtgaatcacaagcccagcaacaccaaggtggacaagaaagttgagcccaaatcttgtgacaaaactcacacat |
| | | gcccaccgtgcccagcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtacgtggacggcgtg |
| | | gaggtgcataatgccaagacaaagccgcgggaggagcagtacaacagcacgtaccgggtggtcagcgtcctcaccgtcctgca |
| | | ccaggactggctgaatggcaaggagtacaagtgcaaggtctccaacaaagccctcccagcccccatcgagaaaaccatctccaa |
| | | agccaaagggcagccccgagaaccacaggtgtacaccctgcccccatcccgggatgagctgaccaagaaccaggtcagcctg |
| | | acctgcctggtcaaaggcttctatcccagcgacatcgccgtggagtgggagagcaatgggcagccggagaacaactacaagac |
| | | cacgcctcccgtgctggactccgacggctccttcttcctctacagcaagctcaccgtggacaagagcaggtggcagcaggggaa |
| | | cgtcttctcatgctccgtgatgcatgaggctctgcacaaccactacacgcagaagagcctctccctgtctccgggtaaa |
| | | SEQ ID NO: 147 |
| | | ----------------------------------------------------------------------------------------- |
| | | cagtctgccctgtctcagcctgtctccgtgtctgggtctcctggagagtcgatcaccatttcctgtactgaagccaccacctggtatca |
| | | acaactcccagggagatcccccaaactcattatttatgacgtgaccaaccggccctcaggcatttcaagtcgtttctctggctccatgt |
| | | ctggtcgcacggcctccctgacaatctccggtctccaggttgacgacgagggtctctatcactgtgcctcacgtgaatttttcggcgg |
| | | ggggaccaagctgaccgtcctcagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagatggcagccccgtc |
| | | aaggtgggagtggagaccaccaaaccctccaaacaaagcaacaacaagtatgcggccagcagctacctgagcctgacgcccg |
| | | agcagtggaagtcccacagaagctacagctgccgggtcacgcatgaagggagcaccgtggagaagacagtggcccctgcaga |
| | | atgctct |
| | | SEQ ID NO: 148 |
| Sequence | N49P71 | cgtgttacattacaacagtctggggctacagtgaggcagcctggggcctcagtcaccgtctcctgcgagacttctggattcaccttc |
| 37 | | atcaaatataccattcactgggtgcgacaggcccctggacagggtcttcagtgggtgggaagaatacacccccgaaccggtgcc |
| | | gtgaagtttgcaccgatatttcagggtaaattttccatgagtcgagacttgtcacgcgacacagcctacctcggattgaccagactga |
| | | cactcgccgacacggctctatttttctgtgcgaggggggcctttgaggcagatttatatgggccaacttacccctttcaccactgggg |
| | | ccaaggaacccaagtcaccgtctccgcggcctccaccaagggcccatcggtcttccccctggcaccctcctccaagagcacctct |
| | | gggggcacagcggccctgggctgcctggtcaaggactacttccccgaaccggtgacggtgtcgtggaactcaggcgccctgac |
| | | cagcggcgtgcacaccttcccggctgtcctacagtcctcaggactctactccctcagcagcgtggtgaccgtgccctccagcagct |
| | | tgggcacccagacctacatctgcaacgtgaatcacaagcccagcaacaccaaggtggacaagaaagttgagcccaaatcttgtg |
| | | acaaaactcacacatgcccaccgtgcccagcacctgaactcctggggggaccgtcagtcttcctcttccccccaaaacccaagga |
| | | caccctcatgatctcccggacccctgaggtcacatgcgtggtggtggacgtgagccacgaagaccctgaggtcaagttcaactgg |
| | | tacgtggacggcgtggaggtgcataatgccaagacaaagccgcgggaggagcagtacaacagcacgtaccgggtggtcagcg |
| | | tcctcaccgtcctgcaccaggactggctgaatggcaaggagtacaagtgcaaggtctccaacaaagccctcccagcccccatcg |
| | | agaaaaccatctccaaagccaaagggcagccccgagaaccacaggtgtacaccctgcccccatcccgggatgagctgaccaag |
| | | aaccaggtcagcctgacctgcctggtcaaaggcttctatcccagcgacatcgccgtggagtgggagagcaatgggcagccgga |
| | | gaacaactacaagaccacgcctcccgtgctggactccgacggctccttcttcctctacagcaagctcaccgtggacaagagcagg |
| | | tggcagcaggggaacgtcttctcatgctccgtgatgcatgaggctctgcacaaccactacacgcagaagagcctctccctgtctcc |
| | | gggtaaa |
| | | SEQ ID NO: 149 |
| | | ----------------------------------------------------------------------------------------- |
| | | tcctgggccctgactcaacccgcctccgtgtctgcgtctcctgggcagtcggtcaccatgtcctgcactggattcggaagttataatc |
| | | ctgactcctggtaccagcaatacccaggcaaagcccccaaactcatcatttatgatgacaataaaagaccctcgggggtctctgatc |
| | | gcttctctgcctccagacttggcagcacatcttcactgacaatctctaacgtccaggccgctgacgacgcccattatgtctgcgcctc |
| | | ctttgagtttttcggcggggggaccaagctgaccgtcctgagtcagcccaaggctgccccctcggtcactctgttcccaccctcctc |
| | | tgaggagcttcaagccaacaaggccacactggtgtgtctcgtaagtgacttctacccgggagccgtgacagtggcctggaaggca |
| | | gatggcagccccgtcaaggtgggagtggagaccaccaaaccctccaaacaaagcaacaacaagtatgcggccagcagctacct |
| | | gagcctgacgcccgagcagtggaagtcccacagaagctacagctgccgggtcacgcatgaagggagcaccgtggagaagac |
| | | agtggcccctgcagaatgctct |
| | | SEQ ID NO: 150 |
| Sequence | N49P72 | cacattcagttgctacagtcggggcctcaagttaagaagtctggggacacagtgagaatctcctgtgagacctctggatataatttcg |
| 38 | | tcgactcccttatccactgggtccgacagaccccggaaaaacgtctcagatggatgggctggatcaatcctctccaaggtggtgtg |
| | | aattacgcgccggaatttcagggcagaatcaggatgaccagggacacgtttatagacacagtttacgtggacttgagcggactgac |
| | | accggccgacacggcctattattactgcgcgcgagggatcgatggcaattcttacccctttcatttctggggccacggaacccggg |
| | | tcaccgtcttctcggcctccaccaagggcccatcggtcttccccctggcaccctcctccaagagcacctctgggggcacagcggc |
| | | cctgggctgcctggtcaaggactacttccccgaaccggtgacggtgtcgtggaactcaggcgccctgaccagcggcgtgcacac |
| | | cttcccggctgtcctacagtcctcaggactctactccctcagcagcgtggtgaccgtgccctccagcagcttgggcacccagacct |
| | | acatctgcaacgtgaatcacaagcccagcaacaccaaggtggacaagaaagttgagcccaaatcttgtgacaaaactcacacatg |
| | | cccaccgtgcccagcacctgaactcctggggggaccgtcagtcttcctcttccccccaaaacccaaggacaccctcatgatctccc |
| | | ggacccctgaggtcacatgcgtggtggtggacgtgagccacgaagaccctgaggtcaagttcaactggtacgtggacggcgtgg |
| | | aggtgcataatgccaagacaaagccgcgggaggagcagtacaacagcacgtaccgggtggtcagcgtcctcaccgtcctgcac |
| | | caggactggctgaatggcaaggagtacaagtgcaaggtctccaacaaagccctcccagcccccatcgagaaaaccatctccaaa |
| | | gccaaagggcagccccgagaaccacaggtgtacaccctgcccccatcccgggatgagctgaccaagaaccaggtcagcctga |
| | | cctgcctggtcaaaggcttctatcccagcgacatcgccgtggagtgggagagcaatgggcagccggagaacaactacaagacc |
| | | acgcctcccgtgctggactccgacggctccttcttcctctacagcaagctcaccgtggacaagagcaggtggcagcaggggaac |
| | | gtcttctcatgctccgtgatgcatgaggctctgcacaaccactacacgcagaagagcctctccctgtctccgggtaaa |
| | | SEQ ID NO: 151 |
| | | ----------------------------------------------------------------------------------------- |
| | | cgatttgccctgactcaacctgcctccgtgtctgggtctcctggacagacgatcaccataacctgcgctggaggcagcgtctcctgg |
| | | ttccatttccctccaggcaaaacccccagactcattatttatgagtcttctaagagaccctcaggggtctctcctcgattctctgggtcc |
| | | cagtctggcagcacggcctccctaataatttctggcctccagtctgatgacgaagggacatacttctgttctattcttgaatttttcggc |
| | | agagggactcttctcaccgtcctgagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagatggcagccccgtc |
| | | aaggtgggagtggagaccaccaaaccctccaaacaaagcaacaacaagtatgcggccagcagctacctgagcctgacgcccg |
| | | agcagtggaagtcccacagaagctacagctgccgggtcacgcatgaagggagcaccgtggagaagacagtggcccctgcaga |
| | | atgctct |
| | | SEQ ID NO: 152 |

TABLE 3
List of mAb variants and the corresponding sequence and mutations. The amino acid
sequence of the variants is determined based on the reference antibody sequence (see
table 2, above for reference antibody sequences) and the mutations described in the
table. See amino acid sequences and corresponding oligonucleotide sequences.

| | Related to | Heavy chain | Light chain | Swaps/ |
|---|---|---|---|---|
| mAb | sequence | mutation | mutations | Other |
| N49P6 | 1 | CH1: 1.4A, 120R | Constant: 1.5G | LC7 swap |
| | | CH3: 12 E, 14M | | |
| N49P6.2 | 1 | CH1: 120R | | |
| | | CH3: 12 E, 14M | | |
| N49P7 | 2 | CH1: 1.4A, 120R | Constant: 1.5G | |
| | | CH3: 12 E, 14M | | |
| N49P7.1 | 2 | CH1: 120R | | |
| | | CH3: 12 E, 14M | | |
| N49P7A | 2 | CH1: 1.4A, 120R | Variable: 42A | |
| | | CH3: 12 E, 14M | Constant: 1.5G | |
| N49P7F | 2 | CH1: 1.4A, 120R | Variable: 42F | |
| | | CH3: 12 E, 14M | Constant: 1.5G | |
| N49P7S | 2 | CH1: 1.4A, 120R | Variable: 42S | |
| | | CH3: 12 E, 14M | Constant: 1.5G | |
| N49P7Y | 2 | CH1: 1.4A, 120R | Variable: 42Y | |
| | | CH3: 12 E, 14M | Constant: 1.5G | |
| N49P7-54TY | 2 | Variable: 59T/62Y | Constant: 1.5G | |
| | | CH1: 1.4A, 120R | | |
| | | CH3: 12 E, 14M | | |
| N49P7LS-1 | 2 | CH1: 1.4A, 120R | Constant: 1.5G | |
| | | CH3: 12 E, 14M, | | |
| | | 107L, 114S | | |
| N49P7LS-2 | 2 | CH1: 1.4A, 120R | Constant: 1.5G | |
| | | CH3: 107L, 114S | | |
| N49P7YTE | 2 | CH1: 1.4A, 120R | Constant: 1.5G | |
| | | CH2: 15.1Y, | | |
| | | 16T, 18E | | |
| N49P7L6 | 2.1 | CH1: 1.4A, 120R | Constant: 1.5G | N49P7 heavy chain |
| | | CH3: 12 E, 14M | | with N49P6 light |
| | | | | chain |
| N49P7L11 | 2.4 | CH1: 1.4A, 120R | | N49P7 heavy chain |
| | | CH3: 12 E, 14M | | with N49P11 light |
| | | | | chain |
| N49P7.1L9 | 2.18 | CH1: 120R | | N49P7.1 heavy |
| | | CH3: 12 E, 14M | | chain with N49P9 |
| | | | | i light chain |
| N49P7.1L19 | 2.7 | CH1: 120R | | N49P7 heavy chain |
| | | CH3: 12 E, 14M | | with N49P19 light |
| | | | | chain |
| R49P7 | 2 | CH1: 1.4A | Constant: 1.5G | Rhesus IgG1, |
| | | | | Rhesus LC3 |
| N49P7.2 | 3 | CH1: 120R | | |
| | | CH3: 12 E, 14M | | |
| N49P11 | 4 | CH1: 120R | | |
| | | CH3: 12 E, 14M | | |
| N49P18 | 5 | CH1: 120R | | |
| | | CH3: 12 E, 14M | | |
| N49P18.2 | 5 | CH1: 120R | | LC7 swap |
| | | CH3: 12 E, 14M | | |
| N49P18.1 | 6 | CH1: 120R | | |
| | | CH3: 12 E, 14M | | |
| N49P19 | 7 | CH1: 120R | | |
| | | CH3: 12 E, 14M | | |
| N49P37 | 8 | CH1: 1.4A, 120R | Constant: 1.5G | |
| | | CH3: 12 E, 14M | | |
| N49P38 | 9 | CH1: 120R | Constant: 1.5G | |
| | | CH3: 12 E, 14M | | |
| N4938.1 | 9 | CH1: 120R | | |
| | | CH3: 12 E, 14M | | |
| N49P55 | 10 | CH1: 120R | | |
| | | CH3: 12 E, 14M | | |
| N49P56 | 11 | CH1: 120R | | |
| | | CH3: 12 E, 14M | | |
| N49P57 | 12 | CH1: 120R | | |
| | | CH3: 12 E, 14M | | |
| N49P58 | 13 | CH1: 120R | | |
| | | CH3: 12 E, 14M | | |
| N49P59 | 14 | CH1: 120R | | |
| | | CH3: 12 E, 14M | | |
| N49P73 | 15 | CH1: 120R | | |
| | | CH3: 12 E, 14M | | |
| N49P74 | 16 | CH1: 120R | | |
| | | CH3: 12 E, 14M | | |
| N49P75 | 17 | CH1: 120R | | |
| | | CH3: 12 E, 14M | | |
| N49P75.1 | 17 | CH1: 1.4A, 120R | Constant: 1.5G | |
| | | CH3: 12 E, 14M | | |
| N49P9 | 18 | CH1: 120R | | |
| | | CH3: 12 E, 14M | | |
| N49P9.1 | 18 | CH1: 120R | | LC2 swap |
| | | CH3: 12 E, 14M | | |
| N49P9.2 | 18 | CH1: 1.4A, 120R | Constant: 1.5G | |
| | | CH3: 12 E, 14M | | |
| N49P9i7 | 18.2 | CH1: 120R | | Part of CDRH3 of |
| | | CH3: 12 E, 14M | | N49P7 swapped for |
| | | | | entire CDRH3 of |
| | | | | N49P9 |
| N49P9i7H1 | 18.2 | CH1: 120R | | CDRH3 of N49P7 |
| | | CH3: 12 E, 14M | | swapped for |
| | | | | CDRH3 of N49P9 |
| N49P9i7H2 | 18.2 | CH1: 1.4S, 120R | Constant: 1.5R | CDRH3 and junction |
| | | CH3: 12 E, 14M | | of N49P7 swapped |
| | | | | for CDRH3 of |
| | | | | N49P9 |
| N49P22 | 19 | CH1: 120R | | |
| | | CH3: 12 E, 14M | | |
| N49P23 | 20 | CH1: 120R | | |
| | | CH3: 12 E, 14M | | |
| N49P9.3 | 21 | CH1: 120R | Constant: 1.5R | |
| | | CH3: 12 E, 14M | | |
| N49P9.4 | 21 | CH1: 120R | | |
| | | CH3: 12 E, 14M | | |
| N49P51 | 22 | CH1: 120R | | |
| | | CH3: 12 E, 14M | | |
| N49P52 | 23 | CH1: 120R | | |
| | | CH3: 12 E, 14M | | |
| N49P53 | 24 | CH1: 120R | | |
| | | CH3: 12 E, 14M | | |
| N49P54 | 25 | CH1: 120R | | |
| | | CH3: 12 E, 14M | | |
| N49P60 | 26 | CH1: 120R | | |
| | | CH3: 12 E, 14M | | |
| N49P61 | 27 | CH1: 120R | | |
| | | CH3: 12 E, 14M | | |
| N49P62 | 28 | CH1: 120R | | |
| | | CH3: 12 E, 14M | | |
| N49P63 | 29 | CH1: 120R | | |
| | | CH3: 12 E, 14M | | |
| N49P64 | 30 | CH1: 120R | | |
| | | CH3: 12 E, 14M | | |
| N49P65 | 31 | CH1: 120R | | |
| | | CH3: 12 E, 14M | | |
| N49P66 | 32 | CH1: 120R | | |
| | | CH3: 12 E, 14M | | |
| N49P67 | 33 | CH1: 120R | | |
| | | CH3: 12 E, 14M | | |
| N49P68 | 34 | CH1: 120R | | |
| | | CH3: 12 E, 14M | | |
| N49P69 | 35 | CH1: 120R | | |
| | | CH3: 12 E, 14M | | |
| N49P7-FR | 2 | CH1: 1.4A, 120R | Constant: 1.5G | QLSQDPDDPDWG |
| | | CH3: 12 E, 14M | | insertion FR3 in 81-85 |
| N49P9-FR | 18 | CH1: 120R | | QLSQDPDDPDWG |
| | | CH3: 12 E, 14M | | insertion FR3 in 81-85 |
| N49P9.3-FR | 21 | CH1: 120R | Constant: 1.5R | QLSQDPDDPDWG |
| | | CH3: 12 E, 14M | | insertion FR3 in 81-85 |
| N49P9.6-FR | 21 | CH1: 1.4A, 120R | Constant: 1.5G | QLSQDPDDPDWG |
| | | CH3: 12 E, 14M | | insertion FR3 in 81-85 |
| N49P9.6-FR-54W | 21 | Variable: 59W | Constant: 1.5G | QLSQDPDDPDWG |
| | | CH1: 1.4A, 120R | | insertion FR3 in 81-85 |
| | | CH3: 12 E, 14M | | |
| N49P9.6-FR-54-F | 21 | Variable: 59F | Constant: 1.5G | QLSQDPDDPDWG |
| | | CH1: 1.4A, 120R | | insertion FR3 in 81-85 |
| | | CH3: 12 E, 14M | | |
| N49P9.6-54W | 21 | Variable: 59W | Constant: 1.5G | |
| | | CH1: 1.4A, 120R | | |
| | | CH3: 12 E, 14M | | |
| N49P9.6-54F | 21 | Variable: 59F | Constant: 1.5G | |
| | | CH1: 1.4A, 120R | | |
| | | CH3: 12 E, 14M | | |
| N49P9.6-FR3-06 | 21 | CH1: 1.4A, 120R | Constant: 1.5G | LFSQDLYYPDRG |
| | | CH3: 12 E, 14M | | insertion FR3 in 81-85 |
| N49P9.6-FR1-D | 21 | Variable: 29D | Constant: 1.5G | |
| | | CH1: 1.4A, 120R | | |
| | | CH3: 12 E, 14M | | |
| N49P9.6-FR1-D-I | 21 | Variable: 29D35I | Constant: 1.5G | |
| | | CH1: 1.4A, 120R | | |
| | | CH3: 12 E, 14M | | |
| N49P9.6-LS | 21 | CH1: 1.4A, 120R | Constant: 1.5G | |
| | | CH3: 12 E, 14M | | |
| | | 107L, 114S | | |
| N49P9.6-YTE | 21 | CH1: 1.4A, 120R | Constant: 1.5G | |
| | | CH2: 15.1Y, | | |
| | | 16T, 18E | | |
| | | CH3: 12 E, 14M | | |
| N49P9.6-FR-LS | 21 | CH1: 1.4A, 120R | Constant: 1.5G | QLSQDPDDPDWG |
| | | CH3: 12 E, 14M | | insertion FR3 in 81-85 |
| | | 107L, 114S | | |
| N49P9.6-FR-YTE | 21 | CH1: 1.4A, 120R | Constant: 1.5G | QLSQDPDDPDWG |
| | | CH2: 15.1Y, | | insertion FR3 in 81-85 |
| | | 16T, 18E | | |
| | | CH3: 12 E, 14M | | |

In some embodiments, the above listed antibodies can be further modified.

In some embodiments, modifications can be made to improve antibody function (binding, neutralization, complement fixation, ADCC, ADCP).

In some embodiments, modifications to the heavy chain can be made: Dual substitution at positions 59 and 62 to T and Y, respectively. Substitution at position 1.4 of CH1 (1^{st} amino acid of the constant region) to G from P or S. Substitution at position 120 of CH1 to R. Substitution at position 12 of CH3 to E, at position 14 of CH3 to M.

In some embodiments, light chain modifications can be made: Substitution at position 1.5 of the light chain constant region (1″ amino acid of the constant region) from R to S or G for those that use LC2, or from S to R or G for those that use LC7.

In some embodiments, modifications to improve antibody half-life can be made. These include the well-recognized “LS” (107L, 114S in CH3) and “YTE” (15.1Y, 16T,18E in CH2) mutations in the Fc. These also include other modifications in the Heavy chain such as: Substitution at position 120 of CH1 to R; Substitution at position 12 of CH3 to E, at position 14 of CH3 to M; Substitution at position 107 of CH3 to L, position 113 of CH3 to S, position 115 to R.

In some embodiments, modifications can be made that involve constant region swapping: Swapping of light chain constant region: light chain constant region swapped to another lambda constant region (LC1-7) for improved stability and function. Another method of swapping that can be used to make monoclonals that are “rhesus-ized,” that is, retain the variable regions in the heavy and light chains, but use constant regions from rhesus lambda chain (such as LC3), as well as rhesus IgG1. These mAbs can also have the half-life extending mutations noted above in the corresponding amino acids of the rhesus IgG1 constant region.

In some embodiments, the swapping of heavy and light chain variable regions can be made: These include taking a native or modified antibody listed in this application and mixing and matching the heavy and light chains from another in his application or N49P series to improve antibody stability or function.

In some embodiments, CDR replacement mutants can be made: These include taking the CDRH1, CDRH2, CDRH3, CDRL1, CDRL2, and/or CDRL3 from one of the parental or modified N49P series antibodies or those in this application and inserting into the corresponding CDR another natural or modified antibody.

In some embodiments, bispecific and trispecific antibodies can be made, For example, an antibody listed in this application would constitute one arm of an IgG molecule, while the other arm(s) would be another anti-HIV monoclonal (or a monoclonal that binds to a cell surface receptor). In some embodiments, the other anti-HIV monoclonal is another antibody listed in this application.

In some embodiments, antibody-drug conjugates can be made: The natural and modified antibodies listed here can be conjugated (covalently or non-covalently) to markers (florescent dye or radionuclides), therapeutic agents, or toxins such as auristatin (a microtubule toxin).

In some embodiments, pharmaceutical compositions can be made: These can include antibodies (pre-made in a variety of vehicles) delivered via injection, delivered in a slow-release depot, or genes encoding antibodies delivered via a viral or other vector.

In some embodiments, antibody-bead conjugates can be made. The modified antibodies listed here can be conjugated to agarose or other beads by a variety of chemical reactions (such as but not limited to Cyanogen Bromide-Activated and NHS esters) for the purpose of creating affinity purification columns that can bind (and purify) gp120 and its mutants, gp160 and its mutants, HIV trimers, or HIV-1 virus.

In some embodiments, the invention provides an expression vector comprising a polynucleotide encoding the VH region and/or the VL region of the anti-HIV antibodies above; or a host cell that comprises an expression vector of the anti-HIV antibodies above; or a host cell comprising a polynucleotide that encodes the VH region and/or the VL region of the anti-HIV antibodies above.

All of the antibodies herein, their modifications, and fragments, can be used for the purpose of HIV prevention, treatment, or cure, and can be done individually or in combination with any number of anti-HIV treatments, including latency reversing agents.

Amino acid and nucleotide sequences of the anti-HIV variant and modified antibodies are shown below. Variable regions within the heavy and light chain in the amino acid sequence are italicized and changes to the amino acid sequence relative to the natural or parental antibody sequence are underlined. CDR residues are in bold.

>N49P6
HEAVY CHAIN
AGLMQSGAVMKNSGASVRVSCQAD [symbol] IHWFRQRRGEGLEWLGW [symbol] NYPRPFQGKVT
MTRDTSTETAYLDVRGLTYDDTAVYYC [symbol] WGRGTQITVVSASTKGPSNPP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 153
GCGGGACTGATGCAGTCTGGGGCTGTGATGAAGAATTCGGGGGCCTCAGTGAGGGTCTCTTGTCA
GGCTGATGGATACGACTTCATTGACTATGTCATTCACTGGTTTCGACAAAGACGTGGAGAAGGTCT
TGAGTGGCTGGGATGGATGAATCCCTCGGGAGGCGGCACAAACTATCCGCGACCATTTCAGGGCA
AAGTCACCATGACCAGGGACACGTCCACCGAGACAGCCTATTTAGATGTCAGAGGACTTACATAT
GACGACACGGCCGTCTATTATTGTGTGAGAGACAGGGCCAACGGTTCGGGAAGAAGACGTTTTGA
GTCGGTGAATTGGTTCCTGGATCTGTGGGGCCGCGGCACCCAAATAACAGTCGTCTCGGCCTCCAC
CAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCACAAGCCCAGC
AACACCAAGGTGGACAAGAGAGTTGAGCCCAAATCTTGTGACAAAACTCACACATGCCCACCGTG
CCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGA
GCAGTACAACAGCACGTACCGGGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCG
GGTAAA
SEQ ID NO: 154
LIGHT CHAIN
QSALTQPRSVSASPGQSVTISCTGT [symbol] VSWCQQKPGQAPKLLIY [symbol] KRPSGVSDRFSGSTSGNTASLTISGL
QADDEGHYFC [symbol] GGGTKLTVLGQPKAAPSVTLFPPSSEELQANKATLVCLVSDFYPGAVTVAWK
ADGSPVKVGVETTKPSKQSNNKYAASSYLSLTPEQWKSHRSYSCRVTHEGSTVEKTVAPAECS
SEQ ID NO: 155
CAGTCTGCCCTAACTCAGCCTCGCTCAGTGTCCGCATCTCCTGGTCAGTCAGTCACCATCTCCTGC
ACTGGAACACACAATTATGTGTCCTGGTGTCAACAGAAACCGGGCCAAGCCCCCAAATTATTAAT
TTACGATTTCAATAAACGGCCCTCAGGGGTCTCTGATCGCTTCTCTGGCTCCACGTCTGGCAACAC
GGCCTCCCTGACCATCTCTGGACTCCAGGCTGACGATGAGGGTCATTATTTTTGTTGGGCGTTTGA
AAATATCGGCGGAGGGACCAAGCTGACCGTCCTGGGTCAGCCCAAGGCTGCCCCCTCGGTCACTC
TGTTCCCACCCTCCTCTGAGGAGCTTCAAGCCAACAAGGCCACACTGGTGTGTCTCGTAAGTGACT
TCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 156
>N49P6.2
HEAVY CHAIN
AGLMQSGAVMKNSGASVRVSCQAD [symbol] IHWFRQRRGEGLEWLGW [symbol] NYPRPFQGKVT
MTRDTSTETAYLDVRGLTYDDTAVYYC [symbol] WGRGTQITVVSASTKGPSNPP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 157
GCGGGACTGATGCAGTCTGGGGCTGTGATGAAGAATTCGGGGGCCTCAGTGAGGGTCTCTTGTCA
GGCTGATGGATACGACTTCATTGACTATGTCATTCACTGGTTTCGACAAAGACGTGGAGAAGGTCT
TGAGTGGCTGGGATGGATGAATCCCTCGGGAGGCGGCACAAACTATCCGCGACCATTTCAGGGCA
AAGTCACCATGACCAGGGACACGTCCACCGAGACAGCCTATTTAGATGTCAGAGGACTTACATAT
GACGACACGGCCGTCTATTATTGTGTGAGAGACAGGGCCAACGGTTCGGGAAGAAGACGTTTTGA
GTCGGTGAATTGGTTCCTGGATCTGTGGGGCCGCGGCACCCAAATAACAGTCGTCTCGGCCTCCAC
CAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCACAAGCCCAGC
AACACCAAGGTGGACAAGAGAGTTGAGCCCAAATCTTGTGACAAAACTCACACATGCCCACCGTG
CCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGA
GCAGTACAACAGCACGTACCGGGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCG
GGTAAA
SEQ ID NO: 158
LIGHT CHAIN
QSALTQPRSVSASPGQSVTISCTGT [symbol] VSWCQQKPGQAPKLLIY [symbol] KRPSGVSDRFSGSTSGNTASLTISGL
QADDEGHYFC [symbol] GGGTKLTVLGQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 159
CAGTCTGCCCTAACTCAGCCTCGCTCAGTGTCCGCATCTCCTGGTCAGTCAGTCACCATCTCCTGC
ACTGGAACACACAATTATGTGTCCTGGTGTCAACAGAAACCGGGCCAAGCCCCCAAATTATTAAT
TTACGATTTCAATAAACGGCCCTCAGGGGTCTCTGATCGCTTCTCTGGCTCCACGTCTGGCAACAC
GGCCTCCCTGACCATCTCTGGACTCCAGGCTGACGATGAGGGTCATTATTTTTGTTGGGCGTTTGA
AAATATCGGCGGAGGGACCAAGCTGACCGTCCTGGGTCAGCCCAAGGCTGCCCCCTCGGTCACTC
TGTTCCCGCCCTCCTCTGAGGAGCTTCAAGCCAACAAGGCCACACTGGTGTGTCTCATAAGTGACT
TCTACCCGGGAGCCGTGACAGTGGCTTGGAAAGCAGATAGCAGCCCCGTCAAGGCGGGAGTGGA
GACCACCACACCCTCCAAACAAAGCAACAACAAGTACGCGGCCAGCAGCTATCTGAGCCTGACGC
CTGAGCAGTGGAAGTCCCACAGAAGCTACAGCTGCCAGGTCACGCATGAAGGGAGCACCGTGGA
GAAGACAGTGGCCCCTACAGAATGTTCA
SEQ ID NO: 160
>N49P7
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 161
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 162
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 163
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 164
>N49P7.1
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSSSTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 165
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCATCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 166
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLRQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 167
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTCGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 168
>N49P7A
HEAVY CHAIN
ADLQSGAVVKKPGDSVRISCEAQ [symbol] IHWIRRAVPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 169
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 170
LIGHT CHAIN
ALTQPRSVSASPGQSVTISCTGT [symbol] VSWAQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 171
GCCCTGACTCAGCCTCGCTCAGTGTCCGCATCTCCTGGGCAGTCCGTCACCATTTCCTGCACTGGA
ACCCACAATTTGGTCTCTTGGGCTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 172
>N49P7F
HEAVY CHAIN
ADLQSGAVVKKPGDSVRISCEAQ [symbol] IHWIRRAVPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 173
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 174
LIGHT CHAIN
ALTQPRSVSASPGQSVTISCTGT [symbol] VSWFQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 175
GCCCTGACTCAGCCTCGCTCAGTGTCCGCATCTCCTGGGCAGTCCGTCACCATTTCCTGCACTGGA
ACCCACAATTTGGTCTCTTGGTTTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 176
>N49P7S
HEAVY CHAI
ADLQSGAVVKKPGDSVRISCEAQ [symbol] IHWIRRAVPGQGPEWMGW [symbol] NIPWKFQGRVSM
TRDTSIETAFLDLRGLKSDDTAVYYCV [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREE MTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 177
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 178
LIGHT CHAIN
ALTQPRSVSASPGQSVTISCTGT [symbol] VSWSQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 179
GCCCTGACTCAGCCTCGCTCAGTGTCCGCATCTCCTGGGCAGTCCGTCACCATTTCCTGCACTGGA
ACCCACAATTTGGTCTCTTGGTCTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 180
>N49P7Y
HEAVY CHAIN
ADLQSGAVVKKPGDSVRISCEAQ [symbol] IHWIRRAVPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 181
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 182
LIGHT CHAIN
ALTQPRSVSASPGQSVTISCTGT [symbol] VSWYQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 183
GCCCTGACTCAGCCTCGCTCAGTGTCCGCATCTCCTGGGCAGTCCGTCACCATTTCCTGCACTGGA
ACCCACAATTTGGTCTCTTGGTATCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 184
>N49P7-54TY
HEAVY CHAIN
ADLVQSGAVVKKPGDSVRISCEAQ [symbol] IHWIRRAPGQGPEWMGW [symbol] NIPWKFQGRVSMT
RDTSIETAFLDLRGLKSDDTAVYYC [symbol] WGRGTAVTIQSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 185
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAACGTACGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 186
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 187
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 188
>N49P7LS-1
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVLHEALHSHYTQKSLSLSPGK
SEQ ID NO: 189
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGCTGCATGAGGCTCTGCACAGCCACTACACGCAGAAGAGCCTCTCCCTGTCCCCGGG
TAAA
SEQ ID NO: 190
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 191
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 192
>N49P7LS-2
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDSDGS
FFLYSKLTVDKSRWQQGNVFSCSVLHEALHSHYTQKSLSLSPGK
SEQ ID NO: 193
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGATGAGCTGACCA
AGAACCAGGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGCTC
CTTCTTCCTCTACAGCAAGCTCACCGTGGACAAGAGCAGGTGGCAGCAGGGGAACGTCTTCTCAT
GCTCCGTGCTGCATGAGGCTCTGCACAGCCACTACACGCAGAAGAGCCTCTCCCTGTCCCCGGGTA
AA
SEQ ID NO: 194
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 195
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 196
>N49P7YTE
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 197
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCT
ATATCACCCGGGAGCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 198
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 199
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 200
>N49P7L6
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 201
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 202
LIGHT CHAIN
QSALTQPRSVSASPGQSVTISCTGT [symbol] VSWCQQKPGQAPKLLIY [symbol] KRPSGVSDRFSGSTSGNTASLTISGL
QADDEGHYFC [symbol] IGGGTKLTVLGQPKAAPSVTLFPPSSEELQANKATLVCLVSDFYPGAVTVAWK
ADGSPVKVGVETTKPSKQSNNKYAASSYLSLTPEQWKSHRSYSCRVTHEGSTVEKTVAPAECS
SEQ ID NO: 203
CAGTCTGCCCTAACTCAGCCTCGCTCAGTGTCCGCATCTCCTGGTCAGTCAGTCACCATCTCCTGC
ACTGGAACACACAATTATGTGTCCTGGTGTCAACAGAAACCGGGCCAAGCCCCCAAATTATTAAT
TTACGATTTCAATAAACGGCCCTCAGGGGTCTCTGATCGCTTCTCTGGCTCCACGTCTGGCAACAC
GGCCTCCCTGACCATCTCTGGACTCCAGGCTGACGATGAGGGTCATTATTTTTGTTGGGCGTTTGA
AAATATCGGCGGAGGGACCAAGCTGACCGTCCTGGGTCAGCCCAAGGCTGCCCCCTCGGTCACTC
TGTTCCCACCCTCCTCTGAGGAGCTTCAAGCCAACAAGGCCACACTGGTGTGTCTCGTAAGTGACT
TCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 204
>N49P7L11
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 205
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 206
LIGHT CHAIN
QCVLTQPRSVSGSPGQSVTISCTGT [symbol] VSWCQHHPGNAPKLLLY [symbol] KRPSGISDRFSGSRSGNTASLTISG
LOPEDEADYFC [symbol] FGGGTKVLVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 207
CAGTGTGTCTTGACTCAGCCTCGCTCAGTGTCCGGATCTCCTGGACAATCAGTCACCATCTCCTGC
ACTGGAACTCACAATTATGTCTCCTGGTGTCAACACCACCCAGGCAACGCCCCCAAATTATTACTT
TATGATTTCGACAAGCGGCCCTCAGGAATCTCTGATCGCTTCTCTGGCTCTAGGTCTGGCAACACG
GCCTCCCTGACCATCTCTGGCCTCCAGCCTGAGGATGAGGCCGATTACTTTTGTTGGGCCTTTGAA
GCCTTTGGCGGAGGGACCAAGGTGCTCGTCCTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 208
>N49P7.1L9
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPAVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 209
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCATCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 210
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 211
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 212
>N49P7.1L19
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 213
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCATCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 214
LIGHT CHAIN
QSALTQPRSVSATPGQSVTISCTGT [symbol] VSWCQQHPGRAPKLLIY [symbol] KRPSGVPDRFSGSGSGGTASLTITG
LQDDDEADYFC [symbol] FGGGTKLTVLRQPKAAPSVTLFPPSSEELQANKATLVCLISDFYPGAVTVAW
KADSSPVKAGVETTTPSKQSNNKYAASSYLSLTPEQWKSHRSYSCQVTHEGSTVEKTVAPTECS
SEQ ID NO: 215
CAGTCTGCCCTGACTCAGCCTCGCTCAGTGTCCGCAACTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTGGACTCCAGGATGACGATGAAGCGGACTATTTTTGTTGGGCCTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 216
>R49P7
HEAVY CHAIN
ADLVQSGAVVKKPGDSVRISCEAQ [symbol] IHWIRRAPGQGPEWMGW [symbol] NIPWKFQGRVSM
TRDTSIETAFLDLRGLKSDDTAVYYC [symbol] WGRGTAVTIQSASTKGPSVFPLAP
SSRSTSESTAALGCLVKDYFPEPVTVSWNSGSLTSGVHTFPAVLQSSGLYSLSSVVTVPSSSLGTQTYVC
NVNHKPSNTKVDKRVEIKTCGGGSKPPTCPPCPAPELLGGPSVFLFPPKPKDTLMISRTPEVTCVVVDV
SQEDPDVKFNWYVNGAEVHHAQTKPRETQYNSTYRVVSVLTVTHQDWLNGKEYTCKVSNKALPAPI
QKTISKDKGQPREPQVYTLPPSREELTKNQVSLTCLVKGFYPSDIVVEWESSGQPENTYKTTPPVLDSD
GSYFLYSKLTVDKSRWQQGNVFSCSVLHEALHSHYTQKSLSLSPGK
SEQ ID NO: 217
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCGACCAA
GGGCCCATCGGTCTTCCCCCTGGCGCCCTCCTCCAGGAGCACCTCCGAGAGCACAGCGGCCCTGG
GCTGCCTGGTCAAGGACTACTTCCCTGAACCCGTGACCGTGTCGTGGAACTCAGGCTCCCTGACCA
GCGGCGTGCACACCTTCCCGGCTGTCCTACAGTCCTCAGGGCTCTACTCCCTCAGCAGCGTGGTGA
CCGTGCCCTCCAGCAGCTTGGGCACCCAGACCTACGTCTGCAACGTAAACCACAAGCCCAGCAAC
ACCAAGGTGGACAAGAGAGTTGAGATAAAAACATGTGGTGGTGGCAGCAAACCTCCCACGTGCCC
ACCGTGCCCAGCACCTGAACTCCTGGGGGGACCGTCAGTCTTCCTCTTCCCCCCAAAACCCAAGGA
CACCCTCATGATCTCCCGGACCCCTGAGGTCACATGCGTGGTGGTAGACGTGAGCCAGGAAGACC
CCGATGTCAAGTTCAACTGGTACGTAAATGGCGCGGAGGTGCATCATGCCCAGACGAAGCCACGG
GAGACGCAGTACAACAGCACATATCGTGTGGTCAGCGTCCTCACCGTCACGCACCAGGACTGGCT
GAACGGCAAGGAGTACACGTGCAAGGTCTCCAACAAAGCCCTCCCGGCCCCCATCCAGAAAACCA
TCTCCAAAGACAAAGGGCAGCCCCGAGAGCCTCAGGTGTACACCCTGCCCCCGTCCCGGGAGGAG
CTGACCAAGAACCAGGTCAGCCTGACCTGCCTGGTCAAAGGCTTCTACCCCAGTGACATCGTCGT
GGAGTGGGAGAGCAGCGGGCAGCCGGAGAACACCTACAAGACCACCCCGCCCGTGCTGGACTCC
GACGGCTCCTACTTCCTCTACAGCAAGCTCACCGTGGACAAGAGCAGGTGGCAGCAGGGGAACGT
CTTCTCATGCTCCGTGCTGCATGAGGCTCTGCACAGCCACTACACGCAGAAGAGCCTCTCCCTGTC
CCCGGGTAAA
SEQ ID NO: 218
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVEVAWKA
DGSAVNAGVETTKPSKQSNNKYAASSYLSLTSDQWKSHKSYSCQVTHEGSTVEKTVAPAECS
SEQ ID NO: 219
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCGAGCGAGGAGCTTCAAGCCAACAAGGCCACACTAGTGTGTCTGATCAGTGACTTCTACCC
GGGAGCCGTGGAAGTGGCCTGGAAGGCAGATGGCAGCGCTGTCAACGCGGGAGTGGAGACCACC
AAACCCTCCAAACAGAGCAACAACAAGTACGCGGCCAGCAGCTACCTGAGCCTGACGTCCGACCA
GTGGAAGTCCCACAAGAGCTACAGCTGCCAGGTCACGCACGAAGGGAGCACCGTGGAGAAGACA
GTGGCCCCTGCAGAATGTTCA
SEQ ID NO: 220
>N49P7.2
HEAVY CHAIN
ADLVQSGAVVKKPGDSVRISCEAQ [symbol] IHWIRRAPGQGLEWMGW [symbol] NIPWQFQGRVSMT
RDTSIETAFLDLRGLKSDDTALYYC [symbol] WGRGTAVTVHSPSTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 221
GCGGACTTGGTGCAGTCTGGGGCTGTGGTGAAGAAGCCTGGGGACTCAGTGAGGATCTCCTGTGA
GGCTCAAGGATACAAATTTCCTGACTACATCATTCACTGGATTCGACGCGCCCCTGGACAAGGCCT
TGAGTGGATGGGGTGGATTAATCCAATGGGCGGACAAGTAAACATTCCATGGCAGTTTCAGGGCA
GGGTCTCCATGACCCGGGACACGTCCATCGAAACAGCATTTCTGGACTTAAGAGGACTAAAGTCT
GACGACACGGCCCTCTATTATTGCGTGAGAGATCGAAGTAATGGATCGGGAAGGCGATTCGAGTC
CTCCAATTGGTTCCTCGATCTGTGGGGCCGCGGCACTGCGGTCACTGTTCATTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 222
LIGHT CHAIN
QSALTQPRSVSASPGQSVTISCTGT [symbol] VSWCQHHPGRAPKLLIY [symbol] KRPSGVPDRFSGSGSGGTASLTISG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 223
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCAGTGGACTCCAGGATGACGATGACGCCGAATATTTTTGTTGGGCGTATGAA
GCTTTTGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 224
>N49P11
HEAVY CHAIN
SAELVQSGAWKKPGTSVKVSCQAY [symbol] IHWLRQAPGQGLEWMGW [symbol] NYAQNFQGRVS
MTRDIYRETAFLEVRDLKTDDTGTYYC [symbol] WGRGTWIRVAPASTKGPSVFPL
APSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 225
TCGGCGGAATTGGTGCAATCTGGGGCTGTGGTGAAGAAGCCTGGGACCTCCGTGAAGGTCTCTTG
TCAGGCTTATGGATACACTTTTACCGACTACCTTATTCATTGGCTTCGACAGGCCCCTGGACAAGG
ACTTGAATGGATGGGATGGATGAATCCTGTTTATGGACAAGTAAATTATGCCCAAAACTTTCAGG
GCAGGGTCTCCATGACCAGGGACATTTACAGGGAAACAGCATTTCTAGAGGTGCGCGACCTGAAG
ACTGACGACACAGGCACTTATTATTGTGTGAGAGACACAGGCGACGGTTCGCGGAGACACTTTGA
CTCCATCAATTGGTTTCTCGATCTTTGGGGCCGCGGGACATGGATAAGGGTCGCCCCAGCCTCCAC
CAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCACAAGCCCAGC
AACACCAAGGTGGACAAGAGAGTTGAGCCCAAATCTTGTGACAAAACTCACACATGCCCACCGTG
CCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGA
GCAGTACAACAGCACGTACCGGGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCG
GGTAAA
SEQ ID NO: 226
LIGHT CHAIN
QCVLTQPRSVSGSPGQSVTISCTGT [symbol] VSWCQHHPGNAPKLLLY [symbol] KRPSGISDRFSGSRSGNTASLTISG
LQPEDEADYFC [symbol] FGGGTKVLVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 227
CAGTGTGTCTTGACTCAGCCTCGCTCAGTGTCCGGATCTCCTGGACAATCAGTCACCATCTCCTGC
ACTGGAACTCACAATTATGTCTCCTGGTGTCAACACCACCCAGGCAACGCCCCCAAATTATTACTT
TATGATTTCGACAAGCGGCCCTCAGGAATCTCTGATCGCTTCTCTGGCTCTAGGTCTGGCAACACG
GCCTCCCTGACCATCTCTGGCCTCCAGCCTGAGGATGAGGCCGATTACTTTTGTTGGGCCTTTGAA
GCCTTTGGCGGAGGGACCAAGGTGCTCGTCCTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 228
>N49P18
HEAVY CHAIN
ADLVQSGAVMKKPGDSVRISCEAR [symbol] IHWIRRAPGQGLEWMGW [symbol] NIPWNFQGRVSMT
RDTSIETAFLDLRGLKSDDTGLYYC [symbol] WGRGTVVTVHSPSTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 229
GCGGACTTGGTGCAGTCTGGGGCTGTGATGAAGAAGCCTGGGGACTCAGTGAGAATCTCCTGTGA
GGCTCGAGGATACACATTCACTGACTACGTCATTCACTGGATTCGACGCGCCCCTGGACAAGGCCT
TGAATGGATGGGGTGGATTGATCCACCTTATGGACAAGTAAATATTCCATGGAATTTTCAGGGCA
GGGTCTCCATGACCCGGGACACGTCCATCGAAACAGCATTTCTGGACTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGGTCACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 230
LIGHT CHAIN
QSALTQPRSVSASPGQSVTISCTGT [symbol] VSWCQHHPGRAPKLLIY [symbol] KRPSGVPDRFSGSGSGGTASLTISG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 231
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAGTTATTAATT
TATGACTTCAATAAGAGGCCCTCAGGGGTCCCTGATCGCTTCTCTGGCTCCGGGTCTGGCGGCACG
GCCTCCCTAACCATCAGTGGACTCCAGGATGACGATGACGCCGAATATTTTTGTTGGGCATATGAA
GCTTTCGGCGGAGGGACCAAGTTGACTGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 232
>N49P18.2
HEAVY CHAIN
ADLVQSGAVMKKPGDSVRISCEAR [symbol] IHWIRRAPGQGLEWMGW [symbol] NIPWNFQGRVSMT
RDTSIETAFLDLRGLKSDDTGLYYC [symbol] WGRGTVVTVHSPSTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 233
GCGGACTTGGTGCAGTCTGGGGCTGTGATGAAGAAGCCTGGGGACTCAGTGAGAATCTCCTGTGA
GGCTCGAGGATACACATTCACTGACTACGTCATTCACTGGATTCGACGCGCCCCTGGACAAGGCCT
TGAATGGATGGGGTGGATTGATCCACCTTATGGACAAGTAAATATTCCATGGAATTTTCAGGGCA
GGGTCTCCATGACCCGGGACACGTCCATCGAAACAGCATTTCTGGACTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGGTCACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 234
LIGHT CHAIN
QSALTQPRSVSASPGQSVTISCTGT [symbol] VSWCQHHPGRAPKLLIY [symbol] KRPSGVPDRFSGSGSGGTASLTISG
LQDDDDAEYFC [symbol] FGGGTKLTVLRQPKAAPSVTLFPPSSEELQANKATLVCLVSDFYPGAVTVAW
KADGSPVKVGVETTKPSKQSNNKYAASSYLSLTPEQWKSHRSYSCRVTHEGSTVEKTVAPAECS
SEQ ID NO: 235
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAGTTATTAATT
TATGACTTCAATAAGAGGCCCTCAGGGGTCCCTGATCGCTTCTCTGGCTCCGGGTCTGGCGGCACG
GCCTCCCTAACCATCAGTGGACTCCAGGATGACGATGACGCCGAATATTTTTGTTGGGCATATGAA
GCTTTCGGCGGAGGGACCAAGTTGACTGTACTTCGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 236
>N49P18.1
HEAVY CHAIN
ADLVQSGAVVKKPGDSVRISCEAQ [symbol] IHWIRRAPGQGLEWMGW [symbol] NIPWNFQGRVSMT
RDTSIETAFLDLRGLKSDDTGLYYC [symbol] WGRGTVVTVHSPSYKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 237
GCGGACTTGGTGCAGTCTGGGGCTGTGGTGAAGAAGCCTGGGGACTCAGTGAGAATCTCCTGTGA
GGCTCAAGGATACACATTCACTGACTACGTCATTCACTGGATTCGACGCGCCCCTGGACAAGGCCT
TGAATGGATGGGGTGGATTAATCCAGGTTATGGACAAGTAAATATTCCATGGAACTTTCAGGGCA
GGGTCTCCATGACCCGAGACACGTCCATCGAAACAGCATTTCTGGACTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGGTCACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 238
LIGHT CHAIN
QSALTQPRSMSASPGQSVTISCTGT [symbol] VSWCQHHPGRPPKLLIY [symbol] KRPSGVPDRFSGSGSGGTASLTISG
LQDDDDAEYIC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 239
CAGTCTGCCCTGACTCAGCCTCGCTCAATGTCCGCATCTCCTGGGCAGTCCGTCACCATTTCCTGC
ACTGGAACCCACAATTTGGTCTCTTGGTGTCAACATCACCCAGGCAGACCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCAGTGGACTCCAGGATGACGATGACGCCGAATACATTTGTTGGGCATATGA
AGCTTTCGGCGGAGGGACCAAGTTGACCGTACTTCGTCAGCCCAAGGCTGCCCCCTCGGTCACTCT
GTTCCCGCCCTCCTCTGAGGAGCTTCAAGCCAACAAGGCCACACTGGTGTGTCTCATAAGTGACTT
CTACCCGGGAGCCGTGACAGTGGCTTGGAAAGCAGATAGCAGCCCCGTCAAGGCGGGAGTGGAG
ACCACCACACCCTCCAAACAAAGCAACAACAAGTACGCGGCCAGCAGCTATCTGAGCCTGACGCC
TGAGCAGTGGAAGTCCCACAGAAGCTACAGCTGCCAGGTCACGCATGAAGGGAGCACCGTGGAG
AAGACAGTGGCCCCTACAGAATGTTCA
SEQ ID NO: 240
>N49P19
HEAVY CHAIN
ADLVQSGAWKNAGASVRVSCEAY [symbol] IHWVRQAPGQGFEWMGY [symbol] NIARKFQGRLSLS
RDRSSETSFLDLSGLRSDDSAVYYC [symbol] WGRGTRVSIFSASTKGPSVFPLAPSS
KSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 241
GCGGACTTGGTGCAGTCTGGGGCTGTGGTGAAAAATGCTGGGGCCTCAGTGAGGGTCTCCTGTGA
GGCTTATGGATACACATTCGTGGACTACTTCATTCATTGGGTCCGACAGGCCCCTGGACAAGGCTT
TGAATGGATGGGATACATGGATCCCTTGAACGGGCGCCCAAACATTGCGCGAAAATTTCAGGGCA
GGCTCTCCCTGAGTCGAGATAGGTCCAGCGAAACTTCATTTCTGGACTTAAGTGGACTGAGGTCTG
ACGACTCGGCCGTCTATTATTGTGTGAGAGACAAGAGTAATGGATCGGGCAGACGGTTTGACTCG
TCTAATTGGTTTCTCGATCTGTGGGGCCGTGGAACCCGGGTCAGTATTTTCTCAGCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
SEQ ID NO: 242
LIGHT CHAIN
QSALTQPRSVSATPGQSVTISCTGT [symbol] VSWCQQHPGRAPKLLIY [symbol] KRPSGVPDRFSGSGSGGTASLTITG
LQDDDEADYFC [symbol] FGGGTKLTVLRQPKAAPSVTLFPPSSEELQANKATLVCLISDFYPGAVTVAW
KADSSPVKAGVETTTPSKQSNNKYAASSYLSLTPEQWKSHRSYSCQVTHEGSTVEKTVAPTECS
SEQ ID NO: 243
CAGTCTGCCCTGACTCAGCCTCGCTCAGTGTCCGCAACTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTGGACTCCAGGATGACGATGAAGCGGACTATTTTTGTTGGGCCTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 244
>N49P37
HEAVY CHAIN
ADLVQSGAVVKKPGDSVRVSCEAY [symbol] IHWIRRAPGRGLEWMGW [symbol] NIPWNFQGRVSM
TRDTSIETAFLDLRGLRSDDTAVYYC [symbol] WGRGTAVTISSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 245
GCGGACTTGGTGCAGTCTGGGGCTGTGGTGAAGAAGCCTGGGGACTCAGTGAGGGTCTCCTGTGA
GGCTTATGGATACACATTCAGTGACTACATCATTCATTGGATTCGACGGGCCCCTGGACGAGGCCT
TGAATGGATGGGATGGATGAATCCGATGGGCGGACAAGTGAATATTCCGTGGAACTTTCAGGGGA
GAGTCTCCATGACCCGGGACACGTCCATCGAAACAGCATTTCTGGACTTAAGAGGACTGAGGTCT
GACGACACGGCCGTCTATTACTGTGTGAGAGATCGCAGCAATGGATCGGGCAAGCGATTTGAGTC
CTCCAATTGGTTCCTCGATCTGTGGGGCCGCGGGACCGCGGTCACTATTTCCTCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 246
LIGHT CHAIN
QSALTQPRSVSAAPGQSVTISCTGT [symbol] VSWCQHHPGRAPKLLIY [symbol] KRPSGVPDRFSGSGSGGTASLTITG
LQDDDEAEYFC [symbol] FGGGTKLTVLGQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 247
CAGTCTGCCCTGACTCAGCCTCGCTCAGTGTCCGCAGCTCCTGGACAGTCCGTCACCATTTCCTGC
ACTGGAACCCACAATTTGGTTTCTTGGTGTCAACATCACCCAGGCAGAGCCCCCAAGTTATTAATT
TATGACTTCAATAAGAGACCCTCAGGTGTCCCTGATCGTTTCTCTGGCTCCGGGTCTGGCGGCACG
GCCTCCCTAACCATCACTGGACTCCAGGATGACGATGAGGCTGAATATTTTTGTTGGGCGTATGAA
GTTTTTGGCGGAGGGACCAAGTTGACCGTGCTTGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 248
>N49P38
HEAVY CHAIN
ADLVQSGAVVKTPGASVRVSCEAY [symbol] IHWVRQAPGQGFEWLGY [symbol] NIARKFQGRLSLSRD
TSIETSFLDLSGLRSDDSAVYYC [symbol] WGRGTRVSISSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 249
GCGGACTTGGTGCAGTCTGGGGCTGTGGTGAAGACGCCTGGGGCCTCAGTGAGGGTCTCCTGTGA
GGCTTATGGATACACATTCATTGACTACATCATTCATTGGGTCCGACAGGCCCCTGGACAAGGTTT
TGAATGGCTGGGATACATTGATCCTATGAACGGGCGCCCAAACATTGCGCGAAAATTTCAGGGCA
GGCTCTCCCTGAGCCGGGATACGTCCATCGAAACATCATTTCTGGACTTAAGTGGACTGAGGTCTG
ACGACTCGGCCGTCTATTATTGTGTGAGAGACAAGAGTAATGGATCGGGCAAACGATTTGACTCC
TCTAATTGGTTTCTCGATCTGTGGGGCCGTGGAACGCGGGTCAGCATTTCTTCAGCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
SEQ ID NO: 250
LIGHT CHAIN
QSALTQPRSVSAAPGQSVTISCTGT [symbol] VSWCQQHPGRAPKLLIY [symbol] KRPSGVPDRFSGSGSGGTASLTITR
LQDDDDADYFC [symbol] FGGGTKLTVLGQPKAAPSVTLFPPSSEELQANKATLVCLISDFYPGAVTVAW
KADSSPVKAGVETTTPSKQSNNKYAASSYLSLTPEQWKSHRSYSCQVTHEGSTVEKTVAPTECS
SEQ ID NO: 251
CAGTCTGCCCTGACTCAGCCTCGCTCAGTGTCCGCAGCTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTAGACTCCAGGATGACGATGACGCTGACTATTTTTGTTGGGCGTATGAT
GCTTTTGGCGGAGGGACCAAGTTGACCGTCCTGGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 252
>N49P38.1
HEAVY CHAIN
ADLVQSGAVVKTPGASVRVSCEAY [symbol] IHWVRQAPGQGFEWLGY [symbol] NIARKFQGRLSLSRD
TSIETSFLDLSGLRSDDSAVYYC [symbol] WGRGTRVSISSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 253
GCGGACTTGGTGCAGTCTGGGGCTGTGGTGAAGACGCCTGGGGCCTCAGTGAGGGTCTCCTGTGA
GGCTTATGGATACACATTCATTGACTACATCATTCATTGGGTCCGACAGGCCCCTGGACAAGGTTT
TGAATGGCTGGGATACATTGATCCTATGAACGGGCGCCCAAACATTGCGCGAAAATTTCAGGGCA
GGCTCTCCCTGAGCCGGGATACGTCCATCGAAACATCATTTCTGGACTTAAGTGGACTGAGGTCTG
ACGACTCGGCCGTCTATTATTGTGTGAGAGACAAGAGTAATGGATCGGGCAAACGATTTGACTCC
TCTAATTGGTTTCTCGATCTGTGGGGCCGTGGAACGCGGGTCAGCATTTCTTCAGCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
SEQ ID NO: 254
LIGHT CHAIN
QSALTQPRSVSAAPGQSVTISCTGT [symbol] VSWCQQHPGRAPKLLIY [symbol] KRPSGVPDRFSGSGSGGTASLTITR
LQDDDDADYFC [symbol] FGGGTKLTVLRQPKAAPSVTLFPPSSEELQANKATLVCLISDFYPGAVTVAW
KADSSPVKAGVETTTPSKQSNNKYAASSYLSLTPEQWKSHRSYSCQVTHEGSTVEKTVAPTECS
SEQ ID NO: 255
CAGTCTGCCCTGACTCAGCCTCGCTCAGTGTCCGCAGCTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTAGACTCCAGGATGACGATGACGCTGACTATTTTTGTTGGGCGTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 256
>N49P55
HEAVY CHAIN
ADLVQSGAVVKKPGASVRVSCEAY [symbol] IHWIRQAPGQGLEWMGW [symbol] NIPWKFQGRVSM
TRDTSIETAFLDLSGLTSDDTAVYYC [symbol] WGRGTPVTISSPSTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 257
GCGGACTTGGTGCAGTCTGGGGCTGTGGTGAAGAAGCCTGGGGCCTCAGTGAGGGTCTCCTGTGA
GGCTTATGGATACACATTCACTGACTACATCATTCATTGGATTCGACAGGCCCCTGGACAAGGCCT
TGAATGGATGGGATGGATGAATCCTATGGGCGGGCGCACAAATATTCCGTGGAAATTTCAGGGCA
GGGTCTCCATGACCCGGGACACGTCCATCGAAACAGCATTTCTGGACTTAAGTGGACTAACGTCTG
ACGACACGGCCGTCTATTATTGCGTGAGAGACAAGAGTAATGGATCGGGCAAACGATTTGACTCC
TCTAATTGGTTCCTCGATCTGTGGGGCCGCGGAACCCCGGTCACTATTTCCTCACCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
SEQ ID NO: 258
LIGHT CHAIN
QSALTQPRSVSAAPGQSVTISCTGT [symbol] VSWCQQHPGRAPKLLIY [symbol] KRPSGVPDRFSGSGSGGTASLSITG
LQDDDEAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 259
CAGTCTGCCCTGACTCAGCCTCGCTCAGTGTCCGCAGCTCCTGGACAGTCCGTCACCATTTCCTGC
ACTGGAACCCACAATTTGGTCTCTTGGTGTCAACAACACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTAAGTATCACTGGACTCCAGGATGACGATGAAGCTGAATATTTTTGTTGGGCGTATGAA
GCTTTTGGCGGAGGGACCAAGTTGACCGTCCTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 260
>N49P56
HEAVY CHAIN
ADLVQSGAVVKKPGASVRVSCEAY [symbol] IHWVRQAPGQGFEWMGY [symbol] NIARKFQGRLSLS
RDTSIETSFLDLSGLRSDDSAVYYC [symbol] WGRGTRVSISSASTKGPSVFPLAPSS
KSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 261
GCGGACTTGGTGCAGTCTGGGGCTGTGGTGAAGAAGCCTGGGGCCTCAGTGCGGGTCTCCTGTGA
GGCTTATGGATATACATTCGTTGACTACCTCATTCATTGGGTCCGACAGGCCCCCGGACAAGGTTT
TGAATGGATGGGATACATGGATCCTATGAACGGGCGCCCAAATATTGCGCGAAAATTTCAGGGCA
GGCTCTCCCTGAGCCGAGATACGTCCATCGAAACATCATTTCTGGACTTAAGTGGACTGAGGTCTG
ACGACTCGGCCGTCTATTATTGTGTGAGAGACAAGAGTGGTGGATCGGGCAAACTATTTGACTCCT
CTAATTGGTTTCTCGATCTGTGGGGCCGTGGAACCCGGGTCAGCATTTCTTCAGCCTCCACCAAGG
GCCCATCGGTCTTCCCCCTGGCACCCTCCTCCAAGAGCACCTCTGGGGGCACAGCGGCCCTGGGCT
GCCTGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGTGACC
GTGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCAG
CACCTGAACTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGT
ACAACAGCACGTACCGGGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAAACCATCTCCAAAGC
CAAAGGGCAGCCCCGAGAACCACAGGTGTACACCCTGCCCCCATCCCGGGAGGAGATGACCAAG
AACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCT
TCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAA
A
SEQ ID NO: 262
LIGHT CHAIN
QSALTQPRSVSAAPGQSVTISCTGT [symbol] VSWCQQHPGRAPKLLIY [symbol] KRPSGVPDRFSGSGSGGTASLTITG
LQDDDDADYFC [symbol] FGGGTKLTVLRQPKAAPSVTLFPPSSEELQANKATLVCLISDFYPGAVTVAW
KADSSPVKAGVETTTPSKQSNNKYAASSYLSLTPEQWKSHRSYSCQVTHEGSTVEKTVAPTECS
SEQ ID NO: 263
CAGTCTGCCCTGACTCAGCCTCGCTCAGTGTCCGCAGCTCCTGGACAGTCAGTCACCATCTCCTGC
ACCGGAACTCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTGGACTCCAGGATGACGATGACGCTGATTATTTTTGTTGGGCGTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 264
>N49P57
HEAVY CHAIN
ADLVQSGAVVKKPGDSVRISCEAQ [symbol] IHWIRRAPGQGLEWMGW [symbol] NIPWNFQGRVSMT
RDTSIETAFLELRGLKSDDTGLYYC [symbol] WGRGTVITVHSPSTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 265
GCGGACTTGGTGCAGTCTGGGGCTGTGGTGAAGAAGCCTGGGGACTCAGTGAGAATCTCCTGTGA
GGCTCAAGGATACACATTCACTGACTACGTCATTCACTGGATTCGACGGGCCCCTGGACAAGGCC
TTGAATGGATGGGGTGGATTAATCCAGGTTATGGACAAGTAAATATTCCATGGAACTTTCAGGGC
AGGGTCTCCATGACCCGAGACACGTCCATCGAAACAGCTTTTCTGGAGTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGATTACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 266
LIGHT CHAIN
QSALTQPRSMSASPGQSVTISCTGT [symbol] VSWCQHHPGRPPKLLIY [symbol] KRPSGVPDRFSGSGSGGTASLTITG
LQDDDDAEYIC [symbol] FGGGTKLTILRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 267
CAGTCTGCCCTGACTCAGCCTCGCTCAATGTCCGCATCTCCTGGGCAGTCCGTCACCATTTCCTGC
ACTGGAACCCACAATTTGGTCTCTTGGTGTCAACATCACCCAGGCAGACCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCACTGGACTCCAGGATGACGATGACGCCGAATACATTTGTTGGGCATATGAA
GCTTTCGGCGGAGGGACCAAGTTGACCATACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 268
>N49P58
HEAVY CHAIN
ADLVQSGAVVKKPGDSVRISCEAQ [symbol] IHWIRRAPGQGLEWMGW [symbol] NIPRNFQGRVSMT
RDTFRETAYLELRGLQSDDKGLYYC [symbol] WGRGTVVNVQSPSTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 269
GCGGACTTGGTGCAGTCTGGGGCTGTGGTGAAGAAGCCTGGGGACTCAGTGAGAATCTCCTGTGA
GGCTCAGGGATATACATTCACCGACTACGTCATTCATTGGATTCGACGGGCCCCTGGACAAGGCCT
TGAATGGATGGGGTGGATGGATCCAAGTTATGGACAAGTCAATATTCCACGGAACTTTCAGGGCA
GGGTCTCCATGACCCGGGACACGTTCAGGGAAACAGCATATCTGGAATTAAGAGGTCTACAGTCT
GACGACAAGGGCCTCTATTATTGTGTGAGAGATCGAAGTCACGGATCGGGAAGGCAATTCGAGTC
CTCCAACTGGTTCCTCGATCTGTGGGGCCGCGGCACTGTGGTCAATGTTCAGTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 270
LIGHT CHAIN
QSALTQPRSVSASPGQSVTISCTGT [symbol] VSWCQHHPGRPPKLLIY [symbol] KRASGVPDRFSGSGSGGTASLTISG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 271
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCTTCAGGGGTCCCTGATCGCTTCTCTGGCTCCGGGTCTGGCGGCACG
GCCTCCCTGACCATTAGTGGACTCCAGGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 272
>N49P59
HEAVY CHAIN
ADLVQSGAVVKKPGDSLRISCEAQ [symbol] IHWIRRAPGQGLEWMGW [symbol] NIPRNFQGRVSM
TRDMYIETAFLDLRGLKSDDTGLYYC [symbol] WGRGTVVTVQSPSTKGPSVFPLA
PSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 273
GCGGACTTGGTGCAGTCTGGGGCTGTGGTGAAGAAGCCTGGGGACTCACTGAGAATCTCCTGTGA
GGCTCAAGGATACACATTCACTGACTACGTCATTCACTGGATTCGACGGGCCCCTGGACAAGGCC
TTGAATGGATGGGATGGATGGATCCAAGTTTTGGACAAATGAACATTCCACGGAACTTTCAGGGC
AGGGTCTCCATGACCCGTGACATGTACATCGAAACAGCATTTCTGGACTTAAGAGGTCTAAAGTCT
GACGACACGGGCCTCTATTATTGCGTGAGAGATCGAAGTCATGGATCGGGAAGGCTATTCGAGTC
CTCCAATTGGTTCCTCGATCTGTGGGGCCGCGGCACTGTGGTCACTGTTCAGTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 274
LIGHT CHAIN
QSALTQPRSVSASPGQSVTISCTGT [symbol] VSWCQHHPGRPPKLLIY [symbol] KRASGVPDRFSGSGSGGTASLTISG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 275
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCTTCAGGGGTCCCTGATCGCTTCTCTGGCTCCGGGTCTGGCGGCACG
GCCTCCCTGACCATTAGTGGACTCCAGGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 276
>N49P73
HEAVY CHAIN
ADLVQSGAVVKKPGDSVRISCEAQ [symbol] IHWIRRAPGQGLEWMGL [symbol] NIPRKFQGRVSMT
RDTSMETAFLDFRGLNFDDTGLYYC [symbol] WGRGTVVTVQSPSTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 277
GCGGACTTGGTGCAGTCTGGGGCTGTGGTGAAGAAGCCTGGGGACTCAGTGAGAATCTCCTGTGA
GGCTCAAGGATACAGATTCACTGACTACGTCATTCATTGGATTCGACGGGCCCCTGGACAAGGCCT
TGAATGGATGGGGTTGATGGATCCAAGTTTTGGACGAATGAATATTCCACGGAAATTTCAGGGCA
GGGTCTCCATGACCCGGGACACGTCCATGGAAACAGCATTTCTGGACTTCAGAGGTCTAAATTTTG
ACGACACGGGCCTCTATTATTGCGTGAGAGATCGAAGTCATGGATCGGGAAGACTATTCGAGTCC
TCCAATTGGTTCCTCGATCTGTGGGGCCGCGGCACTGTGGTCACTGTTCAGTCACCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
SEQ ID NO: 278
LIGHT CHAIN
QSALTQPRSVSASPGQSVTISCTGT [symbol] VSWCQHHPGRPPKLLIY [symbol] KRASGVPDRFSGSGSGGTASLTISG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 279
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCATCAGGGGTCCCTGATCGCTTCTCTGGCTCCGGGTCTGGCGGCACG
GCCTCCCTGACCATCAGTGGACTCCAAGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 280
>N49P74
HEAVY CHAIN
ADLVQSGAVVKKPGDSVRISCEAQ [symbol] IHWIRRAPGQGLEWMGL [symbol] NIPRKFQGRVSMT
RDTSIETAFLDLRGLKSDDTGLYYC [symbol] WGRGTVVTVQSPSTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 281
GCGGACTTGGTGCAGTCTGGGGCTGTGGTGAAGAAGCCTGGGGACTCAGTGAGAATTTCCTGTGA
GGCTCAAGGATACACATTCATTGACTACGTCATTCACTGGATTCGACGGGCCCCTGGACAAGGCCT
TGAATGGATGGGGTTGATGGATCCAACTTATGGACGAATGAATATTCCACGGAAGTTTCAGGGCA
GGGTCTCCATGACCCGGGACACGTCCATCGAAACAGCATTTCTGGACTTAAGAGGTCTAAAATCT
GACGACACGGGCCTCTATTATTGCGTGAGAGATCGAAGTCATGGATCGGGAAGGCTATTCGAGTC
CTCCAACTGGTTCCTGGATCTGTGGGGCCGCGGCACTGTGGTCACTGTTCAGTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 282
LIGHT CHAIN
QSALTQPRSVSASPGQSVTISCTGT [symbol] VSWCQHHPGRPPKLLIY [symbol] KRASGVPDRFSGSGSGGTASLTISG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 283
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCTTCAGGGGTCCCTGATCGCTTCTCTGGCTCCGGGTCTGGCGGCACG
GCCTCCCTGACCATCAGTGGACTCCAAGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 284
>N49P75
HEAVY CHAIN
ADLVQSGAVVKKPGDSVRISCEAQ [symbol] IHWIRRAPGQGLEWMGW [symbol] NIPWNFQGRVSM
TRDTSIETAFLDLRGLKSDDTAVYYC [symbol] WGRGTAVTIHSPSTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 285
GCGGACTTGGTGCAGTCTGGGGCTGTGGTGAAGAAGCCTGGGGACTCAGTGAGGATCTCCTGTGA
GGCTCAAGGATACAGATTTCTTGACTACATCATTCACTGGATTCGACGAGCCCCTGGACAAGGCCT
TGAATGGATGGGATGGATGAATCCAATGGGCGGACAAGTAAACATTCCATGGAACTTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGCGGGACTGCGGTCACTATTCATTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 286
LIGHT CHAIN
QSALTQPRSVSASPGQSVTISCTGT [symbol] VSWCQHHPGRAPKLLIY [symbol] KRPSGVPDRFSGSGSGGTASLTITG
LQDDDDAEYFC [symbol] FGGGTKLTVLRQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAPTECS
SEQ ID NO: 287
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCACTGGACTCCAGGATGACGATGACGCCGAATATTTTTGTTGGGCGTATGAA
GCTTTTGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 288
>N49P75.1
HEAVY CHAIN
ADLVQSGAVVKKPGDSVRISCEAQ [symbol] IHWIRRAPGQGLEWMGW [symbol] NIPWNFQGRVSM
TRDTSIETAFLDLRGLKSDDTAVYYC [symbol] WGRGTAVTIHSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 289
GCGGACTTGGTGCAGTCTGGGGCTGTGGTGAAGAAGCCTGGGGACTCAGTGAGGATCTCCTGTGA
GGCTCAAGGATACAGATTTCTTGACTACATCATTCACTGGATTCGACGAGCCCCTGGACAAGGCCT
TGAATGGATGGGATGGATGAATCCAATGGGCGGACAAGTAAACATTCCATGGAACTTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGCGGGACTGCGGTCACTATTCATTCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 290
LIGHT CHAIN
QSALTQPRSVSASPGQSVTISCTGT [symbol] VSWCQHHPGRAPKLLIY [symbol] KRPSGVPDRFSGSGSGGTASLTITG
LQDDDDAEYFC [symbol] FGGGTKLTVLGQPKAAPSVTLFPPSSEELQANKATLVCLISDFYPGAVTVAW
KADSSPVKAGVETTTPSKQSNNKYAASSYLSLTPEQWKSHRSYSCQVTHEGSTVEKTVAPTECS
SEQ ID NO: 291
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCACTGGACTCCAGGATGACGATGACGCCGAATATTTTTGTTGGGCGTATGAA
GCTTTTGGCGGAGGGACCAAGTTGACCGTACTTGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
SEQ ID NO: 292
>N49P9
HEAVY CHAIN
HVQLVQSGGGVKKIGAAVRISCEVT [symbol] INWVRQAPGQGLEWMGW [symbol] NYSWRFEGRVT
MTRDMDTETAFMELRGLRVDDTAVYYC [symbol] WGQGVRVVVSSPSTKGPSVFPLAPSSKSTS
GGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 293
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGCGAGGGGACCCTCTGGGGAAAATTATCCTTTTCACT
ATTGGGGCCAGGGTGTCCGAGTGGTCGTCTCGTCACCCTCCACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
SEQ ID NO: 294
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 295
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 296
>N49P9.1
HEAVY CHAIN
HVQLVQSGGGVKKIGAAVRISCEVT [symbol] INWVRQAPGQGLEWMGW [symbol] NYSWRFEGRVT
MTRDMDTETAFMELRGLRVDDTAVYYC [symbol] WGQGVRVVVSSPSTKGPSVFPLAPSSKSTS
GGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 297
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGCGAGGGGACCCTCTGGGGAAAATTATCCTTTTCACT
ATTGGGGCCAGGGTGTCCGAGTGGTCGTCTCGTCACCCTCCACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
SEQ ID NO: 298
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
SEQ ID NO: 299
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCGCCCTCCTCTGAGGAGCTTCAAGCCAACAAGGCCACACTGGTGTGTCTCATAAGTGAC
TTCTACCCGGGAGCCGTGACAGTGGCTTGGAAAGCAGATAGCAGCCCCGTCAAGGCGGGAGTGGA
GACCACCACACCCTCCAAACAAAGCAACAACAAGTACGCGGCCAGCAGCTATCTGAGCCTGACGC
CTGAGCAGTGGAAGTCCCACAGAAGCTACAGCTGCCAGGTCACGCATGAAGGGAGCACCGTGGA
GAAGACAGTGGCCCCTACAGAATGTTCA
SEQ ID NO: 300
>N49P9.2
HEAVY CHAIN
HVQLVQSGGGVKKIGAAVRISCEVT [symbol] INWVRQAPGQGLEWMGW [symbol] NYSWRFEGRVT
MTRDMDTETAFMELRGLRVDDTAVYYC [symbol] WGQGVRVVVSSASTKGPSVFPLAPSSKSTS
GGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 301
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGCGAGGGGACCCTCTGGGGAAAATTATCCTTTTCACT
ATTGGGGCCAGGGTGTCCGAGTGGTCGTCTCGTCATCCTCCACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
SEQ ID NO: 302
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLGQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 303
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAGGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 304
>N49P9i7
HEAVY CHAIN
HVQLVQSGGGVKKIGAAVRISCEVT [symbol] INWVRQAPGQGLEWMGW [symbol] NYSWRFEGRVT
MTRDMDTETAFMELRGLRVDDTAVYYC [symbol] WGQGVRVVVSSPSTKGPSVFPLAPSSK
STSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 305
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGTGAGAGATCGCAGTAATGGATCGGGAAAGCGATTC
GAGTCCTCCAATTGGGGCCAGGGTGTCCGAGTGGTCGTCTCGTCACCCTCCACCAAGGGCCCATCG
GTCTTCCCCCTGGCACCCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGACCGTGCCCTC
CAGCAGCTTGGGCACCCAGACCTACATCTGCAACGTGAATCACAAGCCCAGCAACACCAAGGTGG
ACAAGAGAGTTGAGCCCAAATCTTGTGACAAAACTCACACATGCCCACCGTGCCCAGCACCTGAA
CTCCTGGGGGGACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAGCAGTACAACAGC
ACGTACCGGGTGGTCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCCAAAGCCAAAGGGC
AGCCCCGAGAACCACAGGTGTACACCCTGCCCCCATCCCGGGAGGAGATGACCAAGAACCAGGTC
AGCCTGACCTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGTAAA
SEQ ID NO: 306
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 307
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 308
>N49P9i7H1
HEAVY CHAIN
HVQLVQSGGGVKKIGAAVRISCEVT [symbol] INWVRQAPGQGLEWMGW [symbol] NYSWRFEGRVT
MTRDMDTETAFMELRGLRVDDTAVYYC [symbol] WGQGVRVVVSSPSTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 309
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGTGAGAGATCGCAGTAATGGATCGGGAAAGCGATTC
GAGTCCTCCAATTGGTTCCTCGATCTGTGGGGCCAGGGTGTCCGAGTGGTCGTCTCGTCACCCTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 310
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 311
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 312
>N49P9i7H2
HEAVY CHAIN
HVQLVQSGGGVKKIGAAVRISCEVT [symbol] INWVRQAPGQGLEWMGW [symbol] NYSWRFEGRVT
MTRDMDTETAFMELRGLRVDDTAVYYC [symbol] WGRGTAVTIQSSSTKGPSVFPL
APSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 313
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGTGAGAGATCGCAGTAATGGATCGGGAAAGCGATTC
GAGTCCTCCAATTGGTTCCTCGATCTGTGGGGCCGTGGGACTGCGGTCACAATTCAATCATCCTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 314
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLRQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 315
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTACGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 316
>N49P22
HEAVY CHAIN
HIQLLQSGPQVKKSGDTVRISCETS [symbol] IHWVRQTPEKRLRWMGW [symbol] NYAPEFQGRIRMT
RDTFIDTVYVDLSGLTPADTAYYYC [symbol] WGHGTRVTVFSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 317
CACATTCAGTTGCTACAGTCGGGGCCTCAAGTTAAGAAGTCTGGGGACACAGTGAGAATCTCCTG
TGAGACCTCTGGATATAACTTCGTCGACTCCCGTATCCACTGGGTCCGACAGACCCCGGAAAAAC
GTCTCAGATGGATGGGCTGGATCAATCCTCTCCAAGGTGGTGTGAATTACGCGCCGGAATTTCAGG
GCAGAATCAGGATGACCAGGGACACATTTATAGACACAGTTTACGTGGACCTGAGCGGACTGACA
CCGGCCGACACGGCCTATTATTACTGCGCGCGAGGGATCGATGGCAAGTCTTACCCCTTTCATTTC
TGGGGCCACGGAACCCGGGTCACCGTCTTCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 318
LIGHT CHAIN
RFALTQPASVSGSPGQTITITCAGG [symbol] VSWFHFPPGKTPRLIIY [symbol] KRPSGVSPRFSGSQSGSTASLIISGLQSDD
EGTYFC [symbol] FGRGTLVTVLSQPKAAPSVTLFPPSSEELQANKATLVCLVSDFYPGAVTVAWKADGSPV
KVGVETTKPSKQSNNKYAASSYLSLTPEQWKSHRSYSCRVTHEGSTVEKTVAPAECS
SEQ ID NO: 319
CGATTTGCCCTGACTCAACCTGCCTCCGTGTCTGGGTCTCCTGGACAGACGATCACCATAACCTGC
GCTGGAGGCAGCGTCTCCTGGTTTCATTTCCCTCCAGGCAAAACCCCCAGACTCATTATTTATGAG
TCTTCTAAGCGACCCTCTGGGGTCTCTCCTCGATTCTCTGGGTCCCAGTCTGGCAGCACGGCCTCCC
TTATAATTTCTGGCCTCCAGTCTGATGACGAAGGGACATACTTCTGTTCTATTCTTGAATTTTTCGG
CAGAGGGACTCTTGTCACCGTCCTGAGTCAGCCCAAGGCTGCCCCCTCGGTCACTCTGTTCCCACC
CTCCTCTGAGGAGCTTCAAGCCAACAAGGCCACACTGGTGTGTCTCGTAAGTGACTTCTACCCGGG
AGCCGTGACAGTGGCCTGGAAGGCAGATGGCAGCCCCGTCAAGGTGGGAGTGGAGACCACCAAA
CCCTCCAAACAAAGCAACAACAAGTATGCGGCCAGCAGCTACCTGAGCCTGACGCCCGAGCAGTG
GAAGTCCCACAGAAGCTACAGCTGCCGGGTCACGCATGAAGGGAGCACCGTGGAGAAGACAGTG
GCCCCTGCAGAATGCTCT
SEQ ID NO: 320
>N49P23
HEAVY CHAIN
QVRLVQSGAGARKTGASMKLSCSTS [symbol] INWVRQARGQGLEWMGW [symbol] NIEGKFQ
GRVTLTRDIYSDTAYMEMTRLTTGDTGTYYC [symbol] WGQGSLVIVSSASTKGPSVFPLAPSSK
STSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 321
CAGGTGCGCTTGGTGCAGTCTGGGGCTGGGGCGAGGAAGACTGGGGCCTCAATGAAACTTTCCTG
CTCGACCTCTGGATACACCTTCACCACTCATCACGGCCACTTCATAAATTGGGTGCGACAGGCCCG
TGGACAAGGGCTTGAGTGGATGGGGTGGATGAATCCCATGACTGGGCAGATGAATATTGAGGGGA
AATTTCAGGGCAGAGTCACCCTCACTCGAGACATATACAGTGACACGGCTTACATGGAAATGACC
AGACTGACAACTGGCGACACGGGCACTTATTACTGTGCGCGAGGCGATTTCGGACAGAATTATCC
CTTTCATTATTGGGGCCAGGGAAGCCTGGTCATCGTCTCCTCGGCCTCCACCAAGGGCCCATCGGT
CTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCCTCCA
GCAGCTTGGGCACCCAGACCTACATCTGCAACGTGAATCACAAGCCCAGCAACACCAAGGTGGAC
AAGAGAGTTGAGCCCAAATCTTGTGACAAAACTCACACATGCCCACCGTGCCCAGCACCTGAACT
CCTGGGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCAC
GTACCGGGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGAGGAGATGACCAAGAACCAGGTCAG
CCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTACA
GCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTAAA
SEQ ID NO: 322
LIGHT CHAIN
LSALTQPASVSGSPGQSVTISCSGT [symbol] VSWYQQHPDKAPKLIIY [symbol] KRPSGISDRFSASRPDDTASLTISGL
QTGDEATYWC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 323
CTGTCTGCCCTGACTCAGCCTGCCTCCGTGTCTGGGTCTCCTGGGCAGTCGGTCACCATCTCCTGCT
CTGGAACGAACCGTTACCTTGTCTCCTGGTATCAACAACACCCTGACAAAGCCCCCAAACTCATCA
TTTATGACGACAATAAGCGGCCCTCAGGAATTTCTGATCGCTTCTCAGCCTCCAGGCCTGACGACA
CGGCCTCCCTGACAATCTCTGGACTCCAGACTGGGGACGAGGCTACTTATTGGTGTGCCTCATATG
AACGTTTTGGCGGCGGGACGAGGCTGACCGTCCTTAGTCAGCCCAAGGCTGCCCCCTCGGTCACTC
TGTTCCCACCCTCCTCTGAGGAGCTTCAAGCCAACAAGGCCACACTGGTGTGTCTCGTAAGTGACT
TCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 324
>N49P9.3
HEAVY CHAIN
HVQLVQSGGGVKKIGAAVRISCEVS [symbol] INWVRQAPGQGLEWMGW [symbol] NYSWRFQGRVT
MTRDMYTDTAFMELRGLRVDDTAVYYC [symbol] WGQGVRVVVSSPSTKGPSVFPLAPSSKSTS
GGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 325
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGGACATGTACACCGACACGGCCTTCATGGAGTTGAGAGGACTGAG
AGTGGACGACACGGCCGTCTATTATTGCGCGAGGGGACCCTCTGGGGAAAATTATCCTTTTCACTA
TTGGGGCCAGGGTGTCCGAGTGGTCGTCTCGTCACCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
SEQ ID NO: 326
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLRQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 327
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTACGTCAGCCCAAGGCTGCCCCCTCGGTCACTCTGTTCCCA
CCCTCCTCTGAGGAGCTTCAAGCCAACAAGGCCACACTGGTGTGTCTCGTAAGTGACTTCTACCCG
GGAGCCGTGACAGTGGCCTGGAAGGCAGATGGCAGCCCCGTCAAGGTGGGAGTGGAGACCACCA
AACCCTCCAAACAAAGCAACAACAAGTATGCGGCCAGCAGCTACCTGAGCCTGACGCCCGAGCA
GTGGAAGTCCCACAGAAGCTACAGCTGCCGGGTCACGCATGAAGGGAGCACCGTGGAGAAGACA
GTGGCCCCTGCAGAATGCTCT
SEQ ID NO: 328
>N49P9.4
HEAVY CHAIN
HVQLVQSGGGVKKIGAAVRISCEVS [symbol] INWVRQAPGQGLEWMGW [symbol] NYSWRFQGRVT
MTRDMYTDTAFMELRGLRVDDTAVYYC [symbol] WGQGVRVVVSSPSTKGPSVFPLAPSSKSTS
GGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 329
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGGACATGTACACCGACACGGCCTTCATGGAGTTGAGAGGACTGAG
AGTGGACGACACGGCCGTCTATTATTGCGCGAGGGGACCCTCTGGGGAAAATTATCCTTTTCACTA
TTGGGGCCAGGGTGTCCGAGTGGTCGTCTCGTCACCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
SEQ ID NO: 330
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLSQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 331
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAAGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 332
>N49P51
HEAVY CHAIN
HIQLLQSGPQVKKSGDTVRISCETS [symbol] IHWVRQTPEKRLRWMGW [symbol] NYAPEFQGRIRMT
RDTFIDTVYVDLSGLTPADTAYYYC [symbol] WGHGTRVTVFSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 333
CACATTCAGTTGCTACAGTCGGGGCCTCAAGTTAAGAAGTCTGGGGACACAGTGAGAATCTCCTG
TGAGACCTCTGGATATAACTTCGTCGACTCCCGTATCCACTGGGTCCGACAGACCCCGGAAAAAC
GTCTCAGATGGATGGGCTGGATCAATCCTCTCCACGGTGGTGTGAATTACGCGCCGGAATTTCAGG
GCAGAATCAGGATGACCAGGGACACATTTATAGACACAGTTTACGTGGACCTGAGCGGACTGACA
CCGGCCGACACGGCCTATTATTACTGCGCGCGAGGGATCGATGGCAAGTCTTACCCCTTTCATTTC
TGGGGCCACGGAACCCGGGTCACCGTCTTCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 334
LIGHT CHAIN
RFALTQPASVSGSPGQTITITCAGG [symbol] VSWFHFPPGKTPRLIIY [symbol] KRPSGVSPRFSGSQSGSTASLIISGLQSDD
EGTYFC [symbol] FGRGTLVTVLSQPKAAPSVTLFPPSSEELQANKATLVCLVSDFYPGAVTVAWKADGSPV
KVGVETTKPSKQSNNKYAASSYLSLTPEQWKSHRSYSCRVTHEGSTVEKTVAPAECS
SEQ ID NO: 335
CGATTTGCCCTGACTCAACCTGCCTCCGTGTCTGGGTCTCCTGGACAGACGATCACCATAACCTGC
GCTGGAGGCAGCGTCTCCTGGTTTCATTTCCCTCCAGGCAAAACCCCCAGACTCATTATTTATGAG
TCTTCTAAGCGACCCTCTGGGGTCTCTCCTCGATTCTCTGGGTCCCAGTCTGGCAGCACGGCCTCCC
TCATAATTTCTGGCCTCCAGTCTGATGACGAAGGGACATACTTCTGTTCTATTCTTGAATTTTTCGG
CAGAGGGACTCTTGTCACCGTCCTGAGTCAGCCCAAGGCTGCCCCCTCGGTCACTCTGTTCCCACC
CTCCTCTGAGGAGCTTCAAGCCAACAAGGCCACACTGGTGTGTCTCGTAAGTGACTTCTACCCGGG
AGCCGTGACAGTGGCCTGGAAGGCAGATGGCAGCCCCGTCAAGGTGGGAGTGGAGACCACCAAA
CCCTCCAAACAAAGCAACAACAAGTATGCGGCCAGCAGCTACCTGAGCCTGACGCCCGAGCAGTG
GAAGTCCCACAGAAGCTACAGCTGCCGGGTCACGCATGAAGGGAGCACCGTGGAGAAGACAGTG
GCCCCTGCAGAATGCTCT
SEQ ID NO: 336
>N49P52
HEAVY CHAIN
RVTLQQSGAIVRQPGASVTVSCETS [symbol] IYWVRQAPGQGLEWLGR [symbol] KYAPRFQGRLSMTR
DWSLDTAYLGLTGLTLGDTALYFC [symbol] WGQGTLVTVSAASTKGPSVFPLAPSSKST
SGGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 337
CGTGTTACATTACAACAATCTGGGGCTATAGTGAGGCAGCCTGGGGCCTCAGTGACCGTCTCCTGC
GAGACTTCTGGATATACTTTCACCAAGTATTTCATCTACTGGGTGCGACAGGCCCCTGGACAGGGT
CTTGAGTGGCTGGGCAGAATACACCCCCGAACCGGTGCCGTGAAGTATGCACCGAGATTTCAGGG
TAGACTGTCCATGACCAGAGACTGGTCACTCGACACAGCCTACCTCGGATTGACCGGACTGACAC
TCGGCGACACGGCTCTATATTTCTGTGCGAGGGGGGCCTTTGAGGCAGATTCATATGGGTCAAGTT
ATCCCTTTCACCACTGGGGCCAGGGAACCCTAGTCACCGTCTCCGCGGCCTCCACCAAGGGCCCAT
CGGTCTTCCCCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGGT
GGACAAGAGAGTTGAGCCCAAATCTTGTGACAAAACTCACACATGCCCACCGTGCCCAGCACCTG
AACTCCTGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACA
GCACGTACCGGGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGCCAAAGG
GCAGCCCCGAGAACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGTAAA
SEQ ID NO: 338
LIGHT CHAIN
SWALTQPASVSASPGQSVTMSCTGFG [symbol] DSWYQQYPGKAPKLIIY [symbol] KRPSGVSDRFSASRLGSTSSLTIS
NVQAADDAHYVC [symbol] FGGGTKLTVLSQPKAAPSNYLFPPSSEELQANKAYLNCLNSDFYPGANYNAW
KADGSPVKVGVETTKPSKQSNNKYAASSYLSLTPEQWKSHRSYSCRVTHEGSTVEKTVAPAECS
SEQ ID NO: 339
TCCTGGGCCCTGACTCAACCCGCCTCCGTGTCTGCGTCTCCTGGGCAGTCGGTCACCATGTCCTGC
ACTGGATTCGGAAATTATAACCCTGACTCCTGGTACCAACAATACCCAGGCAAAGCCCCCAAACT
CATCATTTATGAAGACAATAAAAGACCCTCGGGGGTCTCTGATCGCTTCTCTGCCTCCAGACTTGG
CAGCACGTCTTCCCTGACAATCTCTAACGTCCAGGCTGCGGACGACGCCCATTATGTCTGCGCCTC
CTTTGAATTTTTCGGCGGAGGGACCAAGCTGACCGTCCTGAGTCAGCCCAAGGCTGCCCCCTCGGT
CACTCTGTTCCCACCCTCCTCTGAGGAGCTTCAAGCCAACAAGGCCACACTGGTGTGTCTCGTAAG
TGACTTCTACCCGGGAGCCGTGACAGTGGCCTGGAAGGCAGATGGCAGCCCCGTCAAGGTGGGAG
TGGAGACCACCAAACCCTCCAAACAAAGCAACAACAAGTATGCGGCCAGCAGCTACCTGAGCCTG
ACGCCCGAGCAGTGGAAGTCCCACAGAAGCTACAGCTGCCGGGTCACGCATGAAGGGAGCACCG
TGGAGAAGACAGTGGCCCCTGCAGAATGCTCT
SEQ ID NO: 340
>N49P53
HEAVY CHAIN
RVTLQQSGATVKQPGASVTVSCETS [symbol] IHWVRQAPGQGLQWVGR [symbol] KYAPIFQGKVSMS
RDLSRDTAYLGLTRLTLADTALFFC [symbol] WGQGTLVIVSAASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 341
CGTGTGACATTACAACAATCTGGGGCTACAGTGAAGCAGCCTGGGGCCTCAGTGACCGTCTCCTG
CGAGACTTCTGGATACACTTTCACCAAGTATACCATTCACTGGGTGCGACAGGCCCCTGGACAGG
GTCTTCAGTGGGTGGGCAGAATACACCCCCGAACCGGTGCCGTGAAGTATGCACCGATATTTCAG
GGTAAAGTGTCCATGAGTCGAGACTTGTCACGCGACACAGCCTACCTCGGATTGACCAGACTGAC
GCTCGCCGACACGGCTCTATTTTTCTGTGCGAGGGGGGCCTTTGAGGCAGATTTAAGTGGGCCAAC
TTACCCCTTTCACCACTGGGGCCAAGGAACCCTAGTCATCGTCTCCGCGGCCTCCACCAAGGGCCC
ATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCG
TGCACACCTTCCCGGCTGTCCTACAGTCCTCAGGACTCTACTCCCTCAGCAGCGTGGTGACCGTGC
CCTCCAGCAGCTTGGGCACCCAGACCTACATCTGCAACGTGAATCACAAGCCCAGCAACACCAAG
GTGGACAAGAGAGTTGAGCCCAAATCTTGTGACAAAACTCACACATGCCCACCGTGCCCAGCACC
TGAACTCCTGGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGAGCAGTACAA
CAGCACGTACCGGGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAGAACC
AGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTT
CCTCTACAGCAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCTCCGGGTAAA
SEQ ID NO: 342
LIGHT CHAIN
SWALTQPASVSASPGQSVTMSCTGF [symbol] DSWYQQYPGKAPKLIIY [symbol] KRPSGVSNRFSASRLGSTSSLTIS
NVQAADDAHYVC [symbol] FGGGTKLIVLSQPKAAPSNYLFPPSSEELQANKAYLNCLNSDFYPGANYNAW
KADGSPVKVGVETTKPSKQSNNKYAASSYLSLTPEQWKSHRSYSCRVTHEGSTVEKTVAPAECS
SEQ ID NO: 343
TCCTGGGCCCTGACTCAACCCGCCTCCGTGTCTGCGTCTCCTGGGCAGTCGGTCACCATGTCCTGC
ACTGGATTCGGAAATTATAACCCTGACTCCTGGTACCAACAATACCCAGGCAAAGCCCCCAAACT
CATCATTTATGAGGACAATAAAAGACCCTCGGGAGTCTCTAATCGCTTCTCTGCCTCCAGACTTGG
CAGCACGTCTTCCCTGACAATCTCTAACGTCCAGGCCGCTGACGACGCCCATTATGTCTGCGCCTC
CTTTGAATTTTTCGGCGGAGGGACCAAGCTGATCGTCCTGAGTCAGCCCAAGGCTGCCCCCTCGGT
CACTCTGTTCCCACCCTCCTCTGAGGAGCTTCAAGCCAACAAGGCCACACTGGTGTGTCTCGTAAG
TGACTTCTACCCGGGAGCCGTGACAGTGGCCTGGAAGGCAGATGGCAGCCCCGTCAAGGTGGGAG
TGGAGACCACCAAACCCTCCAAACAAAGCAACAACAAGTATGCGGCCAGCAGCTACCTGAGCCTG
ACGCCCGAGCAGTGGAAGTCCCACAGAAGCTACAGCTGCCGGGTCACGCATGAAGGGAGCACCG
TGGAGAAGACAGTGGCCCCTGCAGAATGCTCT
SEQ ID NO: 344
>N49P54
HEAVY CHAIN
NVQLMQSGTEVKKSGASVTISCETA [symbol] IHWLRQAPGGGFQWMGW [symbol] NYPQFLQGRVSM
TRDLSTDTVYMVLNGLTPDDTGLYYC [symbol] WGQGTLLTVSPASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 345
AACGTGCAGTTGATGCAGTCTGGGACTGAGGTGAAGAAGTCTGGGGCCTCGGTGACAATCTCTTG
TGAGACCGCTGGATTCAACTTCATCGACTCCGTCATACACTGGCTGCGCCAGGCCCCTGGAGGAG
GATTTCAGTGGATGGGGTGGATCAAGCCTCTTAGAGGTGCCGTCAATTATCCACAGTTTTTGCAGG
GCAGGGTCTCCATGACCCGGGACTTGTCCACCGACACGGTGTACATGGTCTTGAATGGACTGACA
CCTGACGACACAGGCCTTTATTACTGCGCGAAAGGGGCCTTTAGAGGGGGTTCTCCCTTTGGCTTC
TGGGGCCAGGGAACTCTGCTCACCGTCTCCCCAGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 346
LIGHT CHAIN
QSALSQPVSVSGSPGESITISCTGATTWYQQLPGRPPKLIIY [symbol] NRPSGISSRFSGSTSGHTASLTISGLQVDDE
GLYHC [symbol] FGGGTKLTVLSQPKAAPSVTLFPPSSEELQANKATLVCLVSDFYPGAVTVAWKADGSPV
KVGVETTKPSKQSNNKYAASSYLSLTPEQWKSHRSYSCRVTHEGSTVEKTVAPAECS
SEQ ID NO: 347
CAGTCTGCCCTGTCTCAGCCTGTCTCCGTGTCTGGGTCTCCTGGAGAGTCGATCACCATTTCCTGTA
CTGGAGCCACCACCTGGTATCAACAACTCCCAGGCAGACCCCCCAAACTCATCATTTATGACGTCA
CTAACCGGCCCTCAGGCATTTCTAGTCGTTTCTCTGGCTCCACGTCTGGCCACACGGCCTCCCTGA
CAATCTCCGGTCTCCAGGTTGACGACGAGGGTCTGTATCACTGCGCCTCACGTGAATTTTTCGGCG
GAGGGACCAAGCTGACCGTCCTGAGTCAGCCCAAGGCTGCCCCCTCGGTCACTCTGTTCCCACCCT
CCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
SEQ ID NO: 348
>N49P60
HEAVY CHAIN
QVRLVQSGPQVKKTGASVRVSCETS [symbol] IHWLRLGPGEGLQWMGW [symbol] NYENKFRGRVTIT
RDTSTDTVYLDMSRLTPDDTAVYFC [symbol] WGQGTQVTVSPASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 349
CAGGTGCGACTGGTGCAGTCTGGGCCTCAGGTGAAGAAGACTGGGGCCTCAGTGAGGGTCTCCTG
CGAAACCTCTGGATACACGTTCACCTCCTACTTCATCCATTGGTTACGACTGGGCCCCGGAGAGGG
GCTTCAGTGGATGGGGTGGATCAACCCTTTACATGGTGCCGTGAATTATGAAAACAAATTTAGGG
GCAGGGTCACAATCACCAGGGACACGTCCACAGACACAGTGTATTTGGACATGAGCAGACTGACC
CCTGACGACACGGCCGTCTATTTCTGCACAAGAGGAATCGTTGCTGATGGGTGGCCCTATGGCCAC
TGGGGCCAGGGAACCCAAGTCACCGTCTCCCCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 350
LIGHT CHAIN
SWALTQPASVSGSPGQSVAISCAGG [symbol] VSWYQVLPGRAPKLIIY [symbol] KRPSGVSARFSGSQSGNTAYLTISDLQT
EDEGIYFC [symbol] FGGGTKLTVLSQPKAAPSVTLFPPSSEELQANKATLVCLVSDFYPGAVTVAWKADG
SPVKVGVETTKPSKQSNNKYAASSYLSLTPEQWKSHRSYSCRVTHEGSTVEKTVAPAECS
SEQ ID NO: 351
TCCTGGGCCCTGACTCAGCCTGCCTCCGTGTCTGGGTCTCCTGGACAGTCGGTCGCCATCTCCTGC
GCTGGCGGCAGCGTCTCCTGGTACCAGGTGCTCCCAGGCAGAGCCCCCAAACTCATCATTTATGA
GGGCGCTAAGCGACCCTCAGGGGTTTCTGCTCGCTTCTCTGGCTCCCAGTCTGGCAACACGGCTTA
CCTGACAATTTCTGACCTCCAGACTGAGGACGAGGGCATCTACTTCTGCTCTTCACTTCAATTCTTC
GGCGGAGGGACCAAACTGACCGTCCTAAGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 352
>N49P61
HEAVY CHAIN
QVRLQQSGVVVRKPGASVRISCETS [symbol] VHWVRRAPGRGFEWMGW [symbol] DYAPQLRGRISLT
RDIYSETVFIDVSRLTSGDTAIYFC [symbol] WGQGTQLIVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 353
CAGGTGCGACTTCAGCAGTCTGGTGTCGTGGTGAGGAAGCCTGGGGCCTCAGTGAGAATTTCCTG
CGAGACTTCTGGATTCACCTTCATCGACCACATTGTCCATTGGGTGCGGCGGGCCCCTGGACGAGG
CTTTGAATGGATGGGTTGGATCAAGCCTCTTAGGGGTGCCGTAGATTATGCACCCCAACTTCGGGG
CAGGATCTCCCTGACGAGGGACATTTACAGTGAAACCGTCTTTATAGACGTGAGCCGACTGACGT
CTGGCGACACGGCGATATACTTTTGTTGTAAGGCCGCCGCCCCTGAAGAAGCATTCCCCCTTCAAT
ACTGGGGCCAGGGGACCCAACTTATCGTCTCCTCGGCCTCCACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
SEQ ID NO: 354
LIGHT CHAIN
QAALTQPASVSGSPGQSVTISCLYA [symbol] ICWYQLHPGRAPKLLIV [symbol] KRPSGVSPRFSGSKSGTTASLTISGL
QADDEAEYHC [symbol] FGGGTKLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 355
CAGGCTGCCCTGACTCAGCCCGCCTCCGTGTCCGGCTCTCCTGGACAGTCGGTCACCATTTCCTGC
CTTTATGCCAATGTAGATATCTGCTGGTATCAACTACACCCGGGCAGAGCCCCCAAACTTCTAATT
GTTGACAATAATAAGCGGCCCTCAGGAGTCTCTCCTCGCTTCTCTGGCTCCAAGTCTGGCACCACG
GCCTCCCTGACAATCTCTGGACTTCAGGCTGACGACGAGGCTGAATATCACTGCTCTTCAAGAACA
TTTTTTGGCGGGGGGACCAAGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 356
>N49P62
HEAVY CHAIN
QVRLQQSGWVRKPGASVRLSCETS [symbol] VNWVRRAPGRGFEWMGW [symbol] DYAPQHRGRISLT
RDIYTETVFIDLSRLTSGDTAIYFC [symbol] WGQGTQLIVSPASTKGPSVFPLAPSSKSTSGGTA
ALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 357
CAGGTGCGACTTCAGCAGTCTGGTGTCGTGGTGAGGAAGCCTGGGGCCTCAGTGAGACTTTCCTGC
GAGACGTCTGGATTCAAATTCATCGACCACATTGTCAACTGGGTGCGGCGGGCCCCTGGACGAGG
CTTTGAATGGATGGGTTGGATCAAGCCTCTTGGGGGTGTCGCTGATTATGCACCCCAACATCGGGG
CAGGATCTCACTGACGAGGGACATTTACACTGAAACCGTCTTTATAGACCTGAGTCGACTGACGTC
TGGCGACACGGCGATTTATTTCTGTTGTAAGGCCGCCGCCCCTGATGAAGCATTCCCCCTTGAATA
CTGGGGCCAGGGGACCCAACTTATCGTCTCCCCGGCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
SEQ ID NO: 358
LIGHT CHAIN
QAALTQPASVSGSPGQSVTISCLYA [symbol] ICWYQIQPGRLPKLLIV [symbol] RRPSGVSPRFSGSKSGTTASLTISGLQ
ADDEAEYHC [symbol] FGGGTKLTVLSQPKAAPSVTLFPPSSEELQANKATLVCLVSDFYPGAVTVAWKAD
GSPVKVGVETTKPSKQSNNKYAASSYLSLTPEQWKSHRSYSCRVTHEGSTVEKTVAPAECS
SEQ ID NO: 359
CAGGCTGCCCTGACTCAGCCCGCCTCCGTGTCCGGCTCTCCTGGACAGTCGGTCACCATTTCCTGC
CTTTATGCCAATGTAGATATCTGCTGGTATCAAATACAGCCGGGCAGATTACCCAAACTTCTGATT
GTTGACAATAATAGGCGACCCTCAGGAGTCTCTCCTCGCTTCTCTGGCTCCAAGTCTGGCACCACG
GCCTCCCTGACAATCTCTGGACTTCAGGCTGACGACGAGGCTGAATATCACTGCTCTTCAACAACA
TTTTTTGGCGGGGGGACCAAGTTGACCGTCCTCAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 360
>N49P63
HEAVY CHAIN
QVRLVQSGPVMRKPGASVRISCETS [symbol] IVHWVRRAPGRGFEWMGW [symbol] DYAPHLRGRISVT
RDVFSETVFLDLSRLTSGDTAMYFC [symbol] WGQGTQVIVSSASYKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 361
CAGGTGCGACTTGTGCAGTCTGGTCCCGTGATGAGAAAGCCTGGGGCCTCAGTGAGAATTTCTTGC
GAGACATCTGGATTCGCCTTCTTGGACCACATTGTCCACTGGGTGCGGCGGGCCCCTGGACGCGGC
TTTGAATGGATGGGTTGGGTTAAGACCATTGGGGGTGTCGTTGATTATGCACCCCACCTTAGGGGC
AGGATCTCCGTGACGAGAGACGTCTTTAGTGAAACCGTCTTTCTGGACTTGAGCCGACTGACGTCT
GGCGACACGGCGATGTATTTTTGTTCTAAGGCCGCCGCCCCTGACGAAGCCTTCCCCCTTGAATTT
TGGGGCCAGGGGACCCAAGTCATCGTCTCCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 362
LIGHT CHAIN
QAALTQPASVSGSPGQSVHSCLYA [symbol] ICWYQLHPGRAPKLLIL [symbol] KRPSGVSSRFSGSKSGITASLTISDL
QADDEAEYHC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 363
CAGGCTGCCCTGACTCAGCCCGCCTCCGTGTCCGGCTCTCCTGGACAGTCGGTCACCATTTCTTGC
CTTTATGCCAATGTGGATATCTGCTGGTATCAACTTCACCCGGGCAGAGCCCCCAAACTTCTTATT
CTTGACAATAATAAACGGCCCTCAGGAGTCTCTAGTCGCTTCTCCGGTTCCAAGTCTGGCACCACG
GCCTCCCTAACCATCTCTGACCTTCAGGCTGACGACGAGGCTGAATATCACTGCTCTTCAACAACA
TTTTTTGGCGGGGGGACCAGGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 364
>N49P64
HEAVY CHAIN
QVRLVQSGPWRKPGTSVRISCETS [symbol] IVHWVRRAPGRGFEWMGW [symbol] DYAPHLRGRISVT
RDVFSEIVFMELSRLTSGDTAMYFC [symbol] WGQGTQVIVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 365
CAGGTGCGACTTGTGCAGTCTGGTCCCGTGGTGAGAAAGCCTGGGACCTCAGTGAGAATTTCTTGC
GAGACATCTGGATTCGCCTTCTTGGACCACATTGTCCACTGGGTGCGGCGGGCCCCTGGACGCGGC
TTTGAATGGATGGGTTGGGTTAAGACCATTGGGGGTGTCGTTGATTATGCACCCCACCTTAGGGGC
AGGATCTCCGTGACGAGGGACGTATTTAGTGAAATCGTCTTTATGGAGTTGAGTCGACTGACGTCT
GGCGACACGGCGATGTATTTTTGTTCTAAGGCCGCCGCCCCTGACGAAGCCTTCCCCCTTGAATTT
TGGGGCCAGGGGACCCAAGTCATCGTCTCCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 366
LIGHT CHAIN
QAALTQPASVSGSPGQSVTISCLYA [symbol] ICWYQLHPGRAPKLLIV [symbol] KRPSGVSSRFSGSKSGTTASLTISDL
QADDEAEYHC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 367
CAGGCTGCCCTGACTCAGCCCGCCTCCGTGTCCGGCTCTCCTGGACAGTCGGTCACCATTTCCTGC
CTTTATGCCAATGTGGATATCTGCTGGTATCAACTTCACCCGGGCAGAGCCCCCAAACTTCTAATT
GTTGACAATAATAAGCGGCCCTCAGGAGTCTCTAGTCGCTTCTCTGGTTCCAAGTCTGGCACCACG
GCCTCCCTAACAATCTCTGATCTTCAGGCTGACGACGAGGCTGAATATCACTGCTCTTCAACAACA
TTTTTTGGCGGGGGGACCAGGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 368
>N49P65
HEAVY CHAIN
QVQLVQSGAGVKKPGASVRVSCETS [symbol] IHFLRQAPGQGLEWMGW [symbol] NYPRKFQGRVTL
TRDIYTTTVYMQLNGLTPDDTAVYYC [symbol] WGQGSLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 369
CAAGTGCAACTGGTGCAGTCTGGGGCTGGGGTGAAGAAGCCTGGGGCCTCAGTGAGGGTCTCCTG
CGAGACATCCGGATTCAAGTTCACCGAGTACTTTATCCACTTTTTACGACAGGCCCCTGGACAAGG
GCTTGAGTGGATGGGATGGCTCAACCCTCTCAGAGGTGCCGTCAACTATCCACGGAAGTTTCAGG
GCAGAGTCACTTTGACCAGGGACATCTACACCACCACCGTCTACATGCAACTTAACGGTCTGACCC
CTGACGACACGGCCGTCTACTACTGTGCCAGAGCGGTCTTTAATGAAGCTTTCCCCTTTGACTACT
GGGGCCAGGGAAGCCTGGTCACCGTCTCCTCAGCCTCCACCAAGGGCCCATCGGTCTTCCCCCTGG
CACCCTCCTCCAAGAGCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
SEQ ID NO: 370
LIGHT CHAIN
SWAQTQPASVSGSPGQSITISCAGI [symbol] DAWYQQYPGRPPRLILY [symbol] KRPSGVSPRFSASRAGKTASLTISGLQA
DDEAYYHC [symbol] GGVTKLTVLSQPKAAPSVTLFPPSSEELQANKATLVCLVSDFYPGAVTVAWKADG
SPVKVGVETTKPSKQSNNKYAASSYLSLTPEQWKSHRSYSCRVTHEGSTVEKTVAPAECS
SEQ ID NO: 371
TCCTGGGCCCAGACTCAGCCTGCCTCCGTGTCTGGGTCTCCTGGACAGTCGATCACCATCTCCTGC
GCTGGAATCGTCAGTGATGCCTGGTACCAGCAATACCCAGGCAGACCCCCCAGACTCATCCTTTAT
GACGGCGATAAGCGGCCCTCAGGGGTTTCTCCTCGTTTTTCTGCCTCCAGGGCCGGCAAGACGGCC
TCCCTGACAATTTCTGGGCTGCAGGCTGACGACGAGGCTTATTATCACTGCGCGTCAAGGGAATTT
TTTGGAGGCGTGACCAAGTTGACCGTCCTAAGTCAGCCCAAGGCTGCCCCCTCGGTCACTCTGTTC
CCACCCTCCTCTGAGGAGCTTCAAGCCAACAAGGCCACACTGGTGTGTCTCGTAAGTGACTTCTAC
CCGGGAGCCGTGACAGTGGCCTGGAAGGCAGATGGCAGCCCCGTCAAGGTGGGAGTGGAGACCA
CCAAACCCTCCAAACAAAGCAACAACAAGTATGCGGCCAGCAGCTACCTGAGCCTGACGCCCGAG
CAGTGGAAGTCCCACAGAAGCTACAGCTGCCGGGTCACGCATGAAGGGAGCACCGTGGAGAAGA
CAGTGGCCCCTGCAGAATGCTCT
SEQ ID NO: 372
>N49P66
HEAVY CHAIN
QVRLQQSGWVRKPGASVRLSCETS [symbol] VNWVRRAPGRGFEWMGW [symbol] DYAPQHRGRISLT
RDIYTETVFIDLSRLTSGDTAIYFC [symbol] WGQGTQLIVSPASTKGPSVFPLAPSSKSTSGGTA
ALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 373
CAGGTGCGACTTCAGCAGTCTGGTGTCGTGGTGAGGAAGCCTGGGGCCTCAGTGAGACTTTCCTGC
GAGACGTCTGGCTTCAAATTCATCGACCACATTGTCAACTGGGTGCGGCGGGCCCCTGGACGAGG
CTTTGAATGGATGGGTTGGATCAAGCCTCTTGGGGGTGTCGCTGATTATGCACCCCAACATCGGGG
CAGGATCTCACTGACGAGGGACATTTACACTGAAACCGTCTTTATAGACCTGAGTCGACTGACGTC
TGGCGACACGGCGATTTATTTTTGTTGTAAGGCCGCCGCCCCTGATGAAGCATTCCCCCTTGAATA
CTGGGGCCAGGGGACCCAACTTATCGTCTCCCCGGCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
SEQ ID NO: 374
LIGHT CHAIN
QAALTQPASVSGSPGQSVTISCLYA [symbol] ICWYQIQPGRLPKLLIV [symbol] RRPSGVSPRFSGSKSGTTASLTISGLQ
ADDEAEYHC [symbol] FGGGTKLTVLSQPKAAPSVTLFPPSSEELQANKATLVCLVSDFYPGAVTVAWKAD
GSPVKVGVETTKPSKQSNNKYAASSYLSLTPEQWKSHRSYSCRVTHEGSTVEKTVAPAECS
SEQ ID NO: 375
CAGGCTGCCCTGACTCAGCCCGCCTCCGTGTCCGGCTCTCCTGGACAGTCGGTCACCATTTCCTGC
CTTTATGCCAATGTAGATATCTGCTGGTATCAAATACAGCCGGGCAGATTACCCAAACTTCTGATT
GTTGACAATGATAGGCGACCCTCAGGAGTCTCTCCTCGCTTCTCTGGCTCCAAGTCTGGCACCACG
GCCTCCCTGACAATCTCTGGACTTCAGGCTGACGACGAGGCTGAATATCACTGCTCTTCAACAACA
TTTTTTGGCGGGGGGACCAAGTTGACCGTCCTCAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 376
>N49P67
HEAVY CHAIN
QVRLVQSGPVMRKPGASVRISCETS [symbol] VHWVRRAPGRGFEWMGW [symbol] DYAPHLRGRISVT
RDVFSETVFLDLSRLTSGDTAMYFC [symbol] WGQGTQVIVSSASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 377
CAGGTGCGACTTGTGCAGTCTGGTCCCGTGATGAGAAAGCCTGGGGCCTCAGTGAGAATTTCTTGC
GAGACATCTGGATTCGCCTTCTTGGACCACATTGTCCACTGGGTGCGGCGGGCCCCTGGACGCGGC
TTTGAATGGATGGGTTGGGTTAAGACCATTGGGGGTGTCGTTGATTATGCACCCCACCTTAGGGGC
AGGATCTCCGTGACGAGAGACGTCTTTAGTGAAACCGTCTTTCTGGACTTGAGTCGACTGACGTCT
GGCGACACGGCGATGTATTTTTGTTCTAAGGCCGCCGCCCCTGACGAAGCCTTCCCCCTTGAATTT
TGGGGCCAGGGGACCCAAGTCATCGTCTCCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
SEQ ID NO: 378
LIGHT CHAIN
QAALTQPASVSGSPGQSVTISCLYA [symbol] ICWYQLHPGRAPKLLIL [symbol] KRPSGVSSRFSGSKSGTTASLTISDL
QADDEAEYHC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 379
CAGGCTGCCCTGACTCAGCCCGCCTCCGTGTCCGGCTCTCCTGGACAGTCGGTCACCATTTCTTGC
CTTTATGCCAATGTGGATATCTGCTGGTATCAACTTCACCCGGGCAGAGCCCCCAAACTTCTAATT
CTTGACAATAATAAACGGCCCTCAGGAGTCTCTAGTCGCTTCTCCGGTTCCAAGTCTGGCACCACG
GCCTCCCTAACCATCTCTGACCTTCAGGCTGACGACGAGGCTGAATATCACTGCTCTTCAACAACT
TTTTTTGGCGGGGGGACCAGGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
SEQ ID NO: 380
>N49P68
HEAVY CHAIN
HVQLRQSGTEAKKSGASVTISCETA [symbol] IHWLRQAPGGGFQWMGW [symbol] NYPHYLQGRISMT
RDLSSDTVYMVLNRLTPADTGLYYC [symbol] WGQGTLLTVSPASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 381
CACGTGCAGTTGAGGCAGTCTGGGACTGAGGCGAAGAAGTCTGGGGCCTCGGTGACAATCTCTTG
TGAGACCGCTGGATTCAACTTCATCGACTCCGTCATACACTGGCTGCGCCAGGCCCCTGGTGGGGG
ATTTCAGTGGATGGGGTGGATCAAGCCTCTTAGAGGTGGCGTCAATTATCCACATTATTTGCAGGG
CAGAATCTCCATGACCCGGGACTTGTCCAGTGACACGGTTTACATGGTCTTAAATAGACTGACACC
TGCCGACACAGGCCTTTATTACTGCGCGAAAGGGGCCTTTGGGGGGAGTTCTCCCTTTGGCTTCTG
GGGCCAGGGAACTCTGCTCACCGTCTCCCCAGCCTCCACCAAGGGCCCATCGGTCTTCCCCCTGGC
ACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
SEQ ID NO: 382
LIGHT CHAIN
QSALSQPVSVSGSPGESITISCTEATTWYQQLPGKPPKLIIY [symbol] NRPSGISSRFSGSMSGRTASLTISGLQVDDE
GLYHC [symbol] FGGGTKLTVLSQPKAAPSVTLFPPSSEELQANKATLVCLVSDFYPGAVTVAWKADGSPV
KVGVETTKPSKQSNNKYAASSYLSLTPEQWKSHRSYSCRVTHEGSTVEKTVAPAECS
SEQ ID NO: 383
CAGTCTGCCCTGTCTCAGCCTGTCTCCGTGTCTGGGTCTCCTGGAGAGTCGATCACCATTTCCTGTA
CTGAAGCCACCACCTGGTATCAACAACTCCCAGGCAAACCCCCCAAACTCATCATTTATGACGTG
ACCAACCGGCCCTCAGGCATTTCAAGTCGTTTCTCTGGCTCCATGTCTGGTCGCACGGCCTCCCTG
ACAATCTCCGGTCTCCAGGTTGACGACGAGGGTCTCTATCACTGTGCCTCACGTGAATTTTTCGGC
GGGGGGACCAAGCTGACCGTCCTGAGTCAGCCCAAGGCTGCCCCCTCGGTCACTCTGTTCCCACCC
TCCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
SEQ ID NO: 384
>N49P69
HEAVY CHAIN
HVQLMQSGTQAKKSGASVTISCETA [symbol] IHWLRQAPGGGFQWMGW [symbol] NYPPYLQGRISLT
RDLSTDTIYMVLNGLTPADTGFYYC [symbol] WGQGTLLTVSPASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 385
CACGTGCAATTGATGCAGTCTGGGACTCAGGCGAAGAAGTCTGGGGCCTCGGTGACAATTTCTTGT
GAGACCGCTGGATTCAAGTTCATCGACTCCGTCATACACTGGCTGCGCCAGGCCCCTGGAGGGGG
ATTTCAGTGGATGGGGTGGATCAAGCCTCTTGGAGGTGCCGTCAACTATCCACCCTATTTGCAGGG
CAGGATCTCCTTGACCCGTGACTTGTCCACCGACACAATTTACATGGTCTTGAATGGACTGACACC
TGCCGACACAGGCTTTTATTACTGCGCCAAAGGGGCCTTTGGGGGGGGTTCTCCCTTTGGCTTCTG
GGGCCAGGGGACTCTGCTCACCGTCTCCCCAGCCTCCACCAAGGGCCCATCGGTCTTCCCCCTGGC
ACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
SEQ ID NO: 386
LIGHT CHAIN
QSALSQPVSVSGSPGDSITISCFGATTWYQQLPGRPPKLIIY [symbol] NRPSGISGRFSGSMSGQKASLTISGLQVDD
EGLYHC [symbol] FGGGTKLTVLSQPKAAPSVTLFPPSSEELQANKATLVCLVSDFYPGAVTVAWKADGSP
VKVGVETTKPSKQSNNKYAASSYLSLTPEQWKSHRSYSCRVTHEGSTVEKTVAPAECS
SEQ ID NO: 387
CAGTCTGCCCTGTCTCAGCCTGTCTCCGTGTCTGGGTCTCCTGGAGACTCGATCACCATTTCTTGTT
TTGGAGCCACCACCTGGTATCAACAACTCCCAGGCAGACCCCCCAAACTCATCATTTATGACGTGA
CTAACCGGCCCTCAGGCATTTCAGGTCGTTTCTCTGGCTCCATGTCTGGTCAAAAGGCCTCCCTGA
CAATCTCCGGTCTCCAGGTTGACGACGAGGGTCTCTATCACTGCGCCTCACGTGAATTTTTCGGCG
GGGGGACCAAACTGACCGTCCTGAGTCAGCCCAAGGCTGCCCCCTCGGTCACTCTGTTCCCACCCT
CCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
SEQ ID NO: 388
>N49P70
HEAVY CHAIN
HVQLRQSGTEAKKSGASVTISCETA [symbol] IHWLRQAPGGGFQWMGW [symbol] NYPHYLQGRISMT
RDLSSDTVYMVLNRLTPDDTGLYYC [symbol] WGQGTLLTVSPASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 389
CACGTGCAGTTGAGGCAGTCTGGGACTGAGGCGAAGAAGTCTGGGGCCTCGGTGACAATCTCTTG
TGAGACCGCTGGATTCAACTTCATCGACTCCGTCATACACTGGCTGCGCCAGGCCCCTGGTGGGGG
ATTTCAGTGGATGGGGTGGATCAAGCCTCTTAGAGGTGGCGTCAATTATCCACATTATTTGCAGGG
CAGAATCTCCATGACCCGGGACTTGTCCAGTGACACGGTTTACATGGTCTTAAATAGACTGACACC
TGACGACACAGGCCTTTACTACTGCGCGAAAGGGGCCTTTGGGGGGAGTTCTCCCTTTGGCTTCTG
GGGCCAGGGAACTCTGCTCACCGTCTCCCCAGCCTCCACCAAGGGCCCATCGGTCTTCCCCCTGGC
ACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
SEQ ID NO: 390
LIGHT CHAIN
QSALSQPVSVSGSPGESITISCTEATTWYQQLPGRSPKLIIY [symbol] NRPSGISSRFSGSMSGRTASLTISGLQVDDE
GLYHC [symbol] FGGGTKLTVLSQPKAAPSVTLFPPSSEELQANKATLVCLVSDFYPGAVTVAWKADGSPV
KVGVETTKPSKQSNNKYAASSYLSLTPEQWKSHRSYSCRVTHEGSTVEKTVAPAECS
SEQ ID NO: 391
CAGTCTGCCCTGTCTCAGCCTGTCTCCGTGTCTGGGTCTCCTGGAGAGTCGATCACCATTTCCTGTA
CTGAAGCCACCACCTGGTATCAACAACTCCCAGGGAGATCCCCCAAACTCATTATTTATGACGTGA
CCAACCGGCCCTCAGGCATTTCAAGTCGTTTCTCTGGCTCCATGTCTGGTCGCACGGCCTCCCTGA
CAATCTCCGGTCTCCAGGTTGACGACGAGGGTCTCTATCACTGTGCCTCACGTGAATTTTTCGGCG
GGGGGACCAAGCTGACCGTCCTCAGTCAGCCCAAGGCTGCCCCCTCGGTCACTCTGTTCCCACCCT
CCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
SEQ ID NO: 392
>N49P71
HEAVY CHAIN
RVTLQQSGATVRQPGASVTVSCETS [symbol] IHWVRQAPGQGLQWVGR [symbol] KFAPIFQGKFSMS
RDLSRDTAYLGLTRLTLADTALFFC [symbol] WGQGTQVTVSAASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 393
CGTGTTACATTACAACAGTCTGGGGCTACAGTGAGGCAGCCTGGGGCCTCAGTCACCGTCTCCTGC
GAGACTTCTGGATTCACCTTCATCAAATATACCATTCACTGGGTGCGACAGGCCCCTGGACAGGGT
CTTCAGTGGGTGGGAAGAATACACCCCCGAACCGGTGCCGTGAAGTTTGCACCGATATTTCAGGG
TAAATTTTCCATGAGTCGAGACTTGTCACGCGACACAGCCTACCTCGGATTGACCAGACTGACACT
CGCCGACACGGCTCTATTTTTCTGTGCGAGGGGGGCCTTTGAGGCAGATTTATATGGGCCAACTTA
CCCCTTTCACCACTGGGGCCAAGGAACCCAAGTCACCGTCTCCGCGGCCTCCACCAAGGGCCCAT
CGGTCTTCCCCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGGT
GGACAAGAGAGTTGAGCCCAAATCTTGTGACAAAACTCACACATGCCCACCGTGCCCAGCACCTG
AACTCCTGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACA
GCACGTACCGGGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGCCAAAGG
GCAGCCCCGAGAACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGTAAA
SEQ ID NO: 394
LIGHT CHAIN
SWALTQPASVSASPGQSVTMSCTGF [symbol] DSWYQQYPGKAPKLIIY [symbol] KRPSGVSDRFSASRLGSTSSLTIS
NVQAADDAHYVC [symbol] FGGGTKLTVLSQPKAAPSNYLFPPSSEELQANKAYLNCLNSDFYPGANYNAW
KADGSPVKVGVETTKPSKQSNNKYAASSYLSLTPEQWKSHRSYSCRVTHEGSTVEKTVAPAECS
SEQ ID NO: 395
TCCTGGGCCCTGACTCAACCCGCCTCCGTGTCTGCGTCTCCTGGGCAGTCGGTCACCATGTCCTGC
ACTGGATTCGGAAGTTATAATCCTGACTCCTGGTACCAGCAATACCCAGGCAAAGCCCCCAAACT
CATCATTTATGATGACAATAAAAGACCCTCGGGGGTCTCTGATCGCTTCTCTGCCTCCAGACTTGG
CAGCACATCTTCACTGACAATCTCTAACGTCCAGGCCGCTGACGACGCCCATTATGTCTGCGCCTC
CTTTGAGTTTTTCGGCGGGGGGACCAAGCTGACCGTCCTGAGTCAGCCCAAGGCTGCCCCCTCGGT
CACTCTGTTCCCACCCTCCTCTGAGGAGCTTCAAGCCAACAAGGCCACACTGGTGTGTCTCGTAAG
TGACTTCTACCCGGGAGCCGTGACAGTGGCCTGGAAGGCAGATGGCAGCCCCGTCAAGGTGGGAG
TGGAGACCACCAAACCCTCCAAACAAAGCAACAACAAGTATGCGGCCAGCAGCTACCTGAGCCTG
ACGCCCGAGCAGTGGAAGTCCCACAGAAGCTACAGCTGCCGGGTCACGCATGAAGGGAGCACCG
TGGAGAAGACAGTGGCCCCTGCAGAATGCTCT
SEQ ID NO: 396
>N49P72
HEAVY CHAIN
HIQLLQSGPQVKKSGDTVRISCETS [symbol] IHWVRQTPEKRLRWMGW [symbol] NYAPEFQGRIRMT
RDTFIDTVYVDLSGLTPADTAYYYC [symbol] WGHGTRVTVFSASTKGPSVFPEAPSSKSTSGGT
AALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 397
CACATTCAGTTGCTACAGTCGGGGCCTCAAGTTAAGAAGTCTGGGGACACAGTGAGAATCTCCTG
TGAGACCTCTGGATATAATTTCGTCGACTCCCTTATCCACTGGGTCCGACAGACCCCGGAAAAACG
TCTCAGATGGATGGGCTGGATCAATCCTCTCCAAGGTGGTGTGAATTACGCGCCGGAATTTCAGGG
CAGAATCAGGATGACCAGGGACACGTTTATAGACACAGTTTACGTGGACTTGAGCGGACTGACAC
CGGCCGACACGGCCTATTATTACTGCGCGCGAGGGATCGATGGCAATTCTTACCCCTTTCATTTCT
GGGGCCACGGAACCCGGGTCACCGTCTTCTCGGCCTCCACCAAGGGCCCATCGGTCTTCCCCCTGG
CACCCTCCTCCAAGAGCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
SEQ ID NO: 398
LIGHT CHAIN
RFALTQPASVSGSPGQTITITCAGG [symbol] VSWFHFPPGKTPRLIIY [symbol] KRPSGVSPRFSGSQSGSTASLIISGLQSDD
EGTYFC [symbol] FGRGTLLTVLSQPKAAPSVTLFPPSSEELQANKATLVCLVSDFYPGAVTVAWKADGSPV
KVGVETTKPSKQSNNKYAASSYLSLTPEQWKSHRSYSCRVTHEGSTVEKTVAPAECS
SEQ ID NO: 399
CGATTTGCCCTGACTCAACCTGCCTCCGTGTCTGGGTCTCCTGGACAGACGATCACCATAACCTGC
GCTGGAGGCAGCGTCTCCTGGTTCCATTTCCCTCCAGGCAAAACCCCCAGACTCATTATTTATGAG
TCTTCTAAGAGACCCTCAGGGGTCTCTCCTCGATTCTCTGGGTCCCAGTCTGGCAGCACGGCCTCC
CTAATAATTTCTGGCCTCCAGTCTGATGACGAAGGGACATACTTCTGTTCTATTCTTGAATTTTTCG
GCAGAGGGACTCTTCTCACCGTCCTGAGTCAGCCCAAGGCTGCCCCCTCGGTCACTCTGTTCCCAC
CCTCCTCTGAGGAGCTTCAAGCCAACAAGGCCACACTGGTGTGTCTCGTAAGTGACTTCTACCCGG
GAGCCGTGACAGTGGCCTGGAAGGCAGATGGCAGCCCCGTCAAGGTGGGAGTGGAGACCACCAA
ACCCTCCAAACAAAGCAACAACAAGTATGCGGCCAGCAGCTACCTGAGCCTGACGCCCGAGCAGT
GGAAGTCCCACAGAAGCTACAGCTGCCGGGTCACGCATGAAGGGAGCACCGTGGAGAAGACAGT
GGCCCCTGCAGAATGCTCT
SEQ ID NO: 400
>N49P7-FR
HEAVY CHAIN
ADLVQSGAVVKKPGDSVRISCEAQ [symbol] IHWIRRAPGQGPEWMGW [symbol] NIPWKFQGRVSM
TRQLSQDPDDPDWGTAFLDLRGLKSDDTAVYYC [symbol] WGRGTAVTIQSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 501
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGCAATTATCTCAAGACCCAGACGACCCGGACTGGGGCACAGCATTTCTG
GACTTAAGAGGACTAAAGTCTGACGACACGGCCGTCTATTATTGCGTGAGAGATCGCAGTAATGG
ATCGGGAAAGCGATTCGAGTCCTCCAATTGGTTCCTCGATCTGTGGGGCCGTGGGACTGCGGTCAC
AATTCAATCAGCCTCCACCAAGGGCCCATCGGTCTTCCCCCTGGCACCCTCCTCCAAGAGCACCTC
TGGGGGCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTG
AGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGGGTGGTCAGCGTCCTCACCGTCCTGC
ACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCC
ATCCCGGGAGGAGATGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACAAGAGCAGGTGGC
AGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 502
LIGHT CHAIN
ALTQPRSVSASPGQSVTISCTGT [symbol] VSWCQHQPGRAPKLLIY [symbol] KRPSGVPDRFSGSGSGGTASLTITGLQ
DDDDAEYFC [symbol] FGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPTECS
SEQ ID NO: 503
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
SEQ ID NO: 504
>N49P9-FR
HEAVY CHAIN
HVQLVQSGGGVKKIGAAVRISCEVT [symbol] INWVRQAPGQGLEWMGW [symbol] NYSWRFEGRVT
MTRQLSQDPDDPDWGTAFMELRGLRVDDTAVYYC [symbol] WGQGVRVVVSSPSTKGPSVFPL
APSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 505
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGCAATTATCTCAAGACCCAGACGACCCGGACTGGGGCACGGCCTT
CATGGAGTTGAGAGGACTGAGAGTGGACGACACGGCCGTCTATTATTGCGCGAGGGGACCCTCTG
GGGAAAATTATCCTTTTCACTATTGGGGCCAGGGTGTCCGAGTGGTCGTCTCGTCACCCTCCACCA
AGGGCCCATCGGTCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCA
ACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACAACAGCACGTACCGGGTGGTCAGCGTCCTCACCGTCCTGCACCAGGACTGGCTGAATGG
CAAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGAGGAGATGAC
CAAGAACCAGGTCAGCCTGACCTGCCTGGTCAAAGGCTTCTATCCCAGCGACATCGCCGTGGAGT
GGGAGAGCAATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 506
LIGHT CHAIN
ASALTQPASMSASPGQSVTISCSGT [symbol] SAWFQQYPGKPPKLIIF [symbol] KRPSGVPSRFSASRPGDTASLTISNV
QPEDEATYIC [symbol] FGGGTRLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGSTVEKTVAPAECS
SEQ ID NO: 295
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
SEQ ID NO: 296
>N49P9.3-FR
HEAVY CHAIN
HVQLVQSGGGVKKIGAAVRISCEVS [symbol] INWVRQAPGQGLEWMGW [symbol] NYSWRFQGRVT
MTRQLSQDPDDPDWGTAFMELRGLRVDDTAVYYC [symbol] WGQGVRVVVSSPSTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 507
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCACCCTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 508
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLRQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 327
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTACGTCAGCCCAAGGCTGCCCCCTCGGTCACTCTGTTCCCA
CCCTCCTCTGAGGAGCTTCAAGCCAACAAGGCCACACTGGTGTGTCTCGTAAGTGACTTCTACCCG
GGAGCCGTGACAGTGGCCTGGAAGGCAGATGGCAGCCCCGTCAAGGTGGGAGTGGAGACCACCA
AACCCTCCAAACAAAGCAACAACAAGTATGCGGCCAGCAGCTACCTGAGCCTGACGCCCGAGCA
GTGGAAGTCCCACAGAAGCTACAGCTGCCGGGTCACGCATGAAGGGAGCACCGTGGAGAAGACA
GTGGCCCCTGCAGAATGCTCT
SEQ ID NO: 328
>N49P9.6-FR
HEAVY CHAIN
HVQLVQSGGGVKKIGAAVRISCEVS [symbol] INWVRQAPGQGLEWMGW [symbol] NYSWRFQGRVT
MTRQLSQDPDDPDWGTAFMELRGLRVDDTAVYYC [symbol] WGQGVRWVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 509
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 510
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 511
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 512
>N49P9.6-FR-54W
HEAVY CHAIN
HVQLVQSGGGVKKIGAAVRISCEVS [symbol] INWVRQAPGQGLEWMGW [symbol] NYSWRFQGRV
TMTRQLSQDPDDPDWGTAFMELRGLRVDDTAVYYC [symbol] WGQGVRVVVSSASTKGPSVF
PLAPSSKSTSGGTAALGCLVKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 513
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATGGGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 514
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 515
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 516
>N49P9.6-FR-54F
HEAVY CHAIN
HVQLVQSGGGVKKIGAAVRISCEVS [symbol] INWVRQAPGQGLEWMGW [symbol] NYSWRFQGRVT
MTRQLSQDPDDPDWGTAFMELRGLRVDDTAVYYC [symbol] WGQGVRVVVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 517
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATTTGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 518
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 519
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 520
>N49P9.6-FR3-06
HEAVY CHAIN
HVQLVQSGGGVKKIGAAVRISCEVS [symbol] INWVRQAPGQGLEWMGW [symbol] NYSWRFQGRVT
MTRLFSQDLYYPDRGTAFMELRGLRVDDTAVYYC [symbol] WGQGVRVVVSSASYKGPSVFPLA
PSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 521
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCTATTCTCTCAAGACCTATACTACCCGGACCGGGGCACGGCCTT
CATGGAGTTGAGAGGACTGAGAGTGGACGACACGGCCGTCTATTATTGCGCGAGGGGACCCTCTG
GGGAAAATTATCCTTTTCACTATTGGGGCCAGGGTGTCCGAGTGGTCGTCTCGTCAGCGTCCACCA
AGGGCCCATCGGTCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCA
ACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACAACAGCACGTACCGGGTGGTCAGCGTCCTCACCGTCCTGCACCAGGACTGGCTGAATGG
CAAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGAGGAGATGAC
CAAGAACCAGGTCAGCCTGACCTGCCTGGTCAAAGGCTTCTATCCCAGCGACATCGCCGTGGAGT
GGGAGAGCAATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
SEQ ID NO: 522
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 523
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 524
N49P9.6-FR1-D
HEAVY CHAIN
HVQLVQSGGGVKKIGAAVRISCEVS [symbol] INWVRQAPGQGLEWMGW [symbol] NYSWRFQGRVT
MTRQLSQDPDDPDWGTAFMELRGLRVDDTAVYYC [symbol] WGQGVRVVVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 525
CACGTCCAATTGGTGCAGTCTGGAGGTGGGGTGAAGAAGATTGGGGCCGCTGTGAGGATCTCCTG
CGAGGTGTCTGGATACGACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 526
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 527
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 528
>N49P9.6-FR1-D-I
HEAVY CHAIN
HVQLVQSGGGVKKIGAAVRISCEVS [symbol] INWVRQAPGQGLEWMGW [symbol] YSWRFQGRVT
MTRQLSQDPDDPDWGTAFMELRGLRVDDTAVYYC [symbol] WGQGVRVVVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 529
CACGTCCAATTGGTGCAGTCTGGAGGTGGGGTGAAGAAGATTGGGGCCGCTGTGAGGATCTCCTG
CGAGGTGTCTGGATACGACTTCATTGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 530
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 531
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 532
>N49P9.6-FR-LS
HEAVY CHAIN
HVQLVQSGGGVKKIGAAVRISCEVS [symbol] INWVRQAPGQGLEWMGW [symbol] NYSWRFQGRVT
MTRQLSQDPDDPDWGTAFMELRGLRVDDTAVYYC [symbol] WGQGVRVVVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVLHEALHSHYTQKSLSLSPGK
SEQ ID NO: 533
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGCTGCATGAGGCTCTGCACAGCCACTACACGCAGAAGAGCCTCTCCCTGTCTC
CGGGTAAA
SEQ ID NO: 534
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 535
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 536
>N49P9.6-FR-YTE
HEAVY CHAIN
HVQLVQSGGGVKKIGAAVRISCEVS [symbol] INWVRQAPGQGLEWMGW [symbol] NYSWRFQGRVT
MTRQLSQDPDDPDWGTAFMELRGLRVDDTAVYYC [symbol] WGQGVRVVVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLYITREPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 537
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCTATATCACCCGGGAGCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
SEQ ID NO: 538
LIGHT CHAIN
LTQPASMSASPGQSVTISCSGT [symbol] SAWFQQYPGKPPKLIIF [symbol] KRPSGVPSRFSASRPGDTASLTISNVQPE
DEATYIC [symbol] FGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 539
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
SEQ ID NO: 540

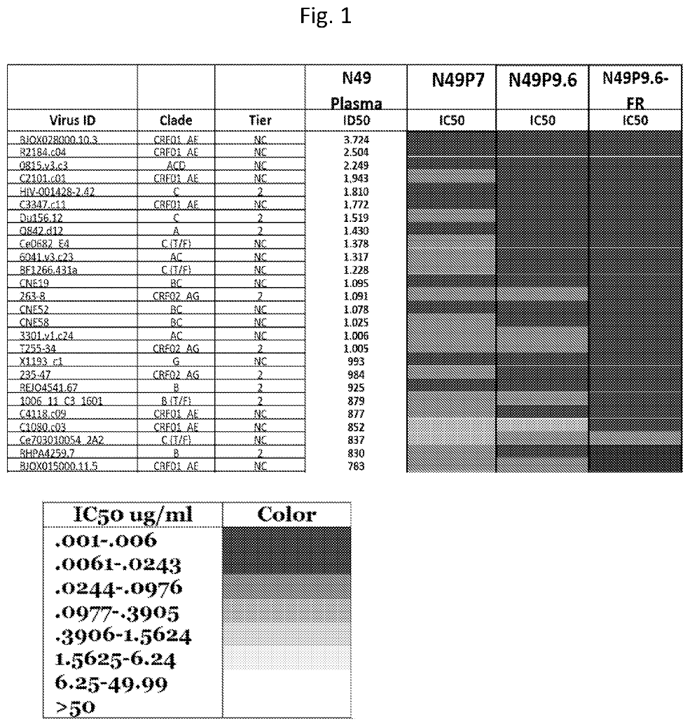

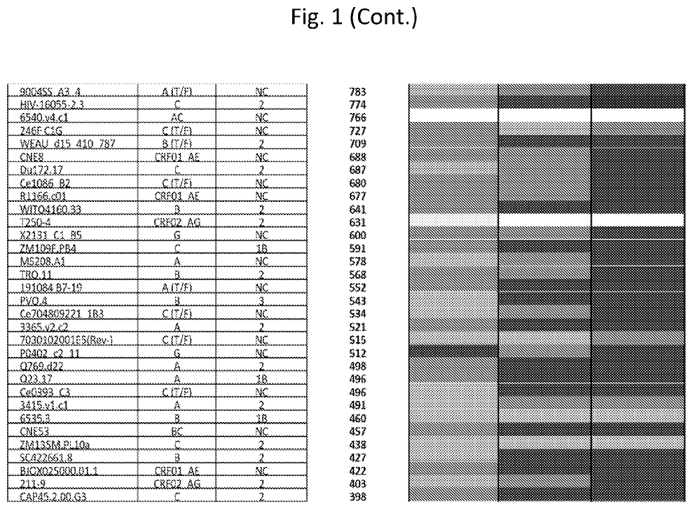

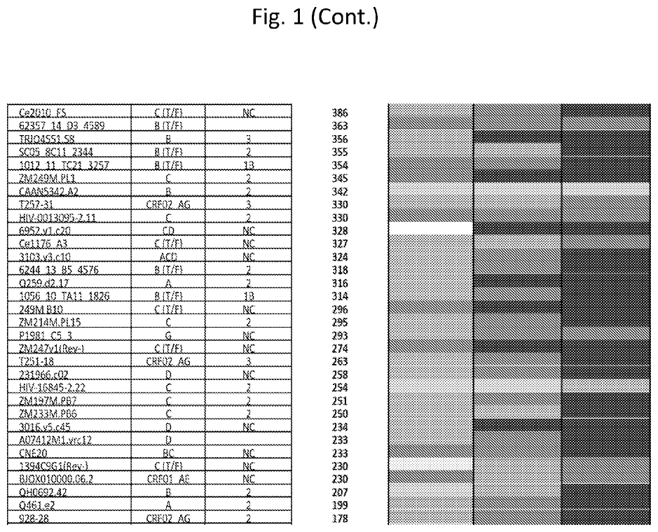

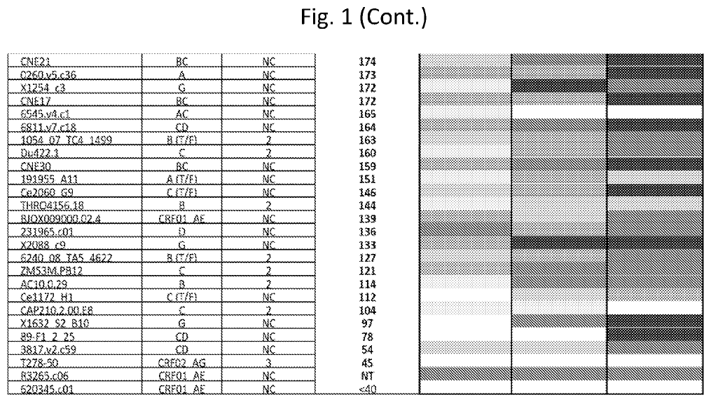

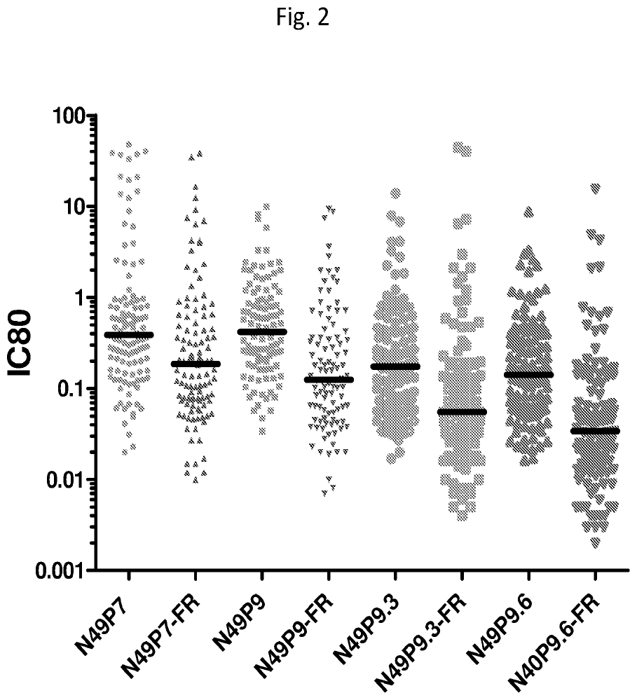

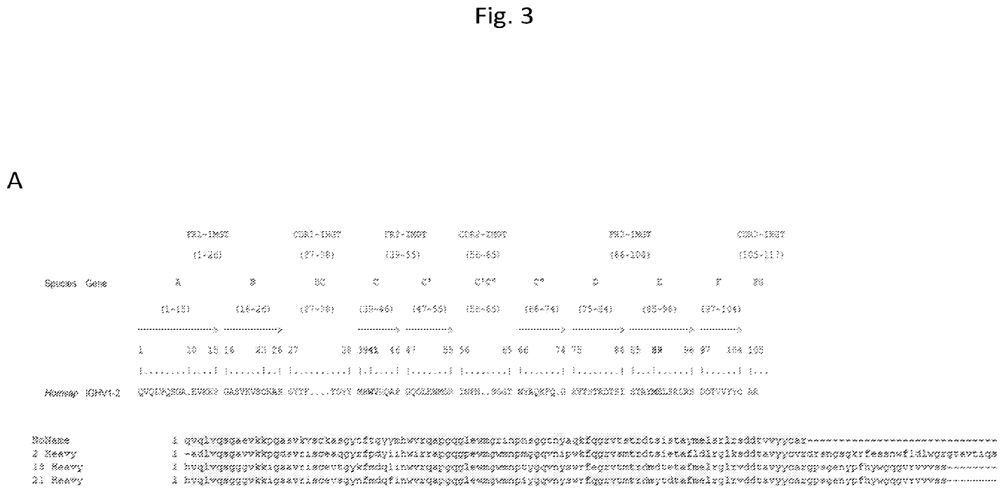

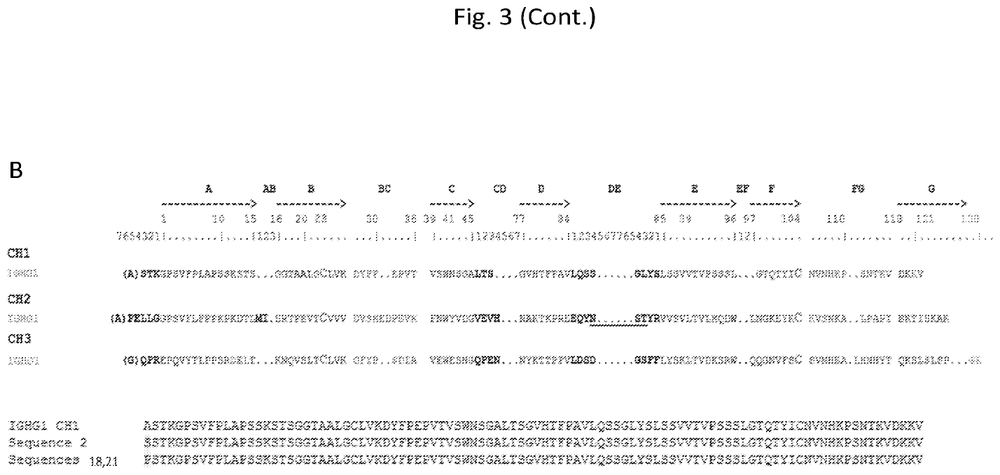

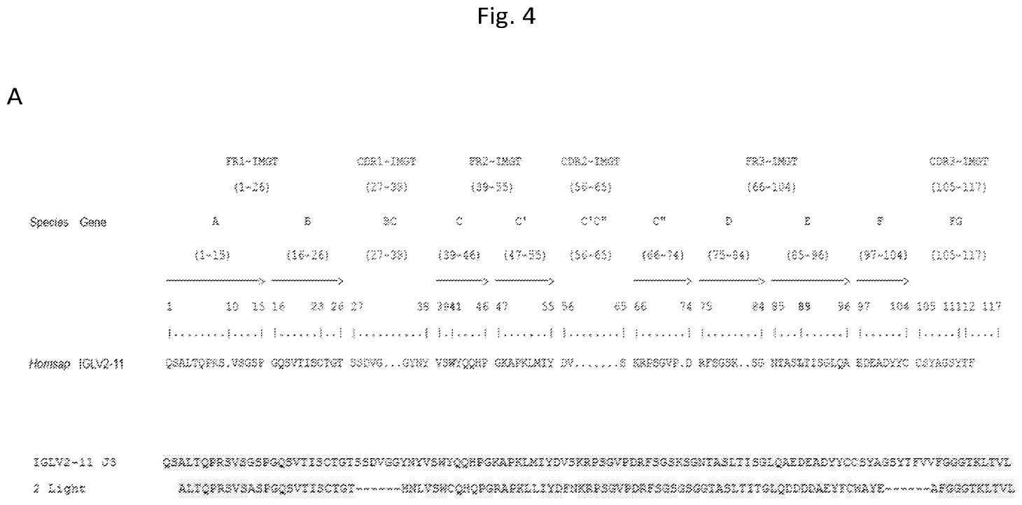

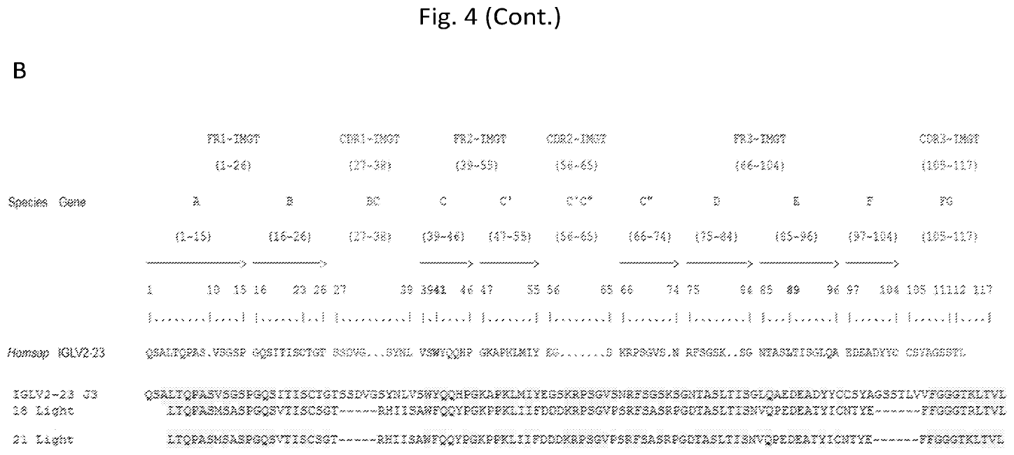

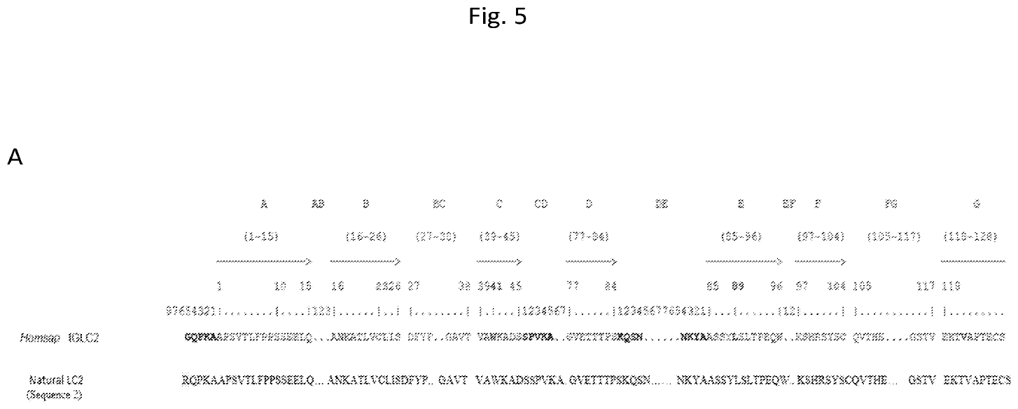

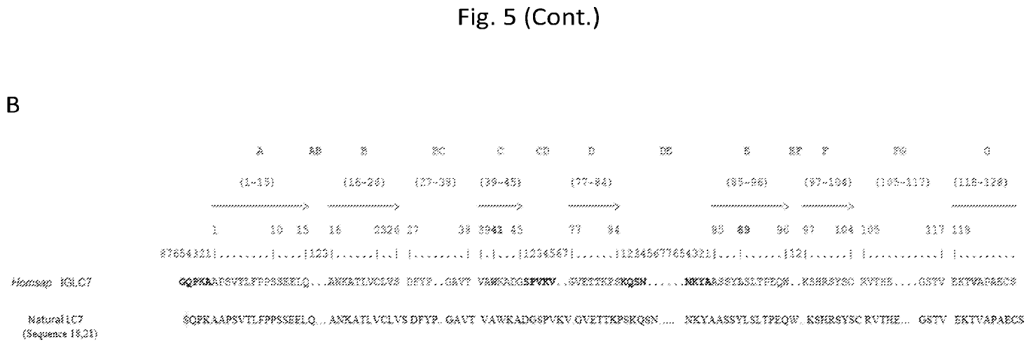

In some embodiments, the antibodies of the invention have a particularly high potency in neutralizing HIV infection in vitro across multiple clades as shown in Table 4, below (see also FIG. 1). Such antibodies are desirable, as only low concentrations are required in order to neutralize a given amount of virus. This facilitates higher levels of protection while administering lower amounts of antibody.

In some embodiments, the anti-HIV antibody neutralizes at least 95%, at least 96%, at least 97%, at least 98%, at least 99% or 100% of the HIV pseudoviruses listed Table 4 (see also FIG. 1) with an IC50 value of less than 50 μg/mL.

In some embodiments, the anti-HIV antibody neutralizes at least 50%, at least 55%, at least 60%, at least 65%, at least 70%, at least 75%, at least 80%, at least 85%, at least 90%, at least 95%, or at least 99% of the HIV pseudoviruses listed in Table 4 with an IC50 value of less than about 1 μg/ml, between about 1-5 μg/ml or greater than about 5 μg/ml.

The neutralization can be performed using a luciferase-based assay in TZM.bl cells as described by M. M. Sajadi et al., J Acquir Immune Defic Syndr 57, 9-15 (2011) and M. Li et al., J Virol 79, 10108-10125 (2005)). This assay measures the reduction in luciferase expression following a single round of virus infection.

TABLE 4
HIV Env pseudovirus panel.

| Virus ID | Clade | Tier |
|---|---|---|
| BJOX028000.10.3 | CRF01_AE (T/F) | NC |
| R2184.c04 | CRF01_AE | NC |
| 0815.v3.c3 | ACD | NC |
| C2101.c01 | CRF01_AE | NC |
| HIV-001428-2.42 | C | 2 |
| C3347.c11 | CRF01_AE | NC |
| Du156.12 | C | 2 |
| Q842.d12 | A | 2 |
| Ce0682_E4 | C (T/F) | NC |
| 6041.v3.c23 | AC | NC |
| BF1266.431a | C (T/F) | NC |
| CNE19 | BC | NC |
| 263-8 | CRF02_AG | 2 |
| CNE52 | BC | NC |
| CNE58 | BC | NC |
| 3301.v1.c24 | AC | NC |
| T255-34 | CRF02_AG | 2 |
| X1193_c1 | G | NC |
| 235-47 | CRF02_AG | 2 |
| REJO4541.67 | B | 2 |
| 1006_11_C3_1601 | B (T/F) | 2 |
| C4118.c09 | CRF01_AE | NC |
| C1080.c03 | CRF01_AE | NC |
| Ce703010054_2A2 | C (T/F) | NC |
| RHPA4259.7 | B | 2 |
| BJOX015000.11.5 | CRF01_AE (T/F) | NC |
| 9004SS_A3_4 | A (T/F) | NC |
| HIV-16055-2.3 | C | 2 |
| 6540.v4.c1 | AC | NC |
| 246F C1G | C (T/F) | NC |
| WEAU_d15_410_787 | B (T/F) | 2 |
| CNE8 | CRF01_AE | NC |
| Du172.17 | C | 2 |
| Ce1086_B2 | C (T/F) | NC |
| R1166.c01 | CRF01_AE | NC |
| WITO4160.33 | B | 2 |
| T250-4 | CRF02_AG | 2 |
| X2131_C1_B5 | G | NC |
| ZM109F.PB4 | C | 1B |
| MS208.A1 | A | NC |
| TRO.11 | B | 2 |
| 191084 B7-19 | A (T/F) | NC |
| PVO.4 | B | 3 |
| Ce704809221_1B3 | C (T/F) | NC |
| 3365.v2.c2 | A | 2 |
| 7030102001E5(Rev-) | C (T/F) | NC |
| P0402_c2_11 | G | NC |
| Q769.d22 | A | 2 |
| Q23.17 | A | 1B |
| Ce0393_C3 | C (T/F) | NC |
| 3415.v1.c1 | A | 2 |
| 6535.3 | B | 1B |
| CNE53 | BC | NC |
| ZM135M.PL10a | C | 2 |
| SC422661.8 | B | 2 |
| BJOX025000.01.1 | CRF01_AE (T/F) | NC |
| 211-9 | CRF02_AG | 2 |
| CAP45.2.00.G3 | C | 2 |
| Ce2010_F5 | C (T/F) | NC |
| 62357_14_D3_4589 | B (T/F) | |
| TRJO4551.58 | B | 3 |
| SC05_8C11_2344 | B (T/F) | 2 |
| 1012_11_TC21_3257 | B (T/F) | 1B |
| ZM249M.PL1 | C | 2 |
| CAAN5342.A2 | B | 2 |
| T257-31 | CRF02_AG | 3 |
| HIV-0013095-2.11 | C | 2 |
| 6952.v1.c20 | CD | NC |
| Ce1176_A3 | C (T/F) | NC |
| 3103.v3.c10 | ACD | NC |
| 6244_13_B5_4576 | B (T/F) | 2 |
| Q259.d2.17 | A | 2 |
| 1056_10_TA11_1826 | B (T/F) | 1B |
| 249M B10 | C (T/F) | NC |
| ZM214M.PL15 | C | 2 |
| P1981_C5_3 | G | NC |
| ZM247v1(Rev-) | C (T/F) | NC |
| T251-18 | CRF02_AG | 3 |
| 231966.c02 | D | NC |
| HIV-16845-2.22 | C | 2 |
| ZM197M.PB7 | C | 2 |
| ZM233M.PB6 | C | 2 |
| 3016.v5.c45 | D | NC |
| A07412M1.vrc12 | D | |
| CNE20 | BC | NC |
| 1394C9G1(Rev-) | C (T/F) | NC |
| BJOX010000.06.2 | CRF01_AE (T/F) | NC |
| QH0692.42 | B | 2 |
| Q461.e2 | A | 2 |
| 928-28 | CRF02_AG | 2 |
| CNE21 | BC | NC |
| 0260.v5.c36 | A | NC |
| X1254_c3 | G | NC |
| CNE17 | BC | NC |
| 6545.v4.c1 | AC | NC |
| 6811.v7.c18 | CD | NC |
| 1054_07_TC4_1499 | B (T/F) | 2 |
| Du422.1 | C | 2 |
| CNE30 | BC | NC |
| 191955_A11 | A (T/F) | NC |
| Ce2060_G9 | C (T/F) | NC |
| THRO4156.18 | B | 2 |
| BJOX009000.02.4 | CRF01_AE | NC |
| 231965.c01 | D | NC |
| X2088_c9 | G | NC |
| 6240_08_TA5_4622 | B (T/F) | 2 |
| ZM53M.PB12 | C | 2 |
| AC10.0.29 | B | 2 |
| Ce1172_H1 | C (T/F) | NC |
| CAP210.2.00.E8 | C | 2 |
| X1632_S2_B10 | G | NC |
| 89-F1_2_25 | CD | NC |
| 3817.v2.c59 | CD | NC |
| T278-50 | CRF02_AG | 3 |
| R3265.c06 | CRF01_AE | NC |
| 620345.c01 | CRF01_AE | NC |

Methods for producing antibodies, such as those disclosed herein, are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains.

Standard techniques of molecular biology may be used to prepare DNA sequences coding for the antibodies or fragments of the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention or fragments thereof. Bacterial, for example E. coli, and other microbial systems may be used, in part, for expression of antibody fragments such as Fab and F(ab′)2 fragments, and especially Fv fragments and single chain antibody fragments, for example, single chain Fvs. Eukaryotic, e.g. mammalian, host cell expression systems may be used for production of larger antibody molecules, including complete antibody molecules. Suitable mammalian host cells include CHO, HEK293T, PER.C6, myeloma or hybridoma cells.

In some embodiments, antibodies according to the invention may be produced by i) expressing a nucleic acid sequence according to the invention in a cell, and ii) isolating the expressed antibody product. Additionally, the method may include iii) purifying the antibody.

For the antibodies of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the antibodies of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer may be used for expression of the antibodies of the invention. For example, suitable promoters and/or enhancers can be selected from the Eukaryotic Promoter Database (EPDB).

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions. Specific expression and purification conditions will vary depending upon the expression system employed.

Following expression, the antibodies and/or antigens of the invention can be isolated and/or purified or concentrated using any suitable technique known in the art. For example, anion or cation exchange chromatography, phosphocellulose chromatography, hydrophobic interaction chromatography, affinity chromatography, immuno-affinity chromatography, hydroxyapatite chromatography, lectin chromatography, molecular sieve chromatography, isoelectric focusing, gel electrophoresis, or any other suitable method or combination of methods can be used.

In some embodiments, the antibodies can be made using recombinant DNA methods as described in U.S. Pat. No. 4,816,567. The polynucleotides encoding a monoclonal antibody can be isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells.

The anti-HIV antibodies can also include insertions, deletions, substitutions, or other selected modifications of particular regions or specific amino acids residues. It should be understood that the antibodies of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. For example, leucine can be replaced with isoleucine or valine, or vice versa; an aspartate with a glutamate or vice versa; a threonine with a serine or vice versa; or a similar conservative replacement of an amino acid with a structurally related amino acid can be made.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

For the purposes of the present invention, it should be appreciated that modified antibodies can comprise any type of variable region that provides for the association of the antibody with the polypeptides of HIV such as gp120.

In some embodiments, the variable regions or domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, in some embodiments the CDRs will be derived from an antibody of different class.

Alterations to the variable region notwithstanding, those skilled in the art will appreciate that the modified antibodies of this invention can comprise antibodies (e.g., full-length antibodies or immunoreactive fragments thereof) in which at least a fraction of one or more of the constant region domains has been deleted or otherwise altered so as to provide desired biochemical characteristics such as increased localization, increased serum half-life or reduced serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. That is, the modified antibodies disclosed herein can comprise alterations or modifications to one or more of the three heavy chain constant domains (CH1, CH2 or CH3) and/or to the light chain constant domain (CL). In some embodiments, modified constant regions wherein one or more domains are partially or entirely deleted are contemplated. In some embodiments, the modified antibodies will comprise domain deleted constructs or variants wherein the entire CH2 domain has been removed (ACH2 constructs). In some embodiments, the omitted constant region domain will be replaced by a short amino acid spacer (e.g. 10 residues) that provides some of the molecular flexibility typically imparted by the absent constant region.

Besides their configuration, it is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and can also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, the anti-HIV antibodies provide for altered effector functions that, in turn, affect the biological profile of the administered antibody. For example, the deletion or inactivation (through point mutations or other means) of a constant region domain can reduce Fc receptor binding of the circulating modified antibody thereby increasing tumor localization. In other cases it may be that constant region modifications, consistent with this invention, moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region in accordance with this invention can easily be made using well known biochemical or molecular engineering techniques well within the purview of the skilled artisan.

In some embodiments, the anti-HIV antibody is an antibody that binds to the same epitope as an antibody selected from the group consisting of N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, and N49P9.6-FR-YTE.

In some embodiments, the anti-HIV antibody comprises a heavy chain comprising an amino acid sequence selected from the group consisting of SEQ ID NOS:501, 505, 507, 509, 513, 517, 521, 525, 529, 533, and 537.

In some embodiments, the anti-HIV antibody comprises an antigen binding fragment of an amino acid sequence selected from the group consisting of SEQ ID NOS:501, 505, 507, 509, 513, 517, 521, 525, 529, 533, and 537.

In some embodiments, the anti-HIV antibody comprises a light chain comprising an amino acid sequence selected from the group consisting of SEQ ID NOS:503, 295, 327, 511, 515, 519, 523, 527, 531, 535, and 539.

In some embodiments, the anti-HIV antibody comprises an antigen binding fragment of an amino acid sequence selected from the group consisting of SEQ ID NOS:503, 295, 327, 511, 515, 519, 523, 527, 531, 535, and 539.

In some embodiments, the anti-HIV antibody comprises a heavy chain comprising an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 and 397, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the anti-HIV antibody comprises an antigen binding fragment of an amino acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 and 397, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the anti-HIV antibody comprises a light chain comprising an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539.

In some embodiments, the anti-HIV antibody comprises an antigen binding fragment of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539.

For any of the light chain sequences described herein, and for any of the antibodies described herein, the first 1, 2, or 3 amino acids in the light chain [by IMGT numbering system] can be deleted, or can be substituted with another amino acid, e.g., a conservative amino acid substitution. In some embodiments there is a deletion of the first 2 or 3 amino acids. In some embodiments, the antibodies can comprise the variable region of such antibodies. For example, the variable region of SEQ ID NO:36 comprises amino acids 1-100 of SEQ ID NO:36. If an antibody has a light chain that has a 2 amino acid deletion in SEQ ID NO:36, the variable region will comprise amino acids 3-100 of SEQ ID NO:36.

In some embodiments, the anti-HIV antibody is selected from the group consisting of:
a. an antibody comprising a heavy chain amino acid sequence comprising SEQ ID NO:1 or an antigen binding fragment thereof, except that the heavy chain amino acid sequence is modified whereby a part or all of the framework 3 region of SEQ ID NO:1 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:2 or an antigen binding fragment thereof;
b. an antibody comprising a heavy chain amino acid sequence comprising SEQ ID NO:3 or an antigen binding fragment thereof, except that the heavy chain amino acid sequence is modified whereby a part or all of the framework 3 region of SEQ ID NO:3 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:4 or an antigen binding fragment thereof;
c. an antibody comprising a heavy chain amino acid sequence comprising SEQ ID NO:5 or an antigen binding fragment thereof, except that the heavy chain amino acid sequence is modified whereby a part or all of the framework 3 region of SEQ ID NO:5 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:6 or an antigen binding fragment thereof;
d. an antibody comprising a heavy chain amino acid sequence comprising SEQ ID NO:7 or an antigen binding fragment thereof, except that the heavy chain amino acid sequence is modified whereby a part or all of the framework 3 region of SEQ ID NO:7 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:8 or an antigen binding fragment thereof;
e. an antibody comprising a heavy chain amino acid sequence comprising SEQ ID NO:9 or an antigen binding fragment thereof, except that the heavy chain amino acid sequence is modified whereby a part or all of the framework 3 region of SEQ ID NO:9 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:10 or an antigen binding fragment thereof;
f. an antibody comprising a heavy chain amino acid sequence comprising SEQ ID NO:11 or an antigen binding fragment thereof, except that the heavy chain amino acid sequence is modified whereby a part or all of the framework 3 region of SEQ ID NO:11 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:12 or an antigen binding fragment thereof;
g. an antibody comprising a heavy chain amino acid sequence comprising SEQ ID NO:13 or an antigen binding fragment thereof, except that the heavy chain amino acid sequence is modified whereby a part or all of the framework 3 region of SEQ ID NO:13 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:14 or an antigen binding fragment thereof;
h. an antibody comprising a heavy chain amino acid sequence comprising SEQ ID NO:15 or an antigen binding fragment thereof, except that the heavy chain amino acid sequence is modified whereby a part or all of the framework 3 region of SEQ ID NO:15 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:16 or an antigen binding fragment thereof;
i. an antibody comprising a heavy chain amino acid sequence comprising SEQ ID NO:17 or an antigen binding fragment thereof, except that the heavy chain amino acid sequence is modified whereby a part or all of the framework 3 region of SEQ ID NO:17 is replaced with an amino acid sequence selected from the group consisting of QLSQDPDDPDWG (SEQ ID NO:541) and comprising SEQ ID NO:18 or an antigen binding fragment thereof;
j. an antibody comprising a heavy chain amino acid sequence comprising SEQ ID NO:19 or an antigen binding fragment thereof, except that the heavy chain amino acid sequence is modified whereby a part or all of the framework 3 region of SEQ ID NO:19 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:20 or an antigen binding fragment thereof;
k. an antibody comprising a heavy chain amino acid sequence comprising SEQ ID NO:21 or an antigen binding fragment thereof, except that the heavy chain amino acid sequence is modified whereby a part or all of the framework 3 region of SEQ ID NO:21 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:22 or an antigen binding fragment thereof;
l. an antibody comprising a heavy chain amino acid sequence comprising SEQ ID NO:23 or an antigen binding fragment thereof, except that the heavy chain amino acid sequence is modified whereby a part or all of the framework 3 region of SEQ ID NO:23 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:24 or an antigen binding fragment thereof;
m. an antibody comprising a heavy chain amino acid sequence comprising SEQ ID NO:25 or an antigen binding fragment thereof, except that the heavy chain amino acid sequence is modified whereby a part or all of the framework 3 region of SEQ ID NO:25 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:26 or an antigen binding fragment thereof;
n. an antibody comprising a heavy chain amino acid sequence comprising SEQ ID NO:27 or an antigen binding fragment thereof, except that the heavy chain amino acid sequence is modified whereby a part or all of the framework 3 region of SEQ ID NO:27 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:28 or an antigen binding fragment thereof;
o. an antibody comprising a heavy chain amino acid sequence comprising SEQ ID NO:29 or an antigen binding fragment thereof, except that the heavy chain amino acid sequence is modified whereby a part or all of the framework 3 region of SEQ ID NO:29 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:30 or an antigen binding fragment thereof;
p. an antibody comprising a heavy chain amino acid sequence comprising SEQ ID NO:31 or an antigen binding fragment thereof, except that the heavy chain amino acid sequence is modified whereby a part or all of the framework 3 region of SEQ ID NO:31 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:32 or an antigen binding fragment thereof;
q. an antibody comprising a heavy chain amino acid sequence comprising SEQ ID NO:33 or an antigen binding fragment thereof, except that the heavy chain amino acid sequence is modified whereby a part or all of the framework 3 region of SEQ ID NO:33 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:34 or an antigen binding fragment thereof;
r. an antibody comprising a heavy chain amino acid sequence comprising SEQ ID NO:35 or an antigen binding fragment thereof, except that the heavy chain amino acid sequence is modified whereby a part or all of the framework 3 region of SEQ ID NO:35 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:36 or an antigen binding fragment thereof;
s. an antibody comprising a heavy chain amino acid sequence comprising SEQ ID NO:37 or an antigen binding fragment thereof, except that the heavy chain amino acid sequence is modified whereby a part or all of the framework 3 region of SEQ ID NO:37 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:38 or an antigen binding fragment thereof;
t. an antibody comprising a heavy chain amino acid sequence comprising SEQ ID NO:39 or an antigen binding fragment thereof, except that the heavy chain amino acid sequence is modified whereby a part or all of the framework 3 region of SEQ ID NO:39 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:40 or an antigen binding fragment thereof;
u. an antibody comprising a heavy chain amino acid sequence comprising SEQ ID NO:41 or an antigen binding fragment thereof, except that the heavy chain amino acid sequence is modified whereby a part or all of the framework 3 region of SEQ ID NO:41 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:42 or an antigen binding fragment thereof;
v. an antibody comprising a heavy chain amino acid sequence comprising SEQ ID NO:43 or an antigen binding fragment thereof, except that the heavy chain amino acid sequence is modified whereby a part or all of the framework 3 region of SEQ ID NO:43 is replaced with an amino acid sequence selected from the group consisting of QLSQDPDDPDWG (SEQ ID NO:541) and comprising SEQ ID NO:44 or an antigen binding fragment thereof;
w. an antibody comprising a heavy chain amino acid sequence comprising SEQ ID NO:45 or an antigen binding fragment thereof, except that the heavy chain amino acid sequence is modified whereby a part or all of the framework 3 region of SEQ ID NO:45 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:46 or an antigen binding fragment thereof;
x. an antibody comprising a heavy chain amino acid sequence comprising SEQ ID NO:47 or an antigen binding fragment thereof, except that the heavy chain amino acid sequence is modified whereby a part or all of the framework 3 region of SEQ ID NO:47 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:48 or an antigen binding fragment thereof;
y. an antibody comprising a heavy chain amino acid sequence comprising SEQ ID NO:49 or an antigen binding fragment thereof, except that the heavy chain amino acid sequence is modified whereby a part or all of the framework 3 region of SEQ ID NO:49 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:50 or an antigen binding fragment thereof;
z. an antibody comprising a heavy chain amino acid sequence comprising SEQ ID NO:51 or an antigen binding fragment thereof, except that the heavy chain amino acid sequence is modified whereby a part or all of the framework 3 region of SEQ ID NO:51 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:52 or an antigen binding fragment thereof;
aa. an antibody comprising a heavy chain amino acid sequence comprising SEQ ID NO:53 or an antigen binding fragment thereof, except that the heavy chain amino acid sequence is modified whereby a part or all of the framework 3 region of SEQ ID NO:53 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:54 or an antigen binding fragment thereof;
bb. an antibody comprising a heavy chain amino acid sequence comprising SEQ ID NO:55 or an antigen binding fragment thereof, except that the heavy chain amino acid sequence is modified whereby a part or all of the framework 3 region of SEQ ID NO:55 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:56 or an antigen binding fragment thereof;
cc. an antibody comprising a heavy chain amino acid sequence comprising SEQ ID NO:57 or an antigen binding fragment thereof, except that the heavy chain amino acid sequence is modified whereby a part or all of the framework 3 region of SEQ ID NO:57 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:58 or an antigen binding fragment thereof;
dd. an antibody comprising a heavy chain amino acid sequence comprising SEQ ID NO:59 or an antigen binding fragment thereof, except that the heavy chain amino acid sequence is modified whereby a part or all of the framework 3 region of SEQ ID NO:59 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:60 or an antigen binding fragment thereof;
ee. an antibody comprising a heavy chain amino acid sequence comprising SEQ ID NO:61 or an antigen binding fragment thereof, except that the heavy chain amino acid sequence is modified whereby a part or all of the framework 3 region of SEQ ID NO:61 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:62 or an antigen binding fragment thereof;
ff. an antibody comprising a heavy chain amino acid sequence comprising SEQ ID NO:63 or an antigen binding fragment thereof, except that the heavy chain amino acid sequence is modified whereby a part or all of the framework 3 region of SEQ ID NO:63 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:64 or an antigen binding fragment thereof;
gg. an antibody comprising a heavy chain amino acid sequence comprising SEQ ID NO:65 or an antigen binding fragment thereof, except that the heavy chain amino acid sequence is modified whereby a part or all of the framework 3 region of SEQ ID NO:65 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:66 or an antigen binding fragment thereof;
hh. an antibody comprising a heavy chain amino acid sequence comprising SEQ ID NO:67 or an antigen binding fragment thereof, except that the heavy chain amino acid sequence is modified whereby a part or all of the framework 3 region of SEQ ID NO:67 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:68 or an antigen binding fragment thereof;
ii. an antibody comprising a heavy chain amino acid sequence comprising SEQ ID NO:69 or an antigen binding fragment thereof, except that the heavy chain amino acid sequence is modified whereby a part or all of the framework 3 region of SEQ ID NO:69 is replaced with an amino acid sequence selected from the group consisting of QLSQDPDDPDWG (SEQ ID NO:541) and comprising SEQ ID NO:70 or an antigen binding fragment thereof;
jj. an antibody comprising a heavy chain amino acid sequence comprising SEQ ID NO:71 or an antigen binding fragment thereof, except that the heavy chain amino acid sequence is modified whereby a part or all of the framework 3 region of SEQ ID NO:71 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:72 or an antigen binding fragment thereof;
kk. an antibody comprising a heavy chain amino acid sequence comprising SEQ ID NO:73 or an antigen binding fragment thereof, except that the heavy chain amino acid sequence is modified whereby a part or all of the framework 3 region of SEQ ID NO:73 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:74 or an antigen binding fragment thereof;
ll. an antibody comprising a heavy chain amino acid sequence comprising SEQ ID NO:75 or an antigen binding fragment thereof, except that the heavy chain amino acid sequence is modified whereby a part or all of the framework 3 region of SEQ ID NO:75 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:76 or an antigen binding fragment thereof;
mm. an antibody comprising a heavy chain amino acid sequence comprising SEQ ID NO:153 or an antigen binding fragment thereof, except that the heavy chain amino acid sequence is modified whereby a part or all of the framework 3 region of SEQ ID NO:153 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:155 or an antigen binding fragment thereof;
nn. an antibody comprising a heavy chain amino acid sequence comprising SEQ ID NO:157 or an antigen binding fragment thereof, except that the heavy chain amino acid sequence is modified whereby a part or all of the framework 3 region of SEQ ID NO:157 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:159 or an antigen binding fragment thereof;
oo. an antibody comprising a heavy chain amino acid sequence comprising SEQ ID NO:161 or an antigen binding fragment thereof, except that the heavy chain amino acid sequence is modified whereby a part or all of the framework 3 region of SEQ ID NO:161 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:163 or an antigen binding fragment thereof;
pp. an antibody comprising a heavy chain amino acid sequence comprising SEQ ID NO:165 or an antigen binding fragment thereof, except that the heavy chain amino acid sequence is modified whereby a part or all of the framework 3 region of SEQ ID NO:165 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:167 or an antigen binding fragment thereof;
qq. an antibody comprising a heavy chain amino acid sequence comprising SEQ ID NO:169 or an antigen binding fragment thereof, except that the heavy chain amino acid sequence is modified whereby a part or all of the framework 3 region of SEQ ID NO:169 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:171 or an antigen binding fragment thereof;
rr. an antibody comprising a heavy chain amino acid sequence comprising SEQ ID NO:173 or an antigen binding fragment thereof, except that the heavy chain amino acid sequence is modified whereby a part or all of the framework 3 region of SEQ ID NO:173 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:175 or an antigen binding fragment thereof;
ss. an antibody comprising a heavy chain amino acid sequence comprising SEQ ID NO:177 or an antigen binding fragment thereof, except that the heavy chain amino acid sequence is modified whereby a part or all of the framework 3 region of SEQ ID NO:177 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:179 or an antigen binding fragment thereof;
tt. an antibody comprising a heavy chain amino acid sequence comprising SEQ ID NO:181 or an antigen binding fragment thereof, except that the heavy chain amino acid sequence is modified whereby a part or all of the framework 3 region of SEQ ID NO:181 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:183 or an antigen binding fragment thereof;
uu. an antibody comprising a heavy chain amino acid sequence comprising SEQ ID NO:185 or an antigen binding fragment thereof, except that the heavy chain amino acid sequence is modified whereby a part or all of the framework 3 region of SEQ ID NO:185 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:187 or an antigen binding fragment thereof;
vv. an antibody comprising a heavy chain amino acid sequence comprising SEQ ID NO:189 or an antigen binding fragment thereof, except that the heavy chain amino acid sequence is modified whereby a part or all of the framework 3 region of SEQ ID NO:189 is replaced with an amino acid sequence selected from the group consisting of QLSQDPDDPDWG (SEQ ID NO:541) and comprising SEQ ID NO:191 or an antigen binding fragment thereof;
ww. an antibody comprising a heavy chain amino acid sequence comprising SEQ ID NO:193 or an antigen binding fragment thereof, except that the heavy chain amino acid sequence is modified whereby a part or all of the framework 3 region of SEQ ID NO:193 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:195 or an antigen binding fragment thereof;
xx. an antibody comprising a heavy chain amino acid sequence comprising SEQ ID NO:197 or an antigen binding fragment thereof, except that the heavy chain amino acid sequence is modified whereby a part or all of the framework 3 region of SEQ ID NO:197 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:199 or an antigen binding fragment thereof;
yy. an antibody comprising a heavy chain amino acid sequence comprising SEQ ID NO:201 or an antigen binding fragment thereof, except that the heavy chain amino acid sequence is modified whereby a part or all of the framework 3 region of SEQ ID NO:201 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:203 or an antigen binding fragment thereof;
zz. an antibody comprising a heavy chain amino acid sequence comprising SEQ ID NO:205 or an antigen binding fragment thereof, except that the heavy chain amino acid sequence is modified whereby a part or all of the framework 3 region of SEQ ID NO:205 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:207 or an antigen binding fragment thereof;
aaa. an antibody comprising a heavy chain amino acid sequence comprising SEQ ID NO:209 or an antigen binding fragment thereof, except that the heavy chain amino acid sequence is modified whereby a part or all of the framework 3 region of SEQ ID NO:209 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:211 or an antigen binding fragment thereof;
bbb. an antibody comprising a heavy chain amino acid sequence comprising SEQ ID NO:213 or an antigen binding fragment thereof, except that the heavy chain amino acid sequence is modified whereby a part or all of the framework 3 region of SEQ ID NO:213 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:215 or an antigen binding fragment thereof;
ccc. an antibody comprising a heavy chain amino acid sequence comprising SEQ ID NO:217 or an antigen binding fragment thereof, except that the heavy chain amino acid sequence is modified whereby a part or all of the framework 3 region of SEQ ID NO:217 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:219 or an antigen binding fragment thereof;
ddd. an antibody comprising a heavy chain amino acid sequence comprising SEQ ID NO:221 or an antigen binding fragment thereof, except that the heavy chain amino acid sequence is modified whereby a part or all of the framework 3 region of SEQ ID NO:221 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:223 or an antigen binding fragment thereof;
eee. an antibody comprising a heavy chain amino acid sequence comprising SEQ ID NO:225 or an antigen binding fragment thereof, except that the heavy chain amino acid sequence is modified whereby a part or all of the framework 3 region of SEQ ID NO:225 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:227 or an antigen binding fragment thereof;
fff. an antibody comprising a heavy chain amino acid sequence comprising SEQ ID NO:229 or an antigen binding fragment thereof, except that the heavy chain amino acid sequence is modified whereby a part or all of the framework 3 region of SEQ ID NO:229 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:231 or an antigen binding fragment thereof;
ggg. an antibody comprising a heavy chain amino acid sequence comprising SEQ ID NO:233 or an antigen binding fragment thereof, except that the heavy chain amino acid sequence is modified whereby a part or all of the framework 3 region of SEQ ID NO:233 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:235 or an antigen binding fragment thereof;
hhh. an antibody comprising a heavy chain amino acid sequence comprising SEQ ID NO:237 or an antigen binding fragment thereof, except that the heavy chain amino acid sequence is modified whereby a part or all of the framework 3 region of SEQ ID NO:237 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:239 or an antigen binding fragment thereof;
iii. an antibody comprising a heavy chain amino acid sequence comprising SEQ ID NO:241 or an antigen binding fragment thereof, except that the heavy chain amino acid sequence is modified whereby a part or all of the framework 3 region of SEQ ID NO:241 is replaced with an amino acid sequence selected from the group consisting of QLSQDPDDPDWG (SEQ ID NO:541) and comprising SEQ ID NO:243 or an antigen binding fragment thereof;
jjj. an antibody comprising a heavy chain amino acid sequence comprising SEQ ID NO:245 or an antigen binding fragment thereof, except that the heavy chain amino acid sequence is modified whereby a part or all of the framework 3 region of SEQ ID NO:245 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:247 or an antigen binding fragment thereof;
kkk. an antibody comprising a heavy chain amino acid sequence comprising SEQ ID NO:249 or an antigen binding fragment thereof, except that the heavy chain amino acid sequence is modified whereby a part or all of the framework 3 region of SEQ ID NO:249 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:251 or an antigen binding fragment thereof;
lll. an antibody comprising a heavy chain amino acid sequence comprising SEQ ID NO:253 or an antigen binding fragment thereof, except that the heavy chain amino acid sequence is modified whereby a part or all of the framework 3 region of SEQ ID NO:253 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:255 or an antigen binding fragment thereof;
mmm. an antibody comprising a heavy chain amino acid sequence comprising SEQ ID NO:257 or an antigen binding fragment thereof, except that the heavy chain amino acid sequence is modified whereby a part or all of the framework 3 region of SEQ ID NO:257 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:259 or an antigen binding fragment thereof;
nnn. an antibody comprising a heavy chain amino acid sequence comprising SEQ ID NO:261 or an antigen binding fragment thereof, except that the heavy chain amino acid sequence is modified whereby a part or all of the framework 3 region of SEQ ID NO:261 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:263 or an antigen binding fragment thereof;
ooo. an antibody comprising a heavy chain amino acid sequence comprising SEQ ID NO:265 or an antigen binding fragment thereof, except that the heavy chain amino acid sequence is modified whereby a part or all of the framework 3 region of SEQ ID NO:265 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:267 or an antigen binding fragment thereof;
ppp. an antibody comprising a heavy chain amino acid sequence comprising SEQ ID NO:269 or an antigen binding fragment thereof, except that the heavy chain amino acid sequence is modified whereby a part or all of the framework 3 region of SEQ ID NO:269 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:271 or an antigen binding fragment thereof;
qqq. an antibody comprising a heavy chain amino acid sequence comprising SEQ ID NO:273 or an antigen binding fragment thereof, except that the heavy chain amino acid sequence is modified whereby a part or all of the framework 3 region of SEQ ID NO:273 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:275 or an antigen binding fragment thereof;
rrr. an antibody comprising a heavy chain amino acid sequence comprising SEQ ID NO:277 or an antigen binding fragment thereof, except that the heavy chain amino acid sequence is modified whereby a part or all of the framework 3 region of SEQ ID NO:277 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:279 or an antigen binding fragment thereof;
sss. an antibody comprising a heavy chain amino acid sequence comprising SEQ ID NO:281 or an antigen binding fragment thereof, except that the heavy chain amino acid sequence is modified whereby a part or all of the framework 3 region of SEQ ID NO:281 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:283 or an antigen binding fragment thereof;
ttt. an antibody comprising a heavy chain amino acid sequence comprising SEQ ID NO:285 or an antigen binding fragment thereof, except that the heavy chain amino acid sequence is modified whereby a part or all of the framework 3 region of SEQ ID NO:285 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:287 or an antigen binding fragment thereof;
uuu. an antibody comprising a heavy chain amino acid sequence comprising SEQ ID NO:289 or an antigen binding fragment thereof, except that the heavy chain amino acid sequence is modified whereby a part or all of the framework 3 region of SEQ ID NO:289 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:291 or an antigen binding fragment thereof;
vvv. an antibody comprising a heavy chain amino acid sequence comprising SEQ ID NO:293 or an antigen binding fragment thereof, except that the heavy chain amino acid sequence is modified whereby a part or all of the framework 3 region of SEQ ID NO:293 is replaced with an amino acid sequence selected from the group consisting of QLSQDPDDPDWG (SEQ ID NO:541) and comprising SEQ ID NO:295 or an antigen binding fragment thereof;
www. an antibody comprising a heavy chain amino acid sequence comprising SEQ ID NO:297 or an antigen binding fragment thereof, except that the heavy chain amino acid sequence is modified whereby a part or all of the framework 3 region of SEQ ID NO:297 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:299 or an antigen binding fragment thereof;
xxx. an antibody comprising a heavy chain amino acid sequence comprising SEQ ID NO:301 or an antigen binding fragment thereof, except that the heavy chain amino acid sequence is modified whereby a part or all of the framework 3 region of SEQ ID NO:301 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:303 or an antigen binding fragment thereof;
yyy. an antibody comprising a heavy chain amino acid sequence comprising SEQ ID NO:305 or an antigen binding fragment thereof, except that the heavy chain amino acid sequence is modified whereby a part or all of the framework 3 region of SEQ ID NO:305 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:307 or an antigen binding fragment thereof;
zzz. an antibody comprising a heavy chain amino acid sequence comprising SEQ ID NO:309 or an antigen binding fragment thereof, except that the heavy chain amino acid sequence is modified whereby a part or all of the framework 3 region of SEQ ID NO:309 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:311 or an antigen binding fragment thereof;
aaaa. an antibody comprising a heavy chain amino acid sequence comprising SEQ ID NO:313 or an antigen binding fragment thereof, except that the heavy chain amino acid sequence is modified whereby a part or all of the framework 3 region of SEQ ID NO:313 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:315 or an antigen binding fragment thereof;
bbbb. an antibody comprising a heavy chain amino acid sequence comprising SEQ ID NO:317 or an antigen binding fragment thereof, except that the heavy chain amino acid sequence is modified whereby a part or all of the framework 3 region of SEQ ID NO:317 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:319 or an antigen binding fragment thereof;
cccc. an antibody comprising a heavy chain amino acid sequence comprising SEQ ID NO:321 or an antigen binding fragment thereof, except that the heavy chain amino acid sequence is modified whereby a part or all of the framework 3 region of SEQ ID NO:321 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:323 or an antigen binding fragment thereof;
dddd. an antibody comprising a heavy chain amino acid sequence comprising SEQ ID NO:325 or an antigen binding fragment thereof, except that the heavy chain amino acid sequence is modified whereby a part or all of the framework 3 region of SEQ ID NO:325 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:327 or an antigen binding fragment thereof;
eeee. an antibody comprising a heavy chain amino acid sequence comprising SEQ ID NO:329 or an antigen binding fragment thereof, except that the heavy chain amino acid sequence is modified whereby a part or all of the framework 3 region of SEQ ID NO:329 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:331 or an antigen binding fragment thereof;
ffff. an antibody comprising a heavy chain amino acid sequence comprising SEQ ID NO:333 or an antigen binding fragment thereof, except that the heavy chain amino acid sequence is modified whereby a part or all of the framework 3 region of SEQ ID NO:333 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:335 or an antigen binding fragment thereof;
gggg. an antibody comprising a heavy chain amino acid sequence comprising SEQ ID NO:337 or an antigen binding fragment thereof, except that the heavy chain amino acid sequence is modified whereby a part or all of the framework 3 region of SEQ ID NO:337 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:339 or an antigen binding fragment thereof;
hhhh. an antibody comprising a heavy chain amino acid sequence comprising SEQ ID NO:341 or an antigen binding fragment thereof, except that the heavy chain amino acid sequence is modified whereby a part or all of the framework 3 region of SEQ ID NO:341 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:343 or an antigen binding fragment thereof;
iiii. an antibody comprising a heavy chain amino acid sequence comprising SEQ ID NO:345 or an antigen binding fragment thereof, except that the heavy chain amino acid sequence is modified whereby a part or all of the framework 3 region of SEQ ID NO:345 is replaced with an amino acid sequence selected from the group consisting of QLSQDPDDPDWG (SEQ ID NO:541) and comprising SEQ ID NO:347 or an antigen binding fragment thereof;
jjjj. an antibody comprising a heavy chain amino acid sequence comprising SEQ ID NO:349 or an antigen binding fragment thereof, except that the heavy chain amino acid sequence is modified whereby a part or all of the framework 3 region of SEQ ID NO:349 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:351 or an antigen binding fragment thereof;
kkkk an antibody comprising a heavy chain amino acid sequence comprising SEQ ID NO:353 or an antigen binding fragment thereof, except that the heavy chain amino acid sequence is modified whereby a part or all of the framework 3 region of SEQ ID NO:353 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:355 or an antigen binding fragment thereof;
llll. an antibody comprising a heavy chain amino acid sequence comprising SEQ ID NO:357 or an antigen binding fragment thereof, except that the heavy chain amino acid sequence is modified whereby a part or all of the framework 3 region of SEQ ID NO:357 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:359 or an antigen binding fragment thereof;
mmmm. an antibody comprising a heavy chain amino acid sequence comprising SEQ ID NO:361 or an antigen binding fragment thereof, except that the heavy chain amino acid sequence is modified whereby a part or all of the framework 3 region of SEQ ID NO:361 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:363 or an antigen binding fragment thereof;
nnnn. an antibody comprising a heavy chain amino acid sequence comprising SEQ ID NO:365 or an antigen binding fragment thereof, except that the heavy chain amino acid sequence is modified whereby a part or all of the framework 3 region of SEQ ID NO:365 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:367 or an antigen binding fragment thereof;
oooo. an antibody comprising a heavy chain amino acid sequence comprising SEQ ID NO:369 or an antigen binding fragment thereof, except that the heavy chain amino acid sequence is modified whereby a part or all of the framework 3 region of SEQ ID NO:369 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:371 or an antigen binding fragment thereof;
pppp. an antibody comprising a heavy chain amino acid sequence comprising SEQ ID NO:373 or an antigen binding fragment thereof, except that the heavy chain amino acid sequence is modified whereby a part or all of the framework 3 region of SEQ ID NO:373 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:375 or an antigen binding fragment thereof;
qqqq. an antibody comprising a heavy chain amino acid sequence comprising SEQ ID NO:377 or an antigen binding fragment thereof, except that the heavy chain amino acid sequence is modified whereby a part or all of the framework 3 region of SEQ ID NO:377 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:379 or an antigen binding fragment thereof;
llll. an antibody comprising a heavy chain amino acid sequence comprising SEQ ID NO:381 or an antigen binding fragment thereof, except that the heavy chain amino acid sequence is modified whereby a part or all of the framework 3 region of SEQ ID NO:381 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:383 or an antigen binding fragment thereof;
ssss. an antibody comprising a heavy chain amino acid sequence comprising SEQ ID NO:385 or an antigen binding fragment thereof, except that the heavy chain amino acid sequence is modified whereby a part or all of the framework 3 region of SEQ ID NO:385 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:387 or an antigen binding fragment thereof;
tttt. an antibody comprising a heavy chain amino acid sequence comprising SEQ ID NO:389 or an antigen binding fragment thereof, except that the heavy chain amino acid sequence is modified whereby a part or all of the framework 3 region of SEQ ID NO:389 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:391 or an antigen binding fragment thereof;
uuuu. an antibody comprising a heavy chain amino acid sequence comprising SEQ ID NO:393 or an antigen binding fragment thereof, except that the heavy chain amino acid sequence is modified whereby a part or all of the framework 3 region of SEQ ID NO:393 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:395 or an antigen binding fragment thereof; and
vvvv. an antibody comprising a heavy chain amino acid sequence comprising SEQ ID NO:397 or an antigen binding fragment thereof, except that the heavy chain amino acid sequence is modified whereby a part or all of the framework 3 region of SEQ ID NO:397 is replaced with an amino acid sequence selected from the group consisting of QLSQDPDDPDWG (SEQ ID NO:541) and comprising SEQ ID NO:399 or an antigen binding fragment thereof.

In some embodiments, the anti-HIV antibody is isolated and/or substantially pure.

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises one or more VL complementary determining regions (CDRs) and wherein the VH region comprises one or more VH complementary determining regions (CDRs), wherein the VL CDRs correspond to the CDRs found within any of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539, wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises one or more VL complementary determining regions (CDRs) and wherein the VH region comprises one or more VH complementary determining regions (CDRs), wherein the VH CDRs correspond to the CDRs found within any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 501, 505, 507, 509, 513, 517, 521, 525, 529, 533, and 537, wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises one or more VL complementary determining regions (CDRs) and wherein the VH region comprises one or more VH complementary determining regions (CDRs), wherein the VL CDRs correspond to the CDRs found within any of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, 319, 323, 327,331, 335, 339, 343, 347, 351, 355, 359, 363, 367,371, 375, 379, 383, 387, 391, 395, 399, 503, 511, 515, 519, 523, 527, 531, 535, and 539, or a variant thereof comprising 1, 2, 3, or 4 conservative amino acid substitutions, and wherein the VH CDRs correspond to the CDRs found within any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 501, 505, 507, 509, 513, 517, 521, 525, 529, 533, and 537 or a variant thereof comprising 1, 2, 3, or 4 conservative amino acid substitutions, wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises an amino acid sequence selected from the group consisting of: amino acids 1-99 of SEQ ID NO:2; amino acids 1-97 of SEQ ID NO:4; amino acids 1-99 of SEQ ID NO:6; amino acids 1-99 of SEQ ID NO:8; amino acids 1-99 of SEQ ID NO:10; amino acids 1-99 of SEQ ID NO:12; amino acids 1-99 of SEQ ID NO:14; amino acids 1-99 of SEQ ID NO:16; amino acids 1-99 of SEQ ID NO:18; amino acids 1-99 of SEQ ID NO:20; amino acids 1-99 of SEQ ID NO:22; amino acids 1-99 of SEQ ID NO:24; amino acids 1-99 of SEQ ID NO:26; amino acids 1-99 of SEQ ID NO:28; amino acids 1-99 of SEQ ID NO:30; amino acids 1-99 of SEQ ID NO:32; amino acids 1-99 of SEQ ID NO:34; amino acids 1-100 of SEQ ID NO:36; amino acids 1-97 of SEQ ID NO:38; amino acids 1-100 of SEQ ID NO:40; amino acids 1-97 of SEQ ID NO:42; amino acids 1-97 of SEQ ID NO:44; amino acids 1-101 of SEQ ID NO:46; amino acids 1-101 of SEQ ID NO:48; amino acids 1-96 of SEQ ID NO:50; amino acids 1-97 of SEQ ID NO:52; amino acids 1-99 of SEQ ID NO:54; amino acids 1-99 of SEQ ID NO:56; amino acids 1-99 of SEQ ID NO:58; amino acids 1-99 of SEQ ID NO:60; amino acids 1-98 of SEQ ID NO:62; amino acids 1-99 of SEQ ID NO:64; amino acids 1-99 of SEQ ID NO:66; amino acids 1-96 of SEQ ID NO:68; amino acids 1-96 of SEQ ID NO:70; amino acids 1-96 of SEQ ID NO:72; amino acids 1-101 of SEQ ID NO:74; amino acids 1-97 of SEQ ID NO:76; amino acids 1-99 of SEQ ID NO:155; amino acids 1-99 of SEQ ID NO:159; amino acids 1-97 of SEQ ID NO:163; amino acids 1-97 of SEQ ID NO:167; amino acids 1-97 of SEQ ID NO:171; amino acids 1-97 of SEQ ID NO:175; amino acids 1-97 of SEQ ID NO:179; amino acids 1-97 of SEQ ID NO:183; amino acids 1-97 of SEQ ID NO:187; amino acids 1-97 of SEQ ID NO:191; amino acids 1-97 of SEQ ID NO:195; amino acids 1-97 of SEQ ID NO:199; amino acids 1-99 of SEQ ID NO:203; amino acids 1-99 of SEQ ID NO:207; amino acids 1-100 of SEQ ID NO:211; amino acids 1-99 of SEQ ID NO:215; amino acids 1-97 of SEQ ID NO:219; amino acids 1-99 of SEQ ID NO:223; amino acids 1-99 of SEQ ID NO:227; amino acids 1-99 of SEQ ID NO:231; amino acids 1-99 of SEQ ID NO:235; amino acids 1-99 of SEQ ID NO:239; amino acids 1-99 of SEQ ID NO:243; amino acids 1-99 of SEQ ID NO:247; amino acids 1-99 of SEQ ID NO:251; amino acids 1-99 of SEQ ID NO:255; amino acids 1-99 of SEQ ID NO:259; amino acids 1-99 of SEQ ID NO:263; amino acids 1-99 of SEQ ID NO:267; amino acids 1-99 of SEQ ID NO:271; amino acids 1-99 of SEQ ID NO:275; amino acids 1-99 of SEQ ID NO:279; amino acids 1-99 of SEQ ID NO:283; amino acids 1-99 of SEQ ID NO:287; amino acids 1-99 of SEQ ID NO:291; amino acids 1-100 of SEQ ID NO:295; amino acids 1-100 of SEQ ID NO:299; amino acids 1-100 of SEQ ID NO:303; amino acids 1-100 of SEQ ID NO:307; amino acids 1-100 of SEQ ID NO:311; amino acids 1-100 of SEQ ID NO:315; amino acids 1-97 of SEQ ID NO:319; amino acids 1-100 of SEQ ID NO:323; amino acids 1-97 of SEQ ID NO:327; amino acids 1-97 of SEQ ID NO:331; amino acids 1-97 of SEQ ID NO:335; amino acids 1-101 of SEQ ID NO:339; amino acids 1-101 of SEQ ID NO:343; amino acids 1-96 of SEQ ID NO:347; amino acids 1-97 of SEQ ID NO:351; amino acids 1-99 of SEQ ID NO:355; amino acids 1-99 of SEQ ID NO:359; amino acids 1-99 of SEQ ID NO:363; amino acids 1-99 of SEQ ID NO:367; amino acids 1-98 of SEQ ID NO:371; amino acids 1-99 of SEQ ID NO:375; amino acids 1-99 of SEQ ID NO:379; amino acids 1-96 of SEQ ID NO:383; amino acids 1-96 of SEQ ID NO:387; amino acids 1-96 of SEQ ID NO:391; amino acids 1-101 of SEQ ID NO:395; amino acids 1-97 of SEQ ID NO:399; amino acids 1-97 of SEQ ID NO:503; amino acids 1-97 of SEQ ID NO: 511; amino acids 1-97 of SEQ ID NO:515; amino acids 1-97 of SEQ ID NO:519; amino acids 1-97 of SEQ ID NO:523; amino acids 1-97 of SEQ ID NO:527; amino acids 1-97 of SEQ ID NO:531; amino acids 1-97 of SEQ ID NO:535; and amino acids 1-97 of SEQ ID NO:539, or a variant thereof comprising 1, 2, 3, or 4 conservative amino acid substitutions, wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VH region comprises an amino acid sequence selected from the group consisting of: amino acids 1-128 of SEQ ID NO:1; amino acids 1-127 of SEQ ID NO:3; amino acids 1-127 of SEQ ID NO:5; amino acids 1-128 of SEQ ID NO:7; amino acids 1-127 of SEQ ID NO:9; amino acids 1-127 of SEQ ID NO:11; amino acids 1-127 of SEQ ID NO:13; amino acids 1-127 of SEQ ID NO:15; amino acids 1-127 of SEQ ID NO:17; amino acids 1-127 of SEQ ID NO:19; amino acids 1-127 of SEQ ID NO:21; amino acids 1-127 of SEQ ID NO:23; amino acids 1-127 of SEQ ID NO:25; amino acids 1-127 of SEQ ID NO:27; amino acids 1-127 of SEQ ID NO:29; amino acids 1-127 of SEQ ID NO:31; amino acids 1-127 of SEQ ID NO:33; amino acids 1-120 of SEQ ID NO:35; amino acids 1-120 of SEQ ID NO:37; amino acids 1-123 of SEQ ID NO:39; amino acids 1-120 of SEQ ID NO:41; amino acids 1-120 of SEQ ID NO:43; amino acids 1-125 of SEQ ID NO:45; amino acids 1-125 of SEQ ID NO:47; amino acids 1-120 of SEQ ID NO:49; amino acids 1-120 of SEQ ID NO:51; amino acids 1-121 of SEQ ID NO:53; amino acids 1-121 of SEQ ID NO:55; amino acids 1-121 of SEQ ID NO:57; amino acids 1-121 of SEQ ID NO:59; amino acids 1-120 of SEQ ID NO:61; amino acids 1-121 of SEQ ID NO:63; amino acids 1-121 of SEQ ID NO:65; amino acids 1-120 of SEQ ID NO:67; amino acids 1-120 of SEQ ID NO:69; amino acids 1-120 of SEQ ID NO:71; amino acids 1-125 of SEQ ID NO:73; amino acids 1-120 of SEQ ID NO:75; amino acids 1-128 of SEQ ID NO:153; amino acids 1-128 of SEQ ID NO:157; amino acids 1-127 of SEQ ID NO:161; amino acids 1-127 of SEQ ID NO:165; amino acids 1-127 of SEQ ID NO:169; amino acids 1-127 of SEQ ID NO:173; amino acids 1-127 of SEQ ID NO:177; amino acids 1-127 of SEQ ID NO:181; amino acids 1-127 of SEQ ID NO:185; amino acids 1-127 of SEQ ID NO:189; amino acids 1-127 of SEQ ID NO:193; amino acids 1-127 of SEQ ID NO:197; amino acids 1-127 of SEQ ID NO:201; amino acids 1-127 of SEQ ID NO:205; amino acids 1-127 of SEQ ID NO:209; amino acids 1-127 of SEQ ID NO:213; amino acids 1-127 of SEQ ID NO:217; amino acids 1-127 of SEQ ID NO:221; amino acids 1-128 of SEQ ID NO:225; amino acids 1-127 of SEQ ID NO:229; amino acids 1-127 of SEQ ID NO:233; amino acids 1-127 of SEQ ID NO:237; amino acids 1-127 of SEQ ID NO:241; amino acids 1-127 of SEQ ID NO:245; amino acids 1-127 of SEQ ID NO:249; amino acids 1-127 of SEQ ID NO:253; amino acids 1-127 of SEQ ID NO:257; amino acids 1-127 of SEQ ID NO:261; amino acids 1-127 of SEQ ID NO:265; amino acids 1-127 of SEQ ID NO:269; amino acids 1-127 of SEQ ID NO:273; amino acids 1-127 of SEQ ID NO:277; amino acids 1-127 of SEQ ID NO:281; amino acids 1-127 of SEQ ID NO:285; amino acids 1-127 of SEQ ID NO:289; amino acids 1-120 of SEQ ID NO:293; amino acids 1-120 of SEQ ID NO:297; amino acids 1-120 of SEQ ID NO:301; amino acids 1-123 of SEQ ID NO:305; amino acids 1-128 of SEQ ID NO:309; amino acids 1-128 of SEQ ID NO:313; amino acids 1-120 of SEQ ID NO:317; amino acids 1-123 of SEQ ID NO:321; amino acids 1-120 of SEQ ID NO:325; amino acids 1-120 of SEQ ID NO:329; amino acids 1-120 of SEQ ID NO:333; amino acids 1-125 of SEQ ID NO:337; amino acids 1-125 of SEQ ID NO:341; amino acids 1-120 of SEQ ID NO:345; amino acids 1-120 of SEQ ID NO:349; amino acids 1-121 of SEQ ID NO:353; amino acids 1-121 of SEQ ID NO:357; amino acids 1-121 of SEQ ID NO:361; amino acids 1-121 of SEQ ID NO:365; amino acids 1-120 of SEQ ID NO:369; amino acids 1-121 of SEQ ID NO:373; amino acids 1-121 of SEQ ID NO:377; amino acids 1-120 of SEQ ID NO:381; amino acids 1-120 of SEQ ID NO:385; amino acids 1-120 of SEQ ID NO:389; amino acids 1-125 of SEQ ID NO:393; and amino acids 1-120 of SEQ ID NO:397, or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VH region comprises an amino acid sequence selected from the group consisting of: amino acids 1-134 of SEQ ID NO:501; amino acids 1-127 of SEQ ID NO: 505; amino acids 1-127 of SEQ ID NO:507; amino acids 1-127 of SEQ ID NO:509; amino acids 1-127 of SEQ ID NO:513; amino acids 1-127 of SEQ ID NO:517; amino acids 1-127 of SEQ ID NO:521; amino acids 1-127 of SEQ ID NO:525; amino acids 1-127 of SEQ ID NO:529; amino acids 1-127 of SEQ ID NO:533; and amino acids 1-127 of SEQ ID NO:537, or a variant thereof comprising 1, 2, 3, or 4 conservative amino acid substitutions,

In some embodiments, the anti-HIV antibody comprises a heavy chain or an antigen binding fragment thereof and a light chain or an antigen binding fragment thereof, wherein the heavy chain or antigen binding fragment thereof comprises a heavy chain variable (VH) region and the light chain or antigen binding fragment thereof comprises a light chain variable (VL) region; wherein the anti-HIV antibody is selected from the group consisting of:
i) an antibody wherein the VH region comprises amino acids 1-128 of SEQ ID NO:1 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:2 or a variant thereof comprising 1, 2, 3, or 4 conservative amino acid substitutions;
ii) an antibody wherein the VH region comprises amino acids 1-127 of SEQ ID NO:3 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:4 or a variant thereof comprising 1, 2, 3, or 4 conservative amino acid substitutions;
iii) an antibody wherein the VH region comprises amino acids 1-127 of SEQ ID NO:5 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:6 or a variant thereof comprising 1, 2, 3, or 4 conservative amino acid substitutions;
iv) an antibody wherein the VH region comprises amino acids 1-128 of SEQ ID NO:7 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:8 or a variant thereof comprising 1, 2, 3, or 4 conservative amino acid substitutions;
v) an antibody wherein the VH region comprises amino acids 1-127 of SEQ ID NO:9 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:10 or a variant thereof comprising 1, 2, 3, or 4 conservative amino acid substitutions;
vi) an antibody wherein the VH region comprises amino acids 1-127 of SEQ ID NO:11 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:12 or a variant thereof comprising 1, 2, 3, or 4 conservative amino acid substitutions;
vii) an antibody wherein the VH region comprises amino acids 1-127 of SEQ ID NO:13 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:14 or a variant thereof comprising 1, 2, 3, or 4 conservative amino acid substitutions;
viii) an antibody wherein the VH region comprises amino acids 1-127 of SEQ ID NO:15 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:16 or a variant thereof comprising 1, 2, 3, or 4 conservative amino acid substitutions;
ix) an antibody wherein the VH region comprises amino acids 1-127 of SEQ ID NO:17 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:18 or a variant thereof comprising 1, 2, 3, or 4 conservative amino acid substitutions;
x) an antibody wherein the VH region comprises amino acids 1-127 of SEQ ID NO:19 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:20 or a variant thereof comprising 1, 2, 3, or 4 conservative amino acid substitutions;
xi) an antibody wherein the VH region comprises amino acids 1-127 of SEQ ID NO:21 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:22 or a variant thereof comprising 1, 2, 3, or 4 conservative amino acid substitutions;
xii) an antibody wherein the VH region comprises amino acids 1-127 of SEQ ID NO:23 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:24 or a variant thereof comprising 1, 2, 3, or 4 conservative amino acid substitutions;
xiii) an antibody wherein the VH region comprises amino acids 1-127 of SEQ ID NO:25 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:26 or a variant thereof comprising 1, 2, 3, or 4 conservative amino acid substitutions;
xiv) an antibody wherein the VH region comprises amino acids 1-127 of SEQ ID NO:27 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:28 or a variant thereof comprising 1, 2, 3, or 4 conservative amino acid substitutions;
xv) an antibody wherein the VH region comprises amino acids 1-127 of SEQ ID NO:29 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:30 or a variant thereof comprising 1, 2, 3, or 4 conservative amino acid substitutions;
xvi) an antibody wherein the VH region comprises amino acids 1-127 of SEQ ID NO:31 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:32 or a variant thereof comprising 1, 2, 3, or 4 conservative amino acid substitutions;
xvii) an antibody wherein the VH region comprises amino acids 1-127 of SEQ ID NO:33 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:34 or a variant thereof comprising 1, 2, 3, or 4 conservative amino acid substitutions;
xviii) an antibody wherein the VH region comprises amino acids 1-120 of SEQ ID NO:35 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:36 or a variant thereof comprising 1, 2, 3, or 4 conservative amino acid substitutions;
xix) an antibody wherein the VH region comprises amino acids 1-120 of SEQ ID NO:37 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:38 or a variant thereof comprising 1, 2, 3, or 4 conservative amino acid substitutions;
xx) an antibody wherein the VH region comprises amino acids 1-123 of SEQ ID NO:39 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:40 or a variant thereof comprising 1, 2, 3, or 4 conservative amino acid substitutions;
xxi) an antibody wherein the VH region comprises amino acids 1-120 of SEQ ID NO:41 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:42 or a variant thereof comprising 1, 2, 3, or 4 conservative amino acid substitutions;
xxii) an antibody wherein the VH region comprises amino acids 1-120 of SEQ ID NO:43 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:44 or a variant thereof comprising 1, 2, 3, or 4 conservative amino acid substitutions;
xxiii) an antibody wherein the VH region comprises amino acids 1-125 of SEQ ID NO:45 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:46 or a variant thereof comprising 1, 2, 3, or 4 conservative amino acid substitutions;
xxiv) an antibody wherein the VH region comprises amino acids 1-125 of SEQ ID NO:47 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:48 or a variant thereof comprising 1, 2, 3, or 4 conservative amino acid substitutions;
xxv) an antibody wherein the VH region comprises amino acids 1-120 of SEQ ID NO:49 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:50 or a variant thereof comprising 1, 2, 3, or 4 conservative amino acid substitutions;
xxvi) an antibody wherein the VH region comprises amino acids 1-120 of SEQ ID NO:51 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:52 or a variant thereof comprising 1, 2, 3, or 4 conservative amino acid substitutions;
xxvii) an antibody wherein the VH region comprises amino acids 1-121 of SEQ ID NO:53 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:54 or a variant thereof comprising 1, 2, 3, or 4 conservative amino acid substitutions;
xxviii) an antibody wherein the VH region comprises amino acids 1-121 of SEQ ID NO:55 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:56 or a variant thereof comprising 1, 2, 3, or 4 conservative amino acid substitutions;
xxix) an antibody wherein the VH region comprises amino acids 1-121 of SEQ ID NO:57 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:58 or a variant thereof comprising 1, 2, 3, or 4 conservative amino acid substitutions;
xxx) an antibody wherein the VH region comprises amino acids 1-121 of SEQ ID NO:59 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:60 or a variant thereof comprising 1, 2, 3, or 4 conservative amino acid substitutions;
xxxi) an antibody wherein the VH region comprises amino acids 1-120 of SEQ ID NO:61 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-98 of SEQ ID NO:62 or a variant thereof comprising 1, 2, 3, or 4 conservative amino acid substitutions;
xxxii) an antibody wherein the VH region comprises amino acids 1-121 of SEQ ID NO:63 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:64 or a variant thereof comprising 1, 2, 3, or 4 conservative amino acid substitutions;
xxxiii) an antibody wherein the VH region comprises amino acids 1-121 of SEQ ID NO:65 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:66 or a variant thereof comprising 1, 2, 3, or 4 conservative amino acid substitutions;
xxxiv) an antibody wherein the VH region comprises amino acids 1-120 of SEQ ID NO:67 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:68 or a variant thereof comprising 1, 2, 3, or 4 conservative amino acid substitutions;
xxxv) an antibody wherein the VH region comprises amino acids 1-120 of SEQ ID NO:69 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:70 or a variant thereof comprising 1, 2, 3, or 4 conservative amino acid substitutions;
xxxvi) an antibody wherein the VH region comprises amino acids 1-120 of SEQ ID NO:71 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:72 or a variant thereof comprising 1, 2, 3, or 4 conservative amino acid substitutions;
xxxvii) an antibody wherein the VH region comprises amino acids 1-125 of SEQ ID NO:73 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:74 or a variant thereof comprising 1, 2, 3, or 4 conservative amino acid substitutions;
xxxviii) an antibody wherein the VH region comprises amino acids 1-120 of SEQ ID NO:75 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:76 or a variant thereof comprising 1, 2, 3, or 4 conservative amino acid substitutions;
xxxix) an antibody wherein the VH region comprises amino acids 1-128 of SEQ ID NO:153 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:155 or a variant thereof comprising 1, 2, 3, or 4 conservative amino acid substitutions;
xl) an antibody wherein the VH region comprises amino acids 1-128 of SEQ ID NO:157 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:159 or a variant thereof comprising 1, 2, 3, or 4 conservative amino acid substitutions;
xli) an antibody wherein the VH region comprises amino acids 1-127 of SEQ ID NO:161 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:163 or a variant thereof comprising 1, 2, 3, or 4 conservative amino acid substitutions;
xlii) an antibody wherein the VH region comprises amino acids 1-127 of SEQ ID NO:165 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:167 or a variant thereof comprising 1, 2, 3, or 4 conservative amino acid substitutions;
xliii) an antibody wherein the VH region comprises amino acids 1-127 of SEQ ID NO:169 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:171 or a variant thereof comprising 1, 2, 3, or 4 conservative amino acid substitutions;
xliv) an antibody wherein the VH region comprises amino acids 1-127 of SEQ ID NO:173 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:175 or a variant thereof comprising 1, 2, 3, or 4 conservative amino acid substitutions;
xlv) an antibody wherein the VH region comprises amino acids 1-127 of SEQ ID NO:177 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:179 or a variant thereof comprising 1, 2, 3, or 4 conservative amino acid substitutions;
xlvi) an antibody wherein the VH region comprises amino acids 1-127 of SEQ ID NO:181 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:183 or a variant thereof comprising 1, 2, 3, or 4 conservative amino acid substitutions;
xlvii) an antibody wherein the VH region comprises amino acids 1-127 of SEQ ID NO:185 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:187 or a variant thereof comprising 1, 2, 3, or 4 conservative amino acid substitutions;
xlviii) an antibody wherein the VH region comprises amino acids 1-127 of SEQ ID NO:189 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:191 or a variant thereof comprising 1, 2, 3, or 4 conservative amino acid substitutions;
xlix) an antibody wherein the VH region comprises amino acids 1-127 of SEQ ID NO:193 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:195 or a variant thereof comprising 1, 2, 3, or 4 conservative amino acid substitutions;
1) an antibody wherein the VH region comprises amino acids 1-127 of SEQ ID NO:197 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:199 or a variant thereof comprising 1, 2, 3, or 4 conservative amino acid substitutions;
li) an antibody wherein the VH region comprises amino acids 1-127 of SEQ ID NO:201 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:203 or a variant thereof comprising 1, 2, 3, or 4 conservative amino acid substitutions;
lii) an antibody wherein the VH region comprises amino acids 1-127 of SEQ ID NO:205 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:207 or a variant thereof comprising 1, 2, 3, or 4 conservative amino acid substitutions;
liii) an antibody wherein the VH region comprises amino acids 1-127 of SEQ ID NO:209 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:211 or a variant thereof comprising 1, 2, 3, or 4 conservative amino acid substitutions;
liv) an antibody wherein the VH region comprises amino acids 1-127 of SEQ ID NO:213 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:215 or a variant thereof comprising 1, 2, 3, or 4 conservative amino acid substitutions;
lv) an antibody wherein the VH region comprises amino acids 1-127 of SEQ ID NO:217 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:219 or a variant thereof comprising 1, 2, 3, or 4 conservative amino acid substitutions;
lvi) an antibody wherein the VH region comprises amino acids 1-127 of SEQ ID NO:221 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:223 or a variant thereof comprising 1, 2, 3, or 4 conservative amino acid substitutions;
lvii) an antibody wherein the VH region comprises amino acids 1-128 of SEQ ID NO:225 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:227 or a variant thereof comprising 1, 2, 3, or 4 conservative amino acid substitutions;
lviii) an antibody wherein the VH region comprises amino acids 1-127 of SEQ ID NO:229 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:231 or a variant thereof comprising 1, 2, 3, or 4 conservative amino acid substitutions;
lix) an antibody wherein the VH region comprises amino acids 1-127 of SEQ ID NO:233 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:235 or a variant thereof comprising 1, 2, 3, or 4 conservative amino acid substitutions;
lx) an antibody wherein the VH region comprises amino acids 1-127 of SEQ ID NO:237 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:239 or a variant thereof comprising 1, 2, 3, or 4 conservative amino acid substitutions;
lxi) an antibody wherein the VH region comprises amino acids 1-127 of SEQ ID NO:241 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:243 or a variant thereof comprising 1, 2, 3, or 4 conservative amino acid substitutions;
lxii) an antibody wherein the VH region comprises amino acids 1-127 of SEQ ID NO:245 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:247 or a variant thereof comprising 1, 2, 3, or 4 conservative amino acid substitutions;
lxiii) an antibody wherein the VH region comprises amino acids 1-127 of SEQ ID NO:249 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:251 or a variant thereof comprising 1, 2, 3, or 4 conservative amino acid substitutions;
lxiv) an antibody wherein the VH region comprises amino acids 1-127 of SEQ ID NO:253 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:255 or a variant thereof comprising 1, 2, 3, or 4 conservative amino acid substitutions;
lxv) an antibody wherein the VH region comprises amino acids 1-127 of SEQ ID NO:257 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:259 or a variant thereof comprising 1, 2, 3, or 4 conservative amino acid substitutions;
lxvi) an antibody wherein the VH region comprises amino acids 1-127 of SEQ ID NO:261 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:263 or a variant thereof comprising 1, 2, 3, or 4 conservative amino acid substitutions;
lxvii) an antibody wherein the VH region comprises amino acids 1-127 of SEQ ID NO:265 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:267 or a variant thereof comprising 1, 2, 3, or 4 conservative amino acid substitutions;
lxviii) an antibody wherein the VH region comprises amino acids 1-127 of SEQ ID NO:269 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:271 or a variant thereof comprising 1, 2, 3, or 4 conservative amino acid substitutions;
lxix) an antibody wherein the VH region comprises amino acids 1-127 of SEQ ID NO:273 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:275 or a variant thereof comprising 1, 2, 3, or 4 conservative amino acid substitutions;
lxx) an antibody wherein the VH region comprises amino acids 1-127 of SEQ ID NO:277 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:279 or a variant thereof comprising 1, 2, 3, or 4 conservative amino acid substitutions;
lxxi) an antibody wherein the VH region comprises amino acids 1-127 of SEQ ID NO:281 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:283 or a variant thereof comprising 1, 2, 3, or 4 conservative amino acid substitutions;
lxxii) an antibody wherein the VH region comprises amino acids 1-127 of SEQ ID NO:285 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:287 or a variant thereof comprising 1, 2, 3, or 4 conservative amino acid substitutions;
lxxiii) an antibody wherein the VH region comprises amino acids 1-127 of SEQ ID NO:289 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:291 or a variant thereof comprising 1, 2, 3, or 4 conservative amino acid substitutions;
lxxiv) an antibody wherein the VH region comprises amino acids 1-120 of SEQ ID NO:293 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:295 or a variant thereof comprising 1, 2, 3, or 4 conservative amino acid substitutions;
lxxv) an antibody wherein the VH region comprises amino acids 1-120 of SEQ ID NO:297 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:299 or a variant thereof comprising 1, 2, 3, or 4 conservative amino acid substitutions;
lxxvi) an antibody wherein the VH region comprises amino acids 1-120 of SEQ ID NO:301 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:303 or a variant thereof comprising 1, 2, 3, or 4 conservative amino acid substitutions;
lxxvii) an antibody wherein the VH region comprises amino acids 1-123 of SEQ ID NO:305 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:307 or a variant thereof comprising 1, 2, 3, or 4 conservative amino acid substitutions;
lxxviii) an antibody wherein the VH region comprises amino acids 1-128 of SEQ ID NO:309 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:311 or a variant thereof comprising 1, 2, 3, or 4 conservative amino acid substitutions;
lxxix) an antibody wherein the VH region comprises amino acids 1-128 of SEQ ID NO:313 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:315 or a variant thereof comprising 1, 2, 3, or 4 conservative amino acid substitutions;
lxxx) an antibody wherein the VH region comprises amino acids 1-120 of SEQ ID NO:317 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:319 or a variant thereof comprising 1, 2, 3, or 4 conservative amino acid substitutions;
lxxxi) an antibody wherein the VH region comprises amino acids 1-123 of SEQ ID NO:321 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:323 or a variant thereof comprising 1, 2, 3, or 4 conservative amino acid substitutions;
lxxxii) an antibody wherein the VH region comprises amino acids 1-120 of SEQ ID NO:325 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:327 or a variant thereof comprising 1, 2, 3, or 4 conservative amino acid substitutions;
lxxxiii) an antibody wherein the VH region comprises amino acids 1-120 of SEQ ID NO:329 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:331 or a variant thereof comprising 1, 2, 3, or 4 conservative amino acid substitutions;
lxxxiv) an antibody wherein the VH region comprises amino acids 1-120 of SEQ ID NO:333 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:335 or a variant thereof comprising 1, 2, 3, or 4 conservative amino acid substitutions;
lxxxv) an antibody wherein the VH region comprises amino acids 1-125 of SEQ ID NO:337 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:339 or a variant thereof comprising 1, 2, 3, or 4 conservative amino acid substitutions;
lxxxvi) an antibody wherein the VH region comprises amino acids 1-125 of SEQ ID NO:341 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:343 or a variant thereof comprising 1, 2, 3, or 4 conservative amino acid substitutions;
lxxxvii) an antibody wherein the VH region comprises amino acids 1-120 of SEQ ID NO:345 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:347 or a variant thereof comprising 1, 2, 3, or 4 conservative amino acid substitutions;
lxxxviii) an antibody wherein the VH region comprises amino acids 1-120 of SEQ ID NO:349 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:351 or a variant thereof comprising 1, 2, 3, or 4 conservative amino acid substitutions;
lxxxix) an antibody wherein the VH region comprises amino acids 1-121 of SEQ ID NO:353 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:355 or a variant thereof comprising 1, 2, 3, or 4 conservative amino acid substitutions;
xc) an antibody wherein the VH region comprises amino acids 1-121 of SEQ ID NO:357 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:359 or a variant thereof comprising 1, 2, 3, or 4 conservative amino acid substitutions;
xci) an antibody wherein the VH region comprises amino acids 1-121 of SEQ ID NO:361 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:363 or a variant thereof comprising 1, 2, 3, or 4 conservative amino acid substitutions;
xcii) an antibody wherein the VH region comprises amino acids 1-121 of SEQ ID NO:365 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:367 or a variant thereof comprising 1, 2, 3, or 4 conservative amino acid substitutions;
xciii) an antibody wherein the VH region comprises amino acids 1-120 of SEQ ID NO:369 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-98 of SEQ ID NO:371 or a variant thereof comprising 1, 2, 3, or 4 conservative amino acid substitutions;
xciv) an antibody wherein the VH region comprises amino acids 1-121 of SEQ ID NO:373 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:375 or a variant thereof comprising 1, 2, 3, or 4 conservative amino acid substitutions;
xcv) an antibody wherein the VH region comprises amino acids 1-121 of SEQ ID NO:377 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:379 or a variant thereof comprising 1, 2, 3, or 4 conservative amino acid substitutions;
xcvi) an antibody wherein the VH region comprises amino acids 1-120 of SEQ ID NO:381 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:383 or a variant thereof comprising 1, 2, 3, or 4 conservative amino acid substitutions;
xcvii) an antibody wherein the VH region comprises amino acids 1-120 of SEQ ID NO:385 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:387 or a variant thereof comprising 1, 2, 3, or 4 conservative amino acid substitutions;
xcviii) an antibody wherein the VH region comprises amino acids 1-120 of SEQ ID NO:389 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:391 or a variant thereof comprising 1, 2, 3, or 4 conservative amino acid substitutions;
xcix) an antibody wherein the VH region comprises amino acids 1-125 of SEQ ID NO:393 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:395 or a variant thereof comprising 1, 2, 3, or 4 conservative amino acid substitutions;
c) an antibody wherein the VH region comprises and amino acids 1-120 of SEQ ID NO:397 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); and wherein the VL region comprises amino acids 1-97 of SEQ ID NO:399 or a variant thereof comprising 1, 2, 3, or 4 conservative amino acid substitutions;
ci) an antibody wherein the VH region comprises and amino acids 1-134 of SEQ ID NO:501 or a variant thereof comprising 1, 2, 3, or 4 conservative amino acid substitutions, and wherein the VL region comprises amino acids 1-97 of SEQ ID NO:503 or a variant thereof comprising 1, 2, 3, or 4 conservative amino acid substitutions;
cii) an antibody wherein the VH region comprises and amino acids 1-127 of SEQ ID NO:505 or a variant thereof comprising 1, 2, 3, or 4 conservative amino acid substitutions, and wherein the VL region comprises amino acids 1-100 of SEQ ID NO:295 or a variant thereof comprising 1, 2, 3, or 4 conservative amino acid substitutions;
ciii) an antibody wherein the VH region comprises and amino acids 1-127 of SEQ ID NO:507 or a variant thereof comprising 1, 2, 3, or 4 conservative amino acid substitutions, and wherein the VL region comprises amino acids 1-100 of SEQ ID NO:327 or a variant thereof comprising 1, 2, 3, or 4 conservative amino acid substitutions;
civ) an antibody wherein the VH region comprises and amino acids 1-127 of SEQ ID NO:509 or a variant thereof comprising 1, 2, 3, or 4 conservative amino acid substitutions, and wherein the VL region comprises amino acids 1-97 of SEQ ID NO:511 or a variant thereof comprising 1, 2, 3, or 4 conservative amino acid substitutions;
cv) an antibody wherein the VH region comprises and amino acids 1-127 of SEQ ID NO:513 or a variant thereof comprising 1, 2, 3, or 4 conservative amino acid substitutions, and wherein the VL region comprises amino acids 1-97 of SEQ ID NO:515 or a variant thereof comprising 1, 2, 3, or 4 conservative amino acid substitutions;
cvi) wherein the VH region comprises and amino acids 1-127 of SEQ ID NO:517 or a variant thereof comprising 1, 2, 3, or 4 conservative amino acid substitutions, and wherein the VL region comprises amino acids 1-97 of SEQ ID NO:519 or a variant thereof comprising 1, 2, 3, or 4 conservative amino acid substitutions; cvii) an antibody wherein the VH region comprises and amino acids 1-127 of SEQ ID NO:521 or a variant thereof comprising 1, 2, 3, or 4 conservative amino acid substitutions, and wherein the VL region comprises amino acids 1-97 of SEQ ID NO:523 or a variant thereof comprising 1, 2, 3, or 4 conservative amino acid substitutions;
cviii) an antibody wherein the VH region comprises and amino acids 1-127 of SEQ ID NO:525 or a variant thereof comprising 1, 2, 3, or 4 conservative amino acid substitutions, and wherein the VL region comprises amino acids 1-97 of SEQ ID NO:527 or a variant thereof comprising 1, 2, 3, or 4 conservative amino acid substitutions;
cix) an antibody wherein the VH region comprises and amino acids 1-127 of SEQ ID NO:529 or a variant thereof comprising 1, 2, 3, or 4 conservative amino acid substitutions, and wherein the VL region comprises amino acids 1-97 of SEQ ID NO:531 or a variant thereof comprising 1, 2, 3, or 4 conservative amino acid substitutions;
cx) an antibody wherein the VH region comprises and amino acids 1-127 of SEQ ID NO:533 or a variant thereof comprising 1, 2, 3, or 4 conservative amino acid substitutions, and wherein the VL region comprises amino acids 1-97 of SEQ ID NO:535 or a variant thereof comprising 1, 2, 3, or 4 conservative amino acid substitutions; and
cxi) an antibody wherein the VH region comprises and amino acids 1-127 of SEQ ID NO:537 or a variant thereof comprising 1, 2, 3, or 4 conservative amino acid substitutions, and wherein the VL region comprises amino acids 1-97 of SEQ ID NO:539 or a variant thereof comprising 1, 2, 3, or 4 conservative amino acid substitutions.

In some embodiments, the anti-HIV antibody is selected from the group consisting of:
i) an antibody comprising a heavy chain variable region, wherein the CDRs comprise amino acid sequences GYDFIDYV (SEQ ID NO:401), MNPSGGGT (SEQ ID NO:402) and VRDRANGSGRRRFESVNWFLDL (SEQ ID NO:403), wherein the heavy chain in the framework 3 region comprises an amino acid sequence selected from the group consisting of QLSQDPDDPDWG (SEQ ID NO:541) and LFSQDLYYPDRG (SEQ ID NO:542);
ii) an antibody comprising a light chain variable region, wherein the CDRs comprise amino acid sequences HNY, DFN and WAFEN (SEQ ID NO:404), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
iii) an antibody comprising a heavy chain variable region, wherein the CDRs comprise amino acid sequences GYRFPDYI (SEQ ID NO:497), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542);
iv) an antibody comprising a light chain variable region, wherein the CDRs comprise amino acid sequences HNL, DFN and WAYEA (SEQ ID NO:408), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
v) an antibody comprising a heavy chain variable region, wherein the CDRs comprise amino acid sequences GYKFPDYI (SEQ ID NO:405), INPMGGQV (SEQ ID NO:406) and VRDRSNGSGRRFESSN (SEQ ID NO:407), wherein the heavy chain in the framework 3 region comprises an amino acid sequence selected from the group consisting of QLSQDPDDPDWG (SEQ ID NO:541) and LFSQDLYYPDRG (SEQ ID NO:542);
vi) an antibody comprising a heavy chain variable region, wherein the CDRs comprise amino acid sequences GYTFTDYL (SEQ ID NO:409), MNPVYGQV (SEQ ID NO:410) and VRDTGDGSRRHFDSINWFLDL (SEQ ID NO:411), wherein the heavy chain in the framework 3 region comprises an amino acid sequence selected from the group consisting of QLSQDPDDPDWG (SEQ ID NO:541) and LFSQDLYYPDRG (SEQ ID
vii) an antibody comprising a light chain variable region, wherein the CDRs comprise amino acid sequences HNY, DFD and WAFEA (SEQ ID NO:412), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
viii) an antibody comprising a heavy chain variable region, wherein the CDRs comprise amino acid sequences GYTFTDYV (SEQ ID NO:413), IDPPYGQV (SEQ ID NO:414) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542);
ix) an antibody comprising a heavy chain variable region, wherein the CDRs comprise amino acid sequences GYTFTDYV (SEQ ID NO:413), INPGYGQV (SEQ ID NO:431) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542);
x) an antibody comprising a heavy chain variable region, wherein the CDRs comprise amino acid sequences GYTFVDYF (SEQ ID NO:416), MDPLNGRP (SEQ ID NO:417) and VRDKSNGSGRRFDSSNWFLDL (SEQ ID NO:418), wherein the heavy chain in the framework 3 region comprises an amino acid sequence selected from the group consisting of QLSQDPDDPDWG (SEQ ID NO:541) and LFSQDLYYPDRG (SEQ ID NO:542);
xi) an antibody comprising a light chain variable region, wherein the CDRs comprise amino acid sequences HNY, DFN and WAYDA (SEQ ID NO:419), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xii) an antibody comprising a heavy chain variable region, wherein the CDRs comprise amino acid sequences GYTFSDYI (SEQ ID NO:420), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542);
xiii) an antibody comprising a light chain variable region, wherein the CDRs comprise amino acid sequences HNL, DFN and WAYEV (SEQ ID NO:422), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xiv) an antibody comprising a heavy chain variable region, wherein the CDRs comprise amino acid sequences GYTFIDYI (SEQ ID NO:423), IDPMNGRP (SEQ ID NO:424) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542);
xv) an antibody comprising a heavy chain variable region, wherein the CDRs comprise amino acid sequences GYTFTDYI (SEQ ID NO:426), MNPMGGRT (SEQ ID NO:427) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542);
xvi) an antibody comprising a heavy chain variable region, wherein the CDRs comprise amino acid sequences GYTFVDYL (SEQ ID NO:428), MDPMNGRP (SEQ ID NO:429) and VRDKSGGSGKLFDSSNWFLDL (SEQ ID NO:430), wherein the heavy chain in the framework 3 region comprises an amino acid sequence selected from the group consisting of QLSQDPDDPDWG (SEQ ID NO:541) and LFSQDLYYPDRG (SEQ ID NO:542);
xvii) an antibody comprising a heavy chain variable region, wherein the CDRs comprise amino acid sequences GYTFTDYV (SEQ ID NO:413), MDPSYGQV (SEQ ID NO:432)and VRDRSHGSGRQFESSNWFLDL (SEQ ID NO:433), wherein the heavy chain in the framework 3 region comprises an amino acid sequence selected from the group consisting of QLSQDPDDPDWG (SEQ ID NO:541) and LFSQDLYYPDRG (SEQ ID NO:542);
xviii) an antibody comprising a heavy chain variable region, wherein the CDRs comprise amino acid sequences GYTFTDYV (SEQ ID NO:413), MDPSFGQM (SEQ ID NO:434)and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542);
xix) an antibody comprising a heavy chain variable region, wherein the CDRs comprise amino acid sequences GYRFTDYV (SEQ ID NO:436), MDPSFGRM (SEQ ID NO:437) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542);
xx) an antibody comprising a heavy chain variable region, wherein the CDRs comprise amino acid sequences GYTFIDYV (SEQ ID NO:438), MDPTYGRM (SEQ ID NO:439)and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxi) an antibody comprising a heavy chain variable region, wherein the CDRs comprise amino acid sequences GYRFLDYI (SEQ ID NO:440), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxii) an antibody comprising a heavy chain variable region, wherein the CDRs comprise amino acid sequences GYKFMDQL (SEQ ID NO:442), MNPTYGQV (SEQ ID NO:443) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxiii) an antibody comprising a light chain variable region, wherein the CDRs comprise amino acid sequences RHII (SEQ ID NO:445), DDD and NTYEF (SEQ ID NO:446), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxiv) an antibody comprising a heavy chain variable region, wherein the CDRs comprise amino acid sequences GYNFVDSR (SEQ ID NO:447), INPLQGGV (SEQ ID NO:448) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxv) an antibody comprising a light chain variable region, wherein the CDRs comprise amino acid sequences S, ESS and SILEF (SEQ ID NO:450), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxvi) an antibody comprising a heavy chain variable region, wherein the CDRs comprise amino acid sequences GYTFTTHHGHF (SEQ ID NO:500), MNPMTGQM (SEQ ID NO:462) and ARGDFGQNYPFHY (SEQ ID NO:463), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxvii) an antibody comprising a light chain variable region, wherein the CDRs comprise amino acid sequences NRYL (SEQ ID NO:464), DDN and ASYER (SEQ ID NO:465), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxviii) an antibody comprising a heavy chain variable region, wherein the CDRs comprise amino acid sequences GYNFMDQF (SEQ ID NO:466), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxix) an antibody comprising a light chain variable region, wherein the CDRs comprise amino acid sequences RHII (SEQ ID NO:445), DDD and NTYEF (SEQ ID NO:446), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxx) an antibody comprising a heavy chain variable region, wherein the CDRs comprise amino acid sequences GYNFVDSR (SEQ ID NO:447), INPLHGGV (SEQ ID NO:468) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxi) an antibody comprising a heavy chain variable region, wherein the CDRs comprise amino acid sequences GYTFTKYF (SEQ ID NO:451), IHPRTGAV (SEQ ID NO:452) and ARGAFEADSYGSSYPFHH (SEQ ID NO:453), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxii) an antibody comprising a light chain variable region, wherein the CDRs comprise amino acid sequences GNYNP (SEQ ID NO:454), EDN and ASFEF (SEQ ID NO:455), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxiii) an antibody comprising a heavy chain variable region, wherein the CDRs comprise amino acid sequences GYTFTKYT (SEQ ID NO:456), IHPRTGAV (SEQ ID NO:452) and ARGAFEADLSGPTYPFHH (SEQ ID NO:457), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxiv) an antibody comprising a heavy chain variable region, wherein the CDRs comprise amino acid sequences GFNFIDSV (SEQ ID NO:458), IKPLRGAV (SEQ ID NO:459) and AKGAFRGGSPFGF (SEQ ID NO:460), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxv) an antibody comprising a light chain variable region, wherein the CDRs comprise amino acid sequences DVT and ASREF (SEQ ID NO:461), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxvi) an antibody comprising a heavy chain variable region, wherein the CDRs comprise amino acid sequences GYTFTSYF (SEQ ID NO:469), INPLHGAV (SEQ ID NO:470) and TRGIVADGWPYGH (SEQ ID NO:471), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxvii) an antibody comprising a light chain variable region, wherein the CDRs comprise amino acid sequences S, EGA and SSLQF (SEQ ID NO:472), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxviii) an antibody comprising a heavy chain variable region, wherein the CDRs comprise amino acid sequences GFTFIDHI (SEQ ID NO:473), IKPLRGAV (SEQ ID NO:459) and CKAAAPEEAFPLQY (SEQ ID NO:474), wherein the heavy chain in the framework 3 region comprises an amino acid sequence selected from the group consisting of QLSQDPDDPDWG (SEQ ID NO:541) and LFSQDLYYPDRG (SEQ ID NO:542);
xxxix) an antibody comprising a light chain variable region, wherein the CDRs comprise amino acid sequences NVD, DNN and SSRTF (SEQ ID NO:475), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xl) an antibody comprising a heavy chain variable region, wherein the CDRs comprise amino acid sequences GFKFIDHI (SEQ ID NO:476), IKPLGGVA (SEQ ID NO:477) and CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542);
xli) an antibody comprising a light chain variable region, wherein the CDRs comprise amino acid sequences NVD, DNN and SSTTF (SEQ ID NO:479), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xlii) an antibody comprising a heavy chain variable region, wherein the CDRs comprise amino acid sequences GFAFLDH (SEQ ID NO:480), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542);
xliii) an antibody comprising a heavy chain variable region, wherein the CDRs comprise amino acid sequences GFAFLDHI (SEQ ID NO:486), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542);
xliv) an antibody comprising a heavy chain variable region, wherein the CDRs comprise amino acid sequences GFKFTEYF (SEQ ID NO:483), LNPLRGAV (SEQ ID NO:484), ARAVFNEAFPFDY (SEQ ID NO:485), wherein the heavy chain in the framework 3 region comprises an amino acid sequence selected from the group consisting of QLSQDPDDPDWG (SEQ ID NO:541) and LFSQDLYYPDRG (SEQ ID NO:542);
xlv) an antibody comprising a light chain variable region, wherein the CDRs comprise amino acid sequences VS, DGD and ASREF (SEQ ID NO:461), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xlvi) an antibody comprising a light chain variable region, wherein the CDRs comprise amino acid sequences NVD, DND and SSTTF (SEQ ID NO:479), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
xlvii) an antibody comprising a heavy chain variable region, wherein the CDRs comprise amino acid sequences GFNFIDSV (SEQ ID NO:458), IKPLRGGV (SEQ ID NO:490) and AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542);
xlviii) an antibody comprising a heavy chain variable region, wherein the CDRs comprise amino acid sequences GFKFIDSV (SEQ ID NO:487), IKPLGGAV (SEQ ID NO:488) and AKGAFGGGSPFGF (SEQ ID NO:489), wherein the heavy chain in the framework 3 region comprises an amino acid sequence selected from the group consisting of QLSQDPDDPDWG (SEQ ID NO:541) and LFSQDLYYPDRG (SEQ ID NO:542);
xlix) an antibody comprising a heavy chain variable region, wherein the CDRs comprise amino acid sequences GFTFIKYT (SEQ ID NO:492), IHPRTGAV (SEQ ID NO:452) and ARGAFEADLYGPTYPFHH (SEQ ID NO:493), wherein the heavy chain in the framework 3 region comprises an amino acid sequence selected from the group consisting of QLSQDPDDPDWG (SEQ ID NO:541) and LFSQDLYYPDRG (SEQ ID NO:542);
l) an antibody comprising a light chain variable region, wherein the CDRs comprise amino acid sequences GSYNP (SEQ ID NO:494), DDN and ASFEF (SEQ ID NO:455), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542); and
li) an antibody comprising a heavy chain variable region, wherein the CDRs comprise amino acid sequences GYNFVDSL (SEQ ID NO:495), INPLQGGV (SEQ ID NO:448) and ARGIDGNSYPFHF (SEQ ID NO:496), wherein the heavy chain in the framework 3 region comprises an amino acid sequence selected from the group consisting of QLSQDPDDPDWG (SEQ ID NO:541) and LFSQDLYYPDRG (SEQ ID NO:542);
lii) an antibody comprising a heavy chain variable region, wherein the CDRs comprise amino acid sequences GYNFMDQF (SEQ ID NO:466), MNPIWGQV (SEQ ID NO:543) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
liii) an antibody comprising a heavy chain variable region, wherein the CDRs comprise amino acid sequences GYNFMDQF (SEQ ID NO:466), MNPIFGQV (SEQ ID NO:544) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
liv) an antibody comprising a heavy chain variable region, wherein the CDRs comprise amino acid sequences GYDFMDQF (SEQ ID NO:545), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and
a. an antibody comprising a heavy chain variable region, wherein the CDRs comprise amino acid sequences GYDFIDQF (SEQ ID NO:546), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody is selected from the group consisting of:
i) an antibody comprising a heavy chain variable region, wherein the CDRs comprise amino acid sequences GYDFIDYV (SEQ ID NO:401), MNPSGGGT (SEQ ID NO:402) and VRDRANGSGRRRFESVNWFLDL (SEQ ID NO:403), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAFEN (SEQ ID NO:404);
ii) an antibody comprising a heavy chain variable region, wherein the CDRs comprise amino acid sequences GYRFPDYI (SEQ ID NO:497), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
iii) an antibody comprising a heavy chain variable region, wherein the CDRs comprise amino acid sequences GYKFPDYI (SEQ ID NO:405), INPMGGQV (SEQ ID NO:406) and VRDRSNGSGRRFESSN (SEQ ID NO:407), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
iv) an antibody comprising a heavy chain variable region, wherein the CDRs comprise amino acid sequences GYTFTDYL (SEQ ID NO:409), MNPVYGQV (SEQ ID NO:410) and VRDTGDGSRRHFDSINWFLDL (SEQ ID NO:411), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFD and WAFEA (SEQ ID NO:412);
v) an antibody comprising a heavy chain variable region, wherein the CDRs comprise amino acid sequences GYTFTDYV (SEQ ID NO:413), IDPPYGQV (SEQ ID NO:414) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
vi) an antibody comprising a heavy chain variable region, wherein the CDRs comprise amino acid sequences GYTFTDYV (SEQ ID NO:413), INPGYGQV (SEQ ID NO:431) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
vii) an antibody comprising a heavy chain variable region, wherein the CDRs comprise amino acid sequences GYTFVDYF (SEQ ID NO:416), MDPLNGRP (SEQ ID NO:417) and VRDKSNGSGRRFDSSNWFLDL (SEQ ID NO:418), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAYDA (SEQ ID NO:419);
viii) an antibody comprising a heavy chain variable region, wherein the CDRs comprise amino acid sequences GYTFSDYI (SEQ ID NO:420), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEV (SEQ ID NO:422);
ix) an antibody comprising a heavy chain variable region, wherein the CDRs comprise amino acid sequences GYTFIDYI (SEQ ID NO:423), IDPMNGRP (SEQ ID NO:424) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAYDA (SEQ ID NO:419);
x) an antibody comprising a heavy chain variable region, wherein the CDRs comprise amino acid sequences GYTFTDYI (SEQ ID NO:426), MNPMGGRT (SEQ ID NO:427) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xi) an antibody comprising a heavy chain variable region, wherein the CDRs comprise amino acid sequences GYTFVDYL (SEQ ID NO:428), MDPMNGRP (SEQ ID NO:429) and VRDKSGGSGKLFDSSNWFLDL (SEQ ID NO:430), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAYDA (SEQ ID NO:419);
xii) an antibody comprising a heavy chain variable region, wherein the CDRs comprise amino acid sequences GYTFTDYV (SEQ ID NO:413), INPGYGQV (SEQ ID NO:431) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xiii) an antibody comprising a heavy chain variable region, wherein the CDRs comprise amino acid sequences GYTFTDYV (SEQ ID NO:413), MDPSYGQV (SEQ ID NO:432)and VRDRSHGSGRQFESSNWFLDL (SEQ ID NO:433), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xiv) an antibody comprising a heavy chain variable region, wherein the CDRs comprise amino acid sequences GYTFTDYV (SEQ ID NO:413), MDPSFGQM (SEQ ID NO:434) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xv) an antibody comprising a heavy chain variable region, wherein the CDRs comprise amino acid sequences GYRFTDYV (SEQ ID NO:436), MDPSFGRM (SEQ ID NO:437) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xvi) an antibody comprising a heavy chain variable region, wherein the CDRs comprise amino acid sequences GYTFIDYV (SEQ ID NO:438), MDPTYGRM (SEQ ID NO:439) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xvii) an antibody comprising a heavy chain variable region, wherein the CDRs comprise amino acid sequences GYRFLDYI (SEQ ID NO:440), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
xviii) an antibody comprising a heavy chain variable region, wherein the CDRs comprise amino acid sequences GYKFMDQL (SEQ ID NO:442), MNPTYGQV (SEQ ID NO:443) and ARGPSGENYPFHY (SEQ ID NO:444), wherein the heavy chain in the framework 3 region comprises an amino acid sequence selected from the group consisting of NO:542); and a light chain variable region, wherein the CDRs comprise amino acid sequences RHII (SEQ ID NO:445), DDD and NTYEF (SEQ ID NO:446);
xix) an antibody comprising a heavy chain variable region, wherein the CDRs comprise amino acid sequences GYNFVDSR (SEQ ID NO:447), INPLQGGV (SEQ ID NO:448) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, ESS and SILEF (SEQ ID NO:450);
xx) an antibody comprising a heavy chain variable region, wherein the CDRs comprise amino acid sequences GYTFTTHHGHF (SEQ ID NO:500), MNPMTGQM (SEQ ID NO:462) and ARGDFGQNYPFHY (SEQ ID NO:463), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NRYL (SEQ ID NO:464), DDN and ASYER (SEQ ID NO:465);
xxi) an antibody comprising a heavy chain variable region, wherein the CDRs comprise amino acid sequences GYNFMDQF (SEQ ID NO:466), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences RHII (SEQ ID NO:445), DDD and NTYEF (SEQ ID NO:446);
xxii) an antibody comprising a heavy chain variable region, wherein the CDRs comprise amino acid sequences GYNFVDSR (SEQ ID NO:447), INPLHGGV (SEQ ID NO:468) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, ESS and SILEF (SEQ ID NO:450);
xxiii) an antibody comprising a heavy chain variable region, wherein the CDRs comprise amino acid sequences GYTFTKYF (SEQ ID NO:451), IHPRTGAV (SEQ ID NO:452) and ARGAFEADSYGSSYPFHH (SEQ ID NO:453), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences GNYNP (SEQ ID NO:454), EDN and ASFEF (SEQ ID NO:455);
xxiv) an antibody comprising a heavy chain variable region, wherein the CDRs comprise amino acid sequences GYTFTKYT (SEQ ID NO:456), IHPRTGAV (SEQ ID NO:452) and ARGAFEADLSGPTYPFHH (SEQ ID NO:457), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences GNYNP (SEQ ID NO:454), EDN and ASFEF (SEQ ID NO:455);
xxv) an antibody comprising a heavy chain variable region, wherein the CDRs comprise amino acid sequences GFNFIDSV (SEQ ID NO:458), IKPLRGAV (SEQ ID NO:459) and AKGAFRGGSPFGF (SEQ ID NO:460), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
xxvi) an antibody comprising a heavy chain variable region, wherein the CDRs comprise amino acid sequences GYTFTSYF (SEQ ID NO:469), INPLHGAV (SEQ ID NO:470) and TRGIVADGWPYGH (SEQ ID NO:471), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, EGA and SSLQF (SEQ ID NO:472);
xxvii) an antibody comprising a heavy chain variable region, wherein the CDRs comprise amino acid sequences GFTFIDHI (SEQ ID NO:473), IKPLRGAV (SEQ ID NO:459) and CKAAAPEEAFPLQY (SEQ ID NO:474), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSRTF (SEQ ID NO:475);
xxviii) an antibody comprising a heavy chain variable region, wherein the CDRs comprise amino acid sequences GFKFIDHI (SEQ ID NO:476), IKPLGGVA (SEQ ID NO:477) and CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
xxix) an antibody comprising a heavy chain variable region, wherein the CDRs comprise amino acid sequences GFAFLDH (SEQ ID NO:480), VKTIGGVV (SEQ ID NO:481) and SKAAAPDEAFPLEF (SEQ ID NO:482), wherein the heavy chain in the framework 3 region comprises an amino acid sequence selected from the group consisting of NO:542); and a light chain variable region, wherein the CDRs comprise amino acid sequences NVD, DNN and SSTTF (SEQ ID NO:479);
xxx) an antibody comprising a heavy chain variable region, wherein the CDRs comprise amino acid sequences GFAFLDHI (SEQ ID NO:486), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
xxxi) an antibody comprising a heavy chain variable region, wherein the CDRs comprise amino acid sequences GFKFTEYF (SEQ ID NO:483), LNPLRGAV (SEQ ID NO:484), ARAVFNEAFPFDY (SEQ ID NO:485), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences VS, DGD and ASREF (SEQ ID NO:461);
xxxii) an antibody comprising a heavy chain variable region, wherein the CDRs comprise amino acid sequences GFKFIDHI (SEQ ID NO:476), IKPLGGVA (SEQ ID NO:477) and CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DND and SSTTF (SEQ ID NO:479);
xxxiii) an antibody comprising a heavy chain variable region, wherein the CDRs comprise amino acid sequences GFAFLDHI (SEQ ID NO:486), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
xxxiv) an antibody comprising a heavy chain variable region, wherein the CDRs comprise amino acid sequences GFNFIDSV (SEQ ID NO:458), IKPLRGGV (SEQ ID NO:490) and AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
xxxv) an antibody comprising a heavy chain variable region, wherein the CDRs comprise amino acid sequences GFKFIDSV (SEQ ID NO:487), IKPLGGAV (SEQ ID NO:488) and AKGAFGGGSPFGF (SEQ ID NO:489), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
xxxvi) an antibody comprising a heavy chain variable region, wherein the CDRs comprise amino acid sequences GFNFIDSV (SEQ ID NO:458), IKPLRGGV (SEQ ID NO:490) and AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
xxxvii) an antibody comprising a heavy chain variable region, wherein the CDRs comprise amino acid sequences GFTFIKYT (SEQ ID NO:492), IHPRTGAV (SEQ ID NO:452) and ARGAFEADLYGPTYPFHH (SEQ ID NO:493), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences GSYNP (SEQ ID NO:494), DDN and ASFEF (SEQ ID NO:455);
xxxviii) an antibody comprising a heavy chain variable region, wherein the CDRs comprise amino acid sequences GYNFVDSL (SEQ ID NO:495), INPLQGGV (SEQ ID NO:448) and ARGIDGNSYPFHF (SEQ ID NO:496), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, ESS and SILEF (SEQ ID NO:450).

In some embodiments, the anti-HIV antibody is selected from the group consisting of:
a. an antibody comprising a heavy chain variable region, wherein CDR H1 comprises GYDFIDYV (SEQ ID NO:401), CDR H2 comprises MNPSGGGT (SEQ ID NO:402) and CDR H3 comprises VRDRANGSGRRRFESVNWFLDL (SEQ ID NO:403), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAFEN (SEQ ID NO:404);
b. an antibody comprising a heavy chain variable region, wherein CDR H1 comprises GYKFPDYI (SEQ ID NO:405), CDR H2 comprises INPMGGQV (SEQ ID NO:406) and CDR H3 comprises VRDRSNGSGRRFESSN (SEQ ID NO:407), wherein the heavy chain in the framework 3 region comprises an amino acid sequence selected from the group consisting of NO:542); and a light chain variable region, wherein CDR L1 comprises HNL, CDR L2 comprises DFN and CDR L3 comprises WAYEA (SEQ ID NO:408);
c. an antibody comprising a heavy chain variable region, wherein CDR H1 comprises GYTFTDYL (SEQ ID NO:409), CDR H2 comprises MNPVYGQV (SEQ ID NO:410) and CDR H3 comprises VRDTGDGSRRHFDSINWFLDL (SEQ ID NO:411), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFD and CDR L3 comprises WAFEA (SEQ ID NO:412);
d. an antibody comprising a heavy chain variable region, wherein CDR H1 comprises GYTFTDYV (SEQ ID NO:413), CDR H2 comprises IDPPYGQV (SEQ ID NO:414) and CDR H3 comprises VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
e. an antibody comprising a heavy chain variable region, wherein CDR H1 comprises GYTFVDYF (SEQ ID NO:416), CDR H2 comprises MDPLNGRP (SEQ ID NO:417) and CDR H3 comprises VRDKSNGSGRRFDSSNWFLDL (SEQ ID NO:418), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
f. an antibody comprising a heavy chain variable region, wherein CDR H1 comprises GYTFSDYI (SEQ ID NO:420), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEV (SEQ ID NO:422);
g. an antibody comprising a heavy chain variable region, wherein CDR H1 comprises GYTFIDYI (SEQ ID NO:423), CDR H2 comprises IDPMNGRP (SEQ ID NO:424) and CDR H3 comprises VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
h. an antibody comprising a heavy chain variable region, wherein CDR H1 comprises GYTFTDYI (SEQ ID NO:426), CDR H2 comprises MNPMGGRT (SEQ ID NO:427) and CDR H3 comprises VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
i. an antibody comprising a heavy chain variable region, wherein CDR H1 comprises GYTFVDYL (SEQ ID NO:428), CDR H2 comprises MDPMNGRP (SEQ ID NO:429) and CDR H3 comprises VRDKSGGSGKLFDSSNWFLDL (SEQ ID NO:430), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
j. an antibody comprising a heavy chain variable region, wherein CDR H1 comprises GYTFTDYV (SEQ ID NO:413), CDR H2 comprises INPGYGQV (SEQ ID NO:431) and CDR H3 comprises VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
k. an antibody comprising a heavy chain variable region, wherein CDR H1 comprises GYTFTDYV (SEQ ID NO:413), CDR H2 comprises MDPSYGQV (SEQ ID NO:432)and CDR H3 comprises VRDRSHGSGRQFESSNWFLDL (SEQ ID NO:433), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
l. an antibody comprising a heavy chain variable region, wherein CDR H1 comprises GYTFTDYV (SEQ ID NO:413), CDR H2 comprises MDPSFGQM (SEQ ID NO:434) and CDR H3 comprises VRDRSHGSGRLFESSNWFLDL (SEQ ID NO:435), wherein the heavy chain in the framework 3 region comprises an amino acid sequence selected from the group consisting of NO:542); and a light chain variable region, wherein CDR L1 comprises HNL, CDR L2 comprises DFN and CDR L3 comprises WAYEA (SEQ ID NO:408);
m. an antibody comprising a heavy chain variable region, wherein CDR H1 comprises GYRFTDYV (SEQ ID NO:436), CDR H2 comprises MDPSFGRM (SEQ ID NO:437) and CDR H3 comprises VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
n. an antibody comprising a heavy chain variable region, wherein CDR H1 comprises GYTFIDYV (SEQ ID NO:438), CDR H2 comprises MDPTYGRM (SEQ ID NO:439) and CDR H3 comprises VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
o. an antibody comprising a heavy chain variable region, wherein CDR H1 comprises GYRFLDYI (SEQ ID NO:440), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
p. an antibody comprising a heavy chain variable region, wherein CDR H1 comprises GYKFMDQL (SEQ ID NO:442), CDR H2 comprises MNPTYGQV (SEQ ID NO:443) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
q. an antibody comprising a heavy chain variable region, wherein CDR H1 comprises GYNFVDSR (SEQ ID NO:447), CDR H2 comprises INPLQGGV (SEQ ID NO:448) and CDR H3 comprises ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises ESS and CDR L3 comprises SILEF (SEQ ID NO:450);
r. an antibody comprising a heavy chain variable region, wherein CDR H1 comprises GYTFTTHHGHF (SEQ ID NO:500), CDR H2 comprises MNPMTGQM (SEQ ID NO:462) and CDR H3 comprises ARGDFGQNYPFHY (SEQ ID NO:463), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NRYL (SEQ ID NO:464), CDR L2 comprises DDN and CDR L3 comprises ASYER (SEQ ID NO:465);
s. an antibody comprising a heavy chain variable region, wherein CDR H1 comprises GYNFMDQF (SEQ ID NO:466), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of NO:542); and a light chain variable region, wherein CDR L1 comprises RHII (SEQ ID NO:445), CDR L2 comprises DDD and CDR L3 comprises NTYEF (SEQ ID NO:446);
t. an antibody comprising a heavy chain variable region, wherein CDR H1 comprises GYNFVDSR (SEQ ID NO:447), CDR H2 comprises INPLHGGV (SEQ ID NO:468) and CDR H3 comprises ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises ESS and CDR L3 comprises SILEF (SEQ ID NO:450);
u. an antibody comprising a heavy chain variable region, wherein CDR H1 comprises GYTFTKYF (SEQ ID NO:451), CDR H2 comprises IHPRTGAV (SEQ ID NO:452) and CDR H3 comprises ARGAFEADSYGSSYPFHH (SEQ ID NO:453), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises GNYNP (SEQ ID NO:454), CDR L2 comprises EDN and CDR L3 comprises ASFEF (SEQ ID NO:455);
v. an antibody comprising a heavy chain variable region, wherein CDR H1 comprises GYTFTKYT (SEQ ID NO:456), CDR H2 comprises IHPRTGAV (SEQ ID NO:452) and CDR H3 comprises ARGAFEADLSGPTYPFHH (SEQ ID NO:457), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises GNYNP (SEQ ID NO:454), CDR L2 comprises EDN and CDR L3 comprises ASFEF (SEQ ID NO:455);
w. an antibody comprising a heavy chain variable region, wherein CDR H1 comprises GFNFIDSV (SEQ ID NO:458), CDR H2 comprises IKPLRGAV (SEQ ID NO:459) and CDR H3 comprises AKGAFRGGSPFGF (SEQ ID NO:460), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises DVT and CDR L2 comprises ASREF (SEQ ID NO:461);
x. an antibody comprising a heavy chain variable region, wherein CDR H1 comprises GYTFTSYF (SEQ ID NO:469), CDR H2 comprises INPLHGAV (SEQ ID NO:470) and CDR H3 comprises TRGIVADGWPYGH (SEQ ID NO:471), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises EGA and CDR L3 comprises SSLQF (SEQ ID NO:472);
y. an antibody comprising a heavy chain variable region, wherein CDR H1 comprises GFTFIDHI (SEQ ID NO:473), CDR H2 comprises IKPLRGAV (SEQ ID NO:459) and CDR H3 comprises CKAAAPEEAFPLQY (SEQ ID NO:474), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSRTF (SEQ ID NO:475);
z. an antibody comprising a heavy chain variable region, wherein CDR H1 comprises GFKFIDHI (SEQ ID NO:476), CDR H2 comprises IKPLGGVA (SEQ ID NO:477) and CDR H3 comprises CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSTTF (SEQ ID NO:479);
aa. an antibody comprising a heavy chain variable region, wherein CDR H1 comprises GFAFLDH (SEQ ID NO:480), CDR H2 comprises VKTIGGVV (SEQ ID NO:481) and CDR H3 comprises SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSTTF (SEQ ID NO:479);
bb. an antibody comprising a heavy chain variable region, wherein CDR H1 comprises GFKFTEYF (SEQ ID NO:483), CDR H2 comprises LNPLRGAV (SEQ ID NO:484) and CDR H3 comprises ARAVFNEAFPFDY (SEQ ID NO:485), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises VS, CDR L2 comprises DGD and CDR L3 comprises ASREF (SEQ ID NO:461);
cc. an antibody comprising a heavy chain variable region, wherein CDR H1 comprises GFKFIDHI (SEQ ID NO:476), CDR H2 comprises IKPLGGVA (SEQ ID NO:477) and CDR H3 comprises CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DND and CDR L3 comprises SSTTF (SEQ ID NO:479);
dd. an antibody comprising a heavy chain variable region, wherein CDR H1 comprises GFAFLDHI (SEQ ID NO:486), CDR H2 comprises VKTIGGVV (SEQ ID NO:481) and CDR H3 comprises SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSTTF (SEQ ID NO:479);
ee. an antibody comprising a heavy chain variable region, wherein CDR H1 comprises GFKFIDSV (SEQ ID NO:487), CDR H2 comprises IKPLGGAV (SEQ ID NO:488) and CDR H3 comprises AKGAFGGGSPFGF (SEQ ID NO:489), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises DVT and CDR L2 comprises ASREF (SEQ ID NO:461);
ff. an antibody comprising a heavy chain variable region, wherein CDR H1 comprises GFNFIDSV (SEQ ID NO:458), CDR H2 comprises IKPLRGGV (SEQ ID NO:490) and CDR H3 comprises AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises DVT and CDR L2 comprises ASREF (SEQ ID NO:461);
gg. an antibody comprising a heavy chain variable region, wherein CDR H1 comprises GFTFIKYT (SEQ ID NO:492), CDR H2 comprises IHPRTGAV (SEQ ID NO:452) and CDR H3 comprises ARGAFEADLYGPTYPFHH (SEQ ID NO:493), wherein the heavy chain in the framework 3 region comprises an amino acid sequence selected from the group consisting of NO:542); and a light chain variable region, wherein CDR L1 comprises GSYNP (SEQ ID NO:494), CDR L2 comprises DDN and CDR L3 comprises ASFEF (SEQ ID NO:455);
hh. an antibody comprising a heavy chain variable region, wherein CDR H1 comprises GYNFVDSL (SEQ ID NO:495), CDR H2 comprises INPLQGGV (SEQ ID NO:448) and CDR H3 comprises ARGIDGNSYPFHF (SEQ ID NO:496), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises ESS and CDR L3 comprises SILEF (SEQ ID NO:450);
ii. an antibody comprising a heavy chain variable region, wherein CDR H1 comprises GYRFPDYI (SEQ ID NO:497), CDR H2 comprises MNPTYGQV (SEQ ID NO:443) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
jj. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAFEN (SEQ ID NO:404);
kk. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFD and CDR L3 comprises WAFEA (SEQ ID NO:412);
ll. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
mm. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
nn. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
oo. an antibody comprising a heavy chain variable region, wherein CDR H1 comprises GYKFMDQL (SEQ ID NO:442), CDR H2 comprises MNPTYGQV (SEQ ID NO:443) and CDR H3 comprises VRDRSNGSGKRFESSN (SEQ ID NO:498), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
pp. an antibody comprising a heavy chain variable region, wherein CDR H1 comprises GYKFMDQL (SEQ ID NO:442), CDR H2 comprises (SEQ ID NO:443) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446); and
qq. an antibody comprising a heavy chain variable region, wherein CDR H1 comprises GYRFLDYI (SEQ ID NO:440), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408).

In some embodiments, the anti-HIV antibody is selected from the group consisting of
b. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
c. an antibody comprising a heavy chain variable region, wherein CDR H1 comprises GYKFMDQL (SEQ ID NO:442), CDR H2 comprises MNPTYGQV (SEQ ID NO:443) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
d. an antibody comprising a heavy chain variable region, wherein CDR H1 comprises GYNFMDQF (SEQ ID NO:466), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
e. an antibody comprising a heavy chain variable region, wherein CDR H1 comprises GYNFMDQF (SEQ ID NO:466), CDR H2 comprises MNPIWGQV (SEQ ID NO:543) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
f. an antibody comprising a heavy chain variable region, wherein CDR H1 comprises GYNFMDQF (SEQ ID NO:466), CDR H2 comprises MNPIFGQV (SEQ ID NO:544) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
g. an antibody comprising a heavy chain variable region, wherein CDR H1 comprises GYDFMDQF (SEQ ID NO:545), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446); and
h. an antibody comprising a heavy chain variable region, wherein CDR H1 comprises GYDFIDQF (SEQ ID NO:546), CDR H1 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446).

In some embodiments, the anti-HIV antibody is a non-naturally occurring antibody. In some embodiments, the anti-HIV antibody is selected from the group consisting of: N49P6; N49P6.2; N49P7; N49P7.1; N49P7A; N49P7S; N49P7F; N49P7Y; N49P7-54TY; N49P7LS-1; N49P7LS-2; N49P7YTE; N49P7L6; N49P7L11; N49P7.1L9; N49P7.1L19 R49P7; N49P7.2; N49P11; N49P18; N49P18.2; N49P18.1; N49P19; N49P37; N49P38; N49P38.1; N49P55; N49P56; N49P57; N49P58; N49P59; N49P73; N49P74; N49P75; N49P75.1; N49P9; N49P9.1; N49P9.2; N49P9i7; N49P9i7H1; N49P9i7H2; N49P22; N49P23; N49P9.3; N49P9.4; N49P51; N49P52; N49P53; N49P54; N49P60; N49P61; N49P62; N49P63; N49P64; N49P65; N49P66; N49P67; N49P68; N49P69; N49P70; N49P71; and N49P72, except that the heavy chain amino acid sequence of the above mentioned antibodies is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

### Polypeptides

In some embodiments, the invention provides isolated polypeptides comprising an individual light chain or heavy chain described herein as well as antigen binding fragments thereof. Polypeptides (e.g., intact antibodies) comprising both a light chain and a heavy chain are also provided.

Also provided are polypeptides that comprise: a polypeptide comprising SEQ ID NOS:501, 505, 507, 509, 513, 517, 521, 525, 529, 533, or 537 or an antigen binding fragment thereof.

Also provided are polypeptides that comprise: a polypeptide having at least about 90% sequence identity to SEQ ID NOS:501, 505, 507, 509, 513, 517, 521, 525, 529, 533, or 537 or an antigen binding fragment thereof.

In some embodiments, the polypeptide comprises a polypeptide having at least about 95%, at least about 96%, at least about 97%, at least about 98%, or at least about 99% sequence identity to SEQ ID NOS:501, 505, 507, 509, 513, 517, 521, 525, 529, 533, or 537 or an antigen binding fragment thereof.

Also provided are polypeptides that comprise: a polypeptide comprising SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 or 397 or an antigen binding fragment thereof, with the exception that the heavy chain amino acid sequence of the above mentioned polypeptides is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

Also provided are polypeptides that comprise: a polypeptide having at least about 90% sequence identity to SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 or 397 with the exception that the heavy chain amino acid sequence of the above mentioned polypeptides is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the polypeptide comprises a polypeptide having at least about 95%, at least about 96%, at least about 97%, at least about 98%, or at least about 99% sequence identity to SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 or 397, with the exception that the heavy chain amino acid sequence of the above mentioned polypeptides is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

### Polynucleotides

In some embodiments, the invention encompasses polynucleotides comprising polynucleotides that encode a polypeptide as described herein, such as a heavy chain or light chain sequence of an HIV antibody or a fragment of such a polypeptide. For example, the invention provides a polynucleotide comprising a nucleic acid sequence that encodes an antibody to gp120 or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the polynucleotides are isolated. In certain embodiments, the polynucleotides are substantially pure.

In some embodiments, the invention provides a polynucleotide comprising a polynucleotide encoding a polypeptide comprising a sequence selected from the group consisting of SEQ ID NOS:501, 505, 507, 509, 513, 517, 521, 525, 529, 533, and 537.

In some embodiments, the invention provides a polynucleotide comprising a polynucleotide encoding a polypeptide comprising the heavy chain variable region found within a sequence selected from the group consisting of SEQ ID NOS:501, 505, 507, 509, 513, 517, 521, 525, 529, 533, and 537.

Also provided is a polynucleotide encoding a polypeptide having at least about 95%, at least about 96%, at least about 97%, at least about 98%, or at least about 99% sequence identity to SEQ ID NOS: 501, 505, 507, 509, 513, 517, 521, 525, 529, 533, and 537.

In some embodiments, the invention provides a polynucleotide comprising a polynucleotide encoding a polypeptide comprising a sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 or 397, with the exception that the heavy chain amino acid sequence of the above mentioned polypeptides is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the invention provides a polynucleotide comprising a polynucleotide encoding a polypeptide comprising the heavy chain variable region found within a sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 or 397, with the exception that the heavy chain amino acid sequence of the above mentioned polypeptides is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

Also provided is a polynucleotide having at least 70%, 75%, 80%, 85%, 86%, 87%, 88%, 89%, 90%, 91%, 92%, 93%, 94%, 95%, 96%, 97%, 98%, or 99% sequence identity to SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 or 397, with the exception that the heavy chain amino acid sequence of the above mentioned polypeptides is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

The invention further provides a polynucleotide comprising a sequence selected from the group consisting of SEQ ID NOS:502, 504, 506, 508, 510, 512, 514, 516, 518, 520, 522, 524, 526, 528, 530, 532, 534, 536, 538, and 540.

Also provided is a polynucleotide having at least 70%, 75%, 80%, 85%, 86%, 87%, 88%, 89%, 90%, 91%, 92%, 93%, 94%, 95%, 96%, 97%, 98%, or 99% sequence identity to SEQ ID NOS: 502, 504, 506, 508, 510, 512, 514, 516, 518, 520, 522, 524, 526, 528, 530, 532, 534, 536, 538, and 540.

In some embodiments the polynucleotides comprise the coding sequence for the mature polypeptide fused in the same reading frame to a polynucleotide which aids, for example, in expression and secretion of a polypeptide from a host cell (e.g. a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g. COS-7 cells) is used.

The present invention further relates to variants of the hereinabove described polynucleotides encoding, for example, fragments, analogs, and derivatives.

The polynucleotide variants can contain alterations in the coding regions, non-coding regions, or both. In some embodiments the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

Vectors and cells comprising the polynucleotides described herein are also provided. The term “vector” means a construct, which is capable of delivering, and expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. “Vector” also includes shuttle and expression vectors. In some embodiments, the vector is a plasmid construct and also includes an origin of replication (e.g., the ColE1 origin of replication) and a selectable marker (e.g., ampicillin or tetracycline resistance), for replication and selection, respectively. An “expression vector” refers to a vector that contains the necessary control sequences or regulatory elements for expression of the antibodies including antibody fragments of the invention, in bacterial or eukaryotic cells.

### Methods

The anti-HIV antibodies of the invention are useful in a variety of applications including, but not limited to, therapeutic treatment methods, such as the treatment, cure, functional cure, or prevention of HIV infection. The methods of use may be in vitro, ex vivo, or in vivo methods.

In some embodiments, the antibodies disclosed herein may be used as neutralizing antibodies, passively administered or given via gene therapies.

In one aspect, the anti-HIV antibodies are useful for detecting the presence of HIV in a biological sample. The term “detecting” as used herein encompasses quantitative or qualitative detection. In certain embodiments, a biological sample comprises a cell or tissue.

Certain other methods can be used to detect binding of anti-HIV antibodies to antigens such as gp120. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain embodiments, the antibodies are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

In certain embodiments, the antibodies are immobilized on an insoluble matrix. Immobilization entails separating the antibody from any antigen that remains free in solution. This conventionally is accomplished by either insolubilizing the antibody before the assay procedure, as by adsorption to a water-insoluble matrix or surface (Bennich et al., U.S. Pat. No. 3,720,760), or by covalent coupling (for example, using glutaraldehyde cross-linking), or by insolubilizing the antibody after formation of a complex between the antibody and antigen, e.g., by immunoprecipitation.

The present invention provides for methods of treating or preventing HIV infection comprising administering a therapeutically effective amount of an antibody as described herein to a subject (e.g., a subject in need of treatment). In some embodiments, the subject is a human.

Subjects at risk for HIV-related diseases or disorders include patients who have come into contact with an infected person or who have been exposed to HIV-1 in some other way. Administration of a prophylactic agent can occur prior to the manifestation of symptoms characteristic of HIV-1-related disease or disorder, such that a disease or disorder is prevented or, alternatively, delayed in its progression.

In some embodiments of the present invention, the subject is administered effective amounts of more than one anti-HIV antibody of the invention. In some embodiments, the subject is administered a pharmaceutical composition comprising a combination of antibodies of the invention, in order to treat or prevent HIV infection. In some embodiments, a combination of antibodies are administered, which can include a combination comprising any one or more of N49P7-FR or an antigen binding fragment thereof, N49P9-FR or an antigen binding fragment thereof, N49P9.3-FR or an antigen binding fragment thereof, N49P9.6-FR or an antigen binding fragment thereof, N49P9.6-FR-54W or an antigen binding fragment thereof, N49P9.6-FR-54F or an antigen binding fragment thereof, N49P9.6-FR3-06 or an antigen binding fragment thereof, N49P9.6-FR1-D or an antigen binding fragment thereof, N49P9.6-FR1-D-I or an antigen binding fragment thereof, N49P9.6 or an antigen binding fragment thereof, N49P9.6-54W or an antigen binding fragment thereof, N49P9.6-54F or an antigen binding fragment thereof, N49P9.6-LS or an antigen binding fragment thereof, N49P9.6-YTE or an antigen binding fragment thereof, N49P9.6-FR-LS or an antigen binding fragment thereof, or N49P9.6-FR-YTE or an antigen binding fragment thereof. In some embodiments, the antibody comprises the VH and VL regions of N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE as described herein. In some embodiments, the antibody comprises the CDRs of the VH and VL regions of N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE as described herein. Such combinations can be selected according to the desired immunity. The composition can further include one or more other broadly neutralizing antibodies.

Methods for preventing an increase in HIV-1 virus titer, virus replication, virus proliferation or an amount of an HIV-1 viral protein in a subject are further provided. In one embodiment, a method includes administering to the subject an amount of an anti-HIV antibody effective to prevent an increase in HIV-1 titer, virus replication or an amount of an HIV-1 protein of one or more HIV strains or isolates in the subject.

For in vivo treatment of human patients, the patient is usually administered or provided a pharmaceutical formulation including an anti-HIV antibody of the invention. When used for in vivo therapy, the antibodies of the invention are administered to the patient in therapeutically effective amounts (i.e., amounts that eliminate or reduce the patient's viral burden). The antibodies can be administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The antibodies may be administered parenterally, when possible, at the target cell site, or intravenously. Intravenous or subcutaneous administration of the antibody is preferred in certain embodiments. Therapeutic compositions of the invention are administered to a patient or subject systemically, parenterally, or locally.

For parenteral administration, the antibodies can be formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. Liposomes are used as carriers. The vehicle contains minor amounts of additives such as substances that enhance isotonicity and chemical stability, e.g., buffers and preservatives. The antibodies are typically formulated in such vehicles at concentrations of about 1 mg/ml to 10 mg/ml.

The dose and dosage regimen depends upon a variety of factors readily determined by a physician, such as the nature of the infection and the characteristics of the particular cytotoxic agent or growth inhibitory agent conjugated to the antibody (when used), e.g., its therapeutic index, the patient, and the patient's history. Generally, a therapeutically effective amount of an antibody is administered to a patient. In particular embodiments, the amount of antibody administered is in the range of about 0.1 mg/kg to about 20 mg/kg of patient body weight. Depending on the type and severity of the infection, about 0.1 mg/kg to about 20 mg/kg body weight (e.g., about 0.1-15 mg/kg/dose) of antibody is an initial candidate dosage for administration to the patient, whether, for example, by one or more separate administrations, or by continuous infusion. The progress of this therapy is readily monitored by conventional methods and assays and based on criteria known to the physician or other persons of skill in the art.

Antibodies of the invention can be coupled to a drug for delivery to a treatment site or coupled to a detectable label to facilitate imaging of a site comprising cells of interest, such as cells infected with HIV. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody of the invention and an epitope of interest (an HIV epitope) can be facilitated by attaching a detectable substance to the antibody. Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, τ3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or .sup.3H. Such labeled reagents may be used in a variety of well-known assays, such as radioimmunoassays, enzyme immunoassays, e.g., ELISA, fluorescent immunoassays, and the like.

The antibodies can be tagged with such labels by known methods. For instance, coupling agents such as aldehydes, carbodiimides, dimaleimide, imidates, succinimides, bid-diazotized benzadine and the like are used to tag the antibodies with the above-described fluorescent, chemiluminescent, and enzyme labels. An enzyme is typically combined with an antibody using bridging molecules such as carbodiimides, periodate, diisocyanates, glutaraldehyde and the like. Various labeling techniques are described in Morrison, Methods in Enzymology 32b, 103 (1974), Syvanen et al., J. Biol. Chem. 284, 3762 (1973) and Bolton and Hunter, Biochem J. 133, 529 (1973).

In one embodiment, the antibodies can be administered as immunoconjugates, conjugated to a second molecule. For example, the second molecule can be a toxin, a label, a radioisotope, a drug, or a chemical compound.

An antibody according to the invention may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like. Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin, TLR agonists (such as TLR7 agonist), or monomethylauristatin E.

Other therapeutic regimens can be combined with the administration of the anti-HIV antibody of the present invention. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

For any application, the antibody, antigen binding fragment, or nucleic acid encoding the antibody or antigen binding fragment can be combined with anti-retroviral therapy. Antiretroviral drugs are broadly classified by the phase of the retrovirus life-cycle that the drug inhibits. The disclosed antibodies can be administered in conjunction with nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. In some examples, a disclosed antibody or active fragment thereof or nucleic acids encoding such is administered in conjunction with IL-15, or conjugated to IL-15.

Single or multiple administrations of the compositions including the antibody, antigen binding fragment, or nucleic acid encoding the antibody or antigen binding fragment, that are disclosed herein, are administered depending on the dosage and frequency as required and tolerated by the patient. In any event, the composition should provide a sufficient quantity of at least one of the antibodies disclosed herein to effectively treat the patient. The dosage can be administered once, but may be applied periodically until either a therapeutic result is achieved or until side effects warrant discontinuation of therapy.

One approach to administration of nucleic acids is direct administration with plasmid DNA, such as with a mammalian expression plasmid. The nucleotide sequence encoding the disclosed antibody, or antibody binding fragments thereof, can be placed under the control of a promoter to increase expression. Another approach is to administer the nucleic acids in the form of mRNA.

In some embodiments, the subject is administered cells that are engineered to express the anti-HIV antibody. In some embodiments, the cells are engineered immune cells, such as B cells. In some embodiments, the cells are engineered, autologous cells.

In another approach to using nucleic acids, an anti-HIV antibody, or antibody binding fragment thereof can also be expressed by attenuated viral hosts or vectors or bacterial vectors. Recombinant vaccinia virus, adeno-associated virus (AAV), herpes virus, retrovirus, cytomegalovirus or other viral vectors can be used to express the antibody. For example, vaccinia vectors and methods useful protocols are described in U.S. Pat. No. 4,722,848. BCG (Bacillus Calmette Guerin) provides another vector for expression of the disclosed antibodies (see Stover, Nature 351:456-460, 1991).

### Compositions

The present invention also encompasses compositions comprising one or more antibodies of the invention. In certain embodiments, the compositions are pharmaceutical compositions. In some embodiments, formulations are prepared for storage and use by combining an antibody with a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

For the treatment or prevention of HIV, the appropriate dosage of an antibody or combination of antibodies of the present invention can depend on a variety of factors, such as the severity and course of the disease, the responsiveness of the disease, whether the antibody or agent is administered for therapeutic or preventative purposes, previous therapy, patient's clinical history, and so on all at the discretion of the treating physician. The antibody or agent can be administered one time or over a series of treatments lasting from several days to several months, or until a cure is effected or a diminution of the disease state is achieved. The administering physician can easily determine optimum dosages, dosing methodologies and repetition rates. In certain embodiments, dosage is from 0.01 μg to 100 mg per kg of body weight, and can be given once or more daily, weekly, monthly or yearly. In certain embodiments, the antibody or combination of antibodies is given once every two weeks or once every three weeks. In certain embodiments, the dosage of the antibody is from about 0.1 mg to about 20 mg per kg of body weight. The treating physician can estimate repetition rates for dosing based on measured residence times and concentrations of the drug in bodily fluids or tissues.

Effective dosages and schedules for administering embodiments of the present invention can be determined empirically. In some embodiments, and effective amount of one or more antibodies are administered to neutralize, treat, prevent or eradicate HIV infection. In some embodiments, compositions comprising one or more nucleic acid molecules of the invention are administered to the subject. In some embodiments, genetic constructs capable of inducing production of antibodies of the present invention may be administered to a patient in need thereof.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 .mu.m so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

In some embodiments, the compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

The compositions can be designed to introduce the antibodies, nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulations can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

The compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradermal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

Dosing schedules (or regimens) can be readily determined for the particular subject and composition. Hence, the composition can be administered one or more times to the subject. Preferably, there is a set time interval between separate administrations of the composition. While this interval varies for every subject, typically it can range from 10 days to several weeks, and is often 2, 4, 6 or 8 weeks. In some embodiments, the interval can be typically from 2 to 6 weeks.

The compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

### Kits

The present invention also includes kits useful in performing diagnostic and prognostic assays using the antibodies of the present invention. Kits of the invention include a suitable container comprising an HIV-1 antibody of the invention in either labeled or unlabeled form. In addition, when the antibody is supplied in a labeled form suitable for an indirect binding assay, the kit further includes reagents for performing the appropriate indirect assay. For example, the kit includes one or more suitable containers including enzyme substrates or derivatizing agents, depending on the nature of the label. Control samples and/or instructions are also included.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Development of Broadly Neutralizing and Potent Anti-HIV Antibodies

Through our efforts to deconvolute ongoing plasma bNAb responses in HIV-infected subjects, we have been isolating lineages of circulating bNAbs in HIV Elite Neutralizers. Plasma polyclonal anti-Env responses in one subject (donor N49) were particularly impressive, demonstrating near pan-neutralizing activity (Sajadi et al., Cell. 2018; 173(7):1783-95 e14. doi: 10.1016/j.cell.2018.03.061). Neutralization breadth was determined using standardized panels of pseudoviruses (representing multiple Tier 1-3 or Clade envelopes) (for detailed data see Sajadi et al., Cell. 2018; 173(7):1783-95 e14. doi: 10.1016/j.cell.2018.03.061). As previously predicted for Elite Neutralizers in our cohort, the N49 plasma bNAbs exhibited basic isoelectric points (Sajadi et al., J Virol. 2012; 86(9):5014-25) and utilized 2\., light chain genes (Sajadi et al., J Infect Dis. 2016; 213(1):156-64). Two related lineages of bNAbs (termed the N49 P series), distinguished by Lambda 2-11 or Lambda 2-23 use, were recovered from NVS 49. All of them recognize the CD4bs. Notably, a number of them have greater breadth than other CD4bs antibodies currently in clinical trials (e.g., 3BNC117 and VRC01). As we already published, one lineage of the bNAbs, exemplified by bNAb N49P7, exhibit near pan-neutralizing activity (Table 4 above). The more recently characterized (unpublished) second lineage of N49 bNAbs includes N49P9, N49P9.3, and N49P9.6. N49P9.6 has breadth comparable to the best CD4bs bNAbs (97%), combined with an overall potency that rivals the best of the PGT series of mAbs (Table 4). N49P9.3 is a clonal variant of N49P9.6 that has one Amino Acid difference in the Heavy Chain sequence.

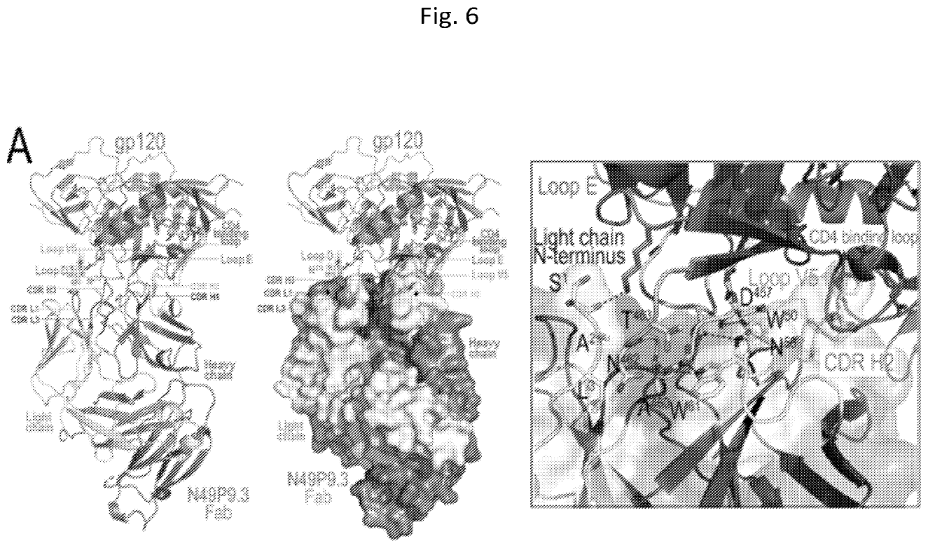

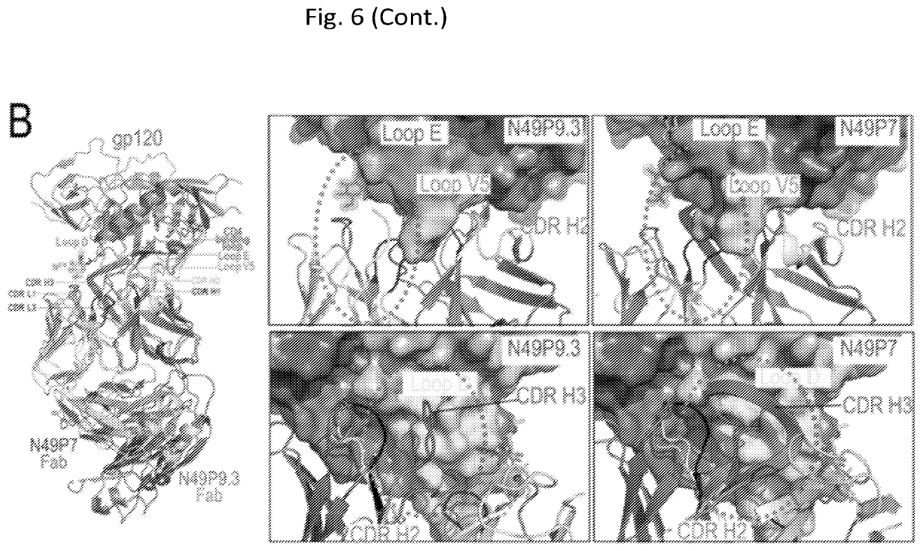

To define the molecular basis for such breadth and potency, we solved the crystal structures of N49P9.3 Fab in complex with HIV-1 93TH057 gp120 core (FIG. 6). These analyses show that N49P9.3 anchors on gp120 antigen within the CD4 binding site of gp120, engaging the CD4-binding loop as well as loops D and V5 (similar to CD4 and other CD4bs bNAbs including N49P7). N49P9.3, in contrast to N49P7, uses relatively short CDRH3 of 11 aa long (compared to 19 aa-long CDR H3 of N49P7) therefore does not reach deeply into Layer 3 of inner domain. Instead N49P9.3 uses CDRH2 to tightly bind (through network of H-bonds) to Loop V5 and H-bonds and hydrophobic contacts of the framework part of light chain (N-terminus, residues 1-3) to loop V4 and Loop E (FIGS. 6A and B). Importantly, bNAb N49P6 and its lineage members test negative for autoreactivity by immunofluorescence and Elisa (DNA, Centromere B, Histone, Jo-1, SSA, SSB, Scl-70, Sm, RNP) by clinically validated assays when tested maximally at 25 ug/ml. The half-life of these mAbs in transgenic mice with 1 copy of the human FcRn gene are comparable to mAbs currently in clinical trials (Table 5).

TABLE 5
t½ of bNAbs in FcRn−/− hFcRn transgenic mice

| | |
|---|---|
| Synagis | 3.14 days |
| VRC01 | 1.53 days |
| VRC01-LS | 2.92 days |
| PGT121 | 2.05 days |
| N49P9.6 | 2.17 days |
| N49P9.6-LS | 3.38 days |
| N49P9.6-FR | 2.68 days |
| N49P9.6-FR-LS | 3.07 days |

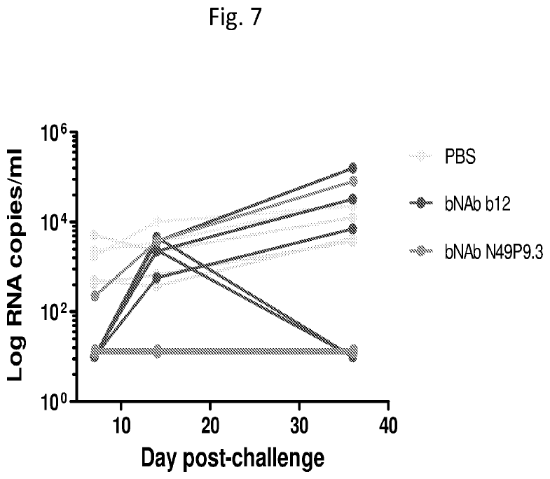

Accordingly, this series of N49 bNAbs was assessed for in vivo antiviral activity in a variety of pilot studies using humanized mouse formats we employ in our laboratories. One assay format examined bNAb efficacy in NOD scid gamma (NSG) mice (NOD.Cg-PrkcdscidIL2rgtmlWij/SzJ (NOD-scid IL2rg−/−)) reconstituted with HIV-infected human PBLs (Hu-PBL). This model can test the ability of a bNAb to suppress HIV-1 replication (which is ongoing in the infected donor cells). Groups of animals (n=5) were treated IP with 10 mg/kg of bNAb N49P9.3 or another anti-CD4bs bNAb, b12 (62). Control animals were treated with PBS. Six hours later the mice were given IP injection with 7.5×10⁶ PBMCs from a heterologous Clade B HIV-1-infected patient not on ARVs (donor LT9). Plasma viral loads were measured periodically by our in-house TaqMan RT-qPCR assay with a 40 HIV-1 RNA copy/ml lower limit of detection (Satheesan et al., J Virol. 2018; 92(7)). As shown in FIG. 7, by the end of the experiment all of the control and bNAb b12-treated animals exhibited plasma viremia at one or more points during the course of the experiment. In comparison, within the bNAb N49P9.3 group only one animal exhibited viremia over the course of the experiment. The bNAb N49P9.3 group exhibited significantly fewer days with detectable viremia versus controls (p<0.005, Fisher's exact test).

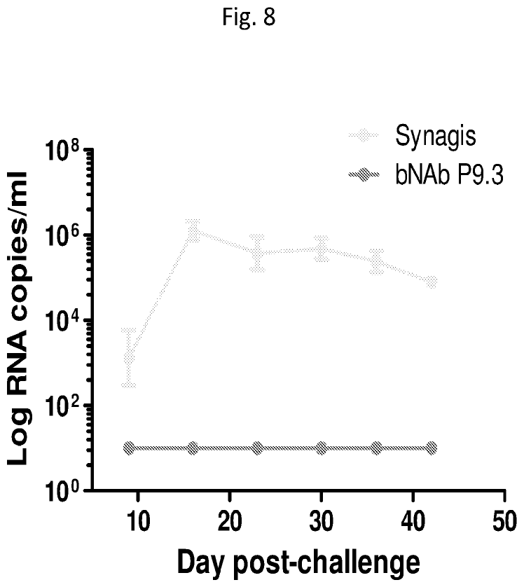

In another assay format, we tested the efficacy of bNAb P9.6 in NSG mice reconstituted with uninfected Hu-PBL (10×10⁶ donor PBMCs/animal). Three weeks later, groups of mice were given 10 mg/kg of either bNAb P9.6 (n=7) or Synagis negative control (n=8) by IP route. Six hours later they were challenged IP with 100 TCID50 of cell-free CCR5-tropic HIV-1 BaL. Plasma viral loads were measured periodically by our in-house TaqMan RT-qPCR assay with a 40 HIV-1 RNA copy/ml lower limit of detection (Satheesan et al., J Virol. 2018; 92(7)). As shown in FIG. 8, all Synagis treated animals became infected and exhibited sustained levels of plasma viremia. None of the bNAb P9.6 treated animals exhibited circulating viral loads at any point in time.

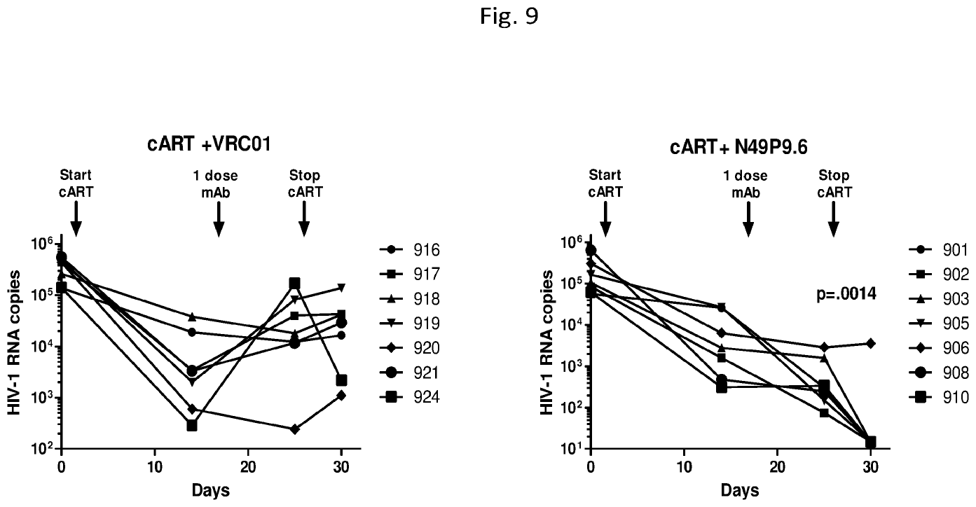

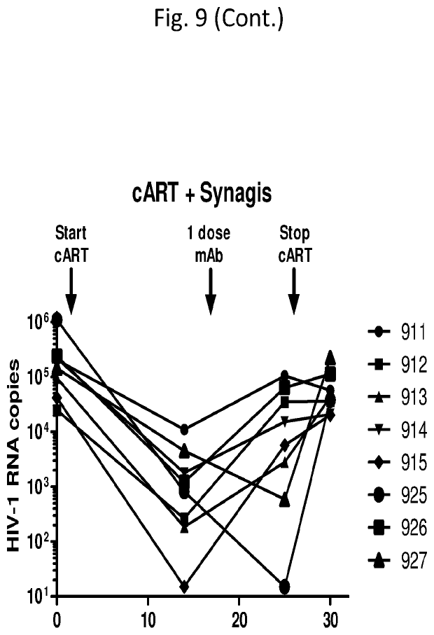

To probe therapeutic effects, HIV-1 infection was established in NSG mice reconstituted with human CD34+ stem cells (Hu-CD34). There is substantially less graft versus host disease (GVHD) in this model versus Hu-PBL mice, allowing for studies of experimental therapeutic interventions. The reconstituted mice were infected by injecting 8000 TCID50 infection of HIV-1 Bal virus IP (based on our titration studies in these mice, this dose consistently results in 100% infection of the mice). On Day 0, 2 weeks after injection of HIV-1, after which all mice were infected, cART was started with 2 drugs (tenofovir and emtricitabine) to induce partial viral suppression in this robust infection model. After 2 weeks of cART, one dose (10 mg/kg) of a test mAb (N49P9.6, VRC01, or Synagis) was administered IP as cART was continued for about 10 days longer (predicting that antibody and cART would “wash out” concurrently). The ability of the mAbs to maintain viral suppression of HIV-1 in conjunction with cART up to Day 30 was then compared. bNAb VRC01 was unable to alter viral rebound in any animal compared to the Synagis control group. However, treatment with N49P9.6 caused a continuous viral load drop to baseline in all but one animal. The difference in Day 30 viral loads between the N49P9.6 and control groups was clearly evident (p=0.0014 by 2-tailed Fisher's exact test) (FIG. 9). The in vitro neutralization potency of VRC01 against the HIV-1 BaL challenge virus was 10-fold lower than that of N49P9.6. This difference in potency is likely responsible for the variance in efficacy. Specifically, given equal doses of the two bNAbs and assuming equivalent pharmacokinetics, N49P9.6 will take longer to drop beneath its lower threshold of efficacy, thus providing a more sustained effect. Although preliminary, these data already suggest that members of the N49P series lineage may deliver greater clinical benefit versus other bNAbs of their class.

Overall, these experiments demonstrate that the impressive in vitro characteristics of N49 P series bNAbs translates to potent in vivo efficacy. Accordingly, we expect that engineered improvements in potency/breadth evident in vitro should provide a superior antiviral effect in vivo.

### Engineering of N49P9.6

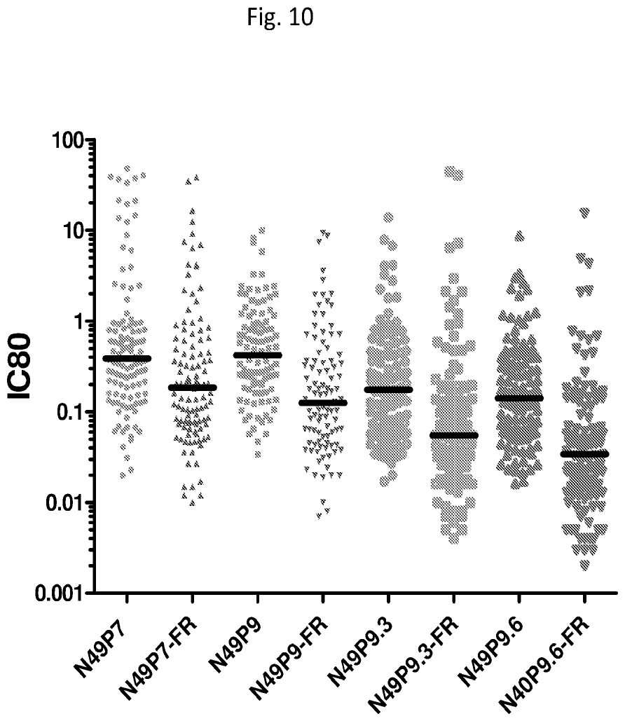

Given the above information, we initiated engineering efforts to improve antigen binding and neutralization potency. CD4 (or CD4bs antibodies) not only bind to the CD4 binding pocket of one gp120, but have additional contacts on the opposing gp120 protomer (Liu et al., Nat Commun. 2019; 10(1):721; Liu et al., Nat Struct Mol Biol. 2017; 24(4):370-8). For CD4bs antibodies FR3 and CDR1 contacts have been described (Liu et al., Nat Commun. 2019; 10(1):721; Liu et al., Nat Struct Mol Biol. 2017; 24(4):370-8). Thus, we inserted a heavy chain framework 3 (FR3) loop from a CD4bs antibody (VRC03) into N49P9.6, which itself has a FR3 deletion, in order to enable the bNAbs to bind to the adjacent protomer in the envelope trimer (Liu et al., Nat Commun. 2019; 10(1):721; Liu et al., Nat Struct Mol Biol. 2017; 24(4):370-8). The constructs were tested for neutralization breadth and potency in the 117 multiclade, pseudovirus panels discussed above. As shown in FIG. 10, one of these new constructs, N49P9.6-FR, demonstrated median potency (IC50 0.01 ug/ml and IC80 0.03 ug/ml) almost an order of magnitude lower (more potent) than N49P9.6, better than any other N49 construct, and equivalent to the more potent members of other bNAb classes. To optimize chances of success, bNAb N49P9.6-FR was be further engineered into an “LS” variant (N49P9.6-FR/LS), which will contains 2 point mutations at positions 428 and 434 (L and S, respectively) in the Fc domain. These alterations extend antibody half-life by promoting stronger binding to FcRn and preventing endosomal degradation of IgG (Zalevsky et al., Nat Biotechnol. 2010; 28(2):157-9). The “LS” version of N49P9.6-FR preserves its breadth and potency when tested in the same 117-pseudovirus neutralization panel (data not shown).

Two engineering strategies were launched. The first was based on observations that CDR1 contacts may naturally occur between N49 P series bNAbs and the adjacent protomer in the envelope trimer(Liu et al., Nat Commun. 2019; 10(1):721; Liu et al., Nat Struct Mol Biol. 2017; 24(4):370-8). It is expected that a bNAb with such binding characteristics will deliver a more potent effect, a concept already supported by N49P9.6-FR. Accordingly, we analyzed all available CD4bs antibodies isolated from donor N49, as well as the antibody database made from the donor's memory B cell and bone marrow. We identified one antibody N49P6 (not to be confused with the similarly named N49P9.6 from which the FR3 variants were made), that contained an aspartate residue in the CDR1 at position 29 (IMGT numbering system) mimicking the contact of CD4 the opposing gp120 protomer. Two variants of N49P9.6 made containing this aspartate, with improvement in potency seen similar to N49P9.6-FR in the 20 pseudovirus panel. These variants are currently undergoing testing with the full pseudovirus panel. In addition, more CDR1 variants have been made and are undergoing testing.

The second engineering strategy involved optimizing the FR3 contacts of N49P9.6-FR. The engineering efforts have been aided by structure based study of resistant variants.

Comparative Assessments of Frontline and Next Generation bNAbs

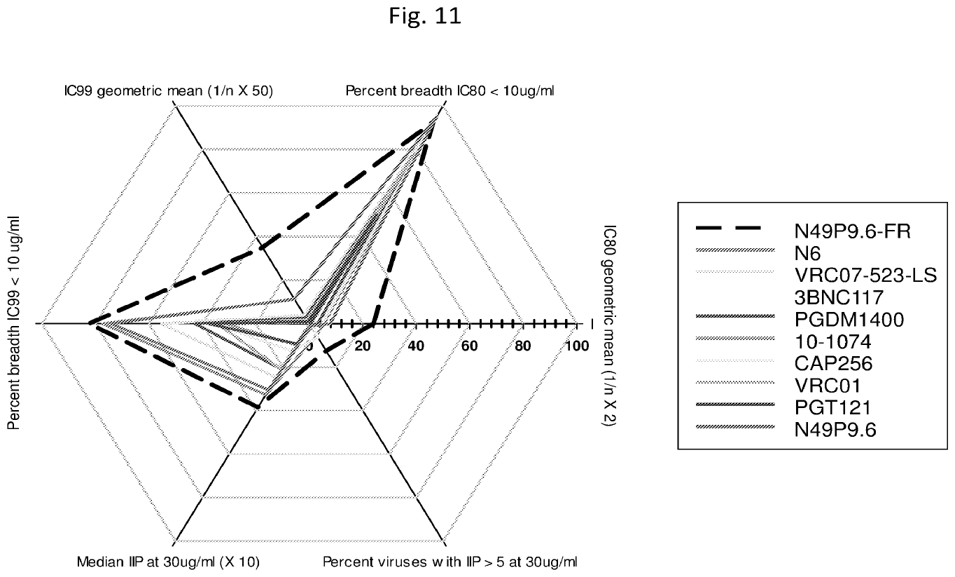

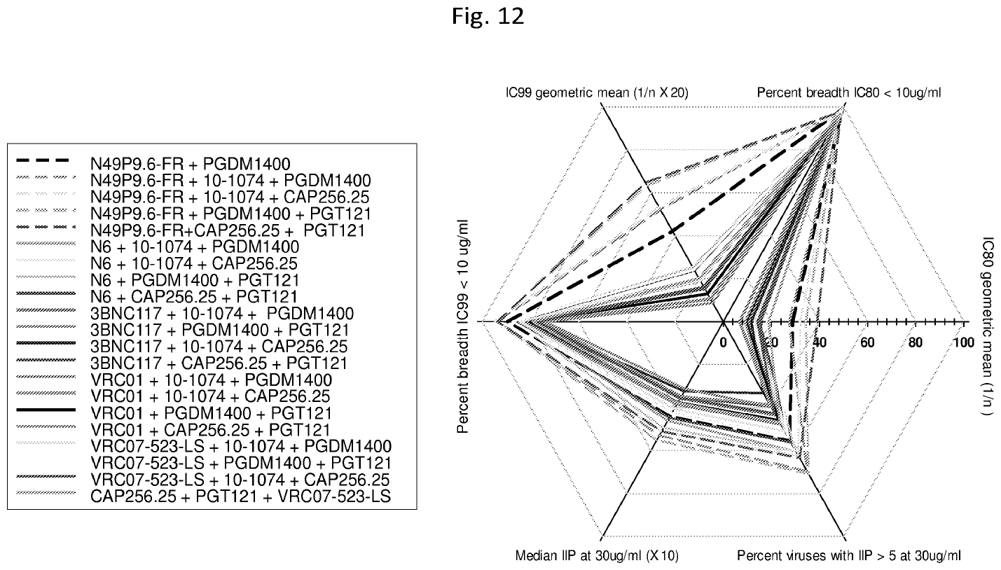

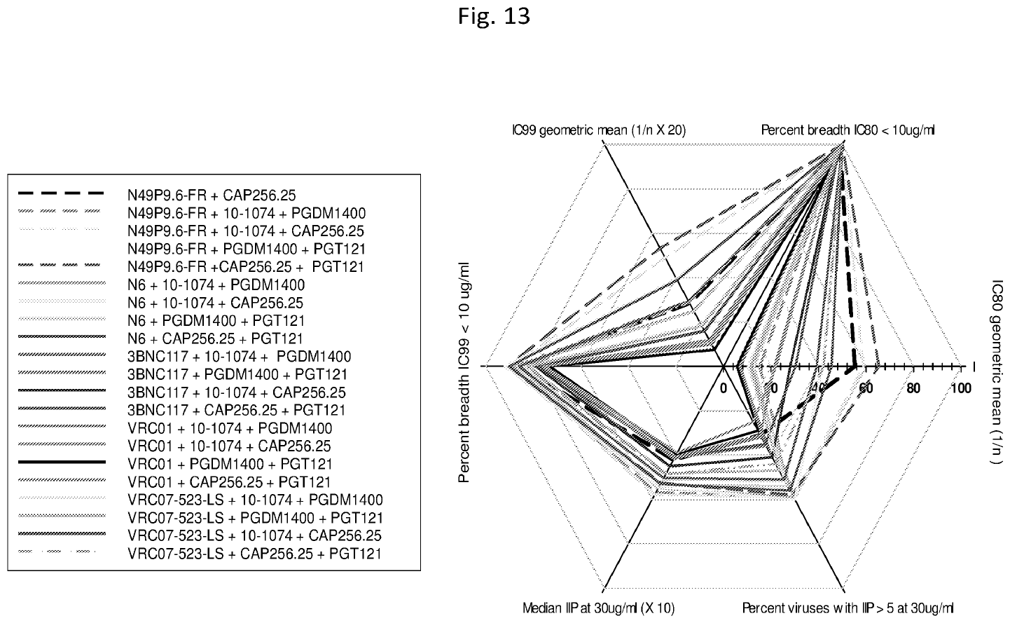

Comparative assessments of front-line bNAb potency and breadth were recently conducted under the aegis of the CA-VIMC, in part based on their highly accurate computational modeling algorithms developed to estimate potencies of bNAb combinations (67). Using various bNAb and pseudovirus panels, they compared IC99 and IC80 geometric means, IC99 and IC80% breadth at <10 ug/ml; median instantaneous inhibition potential (IIP) and % viruses exhibiting IIP >5 at total antibody concentrations of 30 ug/ml. An IIP value reflects the log number reduction of single-round infection events mediated by a drug or a drug combination according to additivity or independence models (Jilek et al., Nat Med. 2012; 18(3):446-51). In the context of cART therapy, it was shown that an IIP >5 for a given drug dose equated with >50% chance of clinical success (Jilek et al., Nat Med. 2012; 18(3):446-51). Results from a global cross-clade, cross-Tier panel of 96 pseudoviruses are shown as radar plots in FIGS. 11 and 12. The data depicted are normalized and adjusted such that higher values are more desirable (e.g. reciprocal IC values are shown) and fit along the constant scale of the radial axis. FIG. 11 shows that N49P9.6-FR is superior to all other bNAbs in clinical development, including ones against the CD4bs, in all categories. FIG. 12 shows that N49P9.6-FR is further distinguished among anti-CD4bs bNAbs (VRC01, VRC07-523-LS, 3BNC117, N6) as forming the basis for the most broad and potent triple antibody combinations. The same superiority in using N49P9.6-FR is seen in a test panel of 100 Clade C pseudoviruses (FIG. 13).

Finally, N49P9.6-FR has been compared with other next generation mAbs, namely 1-18 and LN02 ML85. In side-by-side comparisons, N49P9.6-FR was better alone and in combinations compared to these other next generation mAbs. Given such evidence, further efforts to test and engineer N49P9.6 series bNAbs is clearly warranted.

### Example 2. In Vivo Antiviral Activity of N49 Antibodies

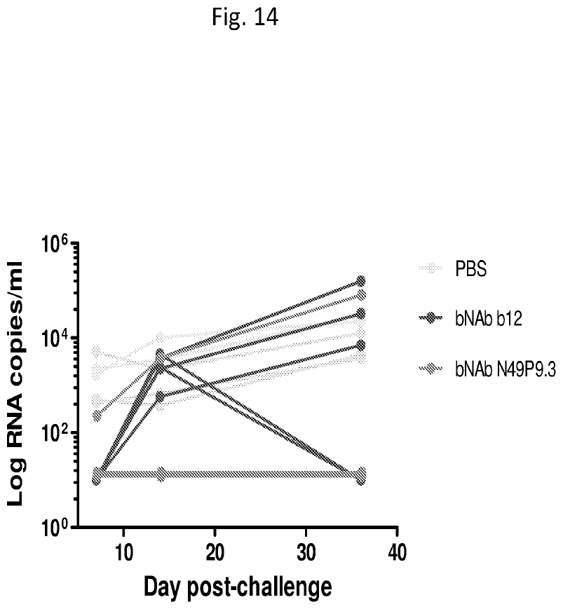

N49 bNAbs were assessed for in vivo antiviral activity in a variety of pilot studies using humanized mouse formats we employ in our laboratories. One assay format examined bNAb efficacy in NOD scid gamma (NSG) mice (NOD.Cg-PrkcdscidIL2rgtmlWij/SzJ (NOD-scid IL2rg−/−)) reconstituted with HIV-infected human PBLs (Hu-PBL). This model can test the ability of a bNAb to suppress HIV-1 replication (which is ongoing in the infected donor cells). Groups of animals (n=5) were treated IP with 10 mg/kg of bNAb N49P9.3 or another anti-CD4bs bNAb, b12. Control animals were treated with PBS. Six hours later the mice were given IP injection with 7.5×10⁶ PBMCs from a heterologous Clade B HIV-1-infected patient not on ARVs (donor LT9). Plasma viral loads were measured periodically by our in-house TaqMan RT-qPCR assay with a 40 HIV-1 RNA copy/ml lower limit of detection. As shown in FIG. 14, by the end of the experiment all of the control and bNAb b12-treated animals exhibited plasma viremia at one or more points during the course of the experiment. In comparison, within the bNAb N49P9.3 group only one animal exhibited viremia over the course of the experiment. The bNAb N49P9.3 group exhibited significantly fewer days with detectable viremia versus controls (p<0.005, Fisher's exact test).

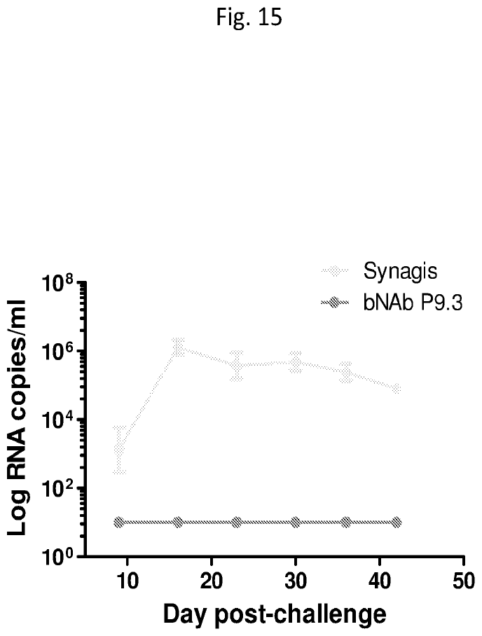

In another assay format, we tested the efficacy of bNAb P9.6 in NSG mice reconstituted with uninfected Hu-PBL (10×10⁶ donor PBMCs/animal). Three weeks later, groups of mice were given 10 mg/kg of either bNAb P9.6 (n=7) or Synagis negative control (n=8) by IP route. Six hours later they were challenged IP with 100 TCID50 of cell-free CCR5-tropic HIV-1 BaL. Plasma viral loads were measured periodically by our in-house TaqMan RT-qPCR assay with a 40 HIV-1 RNA copy/ml lower limit of detection. As shown in FIG. 15, all Synagis treated animals became infected and exhibited sustained levels of plasma viremia. None of the bNAb P9.6 treated animals exhibited circulating viral loads at any point in time.

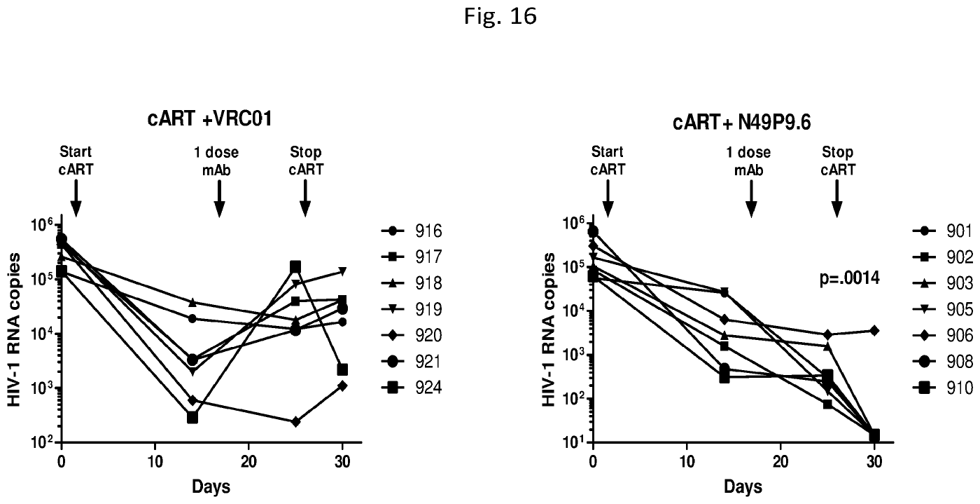

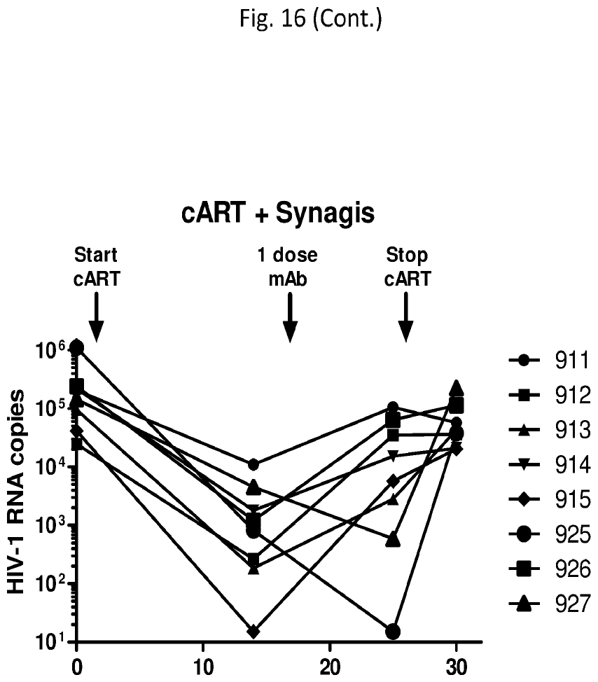

To probe therapeutic effects, HIV-1 infection was established in NSG mice reconstituted with human CD34+ stem cells (Hu-CD34). There is substantially less graft versus host disease (GVHD) in this model versus Hu-PBL mice, allowing for studies of experimental therapeutic interventions. The reconstituted mice were infected by injecting 8000 TCID50 infection of HIV-1 Bal virus IP (based on our titration studies in these mice, this dose consistently results in 100% infection of the mice). On Day 0, 2 weeks after injection of HIV-1, after which all mice were infected, cART was started with 2 drugs (tenofovir and emtricitabine) to induce partial viral suppression in this robust infection model. After 2 weeks of cART, one dose (10 mg/kg) of a test mAb (N49P9.6, VRC01, or Synagis) was administered IP as cART was continued for about 10 days longer (predicting that antibody and cART would “wash out” concurrently). The ability of the mAbs to maintain viral suppression of HIV-1 in conjunction with cART up to Day 30 was then compared. bNAb VRC01 was unable to alter viral rebound in any animal compared to the Synagis control group. However, treatment with N49P9.6 caused a continuous viral load drop to baseline in all but one animal. The difference in Day 30 viral loads between the N49P9.6 and control groups was clearly evident (p=0.0014 by 2-tailed Fisher's exact test) (FIG. 16). The in vitro neutralization potency of VRC01 against the HIV-1 BaL challenge virus was 10-fold lower than that of N49P9.6. This difference in potency is likely responsible for the variance in efficacy. Specifically, given equal doses of the two bNAbs and assuming equivalent pharmacokinetics, N49P9.6 will take longer to drop beneath its lower threshold of efficacy, thus providing a more sustained effect. These data already suggest that members of the N49P series lineage may deliver greater clinical benefit versus other bNAbs of their class. Overall, these experiments demonstrate that the impressive in vitro characteristics of N49 P series bNAbs translates to potent in vivo efficacy.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. An isolated anti-HIV antibody that is capable of neutralizing at least 90% of the HIV pseudoviruses listed in Table 4 with an IC50 value of less than 1 μg/mL.

2. The isolated anti-HIV antibody of claim 1, wherein the antibody is capable of neutralizing at least 95% of the HIV pseudoviruses listed in Table 4 with an IC50 value of less than 1 μg/mL.

3. The isolated anti-HIV antibody of claim 1, wherein the antibody is derived from a N49P series antibody, wherein the N49P series antibody is modified whereby a part or all of the framework 3 region of the heavy chain is replaced with an amino acid sequence selected from the group consisting of QLSQDPDDPDWG (SEQ ID NO:541) and LFSQDLYYPDRG (SEQ ID NO:542).

4. The isolated anti-HIV antibody of claim 1, wherein the antibody is selected from the group consisting of:
a. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
b. an antibody comprising a heavy chain variable region, wherein CDR H1 comprises GYKFMDQL (SEQ ID NO:442), CDR H2 comprises MNPTYGQV (SEQ ID NO:443) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
c. an antibody comprising a heavy chain variable region, wherein CDR H1 comprises GYNFMDQF (SEQ ID NO:466), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
d. an antibody comprising a heavy chain variable region, wherein CDR H1 comprises GYNFMDQF (SEQ ID NO:466), CDR H2 comprises MNPIWGQV (SEQ ID NO:543) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
e. an antibody comprising a heavy chain variable region, wherein CDR H1 comprises GYNFMDQF (SEQ ID NO:466), CDR H2 comprises MNPIFGQV (SEQ ID NO:544) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
f. an antibody comprising a heavy chain variable region, wherein CDR H1 comprises GYDFMDQF (SEQ ID NO:545), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446); and
g. an antibody comprising a heavy chain variable region, wherein CDR H1 comprises GYDFIDQF (SEQ ID NO:546), CDR H1 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446).

5. The isolated anti-HIV antibody of claim 1, wherein the anti-HIV antibody comprises a heavy chain or an antigen binding fragment thereof and a light chain or an antigen binding fragment thereof, wherein the heavy chain or antigen binding fragment thereof comprises a heavy chain variable (VH) region and the light chain or antigen binding fragment thereof comprises a light chain variable (VL) region; wherein the anti-HIV antibody is selected from the group consisting of:
i) an antibody wherein the VH region comprises and amino acids 1-134 of SEQ ID NO:501 or a variant thereof comprising 1, 2, 3, or 4 conservative amino acid substitutions, and wherein the VL region comprises amino acids 1-99 of SEQ ID NO:503 or a variant thereof comprising 1, 2, 3, or 4 conservative amino acid substitutions;
ii) an antibody wherein the VH region comprises and amino acids 1-127 of SEQ ID NO:505 or a variant thereof comprising 1, 2, 3, or 4 conservative amino acid substitutions, and wherein the VL region comprises amino acids 1-100 of SEQ ID NO:295 or a variant thereof comprising 1, 2, 3, or 4 conservative amino acid substitutions;
iii) an antibody wherein the VH region comprises and amino acids 1-127 of SEQ ID NO:507 or a variant thereof comprising 1, 2, 3, or 4 conservative amino acid substitutions, and wherein the VL region comprises amino acids 1-100 of SEQ ID NO:327 or a variant thereof comprising 1, 2, 3, or 4 conservative amino acid substitutions;
iv) an antibody wherein the VH region comprises and amino acids 1-127 of SEQ ID NO:509 or a variant thereof comprising 1, 2, 3, or 4 conservative amino acid substitutions, and wherein the VL region comprises amino acids 1-100 of SEQ ID NO:511 or a variant thereof comprising 1, 2, 3, or 4 conservative amino acid substitutions;
v) an antibody wherein the VH region comprises and amino acids 1-127 of SEQ ID NO:513 or a variant thereof comprising 1, 2, 3, or 4 conservative amino acid substitutions, and wherein the VL region comprises amino acids 1-100 of SEQ ID NO:515 or a variant thereof comprising 1, 2, 3, or 4 conservative amino acid substitutions;
vi) an antibody wherein the VH region comprises and amino acids 1-127 of SEQ ID NO:517 or a variant thereof comprising 1, 2, 3, or 4 conservative amino acid substitutions, and wherein the VL region comprises amino acids 1-100 of SEQ ID NO:519 or a variant thereof comprising 1, 2, 3, or 4 conservative amino acid substitutions;
vii) an antibody wherein the VH region comprises and amino acids 1-127 of SEQ ID NO:521 or a variant thereof comprising 1, 2, 3, or 4 conservative amino acid substitutions, and wherein the VL region comprises amino acids 1-100 of SEQ ID NO:523 or a variant thereof comprising 1, 2, 3, or 4 conservative amino acid substitutions;
viii) an antibody wherein the VH region comprises and amino acids 1-127 of SEQ ID NO:525 or a variant thereof comprising 1, 2, 3, or 4 conservative amino acid substitutions, and wherein the VL region comprises amino acids 1-100 of SEQ ID NO:527 or a variant thereof comprising 1, 2, 3, or 4 conservative amino acid substitutions;
ix) an antibody wherein the VH region comprises and amino acids 1-127 of SEQ ID NO:529 or a variant thereof comprising 1, 2, 3, or 4 conservative amino acid substitutions, and wherein the VL region comprises amino acids 1-100 of SEQ ID NO:531 or a variant thereof comprising 1, 2, 3, or 4 conservative amino acid substitutions;
x) an antibody wherein the VH region comprises and amino acids 1-127 of SEQ ID NO:533 or a variant thereof comprising 1, 2, 3, or 4 conservative amino acid substitutions, and wherein the VL region comprises amino acids 1-100 of SEQ ID NO:535 or a variant thereof comprising 1, 2, 3, or 4 conservative amino acid substitutions; and
xi) an antibody wherein the VH region comprises and amino acids 1-127 of SEQ ID NO:537 or a variant thereof comprising 1, 2, 3, or 4 conservative amino acid substitutions, and wherein the VL region comprises amino acids 1-100 of SEQ ID NO:539 or a variant thereof comprising 1, 2, 3, or 4 conservative amino acid substitutions.

6. The anti-HIV antibody of claim 1, wherein the anti-HIV antibody is selected from the group consisting of:
a. an antibody comprising a heavy chain amino acid sequence comprising SEQ ID NO:501 or an antigen binding fragment thereof and a light chain amino acid sequence comprising SEQ ID NO:503 or an antigen binding fragment thereof;
b. an antibody comprising a heavy chain amino acid sequence comprising SEQ ID NO:505 or an antigen binding fragment thereof and a light chain amino acid sequence comprising SEQ ID NO:295 or an antigen binding fragment thereof;
c. an antibody comprising a heavy chain amino acid sequence comprising SEQ ID NO:507 or an antigen binding fragment thereof and a light chain amino acid sequence comprising SEQ ID NO:327 or an antigen binding fragment thereof;
d. an antibody comprising a heavy chain amino acid sequence comprising SEQ ID NO:509 or an antigen binding fragment thereof and a light chain amino acid sequence comprising SEQ ID NO:511 or an antigen binding fragment thereof;
e. an antibody comprising a heavy chain amino acid sequence comprising SEQ ID NO:513 or an antigen binding fragment thereof and a light chain amino acid sequence comprising SEQ ID NO:515 or an antigen binding fragment thereof;
f. an antibody comprising a heavy chain amino acid sequence comprising SEQ ID NO:517 or an antigen binding fragment thereof and a light chain amino acid sequence comprising SEQ ID NO:519 or an antigen binding fragment thereof;
g. an antibody comprising a heavy chain amino acid sequence comprising SEQ ID NO:521 or an antigen binding fragment thereof and a light chain amino acid sequence comprising SEQ ID NO:523 or an antigen binding fragment thereof;
h. an antibody comprising a heavy chain amino acid sequence comprising SEQ ID NO:525 or an antigen binding fragment thereof and a light chain amino acid sequence comprising SEQ ID NO:527 or an antigen binding fragment thereof;
i. an antibody comprising a heavy chain amino acid sequence comprising SEQ ID NO:529 or an antigen binding fragment thereof and a light chain amino acid sequence comprising SEQ ID NO:531 or an antigen binding fragment thereof;
j. an antibody comprising a heavy chain amino acid sequence comprising SEQ ID NO:533 or an antigen binding fragment thereof and a light chain amino acid sequence comprising SEQ ID NO:535 or an antigen binding fragment thereof; and
k. an antibody comprising a heavy chain amino acid sequence comprising SEQ ID NO:537 or an antigen binding fragment thereof and a light chain amino acid sequence comprising SEQ ID NO:539 or an antigen binding fragment thereof.

7. An isolated host cell expressing the antibody of claim 1.

8. One or more vectors comprising a nucleic acid encoding the antibody of claim 1.

9. The one or more vectors of claim 8, wherein one vector encodes a light chain sequence and another vector encodes a heavy chain sequence.

10. The one or more vectors of claim 8, wherein one vector encodes a light chain sequence and a heavy chain sequence.

11. A cell comprising the one or more vectors of claim 8.

12. An engineered cell that expresses the antibody of claim 1.

13. The cell of claim 12, wherein the cell is an immune cell.

14. The cell of claim 13, wherein the immune cell is a B cell.

15. A pharmaceutical composition comprising one or more antibodies of any of claim 1 and/or cells of claim 11 and a pharmaceutically acceptable carrier.

16. A method for treating or preventing HIV infection in a subject, comprising administering to the subject an effective amount of the composition of claim 15.

17. A method of functionally curing HIV in a subject comprising administering to the subject an effective amount of the composition of claim 15.

18. The method of claim 16, wherein the composition is administered in combination with another therapy.

19. The method of claim 18, wherein the therapy is an anti-retroviral therapy.
